(19) United States
(12) Patent Application Publication
(10) Pub. No.: US 2019/0389841 A1
(43) Pub. Date: Dec. 26, 2019

(54) COMPOUNDS FOR TREATMENT OF TRIPLE NEGATIVE BREAST CANCER AND OVARIAN CANCER

(71) Applicants: UNIVERSITY OF TENNESSEE RESEARCH FOUNDATION, Knoxville, TN (US); VERU INC, MIAMI, FL (US)

(72) Inventors: Wei LI, Germantown, TN (US); Duane D. MILLER, Collierville, TN (US); Shanshan DENG, Memphis, TN (US); Raya KRUTILINA, Memphis, TN (US); Tiffany N. SEAGROVES, Memphis, TN (US); Junming YUE, Memphis, TN (US); Guannan ZHAO, Memphis, TN (US); Wang QINGHUI, Memphis, TN (US); Mitchell S. STEINER, Germantown, TN (US)

(73) Assignees: UNIVERSITY OF TENNESSEE RESEARCH FOUNDATION, Knoxville, TN (US); VERU INC, MIAMI, FL (US)

(21) Appl. No.: 16/413,147

(22) Filed: May 15, 2019

Related U.S. Application Data
(60) Provisional application No. 62/671,824, filed on May 15, 2018.
(60) Provisional application No. 62/741,494, filed on Oct. 4, 2018.
(60) Provisional application No. 62/805,826, filed on Feb. 14, 2019.

(51) Int. Cl. C07D 403/04; A61K 31/4178; A61K 45/06; A61P 35/00

(52) U.S. Cl. CPC C07D 403/04; A61P 35/00; A61K 45/06; A61K 31/4178

(57) ABSTRACT

The present invention encompasses methods of treating triple negative breast cancer and/or ovarian cancer using therapeutically effective amounts of compounds represented by the structure of formula I.

### CROSS REFERENCE TO RELATED APPLICATIONS

This application claims the benefit of U.S. Provisional Application Nos. 62/671,824, filed May 15, 2018; 62/741,494, filed Oct. 4, 2018; and 62/805,826, filed Feb. 14, 2019, hereby incorporated by reference.

### STATEMENT REGARDING FEDERALLY SUPPORTED RESEARCH OR DEVELOPMENT

The invention described herein was made with government support under Grant No. CA148706, awarded by The National Institutes of Health. The government has certain rights in the invention.

### FIELD OF THE INVENTION

The present invention relates to novel methods of treating triple negative breast cancer and/or ovarian cancer by administering to a subject in need thereof a therapeutically effective amount of at least one compound of Formula I or a pharmaceutically acceptable salt thereof, optionally including a pharmaceutically acceptable excipient.

### BACKGROUND OF THE INVENTION

Cancer is the second most common cause of death in the United States, exceeded only by heart disease. In the United States, cancer accounts for 1 of every 4 deaths. The 5-year relative survival rate for all cancer patients diagnosed in 1996-2003 is 66%, up from 50% in 1975-1977 (Cancer Facts & Figures American Cancer Society: Atlanta, Ga. (2008)). This improvement in survival reflects progress in diagnosing at an earlier stage and improvements in treatment. Discovering highly effective anticancer agents with low toxicity is a primary goal of cancer research.

Microtubules are cytoskeletal filaments consisting of α and β-tubulin heterodimers and are involved in a wide range of cellular functions, including shape maintenance, vesicle transport, cell motility, and division. Tubulin is the major structural component of the microtubules and a well verified target for a variety of highly successful anti-cancer drugs. Compounds that are able to interfere with microtubule-tubulin equilibrium in cells are effective in the treatment of cancers. Anticancer drugs like taxol and vinblastine that are able to interfere with microtubule-tubulin equilibrium in cells are extensively used in cancer chemotherapy. There are three major classes of antimitotic agents. Microtubule-stabilizing agents, which bind to fully formed microtubules and prevent the depolymerization of tubulin subunits, are represented by taxanes and epothilones. The other two classes of agents are microtubule-destabilizing agents, which bind to tubulin dimers and inhibit their polymerization into microtubules. Vina alkaloids such as vinblastine bind to the vinca site and represent one of these classes. Colchicine and colchicine-site binders interact at a distinct site on tubulin and define the third class of antimitotic agents.

Both the taxanes and vinca alkaloids are widely used to treat human cancers, while no colchicine-site binders are currently approved for cancer chemotherapy yet. However, colchicine binding agents like combretastatin A-4 (CA-4) and ABT-751, are now under clinical investigation as potential new chemotherapeutic agents (Luo et al., ABT-751, “A novel tubulin-binding agent, decreases tumor perfusion and disrupts tumor vasculature,” Anticancer Drugs 2009, 20(6), 483-92; Mauer et al., “A phase II study of ABT-751 in patients with advanced non-small cell lung cancer,” J. Thorac. Oncol., 2008, 3(6), 631-6; Rustin et al., “A Phase Ib trial of CA4P (combretastatin A-4 phosphate), carboplatin, and paclitaxel in patients with advanced cancer,” Br. J. Cancer, 2010, 102(9), 1355-60).

Unfortunately, microtubule-interacting anticancer drugs in clinical use share two major problems, drug resistance development and neurotoxicity. A common mechanism of drug resistance is because of multidrug resistance proteins (MDRs), namely ATP binding cassette (ABC) transporter protein-mediated drug efflux, limits the efficacy of these drugs (Green et al., “Beta-Tubulin mutations in ovarian cancer using single strand conformation analysis-risk of false positive results from paraffin embedded tissues,” Cancer Letters, 2006, 236(1), 148-54; Wang et al., “Paclitaxel resistance in cells with reduced beta-tubulin,” Biochimica et Biophysica Acta, Molecular Cell Research, 2005, 1744(2), 245-255; Leslie et al., “Multidrug resistance proteins: role of P-glycoprotein, MRP1, MRP2, and BCRP (ABCG2) in tissue defense,” Toxicology and Applied Pharmacology, 2005, 204(3), 216-237).

P-glycoproteins (P-gp protein is encoded by the MDR1 gene) are important members of the ABC superfamily. P-gp prevents the intracellular accumulation of many cancer drugs by actively effluxing drug out of cancer cells, as well as contributing to normal hepatic, renal, or intestinal clearance pathways. Attempts to co-administer P-gp modulators or inhibitors to increase cellular availability by blocking the actions of P-gp have met with limited success (Gottesman et al., “The multidrug transporter, a double-edged sword,” J. Biol. Chem., 1988, 263(25), 12163-6; Fisher et al., “Clinical studies with modulators of multidrug resistance,” Hematology/Oncology Clinics of North America, 1995, 9(2), 363-82).

The other major problem with taxanes, as with many biologically active natural products, is its lipophilicity and lack of solubility in aqueous systems. This leads to the use of emulsifiers like Cremophor EL and Tween 80 in clinical preparations. A number of biologic effects related to these drug formulation vehicles have been described, including acute hypersensitivity reactions and peripheral neuropathies (Hennenfent, et al., “Novel formulations of taxanes: a review. Old wine in a new bottle?” Ann. Oncol., 2006, 17(5), 735-49; ten Tije, et al., “Pharmacological effects of formulation vehicles: implications for cancer chemotherapy,” Clin. Pharmacokinet., 2003, 42(7), 665-85).

Compared to compounds binding the paclitaxel- or vinca alkaloid binding site, colchicine-binding agents usually exhibit relatively simple structures. Thus, providing a better opportunity for oral bioavailability via structural optimization to improve solubility and pharmacokinetic (PK) parameters. In addition, many of these drugs appear to circumvent P-gp-mediated drug resistance. Therefore, these novel colchicine binding site targeted compounds hold great promise as therapeutic agents, particularly since they have improved aqueous solubility and overcome P-gp mediated drug resistance.

Triple negative breast cancer is found in 15% of all cases of breast cancer in the United States. Triple negative breast cancer is defined as tumor that lack expression of estrogen receptor (ER), progesterone receptor (PR), and human epithermal growth factor receptor (HER-2). Triple negative breast cancer is characterized by aggressive clinical behavior and poor prognosis due to rapid resistance to many chemotherapeutic drugs and lack of suitable targets. Currently, there are no approved targeting therapies available. Classic microtubule-targeted drugs (MTDs), such as paclitaxel and its semisynthetic derivatives, have achieved considerable success in the clinical management of breast cancer neoplasms. Anthracyclines and taxanes based chemotherapy is a standard care for triple negative breast cancer. However, eventually most triple negative breast cancer patients will develop drug resistance, tumor relapse, and/or metastasis after a transient response to initial rounds of therapies. There is an urgent need to develop innovative and more effective therapeutic approaches that achieve a more durable response to triple negative breast cancer treatment.

Metastatic ovarian cancer is the most lethal gynecological malignancy in women and chemotherapy is one of the standard treatment options. Even though there are several FDA approved anti-tubulin agents, mainly taxanes, that are included in the effective management of ovarian cancer, drug resistance to taxanes often develops with resulting disease progression.

With the rising incidence of triple negative breast cancer and ovarian cancer and the high resistance to current therapeutic agents, developing more effective drugs for treating such cancers that can effectively circumvent MDR will provide significant benefits to cancer patients.

### SUMMARY OF THE INVENTION

In one embodiment, the invention encompasses methods of treating triple negative breast cancer and/or ovarian cancer in a subject by administering a therapeutically effective amount of a compound of Formula XI to the subject, wherein Formula XI is represented by:

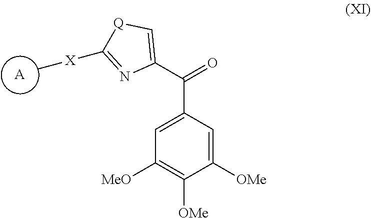

wherein
X is a bond, NH or S;
Q is O, NH or S; and
A is a ring and is substituted or unsubstituted saturated or unsaturated single-, fused- or multiple-ring, aryl or (hetero)cyclic ring system; N-heterocycle; S-heterocycle; O-heterocycle; cyclic hydrocarbon; or mixed heterocycle;
wherein the A ring is optionally substituted by 1-5 substituents which are independently O-alkyl, O-haloalkyl, F, Cl, Br, I, haloalkyl, CN, —CH₂CN, NH₂, hydroxyl, —(CH₂)ᵢNHCH₃, —(CH₂)ᵢNH₂, —(CH₂)ᵢN(CH₃)₂, —OC(O)CF₃, C₁-C₅ linear or branched alkyl, alkylamino, aminoalkyl, —OCH₂Ph, —NHCO-alkyl, COOH, —C(O)Ph, C(O)O-alkyl,
C(O)H, —C(O)NH₂ or NO₂;
i is an integer between 0-5;
wherein if Q is S, then X is not a bond and pharmaceutically acceptable salts thereof.

Another embodiment of the invention encompasses methods of treating triple negative breast cancer and/or ovarian cancer in a subject in need thereof by administering a therapeutically effective amount of a compound of Formula VIII to the subject, wherein Formula VIII is represented by the structure:

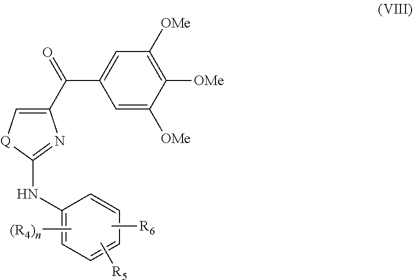

R₄, R₅ and R₆ each independently is hydrogen, O-alkyl, O-haloalkyl, F, Cl, Br, I, haloalkyl, CN, —CH₂CN, NH₂, hydroxyl, —(CH₂)ᵢNHCH₃, —(CH₂)ᵢNH₂, —(CH₂)ᵢN(CH₃)₂, —OC(O)CF₃, C₁-C₅ linear or branched alkyl, alkylamino, aminoalkyl, —OCH₂Ph, —NHCO— alkyl, COOH, —C(O)Ph, C(O)O-alkyl, C(O)H, —C(O)NH₂ or NO₂; Q is S, O or NH;
i is an integer between 0-5; and n is an integer between 1-3 and pharmaceutically acceptable salts thereof.

Yet another embodiment, of the invention encompasses methods of treating triple negative breast cancer and/or ovarian cancer in a subject in need thereof by administering a therapeutically effective amount of a compound of Formula XI(b) to the subject, wherein Formula

XI(b) is represented by the structure:

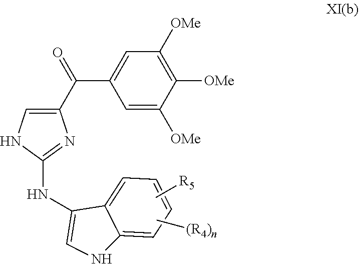

wherein R₄ and R₅ are independently hydrogen, O-alkyl, O-haloalkyl, F, Cl, Br, I, haloalkyl, CN, —CH₂CN, NH₂, hydroxyl, —(CH₂)ᵢNHCH₃, —(CH₂)ᵢNH₂, —(CH₂)ᵢN(CH₃)₂, —OC(O)CF₃, C₁-C₅ linear or branched alkyl, alkylamino, aminoalkyl, —OCH₂Ph, —NHCO— alkyl, COOH, —C(O)Ph, C(O)O-alkyl, C(O)H, —C(O)NH₂ or NO₂;
i is an integer from 0-5; and
n is an integer between 1-4 and pharmaceutically acceptable salts thereof.

One embodiment of the invention encompasses methods of treating triple negative breast cancer and/or ovarian cancer in a subject in need thereof by administering a therapeutically effective amount of a compound of Formula XI(c) to the subject, wherein the compound of Formula XI(c) is represented by the structure:

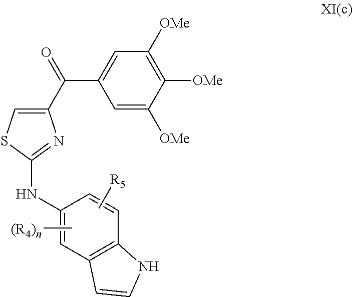

wherein R₄ and R₅ are independently hydrogen, O-alkyl, O-haloalkyl, F, Cl, Br, I, haloalkyl, CN, —CH₂CN, NH₂, hydroxyl, —(CH₂)ᵢNHCH₃, —(CH₂)ᵢNH₂, —(CH₂)ᵢN(CH₃)₂, —OC(O)CF₃, C₁-C₅ linear or branched alkyl, alkylamino, aminoalkyl, —OCH₂Ph, —NHCO— alkyl, COOH, —C(O)Ph, C(O)O-alkyl, C(O)H, —C(O)NH₂ or NO₂;
i is an integer from 0-5; and
n is an integer between 1-4 and pharmaceutically acceptable salts thereof.

Another embodiment of the invention encompasses methods of treating triple negative breast cancer and/or ovarian cancer in a subject in need thereof by administering a compound of Formula XI(e), wherein Formula XI(e) is represented by the structure:

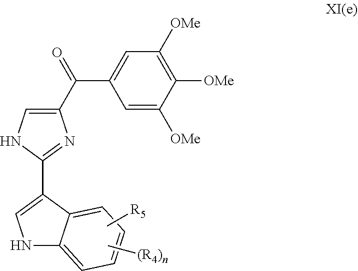

wherein R₄ and R₅ are independently hydrogen, O-alkyl, O-haloalkyl, F, Cl, Br, I, haloalkyl, CN, —CH₂CN, NH₂, hydroxyl, —(CH₂)ᵢNHCH₃, —(CH₂)ᵢNH₂, —(CH₂)ᵢN(CH₃)₂, —OC(O)CF₃, C₁-C₅ linear or branched alkyl, alkylamino, aminoalkyl, —OCH₂Ph, —NHCO— alkyl, COOH, —C(O)Ph, C(O)O-alkyl, C(O)H, —C(O)NH₂ or NO₂;
i is an integer from 0-5; and
n is an integer between 1-4 and pharmaceutically acceptable salts thereof.

Yet another embodiment of the invention encompasses methods of treating triple negative breast cancer and/or ovarian cancer in a subject in need thereof by administering to the subject a therapeutically effective amount of at least one of the following compounds: (2-(phenylamino)thiazol-4-yl)(3,4,5-trimethoxyphenyl)methanone (5a), (2-(p-tolylamino)thiazol-4-yl)(3,4,5-trimethoxyphenyl)methanone (5b), (2-(p-fluorophenylamino)thiazol-4-yl)(3,4,5-trimethoxyphenyl)methanone (5c), (2-(4-chlorophenylamino)thiazol-4-yl)(3,4,5-trimethoxyphenyl)methanone (5d), (2-(phenylamino)-1H-imidazol-4-yl)(3,4,5-trimethoxyphenyl)methanone (5e), 2-(1H-indol-3-yl)-1H-imidazol-4-yl)(3,4,5-trimethoxyphenyl)methanone (17ya); and (2-(1H-indol-5-ylamino)thiazol-4-yl)(3,4,5-trimethoxyphenyl)methanone (55).

In another embodiment, the compound of this invention is its stereoisomer, pharmaceutically acceptable salt, hydrate, N-oxide, or combinations thereof. The invention includes pharmaceutical compositions comprising a compound of this invention and a pharmaceutically acceptable carrier.

### BRIEF DESCRIPTION OF THE DRAWINGS

The subject matter regarded as the invention is particularly pointed out and distinctly claimed in the concluding portion of the specification. The invention, however, both as to organization and method of operation, together with objects, features, and advantages thereof, may best be understood by reference to the following detailed description when read with the accompanying drawings in which:

FIGS. 1A-1B illustrate two graphs of the anti-cancer activity of compound 17ya in vitro. FIG. 1A illustrates compound 17ya tested with MDA-MB-231 cell line as compared to colchicine and paclitaxel. FIG. 1B illustrates compound 17ya tested with MDA-MB-468 cell line and compared to colchicine and paclitaxel.

FIGS. 2A-2B illustrate a comparison of colchicine, paclitaxel and compound 17ya activity in the MDS-MB-231 cell line at 0, 8, 16, and 32 nM concentrations (FIG. 2A), and a bar graph representation of the results (FIG. 2B).

FIG. 3A-3B illustrate the anti-migration of compound 17ya. FIG. 3A illustrates the anti-migration effect of compound 17ya on TNBC cell lines as compared to a control, colchicine (16 nM), and compound 17ya (16 nM) in MDA-MB-231 and MDC-MB-468 cell lines. FIG. 3B illustrates in bar graph form the results of the tests.

FIGS. 4A-4B illustrate a comparison using MBA-MD-231 (16 nM) cell line between control, colchicine, paclitaxel, and compound 17ya at 0 hours, 12 hours, and 24 hours, which is first shown in FIG. 4A. FIG. 4B illustrates the numerical results shown in the bar graph.

FIGS. 5A-5B illustrate a comparison using MBA-MD-468 (16 nM) cell line between control, colchicine, paclitaxel, and compound 17ya at 0 hours, 24 hours, and 48 hours. FIG. 5A illustrates the antimigration effect. FIG. 5B illustrates the numerical results in bar graph.

FIGS. 6A-6B illustrate the anti-invasion of compound 17ya. FIG. 6A illustrates the anti-invasion effect of compound 17ya (40 nM) on TNBC cell lines as compared to a control in MDA-MB-231, and compound 17ya (32 nM) as compared a control and colchicine (32 nM) in MDC-MB-468 cell lines. FIG. 6B illustrates in bar graph form the results of the tests.

FIGS. 7A-7B illustrate the cell apoptosis of compound 17ya (100 nM) on TNBC cells. FIG. 7A illustrates the cell apoptosis of compound 17ya (100 nM) on TNBC cells, cell line MDA-MB-231 as compared against a control at 24 hours, 48 hours, and 72 hours. FIG. 7B illustrates in bar graph form the results of the comparison.

FIG. 8 illustrates the cell apoptosis of compound 17ya in a dose and time-dependent manner after 48 hours on TNBC cells, cell line MDA-MB-231 as compared against a control colchicine (200 nM), and paclitaxel (200 nM) and compound 17ya at 50 nM, 100 nM, 150 nM, and 200 nM. The figure also illustrates in bar graph form the results of the comparison.

FIGS. 9A-9B illustrate with two graphs the inhibition by compound 17ya of TNBC tumor growth in a dose dependent manner without interfering with the body weight of mice. FIG. 9A compares percent tumor growth (volume) over time administered a vehicle, 5 mg/kg compound 17ya, and 10 mg/kg compound 17ya. FIG. 9B illustrates the rat body weight over time (days) when administered the vehicle, 5 mg/kg compound 17ya, and 10 mg/kg compound 17ya.

FIG. 10 illustrates the tumor weight or final tumor weight (as determined in FIG. 9) in bar graph form and size comparison as compound 17ya inhibits TNBC tumor growth in a dose dependent manner.

FIGS. 11A-11B illustrate the anti-cancer activity of compound 17ya as compared to the vehicle and paclitaxel. FIG. 11A illustrates the anti-cancer activity of compound 17ya as compared to the vehicle and paclitaxel at 12.5 mg/kg by measuring tumor weight. FIG. 11B illustrates the anti-cancer activity of compound 17ya as compared to the vehicle and paclitaxel at 12.5 mg/kg by measuring final tumor volume.

FIG. 12 illustrates the anti-metasis of compound 17ya in vivo using H & E sections from lungs as compared to a control, 10 mg/kg paclitaxel, and 10 mg/kg compound 17ya.

FIGS. 13A-B illustrate the effect of compound 17ya on ovarian cancer cells, demonstrating significant inhibition of cell survival. FIG. 13A illustrates the cell survival as determined by colonies/field of 350 SKOV3 cells treated with compound 17ya at 0, 1.25, 2.5, 5, 10, and 30 nM, where **p<0.01 and ***p<0.001. FIG. 13B illustrates the cell survival as determined by colonies/field of OVCAR3 cells treated with compound 17ya at 0, 1.25, 2.5, 5, 10, and 30 nM, where **p<0.01 and ***p<0.001.

FIGS. 14A-14B illustrate inhibition by compound 17ya of ovarian cancer cell migration and invasion. FIG. 14A illustrates the result of cell migration of SKOV3 and OVCAR3 cells treated with compound 17ya (20 nM) and control (vehicle) using transwell plates. Migrated cells were stained with crystal violet and counted, where **p<0.01 and ***p<0.001. FIG. 14B illustrates the invasion results of SKOV3 and OVCAR3 cells treated with compound 17ya (20 nM) and control (vehicle) for 5 h using Matrigel-coated plates (cells were stained with H.E. and counted) and **p<0.01 and ***p<0.001.

FIGS. 15A-15C illustrate inhibition by compound 17ya on ovarian tumor growth and metastasis in vivo. FIG. 15A illustrates the effect of compound 17ya on 2 month old NSG female mice intrabursally injected with 5×105 wildtype SKOV3-Luc2 cells after treatment for five days a week for 4 weeks. FIG. 15B graphically illustrates tumor weight of compound 17ya and control treated ovaries. FIG. 15c illustrates that tumors were not visible in ovaries, liver and spleen of mice treated with compound 17ya.

FIGS. 16A-B graphically illustrate the cell viability after treatment with colchicine, paclitaxel, and compound 17ya. FIG. 16A illustrates the MDA-MD-231 cell viability after treatment with colchicine, paclitaxel, and compound 17ya with IC₅₀ (nM) of 17.46, 3.05, and 8.23, respectively, and SEM of 3.40, 0.42, and 1.34, respectively. FIG. 16B illustrates the MDA-MD-468 cell viability after treatment with colchicine, paclitaxel, and compound 17ya with IC₅₀ (nM) of 9.80, 4.61, and 9.59, respectively, and SEM of 1.45, 0.63, and 1.78, respectively.

FIGS. 17A-B illustrate the cell migration inhibition of compound 17ya and colchicine. FIG. 17A illustrates the effect of colchicine or compound 17ya on cell migration as compared to the control. FIG. 17B illustrates the effect of colchicine, PTX, compound 17ya on cell migration through a Matrigel-coated membrane.

FIG. 18 illustrates the immunofluorescence staining used to visualize the microtubule network comparison between the control, colchicine, paclitaxel, and Veru-111 (compound 17ya) all at 32 nM with cells MDA-MB-231 and MDA-MB-468.

FIG. 19 illustrates the effect of compound 17ya (VERU-111) on apoptosis induction in TNBC cells where MDA-MB-231 cells were treated with 100 nM compound 17ya in a time-dependent manner and the compound induced the cells to apoptosis as compared to the control at 24 h, 48 h, and 72 h with various times and concentrations.

FIG. 20 illustrates the anti-cancer activity of compound 17ya (VERU-111) in orthotopic TNBC mouse model to determine if the potent effect of compound 17ya could be observed in vivo as determined over time, including after 33 days of treatment, where compound 17ya inhibited TNBC tumor growth in a dose dependent manner without interfering the body weight of mice.

FIG. 21 illustrates the comparison of the efficacy of compound 17ya (VERU-111) with paclitaxel in a model since paclitaxel is one of the standard cares for TNBC treatment in clinic, were compound 17ya and paclitaxel significantly regressed the tumor size and tumor weight.

FIG. 22 illustrates H & E staining of tumors in a comparison study of compound 17ya (VERU-111) and paclitaxel in induced TNBC tumor necrosis in the lung tissue where the vehicle group were full of metastasis (indicated by yellow arrow), while the lungs in compound 17ya and paclitaxel group had little, suggesting that compound 17ya significantly reduced metastasis of TNBC.

FIG. 23 illustrates IHC staining of tumors in a comparison study of compound 17ya (VERU-111) and paclitaxel in induced TNBC tumor necrosis in the lung tissue where the vehicle group were full of metastasis (indicated by yellow arrow), while the lungs in compound 17ya and paclitaxel group had little, suggesting that compound 17ya significantly reduced metastasis of TNBC.

FIG. 24 illustrates a brief compound summary showing that compound VERU-111 (compound 17ya) is a novel, oral, next generation tubulin inhibitor targeting α and β subunits of microtubules with low nanomolar inhibition of tubulin polymerization, high oral bioavailability, high brain penetration, and having efficacy against prostate, breast, and other cancers in vivo and in vitro.

FIG. 25 illustrates that VERU-111 (compound 17ya) is built on a proven mechanism-inhibiting microtubule assembly and comparing the microtubules disrupted from spindle shape (control) versus globular shape (compound 17ya).

FIGS. 26A-B illustrate molecular modeling of VERU-111 (compound 17ya) with tubulin complex (compound 17ya=6a). FIG. 26A illustrates the molecular modeling of compound 17ya with the colchine binding site, where the compound is closer in its binding pose to TN-16 than it is to colchine itself. FIG. 26B illustrates that compound 17ya is more linear and penetrates deeper in the binding pocket of the β-tubulin monomer than TN-16 and has hydrogen bonding which results in differential and stronger binding to α and β tubulin.

FIG. 27 illustrates drug-like attributes of VERU-111 (compound 17ya) and VERU-112 (compound 55).

FIG. 28 illustrates pharmacokinetic parameters of VERU-111 (compound 17ya) and VERU-112 (compound 55) in mice, rats, and dogs.

FIG. 29 illustrates a metabolism pathway for VERU-111 (compound 17ya) in human and dogs, where the abundant metabolite M+34 was only found in dog liver microsomes resulting in high clearance in dog in vivo.

FIG. 30 illustrates brain penetration of VERU-111 (compound 17ya) and VERU-112 (compound 55). VERU-112 (compound 55) demonstrated high brain penetration. Brain/plasma concentration ratio was about 20% 4 h after oral treatment. Brain/plasma concentration ratios remained relatively constant over time for VERU-111 (compound 17ya) and VERU-112 (compound 55) suggesting that brain concentrations were in the same pharmacokinetic compartment as plasma and will not accumulate in the brain; perhaps, reducing the possibility for neurotoxicity.

FIG. 31 illustrates compound's activity on p-glycoprotein ATPase (Pgp ATPase). VERU-111 (compound 17ya) was not a substrate for p-glycoprotein, where p<0.05.

FIG. 32 illustrates a summary of VERU-111 (compound 17ya) in vitro and in vivo cytotoxic activities, demonstrating similar or greater potency as paclitaxel and docetaxel in parental lines; while paclitaxel and docetaxel lose activity in taxane-resistant cell lines, VERU-111 (compound 17ya) has potent antiproliferative activity; and the compound is cytotoxic against multiple cancer types: prostate, taxane resistant prostate cancer, breast, triple negative breast, lung, melanoma, glioma, colon, uterine, ovarian, and pancreatic cancers.

FIG. 33 illustrates VERU-111 (compound 17ya) In vitro cytotoxicity after 96 hours (IC₅₀ values—nM). VERU-111 (compound 17ya) has similar potency as paclitaxel and docetaxel in the parental PC-3 cell line. VERU-111 (compound 17ya) retains its potency in the paclitaxel resistant PC-3 cells whereas paclitaxel and docetaxel lose potency.

FIGS. 34A-D illustrate VERU-111 (compound 17ya) (II) and VERU-112 (IAT) inhibited paclitaxel resistant prostate cancer xenograft growth. FIG. 34A illustrates the resistance against PC-3, where treatment was initiated when tumors reached 150-300 mm³. FIG. 34B illustrates the resistance against TxR (PC-3/TxR is taxane resistant), where treatment was initiated when tumors reached 150-300 mm³. FIG. 34C illustrates the resistance against TxR (PC-3/TxR is taxane resistant), where treatment was initiated when tumors reached 150-300 mm³. FIG. 34D illustrates the resistance against TxR (PC-3/TxR is taxane resistant), where treatment was initiated when tumors reached 150-300 mm³.

FIG. 35 illustrates anti-tumor activity of VERU-111 (compound 17ya) and VERU-112 versus docetaxel in vivo. In contrast to the lack of efficacy of Docetaxel in PC-3/TxR tumors, VERU-111 (compound 17ya) was dosed orally and had >100% TGI without an effect on body weight.

FIG. 36 illustrates VERU-111 (compound 17ya) tested in additional xenograft models.

FIG. 37 illustrates anti-cancer activity of VERU-111 (compound 17ya) in triple negative breast cancer (TNBC) in vitro, where in MDA-MB-231 colchine had IC₅₀ of 17.46 (SE 0.05); paclitaxel ha IC₅₀ of 3.05 (SE 0.04); and VERU-111 (compound 17ya) had IC₅₀ of 8.23 (SE 0.05). In MDA-MB-468 colchine had IC₅₀ of 9.80 (SE 0.02); paclitaxel ha IC₅₀ of 4.61 (SE 0.03); and VERU-111 (compound 17ya) had IC₅₀ of 22.96 (SE 0.02).

FIG. 38 illustrates anti-tumor activity of VERU-111 (compound 17ya) in TNBC in vivo, where VERU-111 (compound 17ya) inhibits TNBC tumor growth in a dose dependent manner without interfering the body weight of mice.

FIG. 39 illustrates that VERU-111 (compound 17ya) inhibited triple negative breast cancer xenographs in mice.

FIG. 40 illustrates that VERU-111 (compound 17ya) inhibited triple negative breast cancer metastases in mice.

FIG. 41 illustrates that VERU-111 (compound 17ya) inhibited ovarian cancer in a orthotopic ovarian cancer model (tx 5x/week for 4 weeks).

FIG. 42A-D illustrate VERU-111 (compound 17ya) inhibited pancreatic cancer. FIG. 42A(i-ii) illustrate the dose dependent effect of VERU-111 (compound 17ya) over cell lines Panc-1, AsPC-1, and HPAF-II as percent of cell viability. FIG. 42B(i-ii) illustrate the time dependent effect of VERU-111 (compound 17ya) at 5 nM, 10 nM, and 20 nM as comparted to a control. FIG. 42C illustrates the effect of VERU-111 (compound 17ya) at 1.25 nM, 2.5 nM, and 5 nM as comparted to a control with Panc-1 (Figure C(i)), AsPC-1 (Figure C(ii)), or HPAF-II (Figure C(iii)) cell lines. FIG. 42D illustrates the effect of VERU-111 (compound 17ya) at 1.25 nM, 2.5 nM, and 5 nM as comparted to a control with Panc-1 (Figure D(i)), AsPC-1 (Figure D(ii)), or HPAF-II (Figure D(iii)) cell lines in bar graph form.

FIGS. 43A-E illustrate VERU-111 (compound 17ya) inhibited pancreatic cancer. FIG. 43A(i) illustrates the comparison of Compound 17ya (5 nM, 10 nM, and 20 nM) as compared to a control on Panc-1 cells. FIG. 43A(ii) tabulates the effect in table format. FIG. 43B illustrates the effect of Compound 17ya at 0, 5, 10, and 20 nM against Panc1 and AsPC1 cell lines. FIG. 43C illustrates the cell apoptosis of compound 17ya at 5 nM, 10 nM, and 20 nM as compared to the control. FIG. 43D illustrates the effect against Panc1 and AsPC1 cell lines. FIG. 43E(i) illustrates the inhibiting effect of Compound 17ya on cell proliferation. FIG. 43E(ii) represents the same results in graphical form using mean fluorescence TMRE.

FIG. 44 illustrates VERU-111 (compound 17ya) in preclinical safety (less myelosuppression, less neurotoxicity, maintains body weight), where FIG. 44 illustrates the toxicity tests of liver weight and white blood count (WBC) in mice in the use of VERU-111 (3.3 mpk or 6.7 mpk) and VERU-112 (10 mpk and 30 mpk) as compared to a control and DTX (10 mpk and 20 mpk).

FIG. 45 illustrates VERU-111 (compound 17ya) in preclinical safety (less myelosuppression, less neurotoxicity, maintains body weight), where FIG. 45 illustrates the neurotoxicity tests (hot plate test at 5-52.5° C. and the time require to lick the paw recorded as latency period for pain threshold) in mice in the use of VERU-111 (3.3 mpk or 6.7 mpk) and VERU-112 (10 mpk and 30 mpk) as compared to a control and DTX (10 mpk and 20 mpk).

FIG. 46 illustrates VERU-111 (compound 17ya) as antiproliferative and maintains body weight as contrasted to the lack of efficacy of docetaxel in PC-3/Txr tumors, VERU-111 (compound 17ya) was dosed orally and had >100% TGI without an effect on body weight.

FIG. 47 illustrates nonclinical results in assessment of blockade of HERG potassium channels stably expressed in HEK293 cells and central nervous system safety study in rats with an IC₂₀ of 9.23 nM and the oral administration of VERU-111 (compound 17ya) at doses up to and including 10 mg/kg was not associated with any adverse effects on neurobehavioral function in rats.

FIG. 48 illustrates VERU-111 (compound 17ya) nonclinical results in cardiovascular and respiratory evaluation study in beagle dogs where VERU-111 (compound 17ya) was administered as doses of 2, 4, and 8 mg/kg to dogs and did not produce mortality or effects on blood pressure, heart rate, or the evaluated electrocardiogram or respiratory parameters. Increases in body temperature (<0.7° C. maximum change) were observed at all doses of VERU-111 (compound 17ya) from approximately 3.5 to 11 hours post dose. Vomitus was noted between 4 and 24 hours following the 8 mg dose. Oral administration of VERU-111 (compound 17ya) at doses up to and including 8 mg/kg was not associated with any adverse effects on cardiovascular or respiratory function in dogs.

FIG. 49 illustrates VERU-111 (compound 17ya) pharmacokinetics in dogs were mean (±SD) and CV % for VERU-111 (compound 17ya) pharmacokinetic parameters on days 1 and 7 following oral capsule administration of 5 and 10 mg/kg VERU-111 to male gods.

FIG. 50 illustrates VERU-111 (compound 17ya) 28-day oral capsule toxicity study in beagle dogs that found that it did not impact dog survival, no ophthalmoscopic findings; no changes in hematology, coagulation, and urinalysis parameters; no clinical or macroscopic pathologic observations; at 4 and 8 mg/kg mild observations of inappetence, vomiting emesis, and diarrhea; dogs at 8 mg/kg/day had body weight losses; had QTc prolongation that exceeded 10% change; and reduced thymus organ weights and reduction of lymphocytes in thymus; no observed adverse effect level (NOAEL) was 4 mg/kg/day; and following 28 days of dose at 4 mg/kg/day the mean Cₘₐₓ and AUC_{0-12 hr} values were 23.2 ng/ml and 71.7 hr*ng/mL, respectively.

FIGS. 51A and 51B illustrate VERU-111 (compound 17ya) 28-Day oral capsule toxicity study in dogs-weight. FIG. 51A illustrates the mean body weight in male dogs relative to time (weeks) from the start date. FIG. 51B illustrates the mean body weight in dogs relative to time (weeks) from the start date.

FIG. 52 illustrates VERU-111 (compound 17ya) 28-Day oral capsule toxicity study in dogs-QT interval.

FIG. 53 illustrates VERU-111 (compound 17ya) 28-Day oral capsule toxicity study in dogs-Hematology.

FIG. 54 illustrates VERU-111 (compound 17ya) 28-Day oral capsule toxicity study in dogs-Hematology.

FIG. 55 illustrates VERU-111 (compound 17ya) 28-Day oral capsule toxicity study in dogs-Liver function tests.

FIG. 56 illustrates VERU-111 (compound 17ya) 28-Day oral capsule toxicity study in dogs-Liver function tests.

FIG. 57A-B illustrate compound 17ya 28-day oral capsule toxicokinetics study in beagle dogs. FIG. 57A illustrates individual and mean compound 17ya Cₘₐₓ values on Days 1 and 28 following daily oral capsule administration of 2, 4, and 8 mg/kg compound 17ya to dogs (males and females combined). FIG. 57B illustrates individual and mean compound 17ya AUC_{0-12 hr} values on Days 1 and 28 following daily oral capsule administration of 2, 4, and 8 mg/kg compound 17ya to dogs (males and females combined).

FIGS. 58A-B illustrate the effect of compound 17ya with tubulin-destabilizing colchicine and tubulin-stabilizing agent paclitaxel. FIG. 58A illustrates the effect of compound 17ya, colchicine, and paclitaxel in MDA-MB-231 cell line. FIG. 58B illustrates the effect of compound 17ya, colchicine, and paclitaxel in MDA-MB-486 cell line.

FIGS. 59A-D illustrate the effect of compound 17ya compared to colchicine and paclitaxel in a colony formation assay. FIG. 59A illustrates the effect of compound 17ya, colchicine, and paclitaxel on MDA-MB-231. FIG. 59B illustrates the effect of compound 17ya, colchicine, and paclitaxel on MDA-MB-486. FIG. 59C illustrates in bar graph form the effect of Compound 17ya as compared to paclitaxel and colchicine on the MDA-MD-231 cell line. FIG. 59D illustrates in bar graph form the effect of Compound 17ya as compared to paclitaxel and colchicine on the MDA-MB-468 cell line

FIG. 60 illustrates immunofluorescence staining of the microtubule network comparing a control, compound 17ya, colchicine, and paclitaxel.

FIG. 61 illustrates the effect of compound 17ya to inhibit TNBC cells ability to migrate through a membrane insert in the presence of 16 nM concentration by an average migration rate of 40% in MDA-MB-231 cells and 34% in MDA-MB-468 cells as compared to a control group.

FIG. 62 illustrates the effect of compound 17ya to reduce the TNBC cells capacity to invade through the Matrigel-coated membrane with an average invasion rate of 55% and 36% in MDA-MB-231 and MDA-MB-468 cells, as compared to a control.

FIGS. 63A-B illustrate the results of a scratch assay using compound 17ya, paclitaxel, and colchicine as positive controls on MDA-MB-231 and MDA-MB-486 cell lines. At a dose of 16 nM, compound 17ya, colchicine, and paclitaxel showed effective inhibition of cell migration as illustrated in FIG. 63A for MDA-MB-231. The effect for the same compounds and dose is illustrated in FIG. 63B for MDA-MB-486.

FIG. 64A-B illustrate the effect of compound 17ya, colchicine, and paclitaxel on the accumulation of MDA-MB-231 and MDA-MB-486 cells in the G2 and M phase. FIG. 64A illustrates the effect on the population of cells in the G1 and S phase in a dose dependent manner for compound 17ya, colchicine and paclitaxel, (employed as positive controls) on MDA-MB-231 cells in G2/M phase. FIG. 64B illustrates the effect on the population of cells in the G1 and S phase in a dose dependent manner for compound 17ya, colchicine and paclitaxel, (employed as positive controls) on MDA-MB-486 cells in G2/M phase.

FIGS. 65A-B illustrate the ability of compound 17ya, colchicine, and paclitaxel to initiate apoptotic cell death in a dose dependent manner. FIG. 65A illustrates the effect on MDA-MB-231 cell line. FIG. 65B illustrates the effect on MDA-MB-486 cell line.

FIG. 66A-B illustrate the potency of compound 17ya, colchicine, and paclitaxel to induce TNBC cell apoptosis. FIG. 66A illustrates the results with MDA-MB-231 cell line when treated with 100 nM of compound 17ya for 24, 48, and 72 hours. FIG. 66B illustrates the results with MDA-MB-486 cell line when treated with 100 nM of compound 17ya for 24, 48, and 72 hours.

FIG. 67A-B illustrate the effect of compound 17ya, colchicine, and paclitaxel on apoptotic cell death through regulating caspase-3/PARP pathway, expression of cleaved-caspase-3, and cleaved PARP in TNBC cells. FIG. 67A illustrates the effect on MDA-MB-231 cells after 24 hours of treatment analyzed by Western blotting. FIG. 67B illustrates the effect on MDA-MB-486 cells after 24 hours of treatment analyzed by Western blotting.

FIG. 68 illustrates the effect of compound 17ya and control on increased cleaved-caspase-3 and cleaved-PARP in a time dependent manner on MDA-MB-231 and MDA-MB-486.

FIG. 69 illustrates the effect of compound 17ya and control on the expression of caspase 3/7 activity was evaluated on MDA-MB-231 and MDA-MB-468 cells using the Caspase Glo 3/7 assay system.

FIG. 70 illustrates the effect of vehicle, compound 17ya, and paclitaxel on the percent increase in tumor volume after treatment.

FIG. 71 illustrates the effect of vehicle, compound 17ya, and paclitaxel on mouse body weight after treatment.

FIG. 72 illustrates the final tumor volume after treatment with compound 17ya at 10 mg/kg.

FIG. 73 illustrates the final tumor weight after treatment with compound 17ya at 10 mg/kg.

FIG. 74 illustrates the effect of compound 17ya, control, and paclitaxel on percentage of necrotic cells.

FIG. 75 illustrates the effect of control, compound 17ya, and paclitaxel on Ki67.

FIG. 76 illustrates the effect of control, compound 17ya, and paclitaxel on CD31.

FIG. 77 illustrates the effect of control, compound 17ya, and paclitaxel on cleaved-PARP.

FIG. 78 illustrates the effect of control, compound 17ya, and paclitaxel on cleaved-caspase-3.

It will be appreciated that for simplicity and clarity of illustration, elements shown in the figures have not necessarily been drawn to scale. For example, the dimensions of some of the elements may be exaggerated relative to other elements for clarity. Further, where considered appropriate, reference numerals may be repeated among the figures to indicate corresponding or analogous elements.

### DETAILED DESCRIPTION OF THE INVENTION

The invention encompasses methods of treating triple negative breast cancer and/or ovarian cancer by administering at least one compound of formula (I) in a therapeutically effective amount to a subject in need thereof, wherein the compound of Formula (I) has the formula

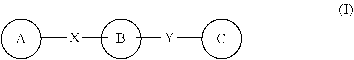

wherein
A and C are each independently substituted or unsubstituted single-, fused- or multiple-ring aryl or (hetero)cyclic ring systems; substituted or unsubstituted, saturated or unsaturated N-heterocycles; substituted or unsubstituted, saturated or unsaturated S-heterocycles; substituted or unsubstituted, saturated or unsaturated O-heterocycles; substituted or unsubstituted, saturated or unsaturated cyclic hydrocarbons; or substituted or unsubstituted, saturated or unsaturated mixed heterocycles;

### B is

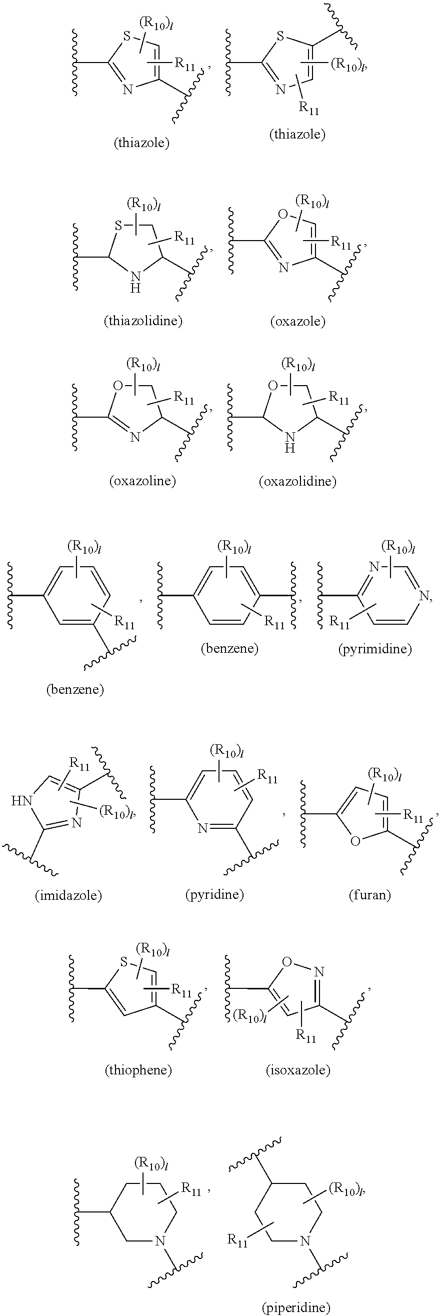

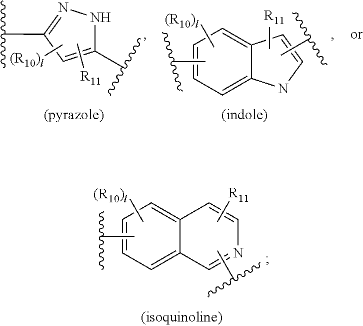

R₁₀ and R₁₁ are independently hydrogen, O-alkyl, O-haloalkyl, F, Cl, Br, I, haloalkyl, CF₃, CN, —CH₂CN, NH₂, hydroxyl, —(CH₂)ᵢNHCH₃, —(CH₂)ᵢNH₂, —(CH₂)ᵢN(CH₃)₂, —OC(O)CF₃, C₁-C₅ linear or branched alkyl, haloalkyl, alkylamino, aminoalkyl, —OCH₂Ph, —NHCO-alkyl, COOH, —C(O)Ph, C(O)O-alkyl, C(O)H, —C(O)NH₂ or NO₂;
X is a bond, NH, C₁ to C₅ hydrocarbon, O, or S;
Y is a bond, —C═O, —C═S, —C═N—NH₂, —C═N—OH, —CH—OH, —C═CH—CN, —C═N—CN, —CH═CH—, —C═C(CH₃)₂, —C═N—OMe, —(C═O)—NH, —NH—(C═O), —(C═O)—O, —O—(C═O), —(CH₂)₁₋₅—(C═O), (C═O)—(CH₂)₁₋₅, —(SO₂)—NH—, —NH—(SO₂)—, SO₂, SO or S;
wherein said A and C rings are optionally substituted by 1-5 substituents which are independently O-alkyl, O-haloalkyl, F, Cl, Br, I, haloalkyl, CF₃, CN, —CH₂CN, NH₂, hydroxyl, —(CH₂)ᵢNHCH₃, —(CH₂)ᵢNH₂, —(CH₂)ᵢN(CH₃)₂, —OC(O)CF₃, C₁-C₅ linear or branched alkyl, haloalkyl, alkylamino, aminoalkyl, —OCH₂Ph, —NHCO-alkyl, COOH, —C(O)Ph, C(O)O-alkyl, C(O)H, —C(O)NH₂ or NO₂;
i is an integer between 0-5;
l in an integer between 0-2;
wherein
if B is a benzene ring, a thiophene ring, a furan ring or an indole ring then X is not a bond or CH₂, and A is not indole;
if B is indole then X is not 0; and
or its pharmaceutically acceptable salt, hydrate, polymorph, metabolite, tautomer or isomer.

The triple negative breast cancer may be taxane-resistance TNBC, taxane-sensitive TNBC, and/or metastasis.

In one embodiment, if B of formula I is a thiazole ring then X is not a bond.

In one embodiment, A in compound of Formula I is indolyl. In another embodiment A is 2-indolyl. In another embodiment A is phenyl. In another embodiment A is pyridyl. In another embodiment A is naphthyl. In another embodiment A is isoquinoline. In another embodiment, C in compound of Formula I is indolyl. In another embodiment C is 2-indolyl. In another embodiment C is 5-indolyl. In another embodiment, B in compound of Formula I is thiazole. In another embodiment, B in compound of Formula I is thiazole; Y is CO and X is a bond. Non limiting examples of compound of formula I are selected from: (2-(1H-Indol-2-yl)thiazol-4-yl)(1H-indol-2-yl)methanone (8) and (2-(1H-indol-2-yl)thiazol-4-yl)(1H-indol-5-yl)methanone (21).

The invention also encompasses a method of treating triple negative breast cancer and/or ovarian cancer in a subject in need thereof by administering at least one compound of formula (Ia) in a therapeutically effective amount to the subject and wherein the compound of formula (Ia) has the structure

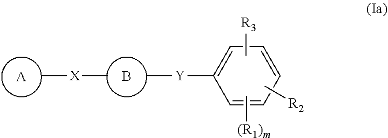

wherein
A is substituted or unsubstituted single-, fused- or multiple-ring, aryl or (hetero)cyclic ring systems; substituted or unsubstituted, saturated or unsaturated N-heterocycles; substituted or unsubstituted, saturated or unsaturated S-heterocycles; substituted or unsubstituted, saturated or unsaturated O-heterocycles; substituted or unsubstituted, saturated or unsaturated cyclic hydrocarbons; or substituted or unsubstituted, saturated or unsaturated mixed heterocycles;

### B is

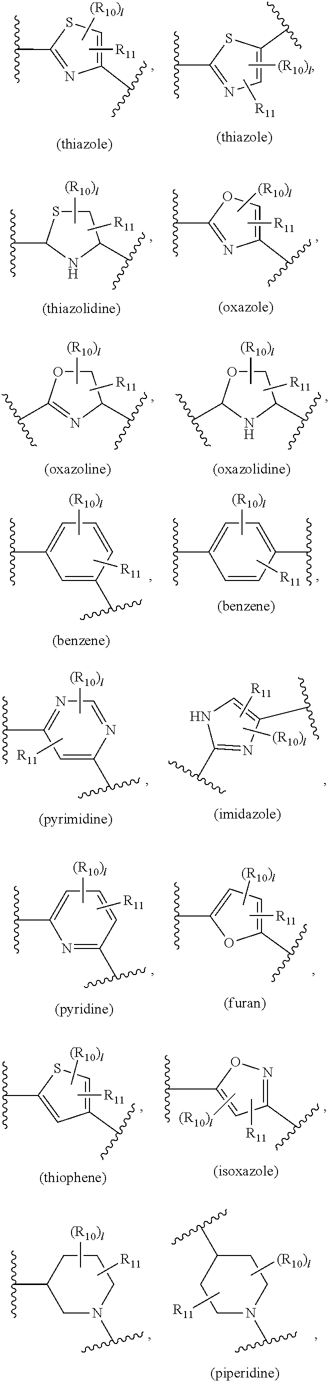

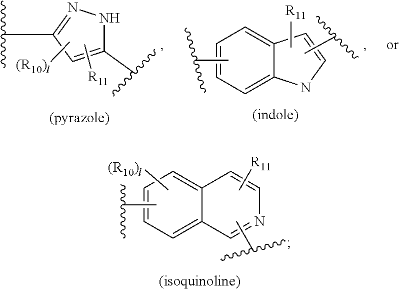

R₁, R₂ and R₃ are independently hydrogen, O-alkyl, O-haloalkyl, F, Cl, Br, I, haloalkyl, CF₃, CN, —CH₂CN, NH₂, hydroxyl, —(CH₂)ᵢNHCH₃, —(CH₂)ᵢNH₂, —(CH₂)ᵢN(CH₃)₂, —OC(O)CF₃, C₁-C₅ linear or branched alkyl, haloalkyl, alkylamino, aminoalkyl, —OCH₂Ph, —NHCO-alkyl, COOH, —C(O)Ph, C(O)O-alkyl, C(O)H, —C(O)NH₂ or NO₂;
R₁₀ and R₁₁ are independently hydrogen, O-alkyl, O-haloalkyl, F, Cl, Br, I, haloalkyl, CF₃, CN, —CH₂CN, NH₂, hydroxyl, —(CH₂)ᵢNHCH₃, —(CH₂)ᵢNH₂, —(CH₂)ᵢN(CH₃)₂, —OC(O)CF₃, C₁-C₅ linear or branched alkyl, haloalkyl, alkylamino, aminoalkyl, —OCH₂Ph, —NHCO-alkyl, COOH, —C(O)Ph, C(O)O-alkyl, C(O)H, —C(O)NH₂ or NO₂;
X is a bond, NH, C₁ to C₅ hydrocarbon, O, or S;
Y is a bond, —C═O, —C═S, —C═N—NH₂, —C═N—OH, —CH—OH, —C═CH—CN, —C═N—CN, —CH═CH—, —C═C(CH₃)₂, —C═N—OMe, —(C═O)—NH, —NH—(C═O), —(C═O)—O, —O—(C═O), —(CH₂)₁₋₅—(C═O), (C═O)—(CH₂)₁₋₅, —(SO₂)—NH—, —NH—(SO₂)—, SO₂, SO or S;
wherein said A ring is optionally substituted by 1-5 substituents which are independently O-alkyl, O-haloalkyl, F, Cl, Br, I, haloalkyl, CF₃, CN, —CH₂CN, NH₂, hydroxyl, —(CH₂)ᵢNHCH₃, —(CH₂)ᵢNH₂, —(CH₂)ᵢN(CH₃)₂, —OC(O)CF₃, C₁-C₅ linear or branched alkyl, haloalkyl, alkylamino, aminoalkyl, —OCH₂Ph, —NHCO-alkyl, COOH, —C(O)Ph, C(O)O-alkyl, C(O)H, —C(O)NH₂ or NO₂;
i is an integer between 0-5;
l is an integer between 0-2;
m is an integer between 1-3;
wherein
if B is a benzene ring, a thiophene ring, a furan ring or an indole ring then X is not a bond or CH₂ and A is not indole;
if B is indole then X is not 0;
or its pharmaceutically acceptable salt, hydrate, polymorph, metabolite, tautomer or isomer.

In one embodiment, if B of formula Ia is a thiazole ring then X is not a bond.

The invention also encompasses methods of treating triple negative breast cancer and/or ovarian cancer by administering at least one compound of formula (II) in a therapeutically effective amount to a subject in need thereof, wherein the compound of Formula (II) has the formula:

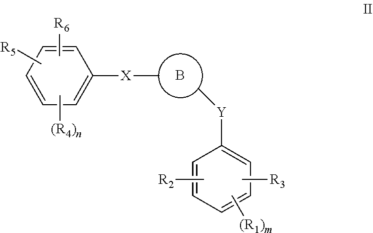

wherein

### B is

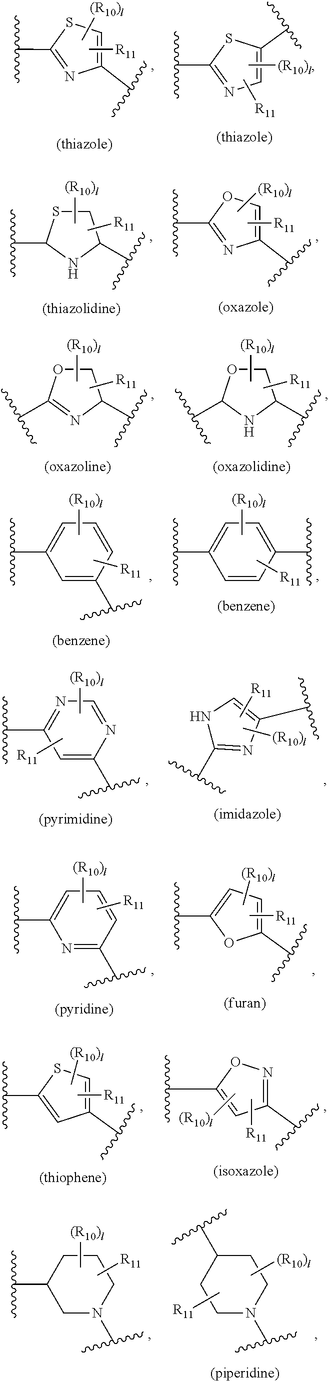

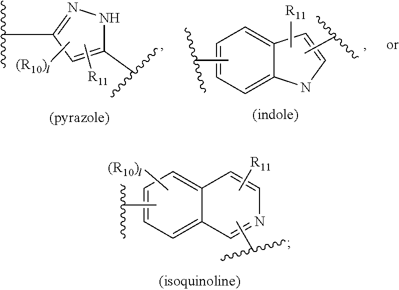

R₁, R₂, R₃, R₄, R₅ and R₆ are independently hydrogen, O-alkyl, O-haloalkyl, F, Cl, Br, I, haloalkyl, CF₃, CN, —CH₂CN, NH₂, hydroxyl, —(CH₂)ᵢNHCH₃, —(CH₂)ᵢNH₂, —(CH₂)ᵢN(CH₃)₂, —OC(O)CF₃, C₁-C₅ linear or branched alkyl, haloalkyl, alkylamino, aminoalkyl, —OCH₂Ph, —NHCO-alkyl, COOH, —C(O)Ph, C(O)O-alkyl, C(O)H, —C(O)NH₂ or NO₂;
R₁₀ and R₁₁ are independently hydrogen, O-alkyl, O-haloalkyl, F, Cl, Br, I, haloalkyl, CF₃, CN, —CH₂CN, NH₂, hydroxyl, —(CH₂)ᵢNHCH₃, —(CH₂)ᵢNH₂, —(CH₂)ᵢN(CH₃)₂, —OC(O)CF₃, C₁-C₅ linear or branched alkyl, haloalkyl, alkylamino, aminoalkyl, —OCH₂Ph, —NHCO-alkyl, COOH, —C(O)Ph, C(O)O-alkyl, C(O)H, —C(O)NH₂ or NO₂;
X is a bond, NH, C₁ to C₅ hydrocarbon, O, or S;
Y is a bond, —C═O, —C═S, —C═N—NH₂, —C═N—OH, —CH—OH, —C═CH—CN, —C═N—CN, —CH═CH—, C═C(CH₃)₂, —C═N—OMe, —(C═O)—NH, —NH—(C═O), —(C═O)—O, —O—(C═O), —(CH₂)₁₋₅—(C═O), (C═O)—(CH₂)₁₋₅, —(SO₂)—NH—, —NH—(SO₂)—, SO₂, SO or S;
i is an integer between 0-5;
l is an integer between 0-2;
n is an integer between 1-3; and
m is an integer between 1-3;
wherein
if B is indole then X is not O;
or its pharmaceutically acceptable salt, hydrate, polymorph, metabolite, tautomer or isomer.

In one embodiment, if B of formula II is a thiazole ring then X is not a bond.

The invention also encompasses methods of treating triple negative breast cancer and/or ovarian cancer by administering at least one compound of formula (III) in a therapeutically effective amount to a subject in need thereof, wherein the compound of Formula (III) has the formula compound of formula (III)

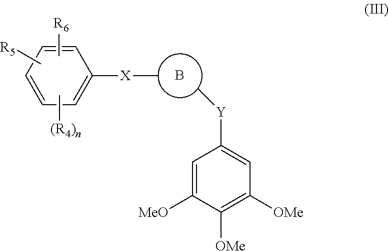

wherein

### B is

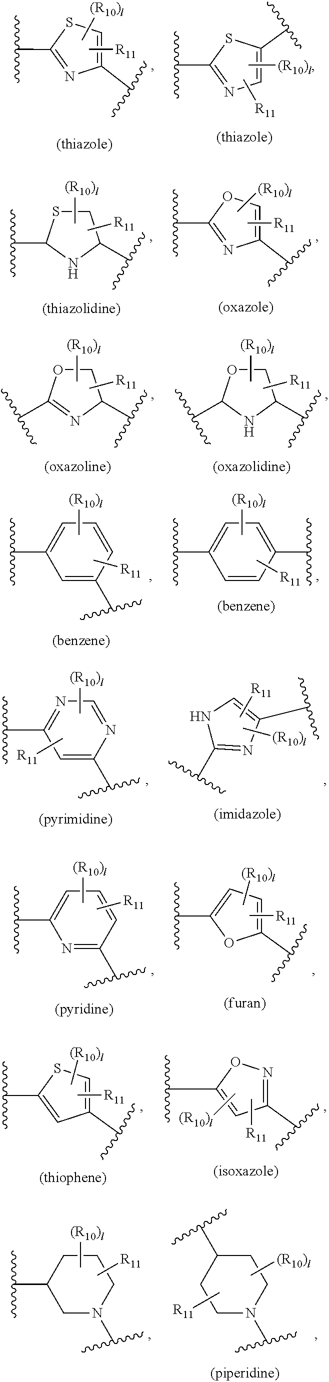

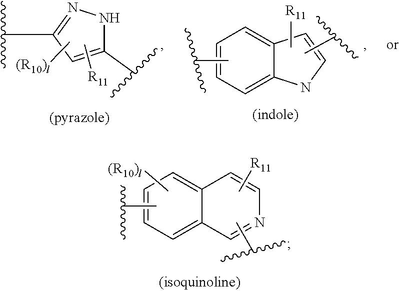

R₄, R₅ and R₆ are independently hydrogen, O-alkyl, O-haloalkyl, F, Cl, Br, I, haloalkyl, CF₃, CN, —CH₂CN, NH₂, hydroxyl, —(CH₂)ᵢNHCH₃, —(CH₂)ᵢNH₂, —(CH₂)ᵢN(CH₃)₂, —OC(O)CF₃, C₁-C₅ linear or branched alkyl, haloalkyl, alkylamino, aminoalkyl, —OCH₂Ph, —NHCO-alkyl, COOH, —C(O)Ph, C(O)O-alkyl, C(O)H, —C(O)NH₂ or NO₂; and
R₁₀ and R₁₁ are independently hydrogen, O-alkyl, O-haloalkyl, F, Cl, Br, I, haloalkyl, CF₃, CN, —CH₂CN, NH₂, hydroxyl, —(CH₂)ᵢNHCH₃, —(CH₂)ᵢNH₂, —(CH₂)ᵢN(CH₃)₂, —OC(O)CF₃, C₁-C₅ linear or branched alkyl, haloalkyl, alkylamino, aminoalkyl, —OCH₂Ph, —NHCO-alkyl, COOH, —C(O)Ph, C(O)O-alkyl, C(O)H, —C(O)NH₂ or NO₂;
X is a bond, NH, C₁ to C₅ hydrocarbon, O, or S;
Y is a bond, —C═O, —C═S, —C═N—NH₂, —C═N—OH, —CH—OH, —C═CH—CN, —C═N—CN, —CH═CH—, C═C(CH₃)₂, —C═N—OMe, —(C═O)—NH, —NH—(C═O), —(C═O)—O, —O—(C═O), —(CH₂)₁₋₅—(C═O), (C═O)—(CH₂)₁₋₅, —(SO₂)—NH—, —NH—(SO₂)—, SO₂, SO or S;
i is an integer between 0-5;
l is an integer between 0-2; and
n is an integer between 1-3;
wherein
if B is indole then X is not 0;
or its pharmaceutically acceptable salt, hydrate, polymorph, metabolite, tautomer or isomer.

In one embodiment, if B of formula III is a thiazole ring then X is not a bond.

The invention encompasses methods of treating triple negative breast cancer by administering at least one compound of formula (IV) in a therapeutically effective amount to a subject in need thereof, wherein the compound of Formula (IV) has the formula

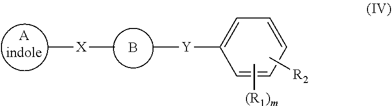

wherein ring A is an indolyl;

### B is

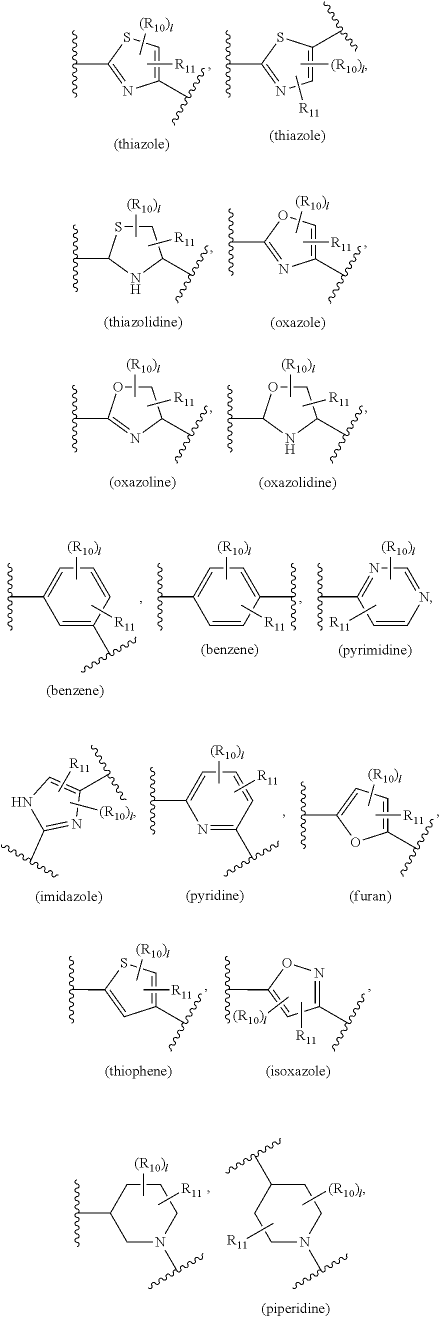

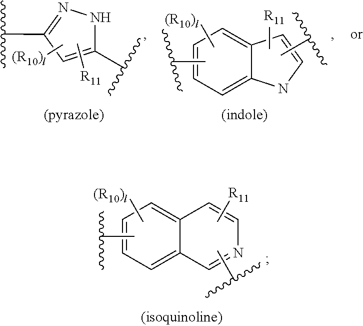

R₁ and R₂ are independently hydrogen, O-alkyl, O-haloalkyl, F, Cl, Br, I, haloalkyl, CF₃, CN, —CH₂CN, NH₂, hydroxyl, —(CH₂)ᵢNHCH₃, —(CH₂)ᵢNH₂, —(CH₂)ᵢN(CH₃)₂, —OC(O)CF₃, C₁-C₅ linear or branched alkyl, haloalkyl, alkylamino, aminoalkyl, —OCH₂Ph, —NHCO-alkyl, COOH, —C(O)Ph, C(O)O-alkyl, C(O)H, —C(O)NH₂ or NO₂;
R₁₀ and R₁₁ are independently hydrogen, O-alkyl, O-haloalkyl, F, Cl, Br, I, haloalkyl, CF₃, CN, —CH₂CN, NH₂, hydroxyl, —(CH₂)ᵢNHCH₃, —(CH₂)ᵢNH₂, —(CH₂)ᵢN(CH₃)₂, —OC(O)CF₃, C₁-C₅ linear or branched alkyl, haloalkyl, alkylamino, aminoalkyl, —OCH₂Ph, —NHCO-alkyl, COOH, —C(O)Ph, C(O)O-alkyl, C(O)H, —C(O)NH₂ or NO₂;
X is a bond, NH, C₁ to C₅ hydrocarbon, O, or S;
Y is a bond, C═O, —C═S, —C═N—NH₂, —C═N—OH, —CH—OH, —C═CH—CN, —C═N—CN, —CH═CH—, C═C(CH₃)₂, —C═N—OMe, —(C═O)—NH, —NH—(C═O), —(C═O)—O, —O—(C═O), —(CH₂)₁₋₅—(C═O), (C═O)—(CH₂)₁₋₅, —(SO₂)—NH—, —NH—(SO₂)—, SO₂, SO or S; wherein said A is optionally substituted by O-alkyl, O-haloalkyl, F, Cl, Br, I, haloalkyl, CF₃, CN, —CH₂CN, NH₂, hydroxyl, —(CH₂)ᵢNHCH₃, —(CH₂)ᵢNH₂, —(CH₂)ᵢN(CH₃)₂, —OC(O)CF₃, C₁-C₅ linear or branched alkyl, haloalkyl, alkylamino, aminoalkyl, —OCH₂Ph, —NHCO-alkyl, COOH, —C(O)Ph, C(O)O-alkyl, C(O)H, —C(O)NH₂ or NO₂; and
i is an integer between 0-5;
l is an integer between 0-2; and
m is an integer between 1-4;
wherein
if B is a benzene ring, a thiophene ring, a furan ring or an indole ring then X is not a bond or CH₂;
or its pharmaceutically acceptable salt, hydrate, polymorph, metabolite, tautomer or isomer.

In one embodiment, if B of formula IV is a thiazole ring then X is not a bond.

In another embodiment, the indolyl of ring A of formula IV is attached to one of its 1-7 positions to X or direct to B if X is a bond (i.e., nothing).

The invention also encompasses methods of treating triple negative breast cancer and/or ovarian cancer by administering at least one compound of formula IV(a) in a therapeutically effective amount to a subject in need thereof, wherein the compound of Formula

IV(a) has the formula:

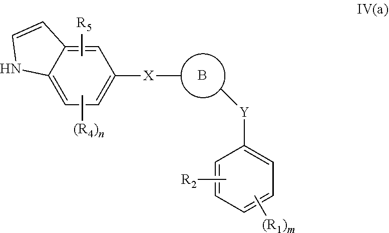

### B is

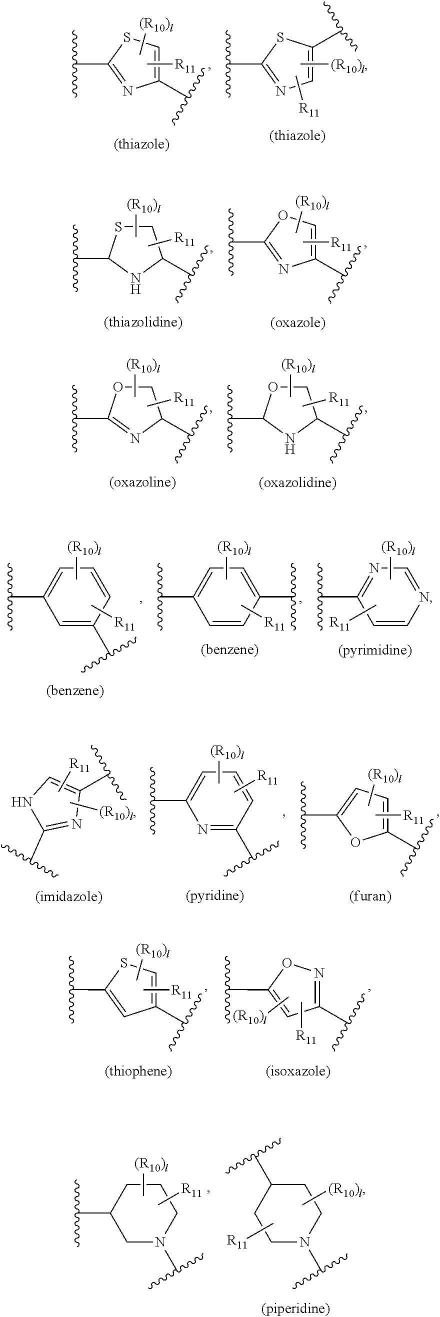

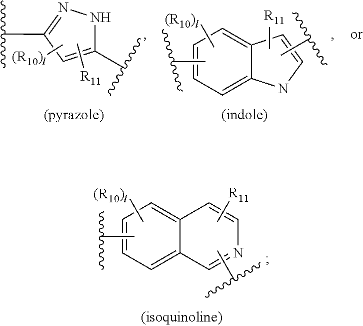

R₂, R₄ and R₅ are independently hydrogen, O-alkyl, O-haloalkyl, F, Cl, Br, I, haloalkyl, CF₃, CN, —CH₂CN, NH₂, hydroxyl, —(CH₂)ᵢNHCH₃, —(CH₂)ᵢNH₂, —(CH₂)ᵢN(CH₃)₂, —OC(O)CF₃, C₁-C₅ linear or branched alkyl, haloalkyl, alkylamino, aminoalkyl, —OCH₂Ph, —NHCO-alkyl, COOH, —C(O)Ph, C(O)O-alkyl, C(O)H, —C(O)NH₂ or NO₂; and
R₁₀ and R₁₁ are independently hydrogen, O-alkyl, O-haloalkyl, F, Cl, Br, I, haloalkyl, CF₃, CN, —CH₂CN, NH₂, hydroxyl, —(CH₂)ᵢNHCH₃, —(CH₂)ᵢNH₂, —(CH₂)ᵢN(CH₃)₂, —OC(O)CF₃, C₁-C₅ linear or branched alkyl, haloalkyl, alkylamino, aminoalkyl, —OCH₂Ph, —NHCO-alkyl, COOH, —C(O)Ph, C(O)O-alkyl, C(O)H, —C(O)NH₂ or NO₂;
X is a bond, NH, C₁ to C₅ hydrocarbon, O, or S;
Y is a bond or C═O, —C═S, —C═N—NH₂, —C═N—OH, —CH—OH, —C═CH—CN, —C═N—CN, —CH═CH—, C═C(CH₃)₂, —C═N—OMe, —(C═O)—NH, —NH—(C═O), —(C═O)—O, —O—(C═O), —(CH₂)₁₋₅—(C═O), (C═O)—(CH₂)₁₋₅, —(SO₂)—NH—, —NH—(SO₂)—, SO₂, SO or S;
i is an integer between 0-5;
l is an integer between 0-2;
n is an integer between 1-2; and
m is an integer between 1-4;
wherein
if B is a benzene ring, a thiophene ring, a furan ring or an indole ring then X is not a bond or CH₂;
or its pharmaceutically acceptable salt, hydrate, polymorph, metabolite, tautomer or isomer.

In one embodiment, if B of formula IVa is a thiazole ring then X is not a bond.

The invention also encompasses methods of treating triple negative breast cancer and/or ovarian cancer by administering at least one compound of formula (V) in a therapeutically effective amount to a subject in need thereof, wherein the compound of Formula (V) has the formula:

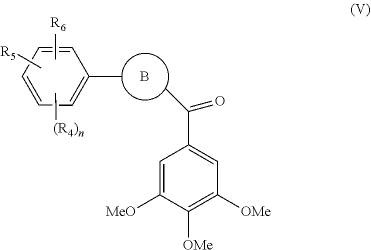

### B is

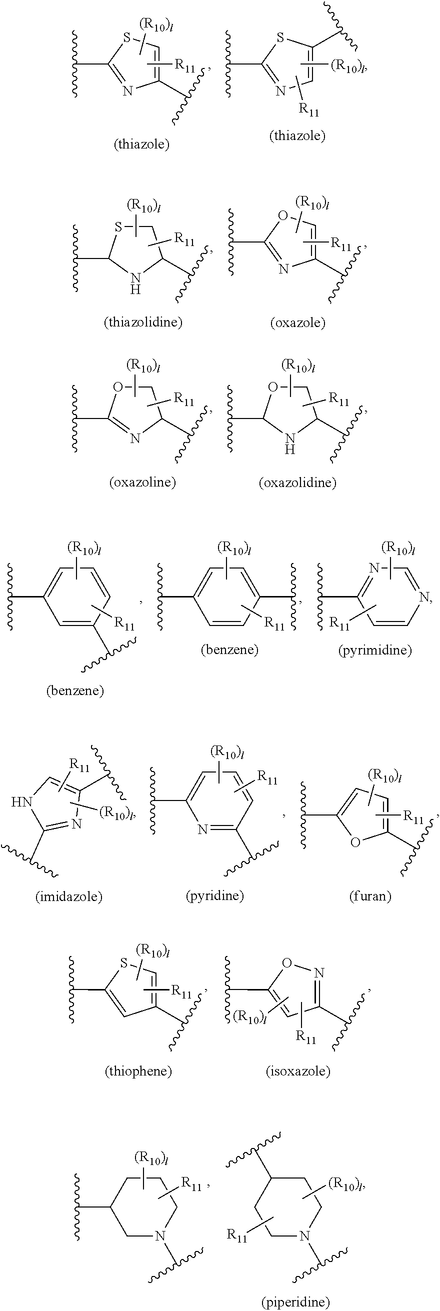

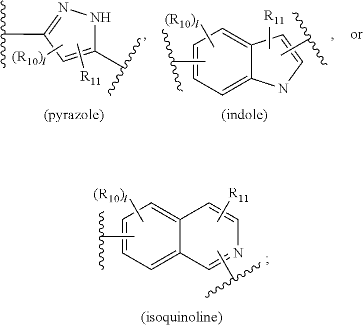

R₄, R₅ and R₆ are independently hydrogen, O-alkyl, O-haloalkyl, F, Cl, Br, I, haloalkyl, CF₃, CN, —CH₂CN, NH₂, hydroxyl, —(CH₂)ᵢNHCH₃, —(CH₂)ᵢNH₂, —(CH₂)ᵢN(CH₃)₂, —OC(O)CF₃, C₁-C₅ linear or branched alkyl, haloalkyl, alkylamino, aminoalkyl, —OCH₂Ph, —NHCO-alkyl, COOH, —C(O)Ph, C(O)O-alkyl, C(O)H, —C(O)NH₂ or NO₂;
R₁₀ and R₁₁ are independently hydrogen, O-alkyl, O-haloalkyl, F, Cl, Br, I, haloalkyl, CF₃, CN, —CH₂CN, NH₂, hydroxyl, —(CH₂)ᵢNHCH₃, —(CH₂)ᵢNH₂, —(CH₂)ᵢN(CH₃)₂, —OC(O)CF₃, C₁-C₅ linear or branched alkyl, haloalkyl, alkylamino, aminoalkyl, —OCH₂Ph, —NHCO-alkyl, COOH, —C(O)Ph, C(O)O-alkyl, C(O)H, —C(O)NH₂ or NO₂;
i is an integer between 1-5;
l is an integer between 0-2; and
n is an integer between 1-3;
or its pharmaceutically acceptable salt, hydrate, polymorph, metabolite, tautomer or isomer.

In another embodiment, B of formula V is not a thiazole

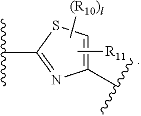

In another embodiment, B of formula V is not an oxazole. In another embodiment, B of formula V is not an oxazoline. In another embodiment, B of formula V is not an imidazole. In another embodiment, B of formula V is not a thiazole, oxazole, oxazoline or imidazole.

Compounds encompassed by the method of the invention include the following compounds:

Formula V

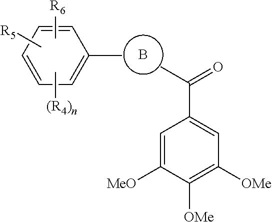

| | | | R₄, R₅ and |
|---|---|---|---|
| Compound | B | | R₆ |
| 1a | 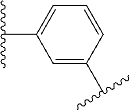 [SMILES: *c1cccc(*)c1] | | H |
| 1b | 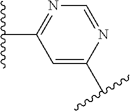 [SMILES: *c1cc(*)ncn1] | | H |
| 1c | 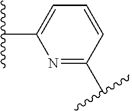 [SMILES: *c1cccc(*)n1] | | H |
| 1d | 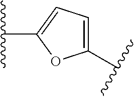 [SMILES: *c1ccc(*)o1] | | H |
| 1e | 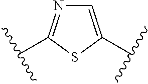 [SMILES: *c1cnc(*)s1] | 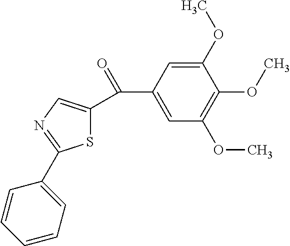 [SMILES: COc1cc(C(=O)c2cnc(-c3ccccc3)s2)cc(OC)c1OC] | H |
| 1f | 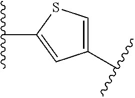 [SMILES: *c1csc(*)c1] | 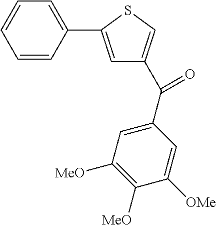 [SMILES: COc1cc(C(=O)c2csc(-c3ccccc3)c2)cc(OC)c1OC] | H |
| 1g | 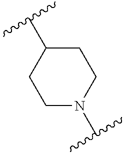 [SMILES: *C1CCN(*)CC1] | 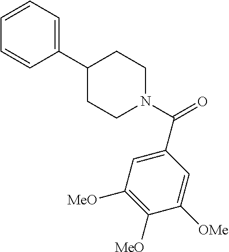 [SMILES: COc1cc(C(=O)N2CCC(c3ccccc3)CC2)cc(OC)c1OC] | H |
| 1h | 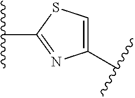 [SMILES: *c1csc(*)n1] | | H |
| 1i | 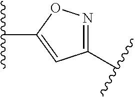 [SMILES: *c1cc(*)on1] | | H |
| 1k | 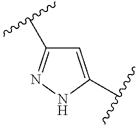 [SMILES: *c1cc(*)[nH]n1] | 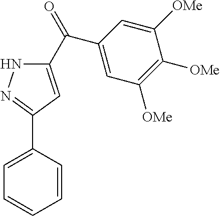 [SMILES: COc1cc(C(=O)c2cc(-c3ccccc3)n[nH]2)cc(OC)c1OC] | H |
| 1l | 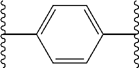 [SMILES: *c1ccc(*)cc1] | 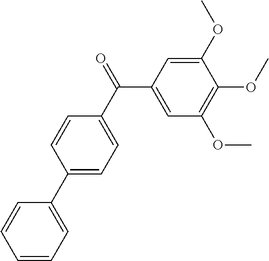 [SMILES: COc1cc(C(=O)c2ccc(-c3ccccc3)cc2)cc(OC)c1OC] | H |
| 35a | 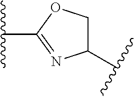 [SMILES: *C1=NC(*)CO1] | 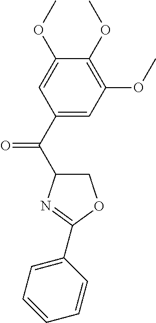 [SMILES: COc1cc(C(=O)C2COC(c3ccccc3)=N2)cc(OC)c1OC] | H |
| 36a | 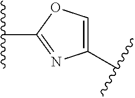 [SMILES: *c1coc(*)n1] | 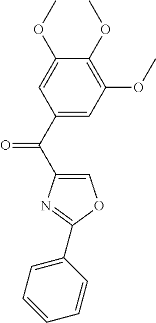 [SMILES: COc1cc(C(=O)c2coc(-c3ccccc3)n2)cc(OC)c1OC] | H |

The invention also encompasses methods of treating triple negative breast cancer and/or ovarian cancer by administering at least one compound of formula (VI) in a therapeutically effective amount to a subject in need thereof, wherein the compound of Formula (VI) has the formula:

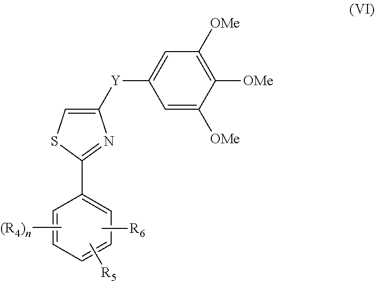

wherein
R₄, R₅ and R₆ are independently hydrogen, O-alkyl, O-haloalkyl, F, Cl, Br, I, haloalkyl, CF₃, CN, —CH₂CN, NH₂, hydroxyl, —(CH₂)ᵢNHCH₃, —(CH₂)ᵢNH₂, —(CH₂)ᵢN(CH₃)₂, —OC(O)CF₃, C₁-C₅ linear or branched alkyl, haloalkyl, alkylamino, aminoalkyl, —OCH₂Ph, —NHCO-alkyl, COOH, —C(O)Ph, C(O)O-alkyl, C(O)H, —C(O)NH₂ or NO₂; and
Y is a bond or C═O, —C═S, —C═N—NH₂, —C═N—OH, —CH—OH, —C═CH—CN, —C═N—CN, —CH═CH—, C═C(CH₃)₂, —C═N—OMe, —(C═O)—NH, —NH—(C═O), —(C═O)—O, —O—(C═O), —(CH₂)₁₋₅—(C═O), (C═O)—(CH₂)₁₋₅, —(SO₂)—NH—, —NH—(SO₂)—, SO₂, SO or S;
n is an integer between 1-3; and
i is an integer from 1-5;
or its pharmaceutically acceptable salt, hydrate, polymorph, metabolite, tautomer or isomer.

The invention encompasses methods with the following compounds:

Formula VI

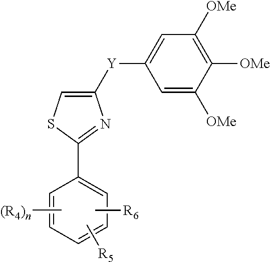

| Compound | Y | R₄, R₅ and R₆ |
|---|---|---|
| 1h | —C═O | H |
| 2a | —C═C(CH₃)₂ | H |
| 2b | —CH—OH | H |
| 2c | —C═CH—CN | H |
| | (cis and trans) | |
| 2d | —C═N—NH₂ | H |
| | (cis and trans) | |
| 2e | —C═N—OH | H |
| | (cis and trans) | |
| 2f | —C═N—OMe | H |
| | (cis and trans) | |
| 2g | —(C═O)—NH— | H |
| 2h | —NH—(C═O)— | H |
| 2i | nothing | H |
| 2j | —C═N—CN | H |
| | (cis and trans) | |
| 2k | C═O | R₄ = R₆ = H |
| | | R₅ = p-F |
| 2l | C═O | R₄ = R₆ = H |
| | | R₅ = p-OH |
| 2m | C═O | R₄ = R₆ = H |
| | | R₅ = p-CH₃ |
| 2n | C═O | R₄ = R₆ = H |
| | | R₅ = p-CH₂—CN |
| 2o | C═O | R₄ = R₆ = H |
| | | R₅ = p-N(CH₃)₂ |
| 2p | C═O | R₄ = m-F; |
| | | R₅ = p-F; |
| | | R₆ = m-F; |
| | | n = 1 |
| 2q | C═O | R₄ = R₆ = H |
| | | R₅ = |
| | | p-CH₂—(C═O)NH₂ |
| 2r | C═O | R₄ = R₆ = H |
| | | R₅ = p-CH₂NH₂ |
| 2s | C═O | R₄ = R₆ = H |
| | | R₅ = p-CH₂NH—CH₃ |
| 2t | C═O | R₄ = m-OMe; |
| | | R₅ = p-OMe; |
| | | R₆ = m-OMe; |
| | | n = 1 |
| 2u | C═O | R₄ = R₆ = H |
| | | R₅ = p-CH₂NMe₂ |

In one embodiment, this invention is directed to compound 3a:

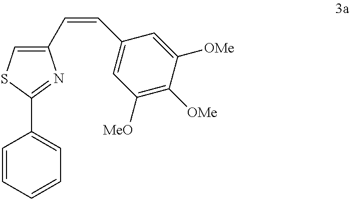 [SMILES: COc1cc(/C=C\c2csc(-c3ccccc3)n2)cc(OC)c1OC]

In one embodiment, this invention is directed to compound 3b:

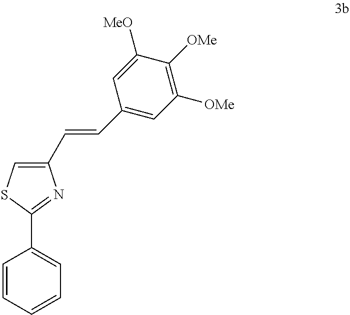 [SMILES: COc1cc(/C=C/c2csc(-c3ccccc3)n2)cc(OC)c1OC]

In one embodiment, this invention is directed to a compound of formula (VII)

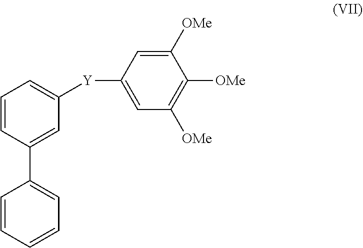

wherein
Y is a bond or C═O, —C═S, —C═N—NH₂, —C═N—OH, —CH—OH, —C═CH—CN, —C═N—CN, —CH═CH—, C═C(CH₃)₂, —C═N—OMe, —(C═O)—NH, —NH—(C═O), —(C═O)—O, —O—(C═O), —(CH₂)₁₋₅—(C═O), (C═O)—(CH₂)₁₋₅, —(SO₂)—NH—, —NH—(SO₂)—, SO₂, SO or S;
or its pharmaceutically acceptable salt, hydrate, polymorph, metabolite, tautomer or isomer.

In one embodiment, this invention is directed to the following compounds:

Formula VII

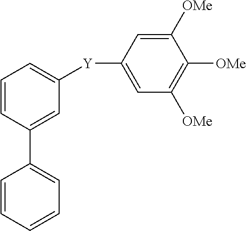

| Compound | Y |
|---|---|
| 4a | S |
| 4b | SO₂ |
| 4c | SO |
| 4d | —(SO₂)—NH— |

In one embodiment, this invention is directed to a compound of formula (VIII)

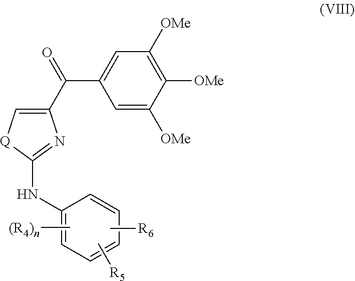

wherein
R₄, R₅ and R₆ are independently hydrogen, O-alkyl, O-haloalkyl, F, Cl, Br, I, haloalkyl, CF₃, CN, —CH₂CN, NH₂, hydroxyl, —(CH₂)ᵢNHCH₃, —(CH₂)ᵢNH₂, —(CH₂)ᵢN(CH₃)₂, —OC(O)CF₃, C₁-C₅ linear or branched alkyl, haloalkyl, alkylamino, aminoalkyl, —OCH₂Ph, —NHCO-alkyl, COOH, —C(O)Ph, C(O)O-alkyl, C(O)H, —C(O)NH₂ or NO₂;

### Q is S, O or NH;

i is an integer between 0-5; and
n is an integer between 1-3;
or its pharmaceutically acceptable salt, hydrate, polymorph, metabolite, tautomer or isomer.

In one embodiment, this invention is directed to the following compounds:

Formula VIII

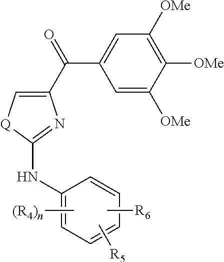

| Compound | R₄ | R₅ | R₆ | Q |
|---|---|---|---|---|
| 5a | H | H | H | S |
| | n = 1 | | | |
| 5b | H | p-CH₃ | H | S |
| | n = 1 | | | |
| 5c | H | p-F | H | S |
| | n = 1 | | | |
| 5d | H | p-Cl | H | S |
| | n = 1 | | | |
| 5e | H | H | H | S |
| | n = 1 | | | |

The invention also encompasses methods of treating triple negative breast cancer and/or ovarian cancer by administering at least one compound of formula (IX) in a therapeutically effective amount to a subject in need thereof, wherein the compound of Formula (IX):

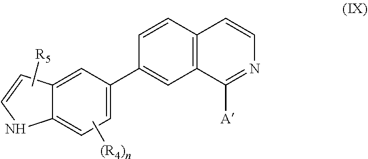

wherein
R₄ and R₅ are independently hydrogen, O-alkyl, O-haloalkyl, F, Cl, Br, I, haloalkyl, CF₃, CN, —CH₂CN, NH₂, hydroxyl, —(CH₂)ᵢNHCH₃, —(CH₂)ᵢNH₂, —(CH₂)ᵢN(CH₃)₂, —OC(O)CF₃, C₁-C₅ linear or branched alkyl, haloalkyl, alkylamino, aminoalkyl, —OCH₂Ph, —NHCO-alkyl, COOH, —C(O)Ph, C(O)O-alkyl, C(O)H, —(O)NH₂ or NO₂;
A′ is halogen; substituted or unsubstituted single-, fused- or multiple-ring, aryl or (hetero)cyclic ring systems; substituted or unsubstituted, saturated or unsaturated N-heterocycles; substituted or unsubstituted, saturated or unsaturated S-heterocycles; substituted or unsubstituted, saturated or unsaturated O-heterocycles; substituted or unsubstituted, saturated or unsaturated cyclic hydrocarbons; or substituted or unsubstituted, saturated or unsaturated mixed heterocycles; wherein said A′ ring is optionally substituted by 1-5 substituents which are independently O-alkyl, O-haloalkyl, F, Cl, Br, I, haloalkyl, CF₃, CN, —CH₂CN, NH₂, hydroxyl, —(CH₂)ᵢNHCH₃, —(CH₂)ᵢNH₂, —(CH₂)ᵢN(CH₃)₂, —OC(O)CF₃, C₁-C₅ linear or branched alkyl, haloalkyl, alkylamino, aminoalkyl, —OCH₂Ph, —NHCO-alkyl, COOH, —C(O)Ph, C(O)O-alkyl, C(O)H, —C(O)NH₂ or NO₂;
i is an integer between 1-5; and
n is an integer between 1-3;
or its pharmaceutically acceptable salt, hydrate, polymorph, metabolite, tautomer or isomer.

In one embodiment, a compound of Formula IX is represented by the structures of the following compounds:

Formula IX

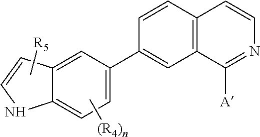

| Compound | A′ | R₄, R₅ |
|---|---|---|
| 6a | 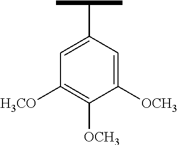 | H |
| 6b | 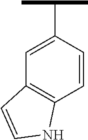 | H |
| 6c | 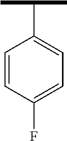 | H |
| 6d | Cl | H |

In another embodiment A′ of formula IX is a halogen. In one embodiment A′ of formula IX is a phenyl. In another embodiment A′ of formula IX is substituted phenyl. In another embodiment the substitution of A′ is halogen. In another embodiment the substitution is 4-F. In another embodiment the substitution is 3,4,5-(OCH₃)₃. In another embodiment, A′ of formula IX is substituted or unsubstituted 5-indolyl. In another embodiment, A′ of formula IX is substituted or unsubstituted 2-indolyl. In another embodiment, A′ of formula IX is substituted or unsubstituted 3-indolyl.

The invention also encompasses methods of treating triple negative breast cancer and/or ovarian cancer by administering at least one compound of formula (IXa) in a therapeutically effective amount to a subject in need thereof, wherein the compound of Formula (IXa):

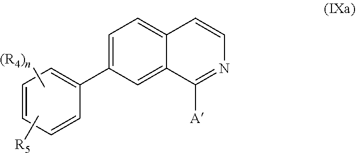

wherein
R₄ and R₅ are independently hydrogen, O-alkyl, O-haloalkyl, F, Cl, Br, I, haloalkyl, CF₃, CN, —CH₂CN, NH₂, hydroxyl, —(CH₂)ᵢNHCH₃, —(CH₂)ᵢNH₂,
—(CH₂)ᵢN(CH₃)₂, —OC(O)CF₃, C₁-C₅ linear or branched alkyl, haloalkyl, alkylamino, aminoalkyl, —OCH₂Ph, —NHCO-alkyl, COOH, —C(O)Ph, C(O)O-alkyl, C(O)H, —(O)NH₂ or NO₂;
A′ is halogen; substituted or unsubstituted single-, fused- or multiple-ring, aryl or (hetero)cyclic ring systems; substituted or unsubstituted, saturated or unsaturated N-heterocycles; substituted or unsubstituted, saturated or unsaturated S-heterocycles; substituted or unsubstituted, saturated or unsaturated O-heterocycles; substituted or unsubstituted, saturated or unsaturated cyclic hydrocarbons; or substituted or unsubstituted, saturated or unsaturated mixed heterocycles; wherein said A′ ring is optionally substituted by 1-5 substituents which are independently O-alkyl, O-haloalkyl, F, Cl, Br, I, haloalkyl, CF₃, CN, —CH₂CN, NH₂, hydroxyl, —(CH₂)ᵢNHCH₃, —(CH₂)ᵢNH₂, —(CH₂)ᵢN(CH₃)₂, —OC(O)CF₃, C₁-C₅ linear or branched alkyl, haloalkyl, alkylamino, aminoalkyl, —OCH₂Ph, —NHCO-alkyl, COOH, —C(O)Ph, C(O)O-alkyl, C(O)H, —C(O)NH₂ or NO₂;
i is an integer between 1-5; and
n is an integer between 1-3;
or its pharmaceutically acceptable salt, hydrate, polymorph, metabolite, tautomer or isomer.

In another embodiment A′ of formula IXa is a halogen. In one embodiment A′ of formula IXa is a phenyl. In another embodiment A′ of formula IXa is substituted phenyl. In another embodiment the substitution of A′ is halogen. In another embodiment the substitution is 4-F. In another embodiment the substitution is 3,4,5-(OCH₃)₃. In another embodiment, A′ of formula IXa is substituted or unsubstituted 5-indolyl. In another embodiment, A′ of formula IXa is substituted or unsubstituted 2-indolyl. In another embodiment, A′ of formula IXa is substituted or unsubstituted 3-indolyl.

In another embodiment, a compound of formula IXa is 1-chloro-7-(4-fluorophenyl)isoquinoline. In another embodiment, a compound of formula IXa is 7-(4-fluorophenyl)-1-(1H-indol-5-yl)isoquinoline. In another embodiment, a compound of formula IXa is 7-(4-fluorophenyl)-1-(3,4,5-trimethoxyphenyl)isoquinoline. In another embodiment, a compound of formula IXa is 1,7-bis(4-fluorophenyl)isoquinoline (40). In another embodiment, a compound of formula IXa is 1,7-bis(3,4,5-trimethoxyphenyl)isoquinoline. In another embodiment, a compound of formula IXa is 1-(4-fluorophenyl)-7-(3,4,5-trimethoxyphenyl)isoquinoline. In another embodiment, a compound of formula IXa is 1-(1H-indol-5-yl)-7-(3,4,5-trimethoxyphenyl)isoquinoline. In another embodiment, a compound of formula IXa is 1-chloro-7-(3,4,5-trimethoxyphenyl)isoquinoline.

The invention also encompasses methods of treating triple negative breast cancer and/or ovarian cancer by administering at least one compound of formula (XI) in a therapeutically effective amount to a subject in need thereof, wherein the compound of Formula (XI) is represented by the structure:

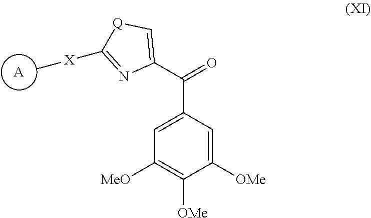

wherein
X is a bond, NH or S;

### Q is O, NH or S; and

A is substituted or unsubstituted single-, fused- or multiple-ring aryl or (hetero)cyclic ring systems; substituted or unsubstituted, saturated or unsaturated N-heterocycles; substituted or unsubstituted, saturated or unsaturated S-heterocycles; substituted or unsubstituted, saturated or unsaturated O-heterocycles; substituted or unsubstituted, saturated or unsaturated cyclic hydrocarbons; or substituted or unsubstituted, saturated or unsaturated mixed heterocycles; wherein said A ring is optionally substituted by 1-5 1-5 substituents which are independently O-alkyl, O-haloalkyl, F, Cl, Br, I, haloalkyl, CF₃, CN, —CH₂CN, NH₂, hydroxyl, —(CH₂)ᵢNHCH₃, —(CH₂)ᵢNH₂, —(CH₂)ᵢN(CH₃)₂, —OC(O)CF₃, C₁-C₅ linear or branched alkyl, haloalkyl, alkylamino, aminoalkyl, —OCH₂Ph, —NHCO-alkyl, COOH, —C(O)Ph, C(O)O-alkyl, C(O)H, —C(O)NH₂ or NO₂; and
i is an integer from 0-5;
or its pharmaceutically acceptable salt, hydrate, polymorph, metabolite, tautomer or isomer.

In one embodiment if Q of Formula XI is S, then X is not a bond.

In one embodiment, A of compound of Formula XI is Ph. In another embodiment, A of compound of Formula XI is substituted Ph. In another embodiment, the substitution is 4-F. In another embodiment, the substitution is 4-Me. In another embodiment, Q of compound of Formula XI is S. In another embodiment, X of compound of Formula XI is NH. Non limiting examples of compounds of Formula XI are selected from: (2-(phenylamino)thiazol-4-yl)(3,4,5-trimethoxyphenyl)methanone (5a), (2-(p-tolylamino)thiazol-4-yl)(3,4,5-trimethoxyphenyl)methanone (5b), (2-(p-fluorophenylamino)thiazol-4-yl)(3,4,5-trimethoxyphenyl)methanone (5c), (2-(4-chlorophenylamino)thiazol-4-yl)(3,4,5-trimethoxyphenyl)methanone (5d), (2-(phenylamino)-1H-imidazol-4-yl)(3,4,5-trimethoxyphenyl)methanone (5e), (2-(phenylamino)thiazol-4-yl)(3,4,5-trimethoxyphenyl)methanone hydrochloride salt (5Ha), (2-(p-tolylamino)thiazol-4-yl)(3,4,5-trimethoxyphenyl)methanone hydrochloride salt (5Hb), (2-(p-fluorophenylamino)thiazol-4-yl)(3,4,5-trimethoxyphenyl)methanone hydrochloride salt (5Hc), (2-(4-chlorophenylamino)thiazol-4-yl)(3,4,5-trimethoxyphenyl)methanone hydrochloride salt (5Hd), (2-(phenylamino)-1H-imidazol-4-yl)(3,4,5-trimethoxyphenyl)methanone hydrochloride salt (5He).

The invention also encompasses methods of treating triple negative breast cancer and/or ovarian cancer by administering at least one compound of formula XI(a) in a therapeutically effective amount to a subject in need thereof, wherein the compound of Formula XI(a) is represented by the structure:

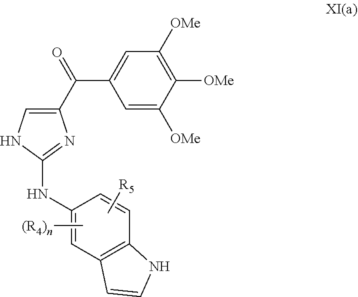

wherein R₄ and R₅ are independently hydrogen, O-alkyl, O-haloalkyl, F, Cl, Br, I, haloalkyl, CF₃, CN, —CH₂CN, NH₂, hydroxyl, —(CH₂)ᵢNHCH₃, —(CH₂)ᵢNH₂, —(CH₂)ᵢN(CH₃)₂, —OC(O)CF₃, C₁-C₅ linear or branched alkyl, haloalkyl, alkylamino, aminoalkyl, —OCH₂Ph, —NHCO-alkyl, COOH, —C(O)Ph, C(O)O-alkyl, C(O)H, —C(O)NH₂ or NO₂;
i is an integer from 0-5; and
n is an integer between 1-4;
or its pharmaceutically acceptable salt, hydrate, polymorph, metabolite, tautomer or isomer.

The invention also encompasses methods of treating triple negative breast cancer and/or ovarian cancer by administering at least one compound of formula XI(b) in a therapeutically effective amount to a subject in need thereof, wherein the compound of Formula XI(b) is represented by the structure:

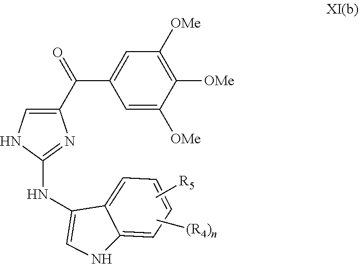

wherein R₄ and R₅ are independently hydrogen, O-alkyl, O-haloalkyl, F, Cl, Br, I, haloalkyl, CF₃, CN, —CH₂CN, NH₂, hydroxyl, —(CH₂)ᵢNHCH₃, —(CH₂)ᵢNH₂, —(CH₂)ᵢN(CH₃)₂, —OC(O)CF₃, C₁-C₅ linear or branched alkyl, haloalkyl, alkylamino, aminoalkyl, —OCH₂Ph, —NHCO-alkyl, COOH, —C(O)Ph, C(O)O-alkyl, C(O)H, —C(O)NH₂ or NO₂;
i is an integer from 0-5; and
n is an integer between 1-4;
or its pharmaceutically acceptable salt, hydrate, polymorph, metabolite, tautomer or isomer.

The invention also encompasses methods of treating triple negative breast cancer and/or ovarian cancer by administering at least one compound of formula XI(c) in a therapeutically effective amount to a subject in need thereof, wherein the compound of Formula

XI(c) is represented by the structure:

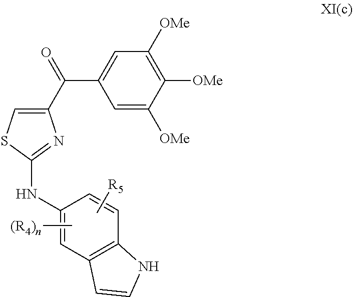

wherein R₄ and R₅ are independently hydrogen, O-alkyl, O-haloalkyl, F, Cl, Br, I, haloalkyl, CF₃, CN, —CH₂CN, NH₂, hydroxyl, —(CH₂)ᵢNHCH₃, —(CH₂)ᵢNH₂, —(CH₂)ᵢN(CH₃)₂, —OC(O)CF₃, C₁-C₅ linear or branched alkyl, haloalkyl, alkylamino, aminoalkyl, —OCH₂Ph, —NHCO-alkyl, COOH, —C(O)Ph, C(O)O-alkyl, C(O)H, —C(O)NH₂ or NO₂;
i is an integer from 0-5; and
n is an integer between 1-4;
or its pharmaceutically acceptable salt, hydrate, polymorph, metabolite, tautomer or isomer.

The invention also encompasses methods of treating triple negative breast cancer and/or ovarian cancer by administering at least one compound of formula XI(d) in a therapeutically effective amount to a subject in need thereof, wherein the compound of Formula XI(d) is represented by the structure:

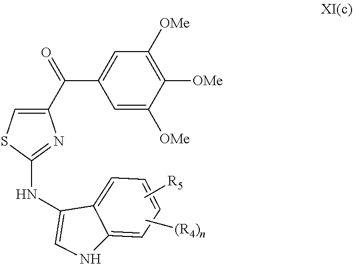

wherein R₄ and R₅ are independently hydrogen, O-alkyl, O-haloalkyl, F, Cl, Br, I, haloalkyl, CF₃, CN, —CH₂CN, NH₂, hydroxyl, —(CH₂)ᵢNHCH₃, —(CH₂)ᵢNH₂, —(CH₂)ᵢN(CH₃)₂, —OC(O)CF₃, C₁-C₅ linear or branched alkyl, haloalkyl, alkylamino, aminoalkyl, —OCH₂Ph, —NHCO-alkyl, COOH, —C(O)Ph, C(O)O-alkyl, C(O)H, —C(O)NH₂ or NO₂;
i is an integer from 0-5; and
n is an integer between 1-4;
or its pharmaceutically acceptable salt, hydrate, polymorph, metabolite, tautomer or isomer.

The invention also encompasses methods of treating triple negative breast cancer and/or ovarian cancer by administering at least one compound of formula XI(e) in a therapeutically effective amount to a subject in need thereof, wherein the compound of Formula XI(e) is represented by the structure:

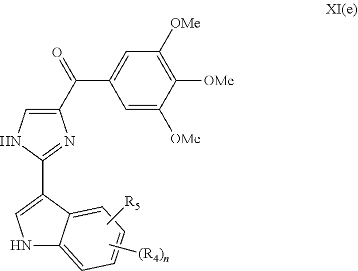

wherein R₄ and R₅ are independently hydrogen, O-alkyl, O-haloalkyl, F, Cl, Br, I, haloalkyl, CF₃, CN, —CH₂CN, NH₂, hydroxyl, —(CH₂)ᵢNHCH₃, —(CH₂)ᵢNH₂, —(CH₂)ᵢN(CH₃)₂, —OC(O)CF₃, C₁-C₅ linear or branched alkyl, haloalkyl, alkylamino, aminoalkyl, —OCH₂Ph, —NHCO-alkyl, COOH, —C(O)Ph, C(O)O-alkyl, C(O)H, —C(O)NH₂ or NO₂;
i is an integer from 0-5; and
n is an integer between 1-4;
or its pharmaceutically acceptable salt, hydrate, polymorph, metabolite, tautomer or isomer.

The invention also encompasses methods of treating triple negative breast cancer and/or ovarian cancer by administering compound 55 in a therapeutically effective amount to a subject in need thereof, wherein compound 55 is represented by the structure:

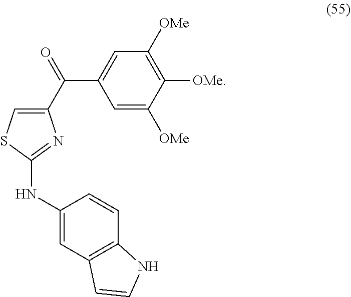 [SMILES: COc1cc(C(=O)c2csc(Nc3ccc4[nH]ccc4c3)n2)cc(OC)c1OC]

The invention also encompasses methods of treating triple negative breast cancer and/or ovarian cancer by administering compound 17ya in a therapeutically effective amount to a subject in need thereof, wherein compound 17ya is represented by the structure:

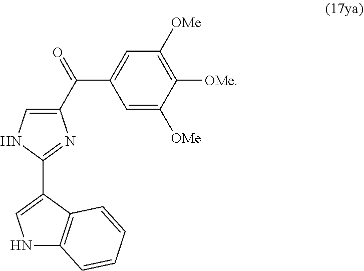 [SMILES: COc1cc(C(=O)c2c[nH]c(-c3c[nH]c4ccccc34)n2)cc(OC)c1OC]

The invention also encompasses methods of treating triple negative breast cancer and/or ovarian cancer by administering at least one compound of the following structures in a therapeutically effective amount to a subject in need thereof, wherein the compound is selected from the following structures:

| com- | |
|---|---|
| pound | structure |
| 8 | 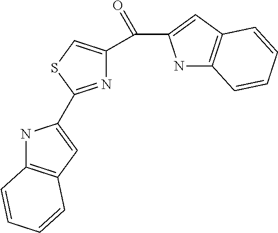 [SMILES: O=C(c1csc(-c2cc3ccccc3[nH]2)n1)c1cc2ccccc2[nH]1] |
| 9 | 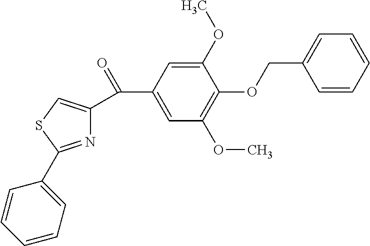 [SMILES: COc1cc(C(=O)c2csc(-c3ccccc3)n2)cc(OC)c1OCc1ccccc1] |
| 10 | 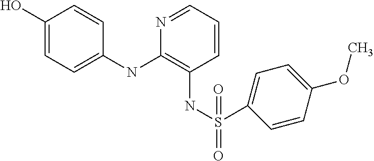 [SMILES: COc1ccc(S(=O)(=O)Nc2cccnc2Nc2ccc(O)cc2)cc1] |
| 11 | 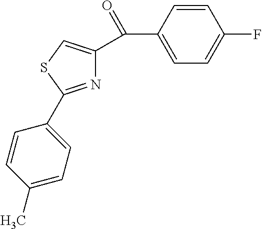 [SMILES: Cc1ccc(-c2nc(C(=O)c3ccc(F)cc3)cs2)cc1] |
| 12 | 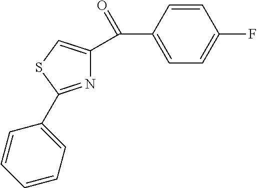 [SMILES: O=C(c1ccc(F)cc1)c1csc(-c2ccccc2)n1] |
| 13 | 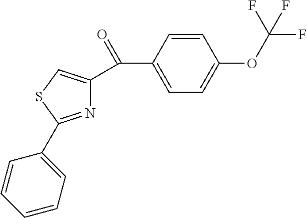 [SMILES: O=C(c1ccc(OC(F)(F)F)cc1)c1csc(-c2ccccc2)n1] |
| 14 | 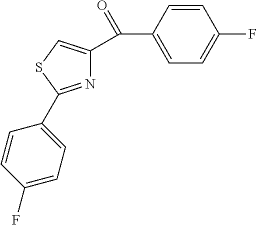 [SMILES: O=C(c1ccc(F)cc1)c1csc(-c2ccc(F)cc2)n1] |
| 16 | 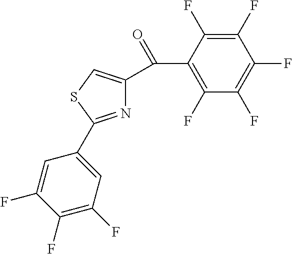 [SMILES: O=C(c1csc(-c2cc(F)c(F)c(F)c2)n1)c1c(F)c(F)c(F)c(F)c1F] |
| 17 | 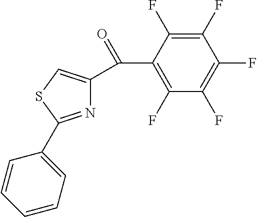 [SMILES: O=C(c1csc(-c2ccccc2)n1)c1c(F)c(F)c(F)c(F)c1F] |
| 18 | 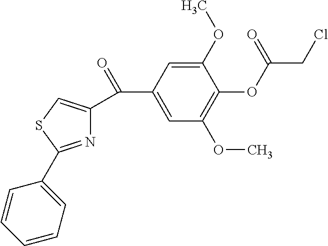 [SMILES: COc1cc(C(=O)c2csc(-c3ccccc3)n2)cc(OC)c1OC(=O)CCl] |
| 19 | 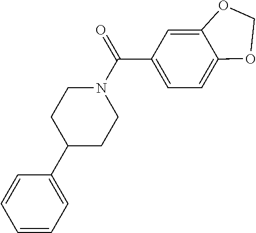 [SMILES: O=C(c1ccc2c(c1)OCO2)N1CCC(c2ccccc2)CC1] |
| 20 | 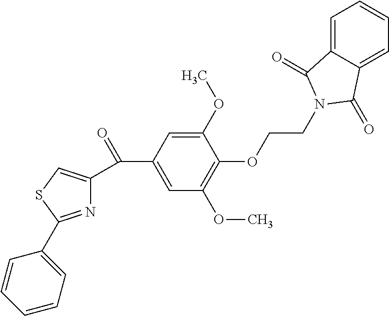 [SMILES: COc1cc(C(=O)c2csc(-c3ccccc3)n2)cc(OC)c1OCCN1C(=O)c2ccccc2C1=O] |
| 21 | 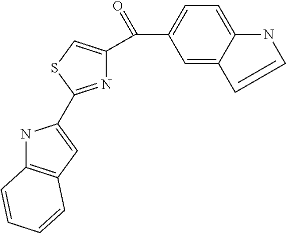 [SMILES: O=C(c1ccc2[nH]ccc2c1)c1csc(-c2cc3ccccc3[nH]2)n1] |
| 22 | 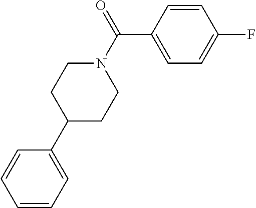 [SMILES: O=C(c1ccc(F)cc1)N1CCC(c2ccccc2)CC1] |
| 23 | 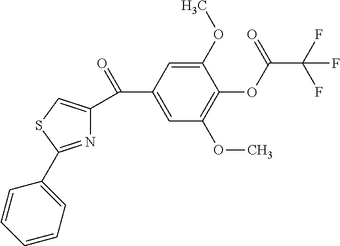 [SMILES: COc1cc(C(=O)c2csc(-c3ccccc3)n2)cc(OC)c1OC(=O)C(F)(F)F] |
| 24 | 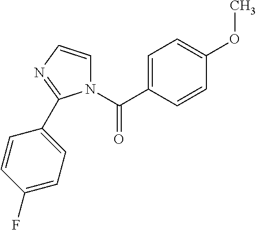 [SMILES: COc1ccc(C(=O)n2ccnc2-c2ccc(F)cc2)cc1] |
| 25 | 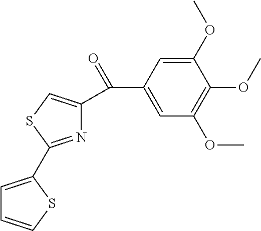 [SMILES: COc1cc(C(=O)c2csc(-c3cccs3)n2)cc(OC)c1OC] |
| 26 | 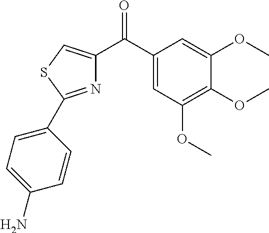 [SMILES: COc1cc(C(=O)c2csc(-c3ccc(N)cc3)n2)cc(OC)c1OC] |
| 27 | 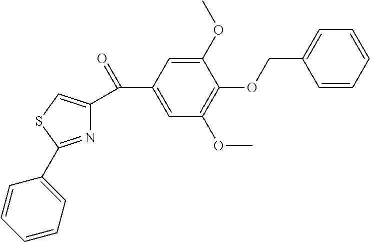 [SMILES: COc1cc(C(=O)c2csc(-c3ccccc3)n2)cc(OC)c1OCc1ccccc1] |
| 28 | 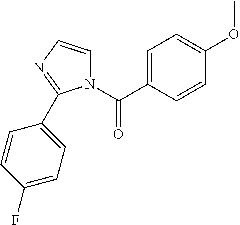 [SMILES: COc1ccc(C(=O)n2ccnc2-c2ccc(F)cc2)cc1] |
| 29 | 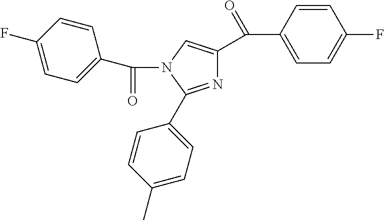 [SMILES: Cc1ccc(-c2nc(C(=O)c3ccc(F)cc3)cn2C(=O)c2ccc(F)cc2)cc1] |
| 30 | 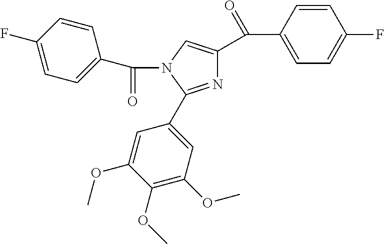 [SMILES: COc1cc(-c2nc(C(=O)c3ccc(F)cc3)cn2C(=O)c2ccc(F)cc2)cc(OC)c1OC] |
| 32 | 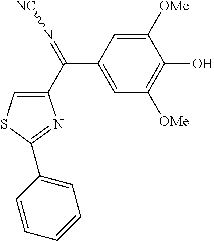 [SMILES: COc1cc(C(=NC#N)c2csc(-c3ccccc3)n2)cc(OC)c1O] |
| 33 | 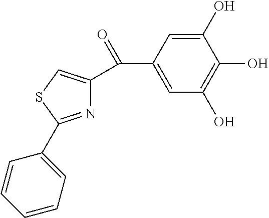 [SMILES: O=C(c1cc(O)c(O)c(O)c1)c1csc(-c2ccccc2)n1] |
| 34 | 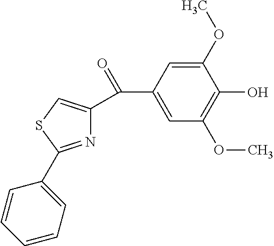 [SMILES: COc1cc(C(=O)c2csc(-c3ccccc3)n2)cc(OC)c1O] |
| 35 | 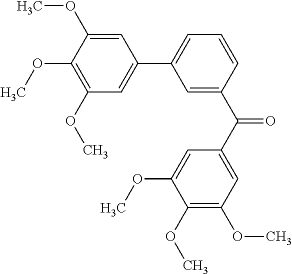 |
| 40 | 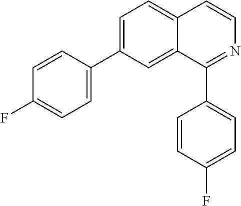 [SMILES: Fc1ccc(-c2ccc3ccnc(-c4ccc(F)cc4)c3c2)cc1] |
| 41 | 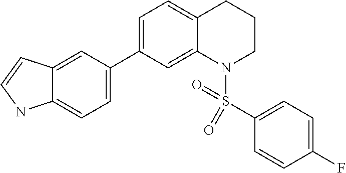 [SMILES: O=S(=O)(c1ccc(F)cc1)N1CCCc2ccc(-c3ccc4[nH]ccc4c3)cc21] |
| 42 | 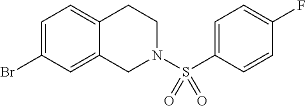 [SMILES: O=S(=O)(c1ccc(F)cc1)N1CCc2ccc(Br)cc2C1] |
| 43 | 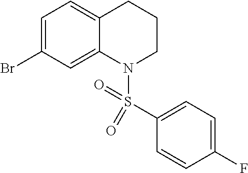 [SMILES: O=S(=O)(c1ccc(F)cc1)N1CCCc2ccc(Br)cc21] |
| 44 | 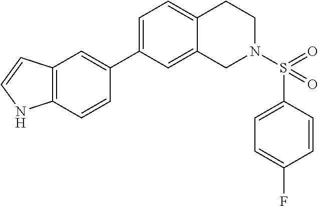 [SMILES: O=S(=O)(c1ccc(F)cc1)N1CCc2ccc(-c3ccc4[nH]ccc4c3)cc2C1] |
| 45 | 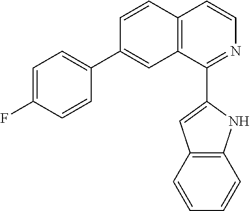 [SMILES: Fc1ccc(-c2ccc3ccnc(-c4cc5ccccc5[nH]4)c3c2)cc1] |
| 46 | 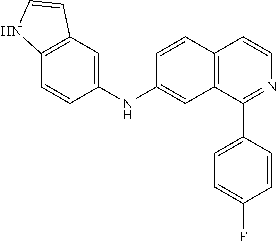 [SMILES: Fc1ccc(-c2nccc3ccc(Nc4ccc5[nH]ccc5c4)cc23)cc1] |
| 47 | 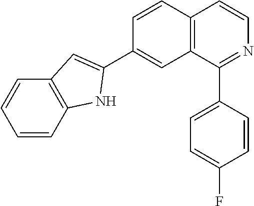 [SMILES: Fc1ccc(-c2nccc3ccc(-c4cc5ccccc5[nH]4)cc23)cc1] |
| 48 | 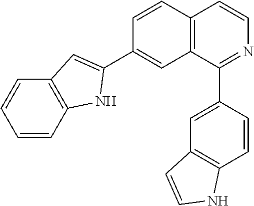 [SMILES: c1ccc2[nH]c(-c3ccc4ccnc(-c5ccc6[nH]ccc6c5)c4c3)cc2c1] |
| 49 | 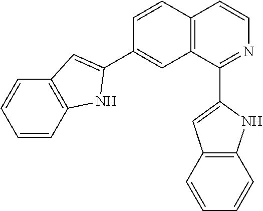 [SMILES: c1ccc2[nH]c(-c3ccc4ccnc(-c5cc6ccccc6[nH]5)c4c3)cc2c1] |
| 50 | 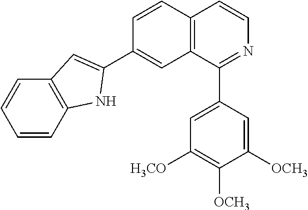 [SMILES: COc1cc(-c2nccc3ccc(-c4cc5ccccc5[nH]4)cc23)cc(OC)c1OC] |
| 51 | 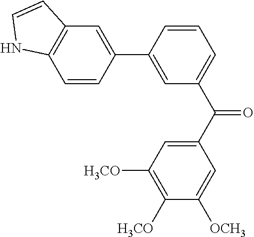 [SMILES: COc1cc(C(=O)c2cccc(-c3ccc4[nH]ccc4c3)c2)cc(OC)c1OC] |
| 52 | 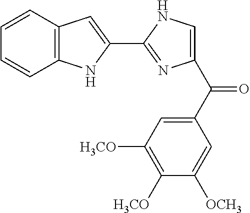 [SMILES: COc1cc(C(=O)c2c[nH]c(-c3cc4ccccc4[nH]3)n2)cc(OC)c1OC] |
| 53 | 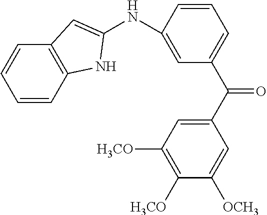 [SMILES: COc1cc(C(=O)c2cccc(Nc3cc4ccccc4[nH]3)c2)cc(OC)c1OC] |
| 54 | 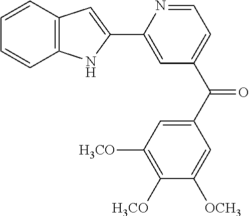 [SMILES: COc1cc(C(=O)c2ccnc(-c3cc4ccccc4[nH]3)c2)cc(OC)c1OC] |

It is well understood that in structures presented in this invention wherein the nitrogen atom has less than 3 bonds, H atoms are present to complete the valence of the nitrogen.

In one embodiment the A, A′ and/or C groups of formula I, I(a), IV, IX, IX(a) and XI are independently substituted and unsubstituted furanyl, indolyl, pyridinyl, phenyl, biphenyl, triphenyl, diphenylmethane, adamantane-yl, fluorene-yl, and other heterocyclic analogs such as, e.g., pyrrolyl, pyrazolyl, imidazolyl, pyridinyl, pyrimidinyl, pyrazinyl, pyridazinyl, triazinyl, tetrazinyl, pyrrolizinyl, indolyl, isoquinolinyl, quinolinyl, isoquinolinyl, benzimidazolyl, indazolyl, quinolizinyl, cinnolinyl, quinalolinyl, phthalazinyl, naphthyridinyl, quinoxalinyl, oxiranyl, oxetanyl, tetrahydrofuranyl, tetrahydropyranyl, dioxanyl, furanyl, pyrylium, benzofuranyl, benzodioxolyl, thiranyl, thietanyl, tetrahydrothiophene-yl, dithiolanyl, tetrahydrothiopyranyl, thiophene-yl, thiepinyl, thianaphthenyl, oxathiolanyl, morpholinyl, thioxanyl, thiazolyl, isothiazolyl, thiadiazolyl, oxazolyl, isoxazolyl, oxadiaziolyl).

In one embodiment, the A, A′ and/or C groups is substituted and unsubstituted phenyl. In another embodiment, the A, A′ and/or C groups is phenyl substituted by Cl, F or methyl. In one embodiment, the A, A′ and/or C groups is substituted and unsubstituted isoquinolinyl. In one embodiment, the A, A′ and/or C groups include substituted and unsubstituted indolyl groups; most preferably, substituted and unsubstituted 3-indolyl and 5-indolyl.

In one embodiment, the A, A′ and/or C groups of formula I, I(a), IV, IX, IX(a) and XI can be substituted or unsubstituted. Thus, although the exemplary groups recited in the preceding paragraph are unsubstituted, it should be appreciated by those of skill in the art that these groups can be substituted by one or more, two or more, three or more, and even up to five substituents (other than hydrogen).

In one embodiment, the most preferred A, A′ and/or C groups are substituted by 3,4,5-trimethoxyphenyl. In another embodiment the A, A′ and/or C groups are substituted by alkoxy. In another embodiment the A, A′ and/or C groups are substituted by methoxy. In another embodiment the A, A′ and/or C groups are substituted by alkyl. In another embodiment the A, A′ and/or C groups are substituted by methyl. In another embodiment the A, A′ and/or C groups are substituted by halogen. In another embodiment, the A, A′ and/or C groups are substituted by F. In another embodiment, the A, A′ and/or C groups are substituted by Cl. In another embodiment, the A, A′ and/or C rings are substituted by Br.

The substituents of these A, A′ and/or C groups of formula I, I(a), IV, IX, IX(a) and XI are independently selected from the group of hydrogen (e.g., no substitution at a particular position), hydroxyl, an aliphatic straight- or branched-chain C₁ to C₁₀ hydrocarbon, alkoxy, haloalkoxy, aryloxy, nitro, cyano, alkyl-CN, halo (e.g., F, Cl, Br, I), haloalkyl, dihaloalkyl, trihaloalkyl, COOH, C(O)Ph, C(O)-alkyl, C(O)O-alkyl, C(O)H, C(O)NH₂, —OC(O)CF₃, OCH₂Ph, amino, aminoalkyl, alkylamino, mesylamino, dialkylamino, arylamino, amido, NHC(O)-alkyl, urea, alkyl-urea, alkylamido (e.g., acetamide), haloalkylamido, arylamido, aryl, and C₅ to C₇ cycloalkyl, arylalkyl, and combinations thereof. Single substituents can be present at the ortho, meta, or para positions. When two or more substituents are present, one of them is preferably, though not necessarily, at the para position.

In one embodiment the B group of formula I, I(a), II, III, IV, IVa and V is selected from substituted or unsubstituted-thiazole, thiazolidine, oxazole, oxazoline, oxazolidine, benzene, pyrimidine, imidazole, pyridine, furan, thiophene, isoxazole, piperidine, pyrazole, indole and isoquinoline, wherein said B ring is linked via any two positions of the ring to X and Y or directly to the A and/or C rings.

In one embodiment the B group of formula I, I(a), II, III, IV, IVa and V is unsubstituted. In another embodiment the B group of formula I, I(a), II, III, IV, IVa and V is:

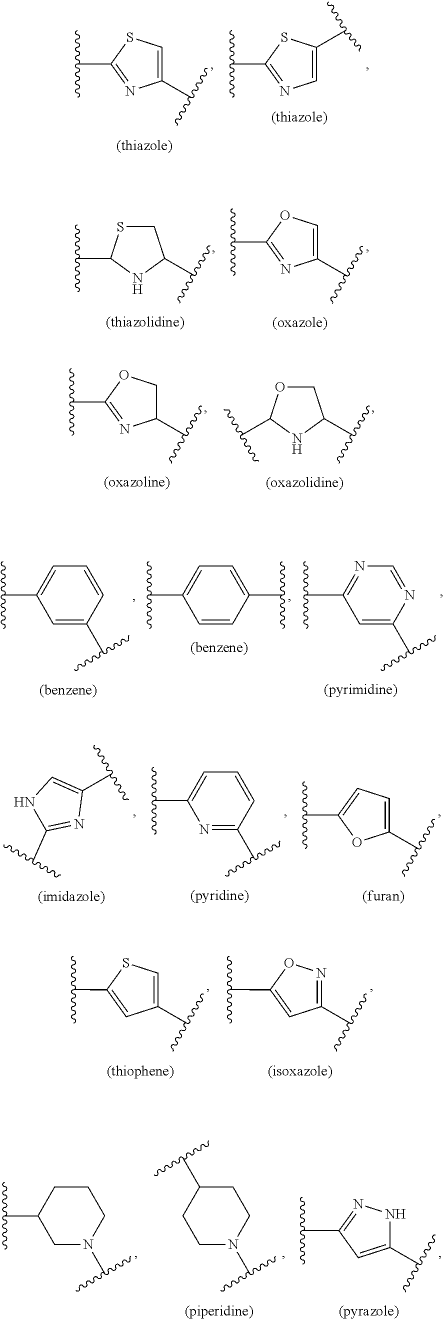

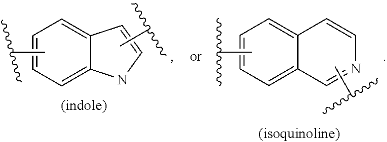

In another embodiment the B group of formula I, I(a), II, III, IV, IVa and V is substituted. In another embodiment the B group of formula I, I(a), II, III, IV, IVa and V is:

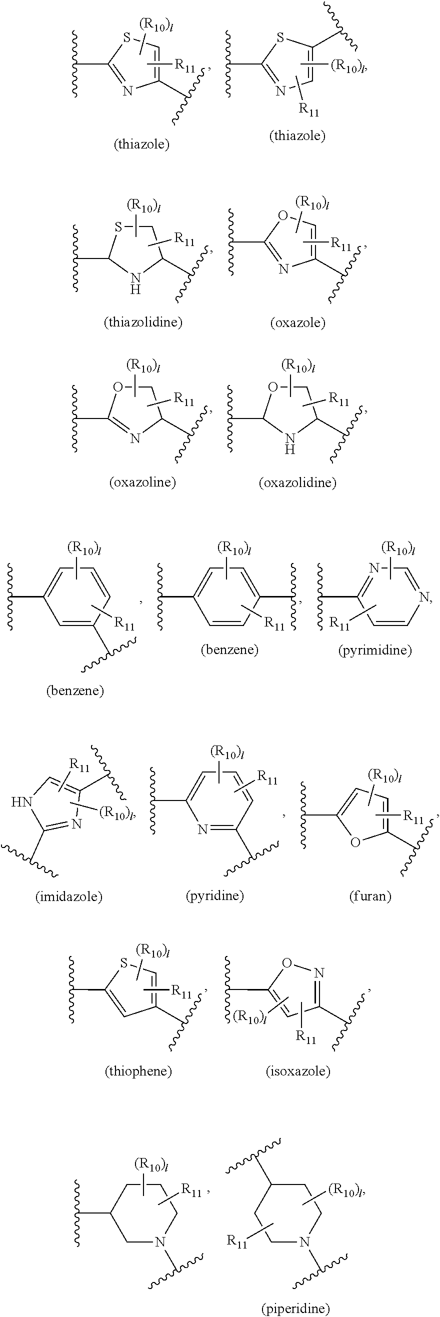

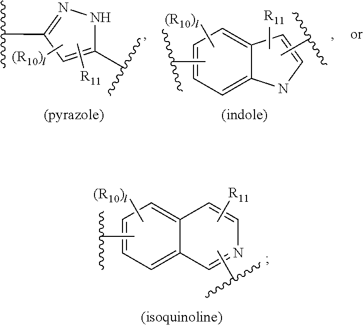

wherein R₁₀ and R₁₁ are independently hydrogen, O-alkyl, O-haloalkyl, F, Cl, Br, I, haloalkyl, CF₃, CN, —CH₂CN, NH₂, hydroxyl, —(CH₂)ᵢNHCH₃, —(CH₂)ᵢNH₂, —(CH₂)ᵢN(CH₃)₂, —OC(O)CF₃, C₁-C₅ linear or branched alkyl, haloalkyl, alkylamino, aminoalkyl, —OCH₂Ph, —NHCO-alkyl, COOH, —C(O)Ph, C(O)O-alkyl, C(O)H, —C(O)NH₂ or NO₂.

In another embodiment the B group is

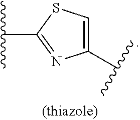 [SMILES: *c1csc(*)n1]

In another embodiment the B group is

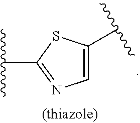 [SMILES: *c1cnc(*)s1]

In another embodiment the B group is

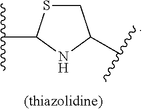 [SMILES: *C1CSC(*)N1]

In another embodiment the B group is

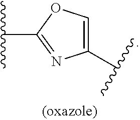 [SMILES: *c1coc(*)n1]

In another embodiment the B group is

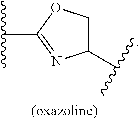 [SMILES: *C1=NC(*)CO1]

In another embodiment the B group is

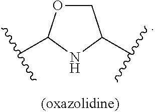 [SMILES: *C1COC(*)N1]

In another embodiment the B group is

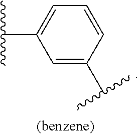 [SMILES: *c1cccc(*)c1]

In another embodiment the B group is

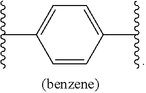 [SMILES: *c1ccc(*)cc1]

In another embodiment the B group is

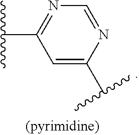 [SMILES: *c1cc(*)ncn1]

In another embodiment the B group is

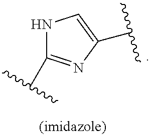 [SMILES: *c1c[nH]c(*)n1]

In another embodiment the B group is

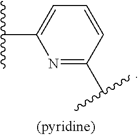 [SMILES: *c1cccc(*)n1]

In another embodiment the B group is

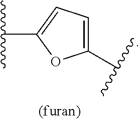 [SMILES: *c1ccc(*)o1]

In another embodiment the B group is

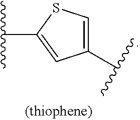 [SMILES: *c1csc(*)c1]

In another embodiment the B group is

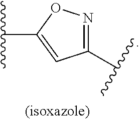 [SMILES: *c1cc(*)on1]

In another embodiment the B group is

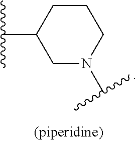 [SMILES: *C1CCCN(*)C1]

In another embodiment the B group is

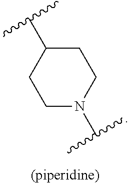 [SMILES: *C1CCN(*)CC1]

In another embodiment the B group is

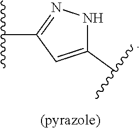 [SMILES: *c1cc(*)[nH]n1]

In another embodiment the B group is

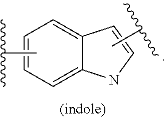

In another embodiment the B group is

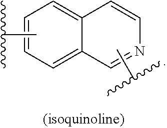

In one embodiment the B group of formula I, I(a), II, III, IV, IVa and V is substituted by R₁₀ and R₁₁. In another embodiment, R₁₀ and R₁₁ are both hydrogens. In another embodiment, R₁₀ and R₁₁ are independently O-alkyl. In another embodiment, R₁₀ and R₁₁ are independently O-haloalkyl. In another embodiment, R₁₀ and R₁₁ are independently F. In another embodiment, R₁₀ and R₁₁ are independently Cl. In another embodiment, R₁₀ and R₁₁ are independently Br. In another embodiment, R₁₀ and R₁₁ are independently I. In another embodiment, R₁₀ and R₁₁ are independently haloalkyl. In another embodiment, R₁₀ and R₁₁ are independently CF₃. In another embodiment, R₁₀ and R₁₁ are independently CN. In another embodiment, R₁₀ and R₁₁ are independently —CH₂CN. In another embodiment, R₁₀ and R₁₁ are independently NH₂. In another embodiment, R₁₀ and R₁₁ are independently hydroxyl. In another embodiment, R₁₀ and R₁₁ are independently —(CH₂)ᵢNHCH₃. In another embodiment, R₁₀ and R₁₁ are independently —(CH₂)ᵢNH₂. In another embodiment, R₁₀ and R₁₁ are independently —(CH₂)ᵢN(CH₃)₂. In another embodiment, R₁₀ and R₁₁ are independently —OC(O)CF₃. In another embodiment, R₁₀ and R₁₁ are independently C₁-C₅ linear or branched alkyl. In another embodiment, R₁₀ and R₁₁ are independently C₁-C₅ linear or branched haloalkyl. In another embodiment, R₁₀ and R₁₁ are independently C₁-C₅ linear or branched alkylamino. In another embodiment, R₁₀ and R₁₁ are independently C₁-C₅ linear or branched aminoalkyl. In another embodiment, R₁₀ and R₁₁ are independently —OCH₂Ph. In another embodiment, R₁₀ and R₁₁ are independently —NHCO-alkyl. In another embodiment, R₁₀ and R₁₁ are independently COOH. In another embodiment, R₁₀ and R₁₁ are independently —C(O)Ph. In another embodiment, R₁₀ and R₁₁ are independently C(O)O-alkyl. In another embodiment, R₁₀ and R₁₁ are independently C(O)H. In another embodiment, R₁₀ and R₁₁ are independently —C(O)NH₂. In another embodiment, R₁₀ and R₁₁ are independently NO₂.

In another embodiment the B group of formula I, I(a), II, III, IV, IVa and V is

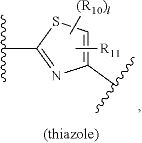

wherein R₁₀ and R₁₁ are independently H and 1 is 1. In another embodiment, R₁₀ and R₁₁ are independently O-alkyl. In another embodiment, R₁₀ and R₁₁ are independently O-haloalkyl. In another embodiment, R₁₀ and R₁₁ are independently F. In another embodiment, R₁₀ and R₁₁ are independently Cl. In another embodiment, R₁₀ and R₁₁ are independently Br. In another embodiment, R₁₀ and R₁₁ are independently I. In another embodiment, R₁₀ and R₁₁ are independently haloalkyl. In another embodiment, R₁₀ and R₁₁ are independently CF₃. In another embodiment, R₁₀ and R₁₁ are independently CN. In another embodiment, R₁₀ and R₁₁ are independently —CH₂CN. In another embodiment, R₁₀ and R₁₁ are independently NH₂. In another embodiment, R₁₀ and R₁₁ are independently hydroxyl. In another embodiment, R₁₀ and R₁₁ are independently —(CH₂)ᵢNHCH₃. In another embodiment, R₁₀ and R₁₁ are independently —(CH₂)ᵢNH₂. In another embodiment, R₁₀ and R₁₁ are independently —(CH₂)ᵢN(CH₃)₂. In another embodiment, R₁₀ and R₁₁ are independently —OC(O)CF₃. In another embodiment, R₁₀ and R₁₁ are independently C₁-C₅ linear or branched alkyl. In another embodiment, R₁₀ and R₁₁ are independently C₁-C₅ linear or branched haloalkyl. In another embodiment, R₁₀ and R₁₁ are independently C₁-C₅ linear or branched alkylamino. In another embodiment, R₁₀ and R₁₁ are independently C₁-C₅ linear or branched aminoalkyl. In another embodiment, R₁₀ and R₁₁ are independently —OCH₂Ph. In another embodiment, R₁₀ and R₁₁ are independently —NHCO-alkyl. In another embodiment, R₁₀ and R₁₁ are independently COOH. In another embodiment, R₁₀ and R₁₁ are independently —C(O)Ph. In another embodiment, R₁₀ and R₁₁ are independently C(O)O-alkyl. In another embodiment, R₁₀ and R₁₁ are independently C(O)H. In another embodiment, R₁₀ and R₁₁ are independently —C(O)NH₂. In another embodiment, R₁₀ and R₁₁ are independently NO₂.

In another embodiment the B group of formula I, I(a), II, III, IV, IVa and V is

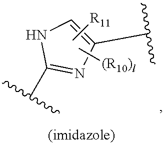

wherein R₁₀ and R₁₁ are independently H and 1 is 1. In another embodiment, R₁₀ and R₁₁ are independently O-alkyl. In another embodiment, R₁₀ and R₁₁ are independently O-haloalkyl. In another embodiment, R₁₀ and R₁₁ are independently F. In another embodiment, R₁₀ and R₁₁ are independently Cl. In another embodiment, R₁₀ and R₁₁ are independently Br. In another embodiment, R₁₀ and R₁₁ are independently I. In another embodiment, R₁₀ and R₁₁ are independently haloalkyl. In another embodiment, R₁₀ and R₁₁ are independently CF₃. In another embodiment, R₁₀ and R₁₁ are independently CN. In another embodiment, R₁₀ and R₁₁ are independently —CH₂CN. In another embodiment, R₁₀ and R₁₁ are independently NH₂. In another embodiment, R₁₀ and R₁₁ are independently hydroxyl. In another embodiment, R₁₀ and R₁₁ are independently —(CH₂)ᵢNHCH₃. In another embodiment, R₁₀ and R₁₁ are independently —(CH₂)ᵢNH₂. In another embodiment, R₁₀ and R₁₁ are independently —(CH₂)ᵢN(CH₃)₂. In another embodiment, R₁₀ and R₁₁ are independently —OC(O)CF₃. In another embodiment, R₁₀ and R₁₁ are independently C₁-C₅ linear or branched alkyl. In another embodiment, R₁₀ and R₁₁ are independently C₁-C₅ linear or branched haloalkyl. In another embodiment, R₁₀ and R₁₁ are independently C₁-C₅ linear or branched alkylamino. In another embodiment, R₁₀ and R₁₁ are independently C₁-C₅ linear or branched aminoalkyl. In another embodiment, R₁₀ and R₁₁ are independently —OCH₂Ph. In another embodiment, R₁₀ and R₁₁ are independently —NHCO-alkyl. In another embodiment, R₁₀ and R₁₁ are independently COOH. In another embodiment, R₁₀ and R₁₁ are independently —C(O)Ph. In another embodiment, R₁₀ and R₁₁ are independently C(O)O-alkyl. In another embodiment, R₁₀ and R₁₁ are independently C(O)H. In another embodiment, R₁₀ and R₁₁ are independently —C(O)NH₂. In another embodiment, R₁₀ and R₁₁ are independently NO₂.

In another embodiment the B group of formula I, I(a), II, III, IV, IVa and V is

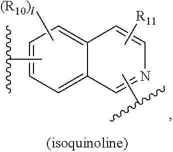

wherein R₁₀ and R₁₁ are independently H and 1 is 1. In another embodiment, R₁₀ and R₁₁ are independently O-alkyl. In another embodiment, R₁₀ and R₁₁ are independently O-haloalkyl. In another embodiment, R₁₀ and R₁₁ are independently F. In another embodiment, R₁₀ and R₁₁ are independently Cl. In another embodiment, R₁₀ and R₁₁ are independently Br. In another embodiment, R₁₀ and R₁₁ are independently I. In another embodiment, R₁₀ and R₁₁ are independently haloalkyl. In another embodiment, R₁₀ and R₁₁ are independently CF₃. In another embodiment, R₁₀ and R₁₁ are independently CN. In another embodiment, R₁₀ and R₁₁ are independently —CH₂CN. In another embodiment, R₁₀ and R₁₁ are independently NH₂. In another embodiment, R₁₀ and R₁₁ are independently hydroxyl. In another embodiment, R₁₀ and R₁₁ are independently —(CH₂)ᵢNHCH₃. In another embodiment, R₁₀ and R₁₁ are independently —(CH₂)ᵢNH₂. In another embodiment, R₁₀ and R₁₁ are independently —(CH₂)ᵢN(CH₃)₂. In another embodiment, R₁₀ and R₁₁ are independently —OC(O)CF₃. In another embodiment, R₁₀ and R₁₁ are independently C₁-C₅ linear or branched alkyl. In another embodiment, R₁₀ and R₁₁ are independently C₁-C₅ linear or branched haloalkyl. In another embodiment, R₁₀ and R₁₁ are independently C₁-C₅ linear or branched alkylamino. In another embodiment, R₁₀ and R₁₁ are independently C₁-C₅ linear or branched aminoalkyl. In another embodiment, R₁₀ and R₁₁ are independently —OCH₂Ph. In another embodiment, R₁₀ and R₁₁ are independently —NHCO-alkyl. In another embodiment, R₁₀ and R₁₁ are independently COOH. In another embodiment, R₁₀ and R₁₁ are independently —C(O)Ph. In another embodiment, R₁₀ and R₁₁ are independently C(O)O-alkyl. In another embodiment, R₁₀ and R₁₁ are independently C(O)H. In another embodiment, R₁₀ and R₁₁ are independently —C(O)NH₂. In another embodiment, R₁₀ and R₁₁ are independently NO₂.

In one embodiment, the X bridge of formula I, Ia, II, III, IV, IVa and XI is a bond. In another embodiment, the X bridge is NH. In another embodiment, the X bridge is C₁ to C₅ hydrocarbon. In another embodiment, the X bridge is CH₂. In another embodiment, the X bridge is —CH₂—CH₂—. In another embodiment, the X bridge is O. In another embodiment, the X bridge is S.

In one embodiment, the Y bridge of formula I, Ia, II, III, IV, IVa, VI, and VII is C═O. In another embodiment, the Y bridge is C═S. In another embodiment, the Y bridge is C═N(NH₂)—. In another embodiment, the Y bridge is —C═NOH. In another embodiment, the Y bridge is —CH—OH. In another embodiment, the Y bridge is —C═CH—(CN). In another embodiment, the Y bridge is —C═N(CN). In another embodiment, the Y bridge is —C═C(CH₃)₂. In another embodiment, the Y bridge is —C═N—OMe. In another embodiment, the Y bridge is —(C═O)NH—. In another embodiment, the Y bridge is —NH(C═O)—. In another embodiment, the Y bridge is —(C═O)-O. In another embodiment, the Y bridge is —O—(C═O). In another embodiment, the Y bridge is —(CH₂)₁₋₅—(C═O). In another embodiment, the Y bridge is —(C═O)—(CH₂)₁₋₅. In another embodiment, the Y bridge is S. In another embodiment, the Y bridge is SO. In another embodiment, the Y bridge is SO₂. In another embodiment, the Y bridge is —CH═CH—. In another embodiment, the Y bridge is —(SO₂)—NH—. In another embodiment, the Y bridge is —NH—(SO₂)—.

In one embodiment, R₁, R₂, R₃, R₄, R₅ and R₆ of formula Ia, II, III, IV, IV(a), V, VI, VIII, IX, IX(a), XI(a), XI(b), XI(c), XI(d) and XI(e) are independently hydrogen. In another embodiment, R₁, R₂, R₃, R₄, R₅ and R₆ are independently O-alkyl. In another embodiment, R₁, R₂, R₃, R₄, R₅ and R₆ are independently O-haloalkyl. In another embodiment, R₁, R₂, R₃, R₄, R₅ and R₆ are independently F. In another embodiment, R₁, R₂, R₃, R₄, R₅ and R₆ are independently Cl. In another embodiment, R₁, R₂, R₃, R₄, R₅ and R₆ are independently Br. In another embodiment, R₁, R₂, R₃, R₄, R₅ and R₆ are independently I. In another embodiment, R₁, R₂, R₃, R₄, R₅ and R₆ are independently haloalkyl. In another embodiment, R₁, R₂, R₃, R₄, R₅ and R₆ are independently CF₃. In another embodiment, R₁, R₂, R₃, R₄, R₅ and R₆ are independently CN. In another embodiment, R₁, R₂, R₃, R₄, R₅ and R₆ are independently —CH₂CN. In another embodiment, R₁, R₂, R₃, R₄, R₅ and R₆ are independently NH₂. In another embodiment, R₁, R₂, R₃, R₄, R₅ and R₆ are independently hydroxyl. In another embodiment, R₁, R₂, R₃, R₄, R₅ and R₆ are independently —(CH₂)ᵢNHCH₃ In another embodiment, R₁, R₂, R₃, R₄, R₅ and R₆ are independently —(CH₂)ᵢNH₂. In another embodiment, R₁, R₂, R₃, R₄, R₅ and R₆ are independently —(CH₂)ᵢN(CH₃)₂. In another embodiment, R₂, R₃, R₄, R₅ and R₆ are independently —OC(O)CF₃. In another embodiment, R₁, R₂, R₃, R₄, R₅ and R₆ are independently C₁-C₅ linear or branched alkyl. In another embodiment, R₁, R₂, R₃, R₄, R₅ and R₆ are independently haloalkyl. In another embodiment, R₁, R₂, R₃, R₄, R₅ and R₆ are independently alkylamino. In another embodiment, R₁, R₂, R₃, R₄, R₅ and R₆ are independently aminoalkyl. In another embodiment, R₁, R₂, R₃, R₄, R₅ and R₆ are independently —OCH₂Ph. In another embodiment, R₁, R₂, R₃, R₄, R₅ and R₆ are independently —NHCO-alkyl. In another embodiment, R₁, R₂, R₃, R₄, R₅ and R₆ are independently COOH. In another embodiment, R₁, R₂, R₃, R₄, R₅ and R₆ are independently —C(O)Ph. In another embodiment, R₁, R₂, R₃, R₄, R₅ and R₆ are independently C(O)O-alkyl. In another embodiment, R₁, R₂, R₃, R₄, R₅ and R₆ are independently C(O)H. In another embodiment, R₁, R₂, R₃, R₄, R₅ and R₆ are independently —C(O)NH₂. In another embodiment, R₁, R₂, R₃, R₄, R₅ and R₆ are independently NO₂.

The invention also encompasses methods of treating triple negative breast cancer and/or ovarian cancer by administering at least one compound of formula XII in a therapeutically effective amount to a subject in need thereof, wherein the compound of Formula XII is represented by the structure:

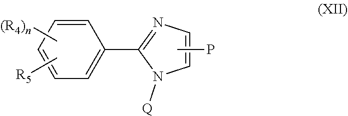

wherein,
P and Q are independently H or

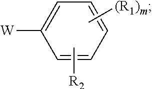

### W is C═O, C═S, SO₂ or S═O;

wherein at least one of Q or P is not hydrogen;
R₁ and R₄ are independently H, O-alkyl, I, Br, Cl, F, alkyl, haloalkyl, aminoalkyl, OCH₂Ph, OH, CN, NO₂, —NHCO-alkyl, COOH, —(CH₂)ᵢNHCH₃, —(CH₂)ᵢNH₂, —(CH₂)ᵢN(CH₃)₂; C(O)O-alkyl or C(O)H; wherein at least one of R₁ and R₄ is not hydrogen;
R₂ and R₅ are independently H, O-alkyl, I, Br, Cl, F, alkyl, haloalkyl, aminoalkyl, OCH₂Ph, OH, CN, NO₂, —NHCO-alkyl, COOH, C(O)O-alkyl or C(O)H;
m is an integer between 1-4;
i is an integer between 0-5; and
n is an integer between 1-4;
or its pharmaceutically acceptable salt, hydrate, polymorph, metabolite, tautomer or isomer.

The invention also encompasses methods of treating triple negative breast cancer and/or ovarian cancer by administering at least one compound of formula XIII in a therapeutically effective amount to a subject in need thereof, wherein the compound of Formula XIII is represented by the structure:

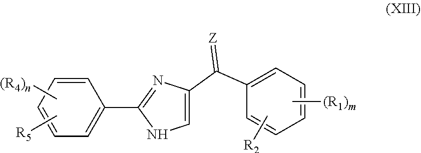

wherein

### Z is O or S;

R₁ and R₄ are independently H, O-alkyl, I, Br, Cl, F, alkyl, haloalkyl, aminoalkyl, OCH₂Ph, OH, CN, NO₂, —NHCO-alkyl, haloalkyl, aminoalkyl, —(CH₂)ᵢNHCH₃, —(CH₂)ᵢNH₂, —(CH₂)ᵢN(CH₃)₂; COOH, C(O)O-alkyl or C(O)H; wherein at least one of R₁ and R₄ is not hydrogen;
R₂ and R₅ are independently H, O-alkyl, I, Br, Cl, F, alkyl, haloalkyl, aminoalkyl, —(CH₂)ᵢNHCH₃, —(CH₂)ᵢNH₂, —(CH₂)ᵢN(CH₃)₂; OCH₂Ph, OH, CN, NO₂, —NHCO-alkyl, COOH, C(O)O-alkyl or C(O)H;
m is an integer between 1-4;
i is an integer between 0-5; and
n is an integer between 1-4;
or its pharmaceutically acceptable salt, hydrate, polymorph, metabolite, tautomer or isomer.

The invention also encompasses methods of treating triple negative breast cancer and/or ovarian cancer by administering at least one compound of formula (XIV) in a therapeutically effective amount to a subject in need thereof, wherein the compound of Formula (XIV) is represented by the structure:

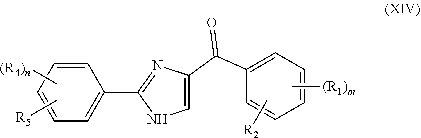

wherein R₁ and R₄ are independently H, O-alkyl, I, Br, Cl, F, alkyl, haloalkyl, aminoalkyl, —(CH₂)ᵢNHCH₃, —(CH₂)ᵢNH₂, —(CH₂)ᵢN(CH₃)₂, OCH₂Ph, OH, CN, NO₂, —NHCO-alkyl, COOH, C(O)O-alkyl or C(O)H; wherein at least one of R₁ and R₄ is not hydrogen;
R₂ and R₅ are independently H, O-alkyl, I, Br, Cl, F, alkyl, haloalkyl, aminoalkyl, OCH₂Ph, OH, CN, NO₂, —NHCO-alkyl, COOH, C(O)O-alkyl or C(O)H;
m is an integer between 1-4;
i is an integer between 0-5; and
n is an integer between 1-4;
or its pharmaceutically acceptable salt, hydrate, polymorph, metabolite, tautomer or isomer.

In one embodiment, R₁ of compound of formula XII, XIII and XIV is OCH₃. In another embodiment, R₁ of compound of formula XII, XIII and XIV is 4-F. In another embodiment, R₁ of compound of formula XII, XIII and XIV is OCH₃ and m is 3. In another embodiment, R₄ of compound of formula XII, XIII and XIV is 4-F. In another embodiment, R₄ of compound of formula XII, XIII and XIV is OCH₃. In another embodiment, R₄ of compound of formula XIV is CH₃. In another embodiment, R₄ of compound of formula XII, XIII and XIV is 4-Cl. In another embodiment, R₄ of compound of formula XII, XIII and XIV is 4-N(Me)₂. In another embodiment, R₄ of compound of formula XII, XIII and XIV is OBn. In another embodiment, R₄ of compound of formula XII, XIII and XIV is 4-Br. In another embodiment, R₄ of compound of formula XII, XIII and XIV is 4-CF₃. Non limiting examples of compounds of formula XIV are selected from: (2-phenyl-1H-imidazol-4-yl)(3,4,5-trimethoxyphenyl)methanone (12aa), (4-fluorophenyl)(2-phenyl-1H-imidazol-4-yl)methanone (12af), (2-(4-fluorophenyl)-1H-imidazol-4-yl)(3,4,5-trimethoxyphenyl)methanone (12ba), (2-(4-methoxyphenyl)-1H-imidazol-4-yl)(3,4,5-trimethoxyphenyl)methanone (12ca), (4-fluorophenyl)(2-(4-methoxyphenyl)-1H-imidazol-4-yl)methanone (12cb), (2-(p-tolyl)-1H-imidazol-4-yl)(3,4,5-trimethoxyphenyl)methanone (12da), (4-fluorophenyl)(2-(p-tolyl)-1H-imidazol-4-yl)methanone (12db), (4-hydroxy-3,5-dimethoxyphenyl)(2-(p-tolyl)-1H-imidazol-4-yl)methanone (12dc), (2-(4-chlorophenyl)-1H-imidazol-4-yl)(3,4,5-trimethoxyphenyl)methanone (12fa), (2-(4-chlorophenyl)-1H-imidazol-4-yl)(4-fluorophenyl)methanone (12fb), (2-(4-chlorophenyl)-1H-imidazol-4-yl)(4-hydroxy-3,5-dimethoxyphenyl)methanone (12fc), (2-(4-(dimethylamino)phenyl)-1H-imidazol-4-yl)(3,4,5-trimethoxyphenyl)methanone (12ga); (2-(4-(dimethylamino)phenyl)-1H-imidazol-4-yl)(4-fluorophenyl)methanone (12gb), (2-(3,4-dimethoxyphenyl)-1H-imidazol-4-yl)(3,4,5-trimethoxyphenyl)methanone (12ha), (2-(4-(benzyloxy)phenyl)-1H-imidazol-4-yl)(4-fluorophenyl)methanone (12jb), (2-(4-bromophenyl)-1H-imidazol-4-yl)(3,4,5-trimethoxyphenyl)methanone (121a), (2-(4-(trifluoromethyl)phenyl)-1H-imidazol-4-yl)(3,4,5-trimethoxyphenyl)methanone (12pa).

The invention also encompasses methods of treating triple negative breast cancer and/or ovarian cancer by administering at least one compound of formula (XIVa) in a therapeutically effective amount to a subject in need thereof, wherein the compound of Formula (XIVa) is represented by the structure:

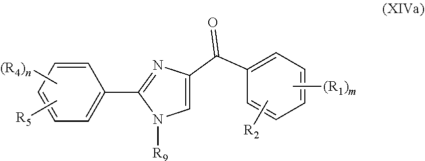

wherein R₁ and R₄ are independently H, O-alkyl, I, Br, Cl, F, alkyl, haloalkyl, aminoalkyl, —(CH₂)ᵢNHCH₃, —(CH₂)ᵢNH₂, —(CH₂)ᵢN(CH₃)₂, OCH₂Ph, OH, CN, NO₂, —NHCO-alkyl, COOH, C(O)O-alkyl or C(O)H; wherein at least one of R₁ and R₄ is not hydrogen;
R₂ and R₅ are independently H, O-alkyl, I, Br, Cl, F, alkyl, haloalkyl, aminoalkyl, —(CH₂)ᵢNHCH₃, —(CH₂)ᵢNH₂, —(CH₂)ᵢN(CH₃)₂, OCH₂Ph, OH, CN, NO₂, —NHCO-alkyl, COOH, C(O)O-alkyl or C(O)H;
R₉ is H, linear or branched, substituted or unsubstituted alkyl, substituted or unsubstituted aryl, CH₂Ph, substituted benzyl, haloalkyl, aminoalkyl, OCH₂Ph, substituted or unsubstituted SO₂-Aryl, substituted or unsubstituted —(C═O)-Aryl or OH;
wherein substitutions are independently selected from the group of hydrogen (e.g., no substitution at a particular position), hydroxyl, an aliphatic straight- or branched-chain C₁ to C₁₀ hydrocarbon, alkoxy, haloalkoxy, aryloxy, nitro, cyano, alkyl-CN, halo (e.g., F, Cl, Br, I), haloalkyl, dihaloalkyl, trihaloalkyl, COOH, C(O)Ph, C(O)-alkyl, C(O)O-alkyl, C(O)H, C(O)NH₂, —OC(O)CF₃, OCH₂Ph, amino, aminoalkyl, alkylamino, mesylamino, dialkylamino, arylamino, amido, NHC(O)-alkyl, urea, alkyl-urea, alkylamido (e.g., acetamide), haloalkylamido, arylamido, aryl, and C₅ to C₇ cycloalkyl, arylalkyl, and combinations thereof;
m is an integer between 1-4;
i is an integer between 0-5; and
n is an integer between 1-4;
or its pharmaceutically acceptable salt, hydrate, polymorph, metabolite, tautomer or isomer.

In one embodiment, R₉ of compound of formula XIVa is CH₃. In another embodiment, R₉ of compound of formula XIVa is CH₂Ph. In another embodiment, R₉ of compound of formula XIVa is (SO₂)Ph. In another embodiment, R₉ of compound of formula XIVa is (SO₂)-Ph-OCH₃. In another embodiment, R₉ of compound of formula XIVa is H. In another embodiment, R₄ of compound of formula XIVa is H. In another embodiment, R₄ of compound of formula XIVa is CH₃. In another embodiment, R₄ of compound of formula XIVa is OCH₃. In another embodiment, R₄ of compound of formula XIVa is OH. In another embodiment, R₄ of compound of formula XIVa is 4-Cl. In another embodiment, R₄ of compound of formula XIVa is 4-N(Me)₂. In another embodiment, R₄ of compound of formula XIVa is OBn. In another embodiment, R₁ of compound of formula XIVa is OCH₃; m is 3 and R₂ is H. In another embodiment, R₁ of compound of formula XIVa is F; m is 1 and R₂ is H. Non limiting examples of compounds of formula XIVa are selected from: (4-fluorophenyl)(2-phenyl-1-(phenylsulfonyl)-1H-imidazol-4-yl)methanone (11af), (4-fluorophenyl)(2-(4-methoxyphenyl)-1-(phenylsulfonyl)-1H-imidazol-4-yl)methanone (11cb), (4-fluorophenyl)(1-(phenylsulfonyl)-2-(p-tolyl)-1H-imidazol-4-yl)methanone (11db), (2-(4-chlorophenyl)-1-(phenylsulfonyl)-1H-imidazol-4-yl)(4-fluorophenyl)methanone (11fb), (2-(4-(dimethylamino)phenyl)-1-(phenylsulfonyl)-1H-imidazol-4-yl)(3,4,5-trimethoxyphenyl)methanone (11ga), (2-(4-(dimethylamino)phenyl)-1-(phenylsulfonyl)-1H-imidazol-4-yl)(4-fluorophenyl)methanone (11gb), (2-(3,4-dimethoxyphenyl)-1-(phenylsulfonyl)-1H-imidazol-4-yl)(3,4,5-trimethoxyphenyl)methanone (11ha), (2-(4-(benzyloxy)phenyl)-1-(phenylsulfonyl)-1H-imidazol-4-yl)(4-fluorophenyl)methanone (11jb), (2-(4-(dimethylamino)phenyl)-1-((4-methoxyphenyl)sulfonyl)-1H-imidazol-4-yl)(4-fluorophenyl)methanone (12gba), (1-benzyl-2-(p-tolyl)-1H-imidazol-4-yl)(3,4,5-trimethoxyphenyl)methanone (12daa), (1-methyl-2-(p-tolyl)-1H-10imidazol-4-yl)(3,4,5-trimethoxyphenyl)methanone (12dab), (4-fluorophenyl)(2-(4-methoxyphenyl)-1-methyl-1H-imidazol-4-yl)methanone (12cba).

The invention also encompasses methods of treating triple negative breast cancer and/or ovarian cancer by administering at least one compound of formula (XV) in a therapeutically effective amount to a subject in need thereof, wherein the compound of Formula (XV) is represented by the structure:

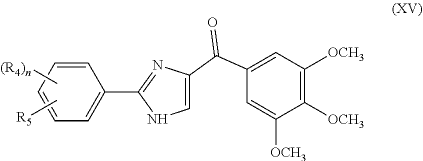

wherein R₄ and R₅ are independently H, O-alkyl, I, Br, Cl, F, alkyl, haloalkyl, aminoalkyl, —(CH₂)ᵢNHCH₃, —(CH₂)ᵢNH₂, —(CH₂)ᵢN(CH₃)₂, OCH₂Ph, OH, CN, NO₂, —NHCO-alkyl, COOH, C(O)O-alkyl or C(O)H;
i is an integer between 0-5; and
n is an integer between is 1-4;
or its pharmaceutically acceptable salt, hydrate, polymorph, metabolite, tautomer or isomer.

In one embodiment, R₄ of compound of formula XV is H. In another embodiment, R₄ of compound of formula XV is F. In another embodiment, R₄ of compound of formula XV is Cl. In another embodiment, R₄ of compound of formula XV is Br. In another embodiment, R₄ of compound of formula XV is I. In another embodiment, R₄ of compound of formula XV is N(Me)₂. In another embodiment, R₄ of compound of formula XV is OBn. In another embodiment, R₄ of compound of formula XV is OCH₃. In another embodiment, R₄ of compound of formula XV is CH₃. In another embodiment, R₄ of compound of formula XV is CF₃. Non limiting examples of compounds of formula XV are selected from: (2-phenyl-1H-imidazol-4-yl)(3,4,5-trimethoxyphenyl)methanone (12aa), (2-(4-fluorophenyl)-1H-imidazol-4-yl)(3,4,5-trimethoxyphenyl)methanone (12ba), (2-(4-methoxyphenyl)-1H-imidazol-4-yl)(3,4,5-trimethoxyphenyl)methanone (12ca), (2-(p-tolyl)-1H-imidazol-4-yl)(3,4,5-trimethoxyphenyl)methanone (12da), (3,4,5-trimethoxyphenyl)(2-(3,4,5-trimethoxyphenyl)-1H-imidazol-4-yl)methanone (12ea), (2-(4-chlorophenyl)-1H-imidazol-4-yl)(3,4,5-trimethoxyphenyl)methanone (12fa), (2-(4-(dimethylamino)phenyl)-1H-imidazol-4-yl)(3,4,5-trimethoxyphenyl)methanone (12ga), (2-(3,4-dimethoxyphenyl)-1H-imidazol-4-yl)(3,4,5-trimethoxyphenyl)methanone (12ha), (2-(2-(trifluoromethyl)phenyl)-1H-imidazol-4-yl)(3,4,5-trimethoxyphenyl)methanone (12la), (2-(4-(benzyloxy)phenyl)-1H-imidazol-4-yl)(3,4,5-trimethoxyphenyl)methanone (12ja), (2-(4-hydroxyphenyl)-1H-imidazol-4-yl)(3,4,5-trimethoxyphenyl)methanone (12ka), (2-(4-bromophenyl)-1H-imidazol-4-yl)(3,4,5-trimethoxyphenyl)methanone (12la), (2-(4-(trifluoromethyl)phenyl)-1H-imidazol-4-yl)(3,4,5-trimethoxyphenyl)methanone (12pa).

The invention also encompasses methods of treating triple negative breast cancer and/or ovarian cancer by administering at least one compound of formula (XVI) in a therapeutically effective amount to a subject in need thereof, wherein the compound of Formula (XVI) is represented by the structure:

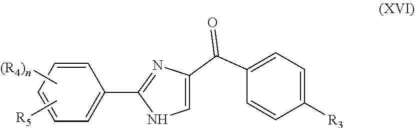

wherein R₄ and R₅ are independently H, O-alkyl, I, Br, Cl, F, alkyl, haloalkyl, aminoalkyl, —(CH₂)ᵢNHCH₃, —(CH₂)ᵢNH₂, —(CH₂)ᵢN(CH₃)₂, OCH₂Ph, OH, CN, NO₂, —NHCO-alkyl, COOH, C(O)O-alkyl or C(O)H;

### R₃ is I, Br, Cl, or F;

i is an integer between 0-5; and
n is an integer between 1-4;
or its pharmaceutically acceptable salt, hydrate, polymorph, metabolite, tautomer or isomer.

In one embodiment, R₃ of compound of formula XVI is halogen. In another embodiment, R₃ is F. In another embodiment, R₃ is Cl. In another embodiment R₃ is Br. In another embodiment R₃ is I. In another embodiment R₄ is H. In another embodiment R₄ is OCH₃. In another embodiment R₄ is OCH₃; n is 3 and R₅ is H. In another embodiment R₄ is CH₃. In another embodiment R₄ is F. In another embodiment R₄ is Cl. In another embodiment R₄ is Br. In another embodiment R₄ is I. In another embodiment R₄ is N(Me)₂. In another embodiment R₄ is OBn. In another embodiment, R₃ is F; R₅ is hydrogen; n is 1 and R₄ is 4-Cl. In another embodiment, R₃ is F; R₅ is hydrogen; n is 1 and R₄ is 4-OCH₃. In another embodiment, R₃ is F; R₅ is hydrogen; n is 1 and R₄ is 4-CH₃. In another embodiment, R₃ is F; R₅ is hydrogen; n is 1 and R₄ is 4-N(Me)₂. In another embodiment, R₃ is F; R₅ is hydrogen; n is 1 and R₄ is 4-OBn. Non limiting examples of compounds of formula XVI are selected from: (4-fluorophenyl)(2-phenyl-1H-imidazol-4-yl)methanone (12af), (4-fluorophenyl)(2-(4-methoxyphenyl)-1H-imidazol-4-yl)methanone (12cb), (4-fluorophenyl)(2-(p-tolyl)-1H-imidazol-4-yl)methanone (12db), 4-fluorophenyl)(2-(3,4,5-trimethoxyphenyl)-1H-imidazol-4-yl)methanone (12eb), (2-(4-chlorophenyl)-1H-imidazol-4-yl)(4-fluorophenyl)methanone (12fb), (2-(4-(dimethylamino)phenyl)-1H-imidazol-4-yl)(4-fluorophenyl)methanone (12gb), (2-(4-(benzyloxy)phenyl)-1H-imidazol-4-yl)(4-fluorophenyl)methanone (12jb).

The invention also encompasses methods of treating triple negative breast cancer and/or ovarian cancer by administering at least one compound of formula (XVII) in a therapeutically effective amount to a subject in need thereof, wherein the compound of Formula (XVII) is represented by the structure:

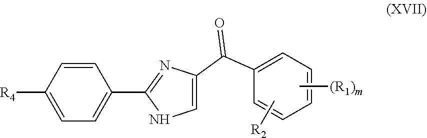

wherein R₄ is H, O-alkyl, I, Br, Cl, F, alkyl, haloalkyl, aminoalkyl, OCH₂Ph, OH, CN, NO₂, —NHCO-alkyl, COOH, C(O)O-alkyl or C(O)H;
wherein R₁ and R₂ are independently H, O-alkyl, I, Br, Cl, F, alkyl, haloalkyl, aminoalkyl, OCH₂Ph, OH, CN, NO₂, —NHCO-alkyl, COOH, C(O)O-alkyl or C(O)H;
and
m is an integer between 1-4;
or its pharmaceutically acceptable salt, hydrate, polymorph, metabolite, tautomer or isomer.
In one embodiment, R₄ of compound of formula XVII is halogen. In another embodiment, R₄ is F. In another embodiment, R₄ is Cl. In another embodiment R₄ is Br. In another embodiment R₄ is I. In another embodiment, R₄ is OCH₃. In another embodiment, R₄ is CH₃. In another embodiment, R₄ is N(Me)₂. In another embodiment, R₄ is CF₃. In another embodiment, R₄ is OH. In another embodiment, R₄ is OBn. In another embodiment, R₁ of compound of formula XVII is halogen. In another embodiment, R₁ of compound of formula XVII is F. In another embodiment, R₁ of compound of formula XVII is Cl. In another embodiment, R₁ of compound of formula XVII is Br. In another embodiment, R₁ of compound of formula XVII is I. In another embodiment, R₁ of compound of formula XVII is OCH₃. In another embodiment, R₁ of compound of formula XVII is OCH₃, m is 3 and R₂ is H. In another embodiment, R₁ of compound of formula XVII is F, m is 1 and R₂ is H. In another embodiment, R₄ is F; R₂ is hydrogen; n is 3 and R₁ is OCH₃. In another embodiment, R₄ is OCH₃; R₂ is hydrogen; n is 3 and R₁ is OCH₃. In another embodiment, R₄ is CH₃; R₂ is hydrogen; n is 3 and R₁ is OCH₃. In another embodiment, R₄ is Cl; R₂ is hydrogen; n is 3 and R₁ is OCH₃. In another embodiment, R₄ is N(Me)₂; R₂ is hydrogen; n is 3 and R₁ is OCH₃. In one embodiment, R₄ of compound of formula XVII is halogen, R₁ is H and R₂ is halogen. In one embodiment, R₄ of compound of formula XVII is halogen, R₁ is halogen and R₂ is H. In one embodiment, R₄ of compound of formula XVII is alkoxy, R₁ is halogen and R₂ is H. In one embodiment, R₄ of compound of formula XVII is methoxy, R₁ is halogen and R₂ is H. Non limiting examples of compounds of formula XVII are selected from: (2-(4-fluorophenyl)-1H-imidazol-4-yl)(3,4,5-trimethoxyphenyl)methanone (12ba), (2-(4-methoxyphenyl)-1H-imidazol-4-yl)(3,4,5-trimethoxyphenyl)methanone (12ca), (4-fluorophenyl)(2-(4-methoxyphenyl)-1H-imidazol-4-yl)methanone (12cb), (2-(p-tolyl)-1H-imidazol-4-yl)(3,4,5-trimethoxyphenyl)methanone (12da), (4-fluorophenyl)(2-(p-tolyl)-1H-imidazol-4-yl)methanone (12db), (4-Hydroxy-3,5-dimethoxyphenyl)(2-(p-tolyl)-1H-imidazol-4-yl)methanone (12dc), (2-(4-chlorophenyl)-1H-imidazol-4-yl)(3,4,5-trimethoxyphenyl)methanone (12fa), (2-(4-chlorophenyl)-1H-imidazol-4-yl)(4-fluorophenyl)methanone (12fb), (2-(4-chlorophenyl)-1H-imidazol-4-yl)(3,4,5-trihydroxyphenyl)methanone (13fa), (2-(4-(dimethylamino)phenyl)-1H-imidazol-4-yl)(3,4,5-trimethoxyphenyl)methanone (12ga), (2-(4-(dimethylamino)phenyl)-1H-imidazol-4-yl)(4-fluorophenyl)methanone (12gb), (2-(4-(benzyloxy)phenyl)-1H-imidazol-4-yl)(4-fluorophenyl)methanone (12jb), (2-(4-hydroxyphenyl)-1H-imidazol-4-yl)(3,4,5-trimethoxyphenyl)methanone (12ka), (2-(4-bromophenyl)-1H-imidazol-4-yl)(3,4,5-trimethoxyphenyl)methanone (121a), (2-(4-(trifluoromethyl)phenyl)-1H-imidazol-4-yl)(3,4,5-trimethoxyphenyl)methanone (12pa).

In another embodiment a compound of formula XVII is represented by the structure of formula 12fb:

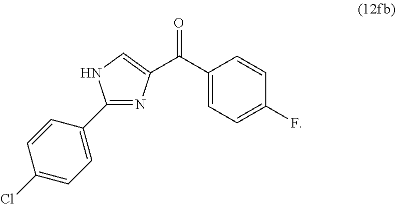 [SMILES: O=C(c1ccc(F)cc1)c1c[nH]c(-c2ccc(Cl)cc2)n1]

In another embodiment a compound of formula XVII is represented by the structure of formula 12cb:

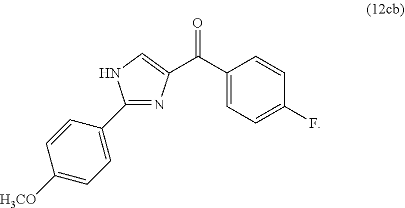 [SMILES: COc1ccc(-c2nc(C(=O)c3ccc(F)cc3)c[nH]2)cc1]

The invention also encompasses methods of treating triple negative breast cancer and/or ovarian cancer by administering at least one compound of formula (XVIII) in a therapeutically effective amount to a subject in need thereof, wherein the compound of Formula (XVIII) is represented by the structure:

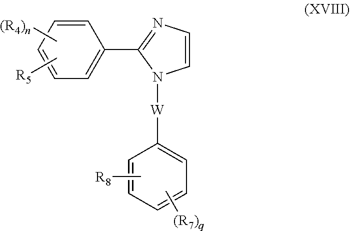

wherein

### W is C═O, C═S, SO₂ or S═O;

R₄ and R₇ are independently H, O-alkyl, I, Br, Cl, F, alkyl, haloalkyl, aminoalkyl, —(CH₂)ᵢNHCH₃, —(CH₂)ᵢNH₂, —(CH₂)ᵢN(CH₃)₂, OCH₂Ph, OH, CN, NO₂, —NHCO-alkyl, COOH, C(O)O-alkyl or C(O)H;
R₅ and R₅ are independently H, O-alkyl, I, Br, Cl, F, alkyl, haloalkyl, aminoalkyl, —(CH₂)ᵢNHCH₃, —(CH₂)ᵢNH₂, —(CH₂)ᵢN(CH₃)₂, OCH₂Ph, OH, CN, NO₂, —NHCO-alkyl, COOH, C(O)O-alkyl or C(O)H;
n is an integer between 1-4;
i is an integer between 0-5; and
q is an integer between 1-4;
or its pharmaceutically acceptable salt, hydrate, polymorph, metabolite, tautomer or isomer.

In one embodiment, W of compound of formula XVIII is C═O. In another embodiment, W of compound of formula XVIII is SO₂. In another embodiment, R₄ of compound of formula XVIII is H. In another embodiment, R₄ of compound of formula XVIII is NO₂. In another embodiment, R₄ of compound of formula XVIII is OBn. In another embodiment, R₇ of compound of formula XVIII is H. In another embodiment, R₇ of compound of formula XVIII is OCH₃. In another embodiment, R₇ of compound of formula XVIII is OCH₃ and q is 3. Non limiting examples of compounds of formula XVII are selected from: (4-methoxyphenyl)(2-phenyl-1H-imidazol-1-yl)methanone (12aba), (2-phenyl-1H-imidazol-1-yl)(3,4,5-trimethoxyphenyl)methanone (12aaa), 2-phenyl-1-(phenylsulfonyl)-1H-imidazole (10a), 2-(4-nitrophenyl)-1-(phenylsulfonyl)-1H-imidazole (10x), 2-(4-(benzyloxy)phenyl)-1-(phenylsulfonyl)-1H-imidazole (10j).

The invention also encompasses methods of treating triple negative breast cancer and/or ovarian cancer by administering at least one compound of formula (XIX) in a therapeutically effective amount to a subject in need thereof, wherein the compound of Formula (XIX) is represented by the structure:

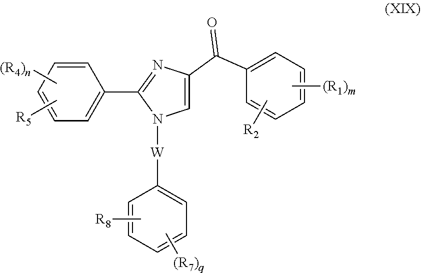

wherein

### W is C═O, C═S, SO₂, S═O;

R₁, R₄ and R₇ are independently H, O-alkyl, I, Br, Cl, F, alkyl, haloalkyl, aminoalkyl, —(CH₂)ᵢNHCH₃, —(CH₂)ᵢNH₂, —(CH₂)ᵢN(CH₃)₂, OCH₂Ph, OH, CN, NO₂, —NHCO-alkyl, COOH, C(O)O-alkyl or C(O)H;
R₂, R₅ and R₈ are independently H, O-alkyl, I, Br, Cl, F, alkyl, haloalkyl, aminoalkyl, —(CH₂)ᵢNHCH₃, —(CH₂)ᵢNH₂, —(CH₂)ᵢN(CH₃)₂, OCH₂Ph, OH, CN, NO₂, —NHCO-alkyl, COOH, C(O)O-alkyl or C(O)H;
m is an integer between 1-4;
n is an integer between 1-4;
i is an integer between 0-5; and
q is 1-4;
or its pharmaceutically acceptable salt, hydrate, polymorph, metabolite, tautomer or isomer.

In one embodiment, R₁, R₄ and R₇ of formula XIX are independently H. In another embodiment, R₁, R₄ and R₇ of formula XIX are independently O-alkyl. In another embodiment, R₁, R₄ and R₇ of formula XIX are independently halogen. In another embodiment, R₁, R₄ and R₇ of formula XIX are independently CN. In another embodiment, R₁, R₄ and R₇ of formula XIX are independently OH. In another embodiment, R₁, R₄ and R₇ of formula XIX are independently alkyl. In another embodiment, R₁, R₄ and R₇ of formula XIX are independently OCH₂Ph. In one embodiment R₂, R₅ and R₅ of formula XIX are independently H. In another embodiment, R₂, R₅ and R₅ of formula XIX are independently O-alkyl. In another embodiment, R₂, R₅ and R₅ of formula XIX are independently halogen. In another embodiment, R₂, R₅ and R₅ of formula XIX are independently CN. In another embodiment, R₂, R₅ and R₅ of formula XIX are independently OH. In another embodiment, R₂, R₅ and R₅ of formula XIX are independently alkyl. In another embodiment, R₂, R₅ and R₅ of formula XIX are independently OCH₂Ph. In another embodiment, R₅, R₂ and R₅ of formula XIX are H, R₄ is 4-N(Me)₂, R₁ is OCH₃, m is 3 and R₇ is OCH₃. In another embodiment, R₅, R₂, R₇ and R₅ of formula XIX are H, R₄ is 4-Br, R₁ is OCH₃, and m is 3. In another embodiment W is SO₂. In another embodiment W is C═O. In another embodiment W is C═S. In another embodiment W is S═O. Non limiting examples of compounds of formula XIX are selected from: (2-(4-(dimethylamino)phenyl)-1-((4-methoxyphenyl)sulfonyl)-1H-imidazol-4-yl)(3,4,5-trimethoxyphenyl)methanone (11gaa); (2-(4-bromophenyl)-1-(phenylsulfonyl)-1H-imidazol-4-yl)(3,4,5-trimethoxyphenyl)methanone (11la), (4-fluorophenyl)(2-(4-methoxyphenyl)-1-(phenylsulfonyl)-1H-imidazol-4-yl)methanone (11cb), (2-(4-chlorophenyl)-1-(phenylsulfonyl)-1H-imidazol-4-yl)(4-fluorophenyl)methanone (11fb), (4-fluorophenyl)(2-phenyl-1-(phenylsulfonyl)-1H-imidazol-4-yl)methanone (11af), (4-fluorophenyl)(1-(phenylsulfonyl)-2-(p-tolyl)-1H-imidazol-4-yl)methanone (11db), (2-(4-(dimethylamino)phenyl)-1-(phenylsulfonyl)-1H-imidazol-4-yl)(3,4,5-trimethoxyphenyl)methanone (11ga), (2-(4-(dimethylamino)phenyl)-1-(phenylsulfonyl)-1H-imidazol-4-yl)(4-fluorophenyl)methanone (11gb), (2-(3,4-dimethoxyphenyl)-1-(phenylsulfonyl)-1H-imidazol-4-yl)(3,4,5-trimethoxyphenyl)methanone (11ha), (2-(4-(benzyloxy)phenyl)-1-(phenylsulfonyl)-1H-imidazol-4-yl)(4-fluorophenyl)methanone (11jb), (2-(4-(dimethylamino)phenyl)-1-((4-methoxyphenyl)sulfonyl)-1H-imidazol-4-yl)(4-fluorophenyl)methanone (12gba).

In another embodiment a compound of formula XIX is represented by the structure of formula 11cb:

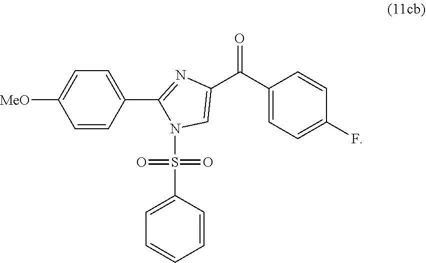 [SMILES: COc1ccc(-c2nc(C(=O)c3ccc(F)cc3)cn2S(=O)(=O)c2ccccc2)cc1]

In another embodiment a compound of formula XIX is represented by the structure of formula 11fb:

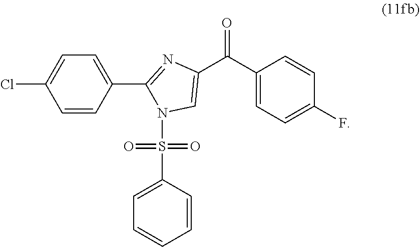 [SMILES: O=C(c1ccc(F)cc1)c1cn(S(=O)(=O)c2ccccc2)c(-c2ccc(Cl)cc2)n1]

The invention also encompasses methods of treating triple negative breast cancer and/or ovarian cancer by administering at least one compound of formula (XX) in a therapeutically effective amount to a subject in need thereof, wherein the compound of Formula (XX) is represented by the structure:

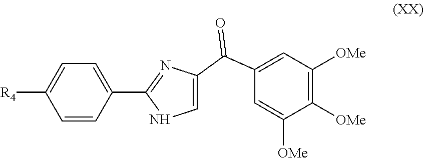

wherein
R₄ is H, O-alkyl, I, Br, Cl, F, alkyl, haloalkyl, aminoalkyl, —(CH₂)ᵢNHCH₃, —(CH₂)ᵢNH₂, —(CH₂)ᵢN(CH₃)₂, OCH₂Ph, OH, CN, NO₂, —NHCO-alkyl, COOH, C(O)O-alkyl or C(O)H; and
i is an integer between 0-5;
or its pharmaceutically acceptable salt, hydrate, polymorph, metabolite, tautomer or isomer.

In one embodiment, R₄ of compound of formula XX is H. In another embodiment, R₄ of compound of formula XX is halogen. In another embodiment, R₄ is F. In another embodiment, R₄ is Cl. In another embodiment R₄ is Br. In another embodiment R₄ is I. In another embodiment, R₄ is alkyl. In another embodiment, R₄ is methyl. Non limiting examples of compounds of formula XX are selected from: (2-phenyl-1H-imidazol-4-yl)(3,4,5-trimethoxyphenyl)methanone (12aa), (2-(4-fluorophenyl)-1H-imidazol-4-yl)(3,4,5-trimethoxyphenyl)methanone (12ba), (2-(4-methoxyphenyl)-1H-imidazol-4-yl)(3,4,5-trimethoxyphenyl)methanone (12ca), (2-(p-tolyl)-1H-imidazol-4-yl)(3,4,5-trimethoxyphenyl)methanone (12da), (2-(4-chlorophenyl)-1H-imidazol-4-yl)(3,4,5-trimethoxyphenyl)methanone (12fa), (2-(4-(dimethylamino)phenyl)-1H-imidazol-4-yl)(3,4,5-trimethoxyphenyl)methanone (12ga), (2-(2-(trifluoromethyl)phenyl)-1H-imidazol-4-yl)(3,4,5-trimethoxyphenyl)methanone (12la), (2-(4-(benzyloxy)phenyl)-1H-imidazol-4-yl)(3,4,5-trimethoxyphenyl)methanone (12ja), (2-(4-hydroxyphenyl)-1H-imidazol-4-yl)(3,4,5-trimethoxyphenyl)methanone (12ka), (2-(4-bromophenyl)-1H-imidazol-4-yl)(3,4,5-trimethoxyphenyl)methanone (121a), (2-(4-(trifluoromethyl)phenyl)-1H-imidazol-4-yl)(3,4,5-trimethoxyphenyl)methanone (12pa).

In another embodiment a compound of formula XX is represented by the structure of formula 12da:

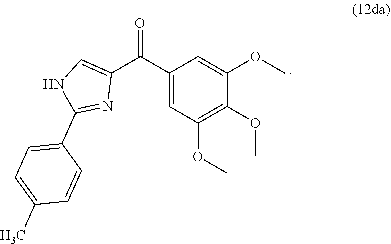 [SMILES: COc1cc(C(=O)c2c[nH]c(-c3ccc(C)cc3)n2)cc(OC)c1OC ; "."]

In another embodiment a compound of formula XX is represented by the structure of formula 12fa:

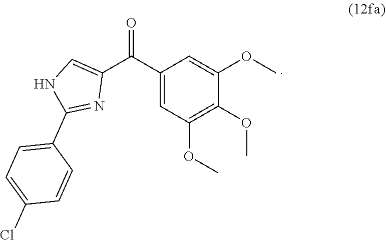 [SMILES: COc1cc(C(=O)c2c[nH]c(-c3ccc(Cl)cc3)n2)cc(OC)c1OC ; "."]

The invention also encompasses methods of treating triple negative breast cancer and/or ovarian cancer by administering at least one compound of formula (XXI) in a therapeutically effective amount to a subject in need thereof, wherein the compound of Formula (XXI) is represented by the structure:

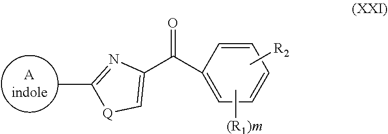

wherein
A is indolyl;

### Q is NH, O or S;

R₁ and R₂ are independently H, O-alkyl, I, Br, Cl, F, alkyl, haloalkyl, aminoalkyl, —(CH₂)ᵢNHCH₃, —(CH₂)ᵢNH₂, —(CH₂)ᵢN(CH₃)₂, OCH₂Ph, OH, CN, NO₂, —NHCO-alkyl, COOH, C(O)O-alkyl or C(O)H; and
wherein said A is optionally substituted by substituted or unsubstituted O-alkyl, O-haloalkyl, F, Cl, Br, I, haloalkyl, CF₃, CN, —CH₂CN, NH₂, hydroxyl, —(CH₂)ᵢNHCH₃, —(CH₂)ᵢNH₂, —(CH₂)ᵢN(CH₃)₂, —OC(O)CF₃, substituted or unsubstituted —SO₂-aryl, substituted or unsubstituted C₁-C₅ linear or branched alkyl, substituted or unsubstituted haloalkyl, substituted or unsubstituted alkylamino, substituted or unsubstituted aminoalkyl, —OCH₂Ph, substituted or unsubstituted —NHCO-alkyl, COOH, substituted or unsubstituted —C(O)Ph, substituted or unsubstituted C(O)O— alkyl, C(O)H, —C(O)NH₂, NO₂ or combination thereof;
i is an integer between 0-5; and
m is an integer between 1-4;
or its pharmaceutically acceptable salt, hydrate, polymorph, metabolite, tautomer or isomer..

In one embodiment, R₁ of compound of formula XXI is OCH₃; m is 3 and R₂ is hydrogen. In another embodiment, R₁ is F; m is 1 and R₂ is hydrogen. In one embodiment, Q of formula XXI is O. In another embodiment Q of formula XXI is NH. In another embodiment, Q of formula XXI is S.

In one embodiment, A ring of compound of formula XXI is substituted 5-indolyl. In another embodiment the substitution is —(C═O)-Aryl. In another embodiment, the aryl is 3,4,5-(OCH₃)₃-Ph.

In another embodiment, A ring of compound of formula XXI is 3-indolyl. In another embodiment, A ring of compound of formula XXI is 5-indolyl. In another embodiment, A ring of compound of formula XXI is 2-indolyl. Non limiting examples of compounds of formula XXI are selected from: (5-(4-(3,4,5-trimethoxybenzoyl)-1H-imidazol-2-yl)-1H-indol-2-yl)(3,4,5-trimethoxyphenyl)methanone (15xaa); (1-(phenylsulfonyl)-2-(1-(phenylsulfonyl)-2-(3,4,5-trimethoxybenzoyl)-1H-indol-5-yl)-1H-imidazol-4-yl)(3,4,5-trimethoxyphenyl)methanone (16xaa); 2-(1H-indol-3-yl)-1H-imidazol-4-yl)(3,4,5-trimethoxyphenyl)methanone (17ya); (2-(1H-indol-2-yl)thiazol-4-yl)(3,4,5-trimethoxyphenyl)methanone (62a); and (2-(1H-indol-5-yl)thiazol-4-yl)(3,4,5-trimethoxyphenyl)methanone (66a).

The invention also encompasses methods of treating triple negative breast cancer and/or ovarian cancer by administering at least one compound of formula (XXIa) in a therapeutically effective amount to a subject in need thereof, wherein the compound of Formula (XXIa) is represented by the structure:

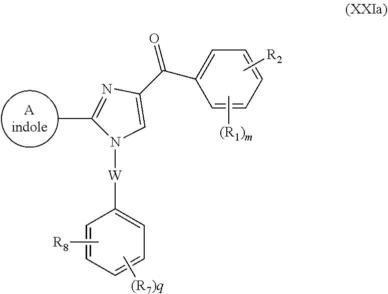

wherein

### W is C═O, C═S, SO₂, S═O;

A is indolyl;
R₁ and R₂ are independently H, O-alkyl, I, Br, Cl, F, alkyl, haloalkyl, aminoalkyl, —(CH₂)ᵢNHCH₃, —(CH₂)ᵢNH₂, —(CH₂)ᵢN(CH₃)₂, OCH₂Ph, OH, CN, NO₂, —NHCO-alkyl, COOH, C(O)O-alkyl or C(O)H;
R₇ and R₅ are independently H, O-alkyl, I, Br, Cl, F, alkyl, haloalkyl, aminoalkyl, —(CH₂)ᵢNHCH₃, —(CH₂)ᵢNH₂, —(CH₂)ᵢN(CH₃)₂, OCH₂Ph, OH, CN, NO₂, —NHCO-alkyl, COOH, C(O)O-alkyl or C(O)H;
wherein said A is optionally substituted by substituted or unsubstituted O-alkyl, O-haloalkyl, F, Cl, Br, I, haloalkyl, CF₃, CN, —CH₂CN, NH₂, hydroxyl, —(CH₂)ᵢNHCH₃, —(CH₂)ᵢNH₂, —(CH₂)ᵢN(CH₃)₂, —OC(O)CF₃, substituted or unsubstituted —SO₂-aryl, substituted or unsubstituted C₁-C₅ linear or branched alkyl, substituted or unsubstituted haloalkyl, substituted or unsubstituted alkylamino, substituted or unsubstituted aminoalkyl, —OCH₂Ph, substituted or unsubstituted —NHCO-alkyl, COOH, substituted or unsubstituted —C(O)Ph, substituted or unsubstituted C(O)O— alkyl, C(O)H, —C(O)NH₂, NO₂ or combination thereof;
i is an integer between 0-5; and
m is an integer between 1-4;
q is an integer between 1-4;
or its pharmaceutically acceptable salt, hydrate, polymorph, metabolite, tautomer or isomer.

In one embodiment, R₁ of compound of formula XXIa is OCH₃; m is 3 and R₂ is hydrogen. In another embodiment, R₁ is F; m is 1 and R₂ is hydrogen. In another embodiment, A ring of compound of formula XXIa is substituted 5-indolyl. In another embodiment, A ring of compound of formula XXIa is 3-indolyl. Non limiting examples of compounds of formula XXIa are selected from: (1-(phenylsulfonyl)-2-(1-(phenylsulfonyl)-2-(3,4,5-trimethoxybenzoyl)-1H-indol-5-yl)-1H-imidazol-4-yl)(3,4,5-trimethoxyphenyl)methanone (16xaa); (1-(phenylsulfonyl)-2-(1-(phenylsulfonyl)-1H-indol-3-yl)-1H-imidazol-4-yl)(3,4,5-trimethoxyphenyl)methanone (17yaa).

The invention also encompasses methods of treating triple negative breast cancer and/or ovarian cancer by administering at least one compound of formula (XXII) in a therapeutically effective amount to a subject in need thereof, wherein the compound of Formula (XXII) is represented by the structure:

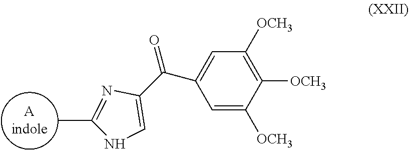

wherein
A is indolyl;
wherein said A is optionally substituted by substituted or unsubstituted O-alkyl, O-haloalkyl, F, Cl, Br, I, haloalkyl, CF₃, CN, —CH₂CN, NH₂, hydroxyl, —(CH₂)ᵢNHCH₃, —(CH₂)ᵢNH₂, —(CH₂)ᵢN(CH₃)₂, —OC(O)CF₃, substituted or unsubstituted —SO₂-aryl, substituted or unsubstituted C₁-C₅ linear or branched alkyl, substituted or unsubstituted haloalkyl, substituted or unsubstituted alkylamino, substituted or unsubstituted aminoalkyl, —OCH₂Ph, substituted or unsubstituted —NHCO-alkyl, COOH, substituted or unsubstituted —C(O)Ph, substituted or unsubstituted C(O)O— alkyl, C(O)H, —C(O)NH₂, NO₂ or combination thereof;
i is an integer between 0-5;
or its pharmaceutically acceptable salt, hydrate, polymorph, metabolite, tautomer or isomer.

In one embodiment, A ring of compound of formula XXII is substituted 5-indolyl. In another embodiment the substitution is —(C═O)-Aryl. In another embodiment, the aryl is 3,4,5-(OCH₃)₃-Ph.

In another embodiment, A ring of compound of formula XXII is 3-indolyl. Non limiting examples of compounds of formula XXII are selected from: (5-(4-(3,4,5-trimethoxybenzoyl)-1H-imidazol-2-yl)-1H-indol-2-yl)(3,4,5-trimethoxyphenyl)methanone (15xaa); (2-(1H-indol-3-yl)-1H-imidazol-4-yl)(3,4,5-trimethoxyphenyl)methanone (17ya),

In another embodiment a compound of formula XXI or XXII is represented by the structure of formula 17ya:

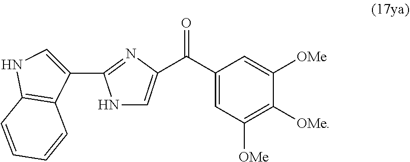 [SMILES: COc1cc(C(=O)c2c[nH]c(-c3c[nH]c4ccccc34)n2)cc(OC)c1OC]

In one embodiment, Q of compound of formula XII is H and P is

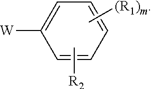

In another embodiment, P of compound of formula XII is H and Q is

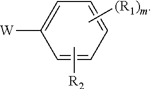

In another embodiment, P of compound of formula XII is

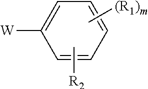

and Q is SO₂-Ph. In one embodiment. Q of compound of formula XII is H and P is

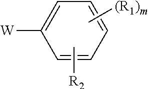

wherein W is C═O. In another embodiment W of compound of formula XII, XVIII, XIX, or XXIa is C═O. In another embodiment, W of compound of formula XII, XVIII, XIX, or XXIa is SO₂. In another embodiment, W of compound of formula XII, XVIII, XIX, or XXIa is C═S. In another embodiment, W of compound of formula XII, XVIII, XIX, or XXIa is S═O.

In one embodiment, Z of compound of formula XIII is oxygen. In another embodiment, Z of compound of formula XIII is sulfur.

In one embodiment, R₅ of compound of formula XII-XVI, XVIII, or XIX is hydrogen, n is 1 and R₄ is in the para position.

In one embodiment, R₄ of compound of formula XII—XX is alkyl. In another embodiment, R₄ of compound of formula XII—XX is H. In another embodiment, R₄ of compound of formula XII—XX is methyl (CH₃). In another embodiment, R₄ of compound of formula XII—XX is O-alkyl. In another embodiment, R₄ of compound of formula XII—XX is OCH₃. In another embodiment, R₄ of compound of formula XII—XX is I. In another embodiment, R₄ of compound of formula XII—XX is Br. In another embodiment, R₄ of compound of formula XII—XX is F. In another embodiment, R₄ of compound of formula XII—XX is Cl. In another embodiment, R₄ of compound of formula XII—XX is N(Me)₂. In another embodiment, R₄ of compound of formula XII—XX is OBn. In another embodiment, R₄ of compound of formula XII—XX is OH. In another embodiment, R₄ of compound of formula XII—XX is CF₃.

In one embodiment, R₂ of compound of formula XII, XIII, XIV, XIVa, XVII, XIX, XXI or XXIa is hydrogen; R₁ is OCH₃ and m is 3. In another embodiment, R₂ of compound of formula XII, XIII, XIV, XIVa, XVII, XIX, XXI or XXIa is hydrogen; m is 1 and R₁ is in the para position. In another embodiment, R₂ of compound of formula XII, XIII, XIV, XIVa, XVII, XIX, XXI or XXIa is hydrogen; m is 1 and R₁ is I. In another embodiment, R₂ of compound of formula XII, XIII, XIV, XIVa, XVII, XIX, XXI or XXIa is hydrogen; m is 1 and R₁ is Br. In another embodiment, R₂ of compound of formula XII, XIII, XIV, XIVa, XVII, XIX, XXI or XXIa is hydrogen; m is 1 and R₁ is F. In another embodiment, R₂ of compound of formula XII, XIII, XIV, XIVa, XVII, XIX, XXI or XXIa is hydrogen; m is 1 and R₁ is Cl. In another embodiment, R₁ of compound of formula XII, XIII, XIV, XIVa, XVII, XIX, XXI or XXIa is I. In another embodiment, R₁ of compound of formula XII, XIII, XIV, XIVa, XVII, XIX, XXI or XXIa is Br. In another embodiment, R₁ of compound of formula XII, XIII, XIV, XIVa, XVII, XIX, XXI or XXIa is Cl. In another embodiment, R₁ of compound of formula XII, XIII, XIV, XIVa, XVII, XIX, XXI or XXIa is F.

In one embodiment Q of compound of formula XII is H and P is

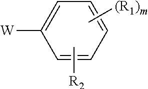

wherein W is C═O. Non-limiting examples of compounds of formula XII—XVII and XX-XXII are selected from (2-phenyl-1H-imidazol-4-yl)(3,4,5-trimethoxyphenyl)methanone (12aa); (4-methoxyphenyl)(2-phenyl-1H-imidazol-4-yl)methanone (12ab); (3-methoxyphenyl)(2-phenyl-1H-imidazol-4-yl)methanone (12ac); (3,5-dimethoxyphenyl)(2-phenyl-1H-imidazol-4-yl)methanone (12ad); (3,4-dimethoxyphenyl)(2-phenyl-1H-imidazol-4-yl)methanone (12ae); (4-fluorophenyl)(2-phenyl-1H-imidazol-4-yl)methanone (12af); (3-fluorophenyl)(2-phenyl-1H-imidazol-4-yl)methanone (12ag); (2-phenyl-1H-imidazol-4-yl)(p-tolyl)methanone (12ah); (2-phenyl-1H-imidazol-4-yl)(m-tolyl)methanone (12ai); (2-(4-fluorophenyl)-1H-imidazol-4-yl)(3,4,5-trimethoxyphenyl)methanone (12ba); (2-(4-methoxyphenyl)-1H-imidazol-4-yl)(3,4,5-trimethoxyphenyl)methanone (12ca); (4-fluorophenyl)(2-(4-methoxyphenyl)-1H-imidazol-4-yl)methanone (12cb); (2-(p-tolyl)-1H-imidazol-4-yl)(3,4,5-trimethoxyphenyl)methanone (12da); (4-fluorophenyl)(2-(p-tolyl)-1H-imidazol-4-yl)methanone (12db); (4-fluorophenyl)(2-(p-tolyl)-1H-imidazol-4-yl)methanone hydrochloride (12db-HO); (4-hydroxy-3,5-dimethoxyphenyl)(2-(p-tolyl)-1H-imidazol-4-yl)methanone (12dc); (3,4,5-trimethoxyphenyl)(2-(3,4,5-trimethoxyphenyl)-1H-imidazol-4-yl)methanone (12ea); (4-fluorophenyl)(2-(3,4,5-trimethoxyphenyl)-1H-imidazol-4-yl)methanone (12eb); (2-(4-chlorophenyl)-1H-imidazol-4-yl)(3,4,5-trimethoxyphenyl)methanone (12fa); (2-(4-chlorophenyl)-1H-imidazol-4-yl)(4-fluorophenyl)methanone (12fb); (2-(4-chlorophenyl)-1H-imidazol-4-yl)(4-hydroxy-3,5-dimethoxyphenyl)methanone (12fc); (2-(4-(dimethylamino)phenyl)-1H-imidazol-4-yl)(3,4,5-trimethoxyphenyl)methanone (12ga); (2-(4-(dimethylamino)phenyl)-1H-imidazol-4-yl)(4-fluorophenyl)methanone (12gb); (2-(3,4-dimethoxyphenyl)-1H-imidazol-4-yl)(3,4,5-trimethoxyphenyl)methanone (12ha); (2-(3,4-dimethoxyphenyl)-1H-imidazol-4-yl)(4-fluorophenyl)methanone (12hb); (2-(2-(trifluoromethyl)phenyl)-1H-imidazol-4-yl)(3,4,5-trimethoxyphenyl)methanone (12la); (4-fluorophenyl)(2-(2-(trifluoromethyl)phenyl)-1H-imidazol-4-yl)methanone (12lb); (2-(4-(benzyloxy)phenyl)-1H-imidazol-4-yl)(3,4,5-trimethoxyphenyl)methanone (12ja); (2-(4-(benzyloxy)phenyl)-1H-imidazol-4-yl)(4-fluorophenyl)methanone (12jb); (2-(4-hydroxyphenyl)-1H-imidazol-4-yl)(3,4,5-trimethoxyphenyl)methanone (12ka); (2-(4-(hydroxyphenyl)-1H-imidazol-4-yl)(4-fluorophenyl)methanone (12kb); (2-(4-bromophenyl)-1H-imidazol-4-yl)(3,4,5-trimethoxyphenyl)methanone (121a); (2-(4-(trifluoromethyl)phenyl)-1H-imidazol-4-yl)(3,4,5-trimethoxyphenyl)methanone (12pa); (3,4,5-trihydroxyphenyl)(2-(3,4,5-trihydroxyphenyl)-1H-imidazol-4-yl)methanone (13ea); (2-(4-chlorophenyl)-1H-imidazol-4-yl)(3,4,5-trihydroxyphenyl)methanone (13fa); and 2-(3,4-dihydroxyphenyl)-1H-imidazol-4-yl)(3,4,5-trihydroxyphenyl)methanone (13ha).

In one embodiment, P of compound of formula XII is

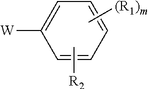

and Q is SO₂-Ph. Non-limiting examples of compound of formula XII wherein P of compound of formula XII is

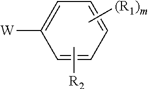

and Q is SO₂-Ph are selected from (4-methoxyphenyl)(2-phenyl-1-(phenylsulfonyl)-1H-imidazol-4-yl)methanone (11ab); (3-methoxyphenyl)(2-phenyl-1-(phenylsulfonyl)-1H-imidazol-4-yl)methanone (11ac); (2-phenyl-1-(phenylsulfonyl)-1H-imidazol-4-yl)(p-tolyl)methanone (11ah); (4-fluorophenyl)(2-phenyl-1-(phenylsulfonyl)-1H-imidazol-4-yl)methanone (11af); (3-fluorophenyl)(2-phenyl-1-(phenylsulfonyl)-1H-imidazol-4-yl)methanone (11ag); (4-fluorophenyl)(2-(4-methoxyphenyl)-1-(phenylsulfonyl)-1H-imidazol-4-yl)methanone (11cb); (1-(phenylsulfonyl)-2-(p-tolyl)-1H-imidazol-4-yl)(3,4,5-trimethoxyphenyl)methanone (11da); (4-fluorophenyl)(1-(phenylsulfonyl)-2-(p-tolyl)-1H-imidazol-4-yl)methanone (11db); (1-(phenylsulfonyl)-2-(3,4,5-trimethoxyphenyl)-1H-imidazol-4-yl)(3,4,5-trimethoxyphenyl)methanone (ilea); (4-fluorophenyl)(1-(phenylsulfonyl)-2-(3,4,5-trimethoxyphenyl)-1H-imidazol-4-yl)methanone (11eb); (2-(4-chlorophenyl)-1-(phenylsulfonyl)-1H-imidazol-4-yl)(4-fluorophenyl)methanone (11fb); (2-(4-(dimethylamino)phenyl)-1-(phenylsulfonyl)-1H-imidazol-4-yl)(3,4,5-trimethoxyphenyl)methanone (11ga); (2-(4-(dimethylamino)phenyl)-1-(phenylsulfonyl)-1H-imidazol-4-yl)(4-fluorophenyl)methanone (11gb); (2-(3,4-dimethoxyphenyl)-1-(phenylsulfonyl)-1H-imidazol-4-yl)(3,4,5-trimethoxyphenyl)methanone (11ha); (2-(3,4-dimethoxyphenyl)-1-(phenylsulfonyl)-1H-imidazol-4-yl)(4-fluorophenyl)methanone (11hb); (1-(phenylsulfonyl)-2-(2-(trifluoromethyl)phenyl)-1H-imidazol-4-yl)(3,4,5-trimethoxyphenyl)methanone (11ia); (1-(phenylsulfonyl)-2-(2-(trifluoromethyl)phenyl)-1H-imidazol-4-yl)(4-fluorophenyl)methanone (11ib); and (2-(4-(benzyloxy)phenyl)-1-(phenylsulfonyl)-1H-imidazol-4-yl)(4-fluorophenyl)methanone (11jb); (2-(4-bromophenyl)-1-(phenylsulfonyl)-1H-imidazol-4-yl)(3,4,5-trimethoxyphenyl)methanone (11la); (1-(phenylsulfonyl)-2-(4-(trifluoromethyl)phenyl)-1H-imidazol-4-yl)(3,4,5-trimethoxyphenyl)methanone (11pa).

In one embodiment, R₄ and R₅ of compounds of formula XIII-XVI are hydrogens. Non-limiting examples of compounds of formula XIII-XVI wherein R₄ and R₅ are hydrogens are selected from (2-phenyl-1H-imidazol-4-yl)(3,4,5-trimethoxyphenyl)methanone (12aa); (4-methoxyphenyl)(2-phenyl-1H-imidazol-4-yl)methanone (12ab); (3-methoxyphenyl)(2-phenyl-1H-imidazol-4-yl)methanone (12ac); (3,5-dimethoxyphenyl)(2-phenyl-1H-imidazol-4-yl)methanone (12ad); (3,4-dimethoxyphenyl)(2-phenyl-1H-imidazol-4-yl)methanone (12ae); (4-fluorophenyl)(2-phenyl-1H-imidazol-4-yl)methanone (12af); (3-fluorophenyl)(2-phenyl-1H-imidazol-4-yl)methanone (12ag); (2-phenyl-1H-imidazol-4-yl)(p-tolyl)methanone (12ah); and (2-phenyl-1H-imidazol-4-yl)(m-tolyl)methanone (12ai).

In one embodiment, P of compound of formula XII is H and Q is

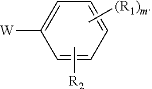

In another embodiment W is C═O. In another embodiment, W of compound of formula XVIII is C═O. Non-limiting examples of compound of formula XVIII wherein W is C═O are selected from (4-methoxyphenyl)(2-phenyl-1H-imidazol-1-yl)methanone (12aba) and (2-phenyl-1H-imidazol-1-yl)(3,4,5-trimethoxyphenyl)methanone (12aaa).

In another embodiment, W of compound of formula XVIII is SO₂. Non-limiting examples of compound of formula XVIII wherein W is SO₂ are selected from 2-phenyl-1-(phenylsulfonyl)-1H-imidazole (10a); 2-(4-nitrophenyl)-1-(phenylsulfonyl)-1H-imidazole (10x) and 2-(4-(benzyloxy)phenyl)-1-(phenylsulfonyl)-1H-imidazole (10j).

The present invention further encompasses methods of treating prostate cancer, taxane resistant prostate cancer, breast cancer, triple negative breast cancer, lung cancer, melanoma, glioma, colon cancer, uterine cancer, ovarian cancer, and pancreatic cancer using a compound as described herein, for example, a compound of formulas VIII, XI, XI(b), XI(c), XI((e) and compounds 5a, 5b, 5c, 5d, 5e, 17ya, and 55. The present invention further encompasses methods of treating prostate cancer, taxane resistant prostate cancer, breast cancer, triple negative breast cancer, lung cancer, melanoma, glioma, colon cancer, uterine cancer, ovarian cancer, and pancreatic cancer using a compound as described herein. The present invention further encompasses methods of treating prostate cancer using a compound as described herein. The present invention further encompasses methods of treating taxane resistant prostate cancer using a compound as described herein. The present invention further encompasses methods of treating lung cancer using a compound as described herein. The present invention further encompasses methods of treating breast cancer using a compound as described herein. The present invention further encompasses methods of treating melanoma using a compound as described herein. The present invention further encompasses methods of treating glioma using a compound as described herein. The present invention further encompasses methods of treating colon cancer using a compound as described herein. The present invention further encompasses methods of treating prostate cancer, taxane resistant prostate cancer, breast cancer, triple negative breast cancer, lung cancer, melanoma, glioma, colon cancer, uterine cancer, ovarian cancer, and pancreatic cancer using a compound as described herein. The present invention further encompasses methods of treating uterine cancer using a compound as described herein. The present invention further encompasses methods of treating pancreatic cancer using a compound as described herein. In one embodiment, the compound is a compound of formulas VIII, XI, XI(b), XI(c), and XI((e) and compounds 5a, 5b, 5c, 5d, 5e, 17ya, and 55. In one embodiment, the compound is a compound of formula XI. In one embodiment, the compound is a compound of formula XI(e). In one embodiment, the compound is compound 17ya. In another embodiment, the compound is compound 55.

As used herein, “single-, fused- or multiple-ring, aryl or (hetero)cyclic ring systems” can be any such ring, including but not limited to phenyl, biphenyl, triphenyl, naphthyl, cycloalkyl, cycloalkenyl, cyclodienyl, fluorene, adamantane, etc.

“Saturated or unsaturated N-heterocycles” can be any such N-containing heterocycle, including but not limited to aza- and diaza-cycloalkyls such as aziridinyl, azetidinyl, diazatidinyl, pyrrolidinyl, piperidinyl, piperazinyl, and azocanyl, pyrrolyl, pyrazolyl, imidazolyl, pyridinyl, pyrimidinyl, pyrazinyl, pyridazinyl, triazinyl, tetrazinyl, pyrrolizinyl, indolyl, quinolinyl, isoquinolinyl, benzimidazolyl, indazolyl, quinolizinyl, cinnolinyl, quinololinyl, phthalazinyl, naphthyridinyl, quinoxalinyl, etc.

“Saturated or unsaturated O-Heterocycles” can be any such O-containing heterocycle including but not limited to oxiranyl, oxetanyl, tetrahydrofuranyl, tetrahydropyranyl, dioxanyl, furanyl, pyrylium, benzofuranyl, benzodioxolyl, etc.

“Saturated or unsaturated S-heterocycles” can be any such S-containing heterocycle, including but not limited to thiranyl, thietanyl, tetrahydrothiophene-yl, dithiolanyl, tetrahydrothiopyranyl, thiophene-yl, thiepinyl, thianaphthenyl, etc.

“Saturated or unsaturated mixed heterocycles” can be any heterocycle containing two or more S-, N-, or O-heteroatoms, including but not limited to oxathiolanyl, morpholinyl, thioxanyl, thiazolyl, isothiazolyl, thiadiazolyl, oxazolyl, isoxazolyl, oxadiaziolyl, etc.

As used herein, “aliphatic straight- or branched-chain hydrocarbon” refers to both alkylene groups that contain a single carbon and up to a defined upper limit, as well as alkenyl groups and alkynyl groups that contain two carbons up to the upper limit, whether the carbons are present in a single chain or a branched chain. Unless specifically identified, a hydrocarbon can include up to about 30 carbons, or up to about 20 hydrocarbons, or up to about 10 hydrocarbons. Alkenyl and alkynyl groups can be mono-unsaturated or polyunsaturated. In another embodiment, an alkyl includes C₁-C₆ carbons. In another embodiment, an alkyl includes C₁-C₈ carbons. In another embodiment, an alkyl includes C₁-C₁₀ carbons. In another embodiment, an alkyl is a C₁-C₁₂ carbons. In another embodiment, an alkyl is a C₁-C₅ carbons.

As used herein, the term “alkyl” can be any straight- or branched-chain alkyl group containing up to about 30 carbons unless otherwise specified. In another embodiment, an alkyl includes C₁-C₆ carbons. In another embodiment, an alkyl includes C₁-C₈ carbons. In another embodiment, an alkyl includes C₁-C₁₀ carbons. In another embodiment, an alkyl is a C₁-C₁₂ carbons. In another embodiment, an alkyl is a C₁-C₂₀ carbons. In another embodiment, cyclic alkyl group has 3-8 carbons. In another embodiment, branched alkyl is an alkyl substituted by alkyl side chains of 1 to 5 carbons.

The alkyl group can be a sole substituent or it can be a component of a larger substituent, such as in an alkoxy, haloalkyl, arylalkyl, alkylamino, dialkylamino, alkylamido, alkylurea, etc. Preferred alkyl groups are methyl, ethyl, and propyl, and thus halomethyl, dihalomethyl, trihalomethyl, haloethyl, dihaloethyl, trihaloethyl, halopropyl, dihalopropyl, trihalopropyl, methoxy, ethoxy, propoxy, arylmethyl, arylethyl, arylpropyl, methylamino, ethylamino, propylamino, dimethylamino, diethylamino, methylamido, acetamido, propylamido, halomethylamido, haloethylamido, halopropylamido, methyl-urea, ethyl-urea, propyl-urea, etc.

As used herein, the term “aryl” refers to any aromatic ring that is directly bonded to another group. The aryl group can be a sole substituent, or the aryl group can be a component of a larger substituent, such as in an arylalkyl, arylamino, arylamido, etc. Exemplary aryl groups include, without limitation, phenyl, tolyl, xylyl, furanyl, naphthyl, pyridinyl, pyrimidinyl, pyridazinyl, pyrazinyl, triazinyl, thiazolyl, oxazolyl, isooxazolyl, pyrazolyl, imidazolyl, thiophene-yl, pyrrolyl, phenylmethyl, phenylethyl, phenylamino, phenylamido, etc.

As used herein, the term “aminoalkyl” refers to an amine group substituted by an alkyl group as defined above. Aminoalkyl refers to monoalkylamine, dialkylamine or trialkylamine. Nonlimiting examples of aminoalkyl groups are —N(Me)₂, —NHMe, —NH₃.

A “haloalkyl” group refers, in another embodiment, to an alkyl group as defined above, which is substituted by one or more halogen atoms, e.g. by F, Cl, Br or I. Nonlimiting examples of haloalkyl groups are CF₃, CF₂CF₃, CH₂CF₃.

In one embodiment, this invention provides a compound used in this invention or its isomer, metabolite, pharmaceutically acceptable salt, pharmaceutical product, tautomer, hydrate, N-oxide, polymorph, or crystal or combinations thereof. In one embodiment, this invention provides an isomer of the compound of this invention. In another embodiment, this invention provides a metabolite of the compound of this invention. In another embodiment, this invention provides a pharmaceutically acceptable salt of the compound of this invention. In another embodiment, this invention provides a pharmaceutical product of the compound of this invention. In another embodiment, this invention provides a tautomer of the compound of this invention. In another embodiment, this invention provides a hydrate of the compound of this invention. In another embodiment, this invention provides an N-oxide of the compound of this invention. In another embodiment, this invention provides a polymorph of the compound of this invention. In another embodiment, this invention provides a crystal of the compound of this invention. In another embodiment, this invention provides composition comprising a compound of this invention, as described herein, or, in another embodiment, a combination of an isomer, metabolite, pharmaceutically acceptable salt, pharmaceutical product, tautomer, hydrate, N-oxide, polymorph, or crystal of the compound of this invention.

In one embodiment, the term “isomer” includes, but is not limited to, optical isomers and analogs, structural isomers and analogs, conformational isomers and analogs, and the like.

In one embodiment, the compounds of this invention are the pure (E)-isomers. In another embodiment, the compounds of this invention are the pure (Z)-isomers. In another embodiment, the compounds of this invention are a mixture of the (E) and the (Z) isomers. In one embodiment, the compounds of this invention are the pure (R)-isomers. In another embodiment, the compounds of this invention are the pure (S)-isomers. In another embodiment, the compounds of this invention are a mixture of the (R) and the (S) isomers.

The compounds of the present invention can also be present in the form of a racemic mixture, containing substantially equivalent amounts of stereoisomers. In another embodiment, the compounds of the present invention can be prepared or otherwise isolated, using known procedures, to obtain a stereoisomer substantially free of its corresponding stereoisomer (i.e., substantially pure). By substantially pure, it is intended that a stereoisomer is at least about 95% pure, more preferably at least about 98% pure, most preferably at least about 99% pure.

Compounds of the present invention can also be in the form of a hydrate, which means that the compound further includes a stoichiometric or non-stoichiometric amount of water bound by non-covalent intermolecular forces.

Compounds of the present invention may exist in the form of one or more of the possible tautomers and depending on the particular conditions it may be possible to separate some or all of the tautomers into individual and distinct entities. It is to be understood that all of the possible tautomers, including all additional enol and keto tautomers and/or isomers are hereby covered. For example, the following tautomers, but not limited to these, are included.

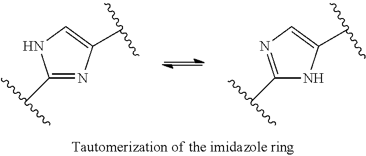 [SMILES: *c1c[nH]c(*)n1 ; *c1cnc(*)[nH]1]

The invention includes “pharmaceutically acceptable salts” of the compounds of this invention, which may be produced, by reaction of a compound of this invention with an acid or base. Certain compounds, particularly those possessing acid or basic groups, can also be in the form of a salt, preferably a pharmaceutically acceptable salt. The term “pharmaceutically acceptable salt” refers to those salts that retain the biological effectiveness and properties of the free bases or free acids, which are not biologically or otherwise undesirable. The salts are formed with inorganic acids such as hydrochloric acid, hydrobromic acid, sulfuric acid, nitric acid, phosphoric acid and the like, and organic acids such as acetic acid, propionic acid, glycolic acid, pyruvic acid, oxylic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid, N-acetylcysteine and the like. Other salts are known to those of skill in the art and can readily be adapted for use in accordance with the present invention.

Suitable pharmaceutically-acceptable salts of amines of compounds the compounds of this invention may be prepared from an inorganic acid or from an organic acid. In one embodiment, examples of inorganic salts of amines are bisulfates, borates, bromides, chlorides, hemisulfates, hydrobromates, hydrochlorates, 2-hydroxyethylsulfonates (hydroxyethanesulfonates), iodates, iodides, isothionates, nitrates, persulfates, phosphate, sulfates, sulfamates, sulfanilates, sulfonic acids (alkylsulfonates, arylsulfonates, halogen substituted alkylsulfonates, halogen substituted arylsulfonates), sulfonates and thiocyanates.

In one embodiment, examples of organic salts of amines may be selected from aliphatic, cycloaliphatic, aromatic, araliphatic, heterocyclic, carboxylic and sulfonic classes of organic acids, examples of which are acetates, arginines, aspartates, ascorbates, adipates, anthranilates, algenates, alkane carboxylates, substituted alkane carboxylates, alginates, benzenesulfonates, benzoates, bisulfates, butyrates, bicarbonates, bitartrates, citrates, camphorates, camphorsulfonates, cyclohexylsulfamates, cyclopentanepropionates, calcium edetates, camsylates, carbonates, clavulanates, cinnamates, dicarboxylates, digluconates, dodecylsulfonates, dihydrochlorides, decanoates, enanthuates, ethanesulfonates, edetates, edisylates, estolates, esylates, fumarates, formates, fluorides, galacturonates gluconates, glutamates, glycolates, glucorate, glucoheptanoates, glycerophosphates, gluceptates, glycollylarsanilates, glutarates, glutamate, heptanoates, hexanoates, hydroxymaleates, hydroxycarboxlic acids, hexylresorcinates, hydroxybenzoates, hydroxynaphthoates, hydrofluorates, lactates, lactobionates, laurates, malates, maleates, methylenebis(beta-oxynaphthoate), malonates, mandelates, mesylates, methane sulfonates, methylbromides, methylnitrates, methylsulfonates, monopotassium maleates, mucates, monocarboxylates, naphthalenesulfonates, 2-naphthalenesulfonates, nicotinates, nitrates, napsylates, N-methylglucamines, oxalates, octanoates, oleates, pamoates, phenylacetates, picrates, phenylbenzoates, pivalates, propionates, phthalates, phenylacetate, pectinates, phenylpropionates, palmitates, pantothenates, polygalacturates, pyruvates, quinates, salicylates, succinates, stearates, sulfanilate, subacetates, tartrates, theophyllineacetates, p-toluenesulfonates (tosylates), trifluoroacetates, terephthalates, tannates, teoclates, trihaloacetates, triethiodide, tricarboxylates, undecanoates and valerates.

In one embodiment, examples of inorganic salts of carboxylic acids or hydroxyls may be selected from ammonium, alkali metals to include lithium, sodium, potassium, cesium; alkaline earth metals to include calcium, magnesium, aluminium; zinc, barium, cholines, quaternary ammoniums.

In another embodiment, examples of organic salts of carboxylic acids or hydroxyl may be selected from arginine, organic amines to include aliphatic organic amines, alicyclic organic amines, aromatic organic amines, benzathines, t-butylamines, benethamines (N-benzylphenethylamine), dicyclohexylamines, dimethylamines, diethanolamines, ethanolamines, ethylenediamines, hydrabamines, imidazoles, lysines, methylamines, meglamines, N-methyl-D-glucamines, N,N′-dibenzylethylenediamines, nicotinamides, organic amines, ornithines, pyridines, picolies, piperazines, procain, tris(hydroxymethyl)methylamines, triethylamines, triethanolamines, trimethylamines, tromethamines and ureas.

In one embodiment, the salts may be formed by conventional means, such as by reacting the free base or free acid form of the product with one or more equivalents of the appropriate acid or base in a solvent or medium in which the salt is insoluble or in a solvent such as water, which is removed in vacuo or by freeze drying or by exchanging the ions of an existing salt for another ion or suitable ion-exchange resin.

The compounds used in the method of the invention are synthesized according to published methods. In particular, the compounds are synthesized according to the methods described in PCT publication Nos. WO 2010/74776, published Jul. 1, 2010; WO 2011/19059, published Sep. 9, 2010; and WO 2012/027481, published Mar. 1, 2012, hereby incorporated by reference.

### Pharmaceutical Composition

Another aspect of the present invention relates to a pharmaceutical composition for use in treating triple negative breast cancer and/or ovarian cancer including a pharmaceutically acceptable carrier and at least one compound described above. Typically, the pharmaceutical composition of the present invention will include a compound or its pharmaceutically acceptable salt, as well as a pharmaceutically acceptable carrier. The term “pharmaceutically acceptable carrier” refers to any suitable adjuvants, carriers, excipients, or stabilizers, and can be in solid or liquid form such as, tablets, capsules, powders, solutions, suspensions, or emulsions.

Typically, the composition will contain from about 0.01 to 99 percent, preferably from about 20 to 75 percent of active compound(s), together with the adjuvants, carriers and/or excipients. While individual needs may vary, determination of optimal ranges of effective amounts of each component is within the skill of the art. Typical dosages comprise about 0.01 to about 100 mg/kg body wt. The preferred dosages comprise about 0.1 to about 100 mg/kg body wt. The most preferred dosages comprise about 1 to about 100 mg/kg body wt. Treatment regimen for the administration of the compounds of the present invention can also be determined readily by those with ordinary skill in art. That is, the frequency of administration and size of the dose can be established by routine optimization, preferably while minimizing any side effects.

The solid unit dosage forms can be of the conventional type. The solid form can be a capsule and the like, such as an ordinary gelatin type containing the compounds and a carrier, for example, lubricants and inert fillers such as, lactose, sucrose, or cornstarch. The compounds may be tabulated with conventional tablet bases such as lactose, sucrose, or cornstarch in combination with binders like acacia, cornstarch, or gelatin, disintegrating agents, such as cornstarch, potato starch, or alginic acid, and a lubricant, like stearic acid or magnesium stearate.

The tablets, capsules, and the like can also contain a binder such as gum tragacanth, acacia, corn starch, or gelatin; excipients such as dicalcium phosphate; a disintegrating agent such as corn starch, potato starch, alginic acid; a lubricant such as magnesium stearate; and a sweetening agent such as sucrose, lactose, or saccharin. When the dosage unit form is a capsule, it can contain, in addition to materials of the above type, a liquid carrier such as a fatty oil.

Various other materials may be present as coatings or to modify the physical form of the dosage unit. For instance, tablets can be coated with shellac, sugar, or both. A syrup can contain, in addition to active ingredient, sucrose as a sweetening agent, methyl and propylparabens as preservatives, a dye, and flavoring such as cherry or orange flavor.

For oral therapeutic administration, the active compounds can be incorporated with excipients and used in the form of tablets, capsules, elixirs, suspensions, syrups, and the like. Such compositions and preparations should contain at least 0.1% of active compound. The percentage of the compound in these compositions can, of course, be varied and can conveniently be between about 2% to about 60% of the weight of the unit. The amount of active compound in such therapeutically useful compositions is such that a suitable dosage will be obtained. Preferred compositions according to the present invention are prepared so that an oral dosage unit contains between about 1 mg and 800 mg of active compound.

The active compounds or formulations thereof may be orally administered, for example, with an inert diluent, or with an assimilable edible carrier, or they can be enclosed in hard or soft shell capsules, or they can be compressed into tablets, or they can be incorporated directly with the food of the diet.

The pharmaceutical forms suitable for injectable use include sterile aqueous solutions or dispersions and sterile powders for the extemporaneous preparation of sterile injectable solutions or dispersions. In all cases, the form should be sterile and should be fluid to the extent that easy syringability exists. It should be stable under the conditions of manufacture and storage and should be preserved against the contaminating action of microorganisms, such as bacteria and fungi. The carrier can be a solvent or dispersion medium containing, for example, water, ethanol, polyol (e.g., glycerol, propylene glycol, and liquid polyethylene glycol), suitable mixtures thereof, and vegetable oils.

The compounds or pharmaceutical compositions of the present invention may also be administered in injectable dosages by solution or suspension of these materials in a physiologically acceptable diluent with a pharmaceutical adjuvant, carrier or excipient. Such adjuvants, carriers and/or excipients include, but are not limited to, sterile liquids, such as water and oils, with or without the addition of a surfactant and other pharmaceutically and physiologically acceptable components. Illustrative oils are those of petroleum, animal, vegetable, or synthetic origin, for example, peanut oil, soybean oil, or mineral oil. In general, water, saline, aqueous dextrose and related sugar solution, and glycols, such as propylene glycol or polyethylene glycol, are preferred liquid carriers, particularly for injectable solutions.

The active compounds or formulations thereof may also be administered parenterally. Solutions or suspensions of these active compounds can be prepared in water suitably mixed with a surfactant such as hydroxypropylcellulose. Dispersions can also be prepared in glycerol, liquid polyethylene glycols, and mixtures thereof in oils. Illustrative oils are those of petroleum, animal, vegetable, or synthetic origin, for example, peanut oil, soybean oil, or mineral oil. In general, water, saline, aqueous dextrose and related sugar solution, and glycols such as, propylene glycol or polyethylene glycol, are preferred liquid carriers, particularly for injectable solutions. Under ordinary conditions of storage and use, these preparations contain a preservative to prevent the growth of microorganisms.

For use as aerosols, the compounds or formulations thereof in solution or suspension may be packaged in a pressurized aerosol container together with suitable propellants, for example, hydrocarbon propellants like propane, butane, or isobutane with conventional adjuvants. The materials of the present invention also may be administered in a non-pressurized form such as in a nebulizer or atomizer.

The compounds used in the methods of the invention are administered in combination with an anti-cancer agent. In one embodiment, the anti-cancer agent is a monoclonal antibody. In some embodiments, the monoclonal antibodies are used for diagnosis, monitoring, or treatment of cancer. In one embodiment, monoclonal antibodies react against specific antigens on cancer cells. In one embodiment, the monoclonal antibody acts as a cancer cell receptor antagonist. In one embodiment, monoclonal antibodies enhance the patient's immune response. In one embodiment, monoclonal antibodies act against cell growth factors, thus blocking cancer cell growth. In one embodiment, anti-cancer monoclonal antibodies are conjugated or linked to anti-cancer drugs, radioisotopes, other biologic response modifiers, other toxins, or a combination thereof. In one embodiment, anti-cancer monoclonal antibodies are conjugated or linked to a compound as described hereinabove.

Yet another aspect of the present invention relates to a method of treating triple negative breast cancer and/or ovarian cancer that includes selecting a subject in need of treatment the cancer and administering to the subject a pharmaceutical composition comprising at least one compound and a pharmaceutically acceptable carrier under conditions effective to treat the cancer.

When administering the compounds, they can be administered systemically or, alternatively, they can be administered directly to a specific site where cancer cells or precancerous cells are present. Thus, administering can be accomplished in any manner effective for delivering the compounds or the pharmaceutical compositions to the cancer cells or precancerous cells. Exemplary modes of administration include, without limitation, administering the compounds or compositions orally, topically, transdermally, parenterally, subcutaneously, intravenously, intramuscularly, intraperitoneally, by intranasal instillation, by intracavitary or intravesical instillation, intraocularly, intraarterially, intralesionally, or by application to mucous membranes, such as, that of the nose, throat, and bronchial tubes.

### Biological Activity

The invention encompasses compounds and compositions for use in treating triple negative breast cancer and/or ovarian cancer. At least one compound or a composition comprising the same will have utility in inhibiting, suppressing, enhancing or stimulating a desired response in a subject, as will be understood by one skilled in the art. The compositions may further comprise additional active ingredients, whose activity is useful for the particular application for which the compound of this invention is being administered.

Drug resistance is the major cause of cancer chemotherapy failure. One major contributor to multidrug resistance is overexpression of P-glycoprotein (P-gp). This protein is a clinically important transporter protein belonging to the ATP-binding cassette family of cell membrane transporters. It can pump substrates including anticancer drugs out of tumor cells through an ATP-dependent mechanism. The TNBC may be taxane-resistant TNBC, taxane-sensitive TNBC, and/or metastasis.

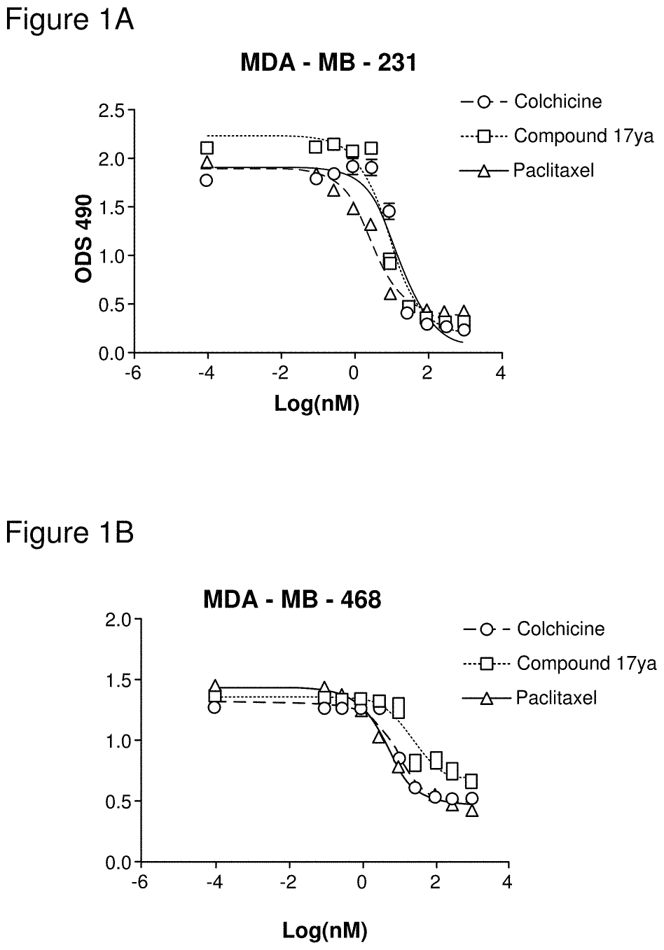

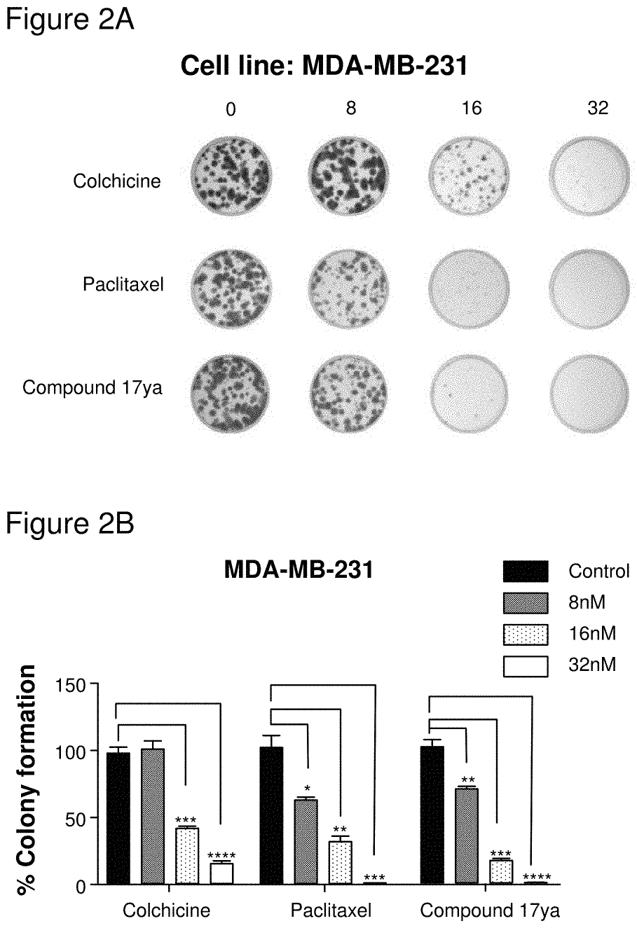

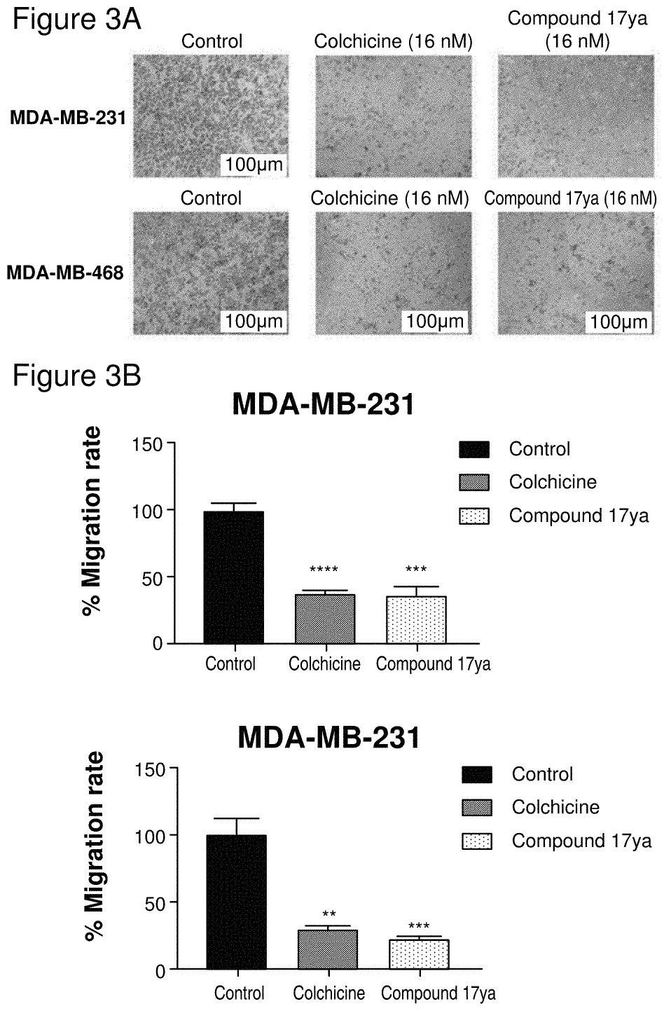

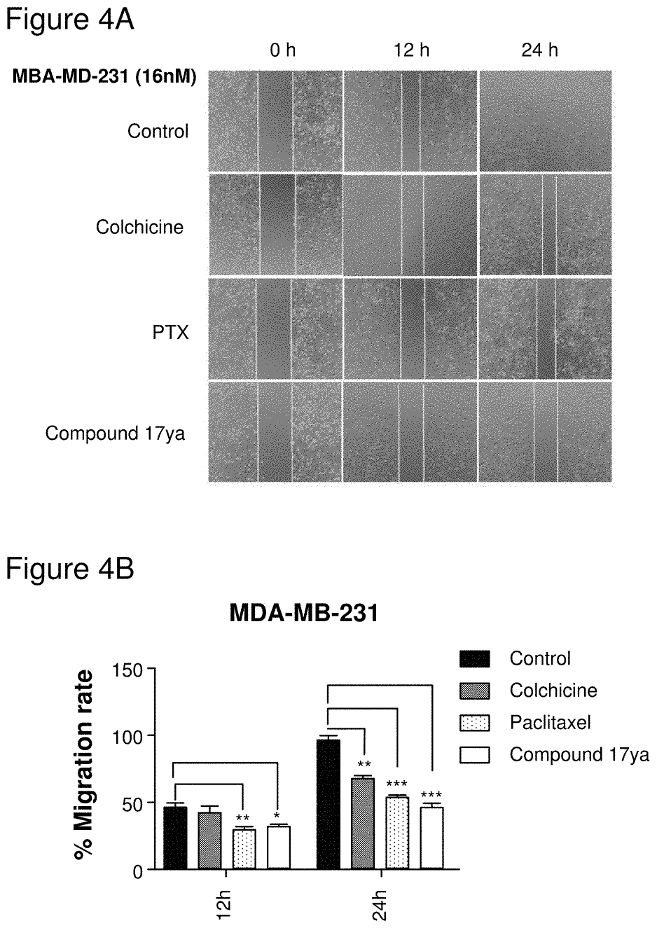

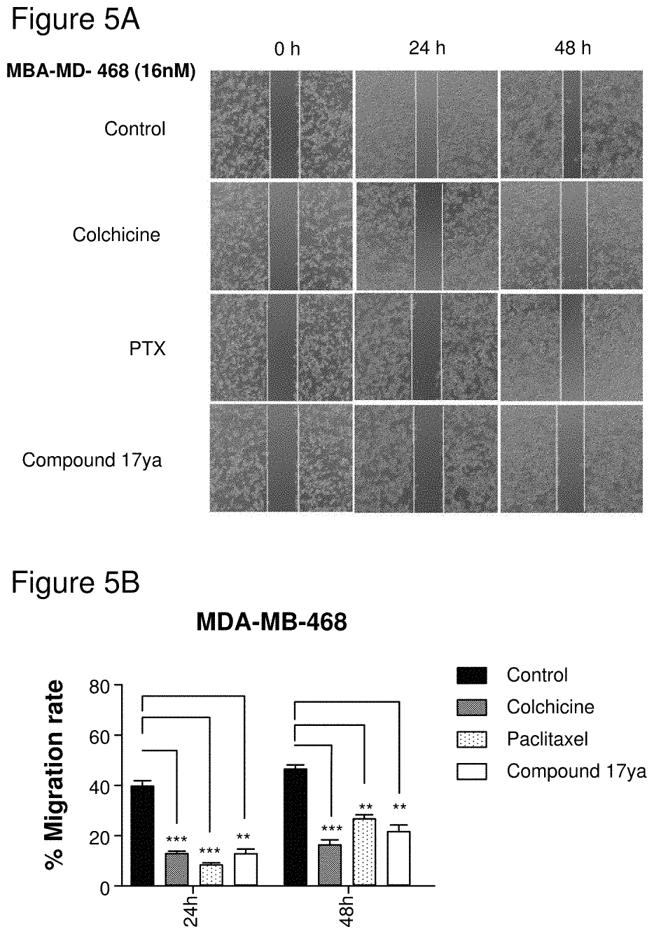

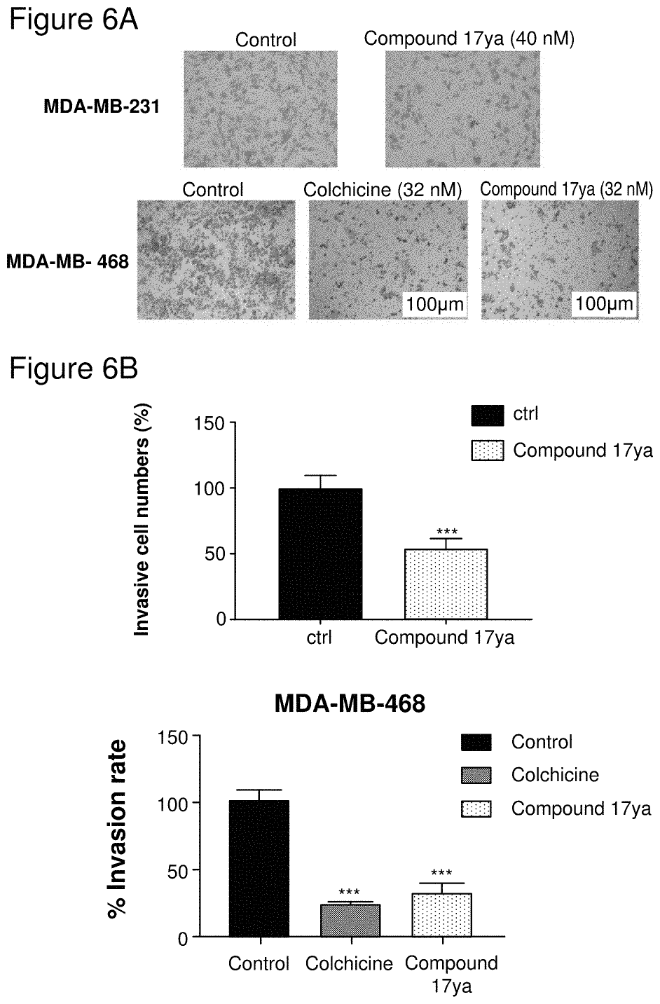

The method of treating TNBC was illustrated by the in vitro studies of compound 17ya determining the anticancer activity of the compound. Compound 17ya anti-TNBC activity was compared to colchicine and paclitaxel against cell lines MDA-MB-231 and MDA-MB-468. In tests with MDA-MB-231, the IC₅₀ (nM) was determined to be 17.46, 3.05, and 8.23 for colchicine, paclitaxel, and compound 17ya, respectively, and the SE was 0.05, 0.04, and 0.05, respectively. In tests with MDA-MB-468, the IC₅₀ (nM) was determined to be 9.80, 4.61, and 22.96 for colchicine, paclitaxel, and compound 17ya, respectively, and the SE was 0.02, 0.03, and 0.02, respectively. FIG. 1 graphically illustrates the results of the anti-cancer activity of compound 17ya in vitro as compared to colchicine and paclitaxel for cell lines MDA-MB-231 and MDA-MB-468. FIG. 2 illustrates the anti-migration of compound 17ya (16 nM) on TNBC cells as compared to colchicine (16 nM) and a control in cell lines MDA-MB-231 and MDA-MB-468. The anti-invasion properties of compound 17ya (40 nM) was also determined in TNBC cell lines MDA-MB-231 and MDA-MB-468 as compared to a control, colchicine (32 nM) and paclitaxel (32 nM).

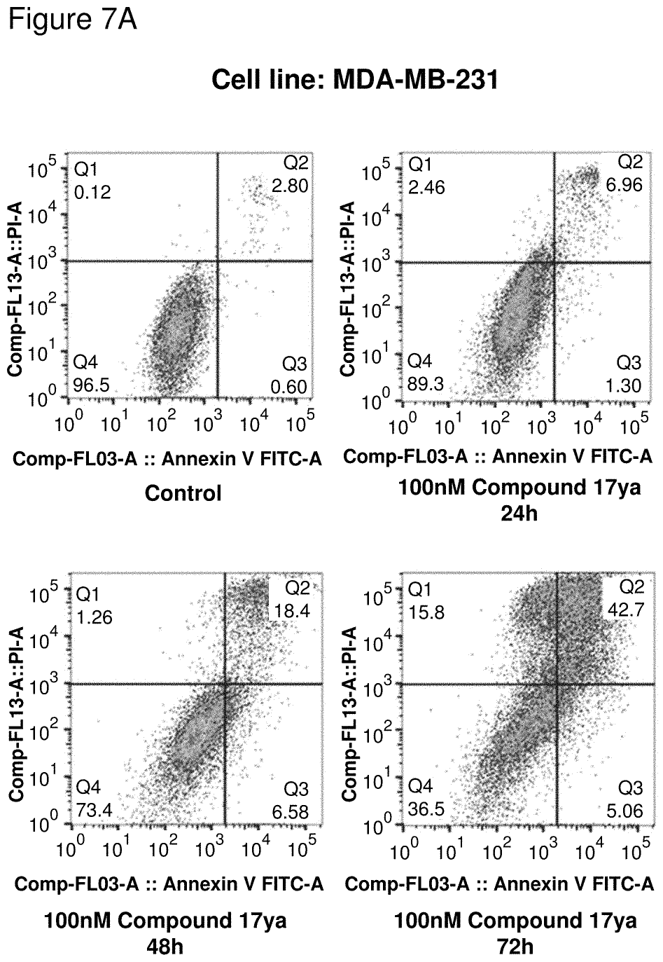

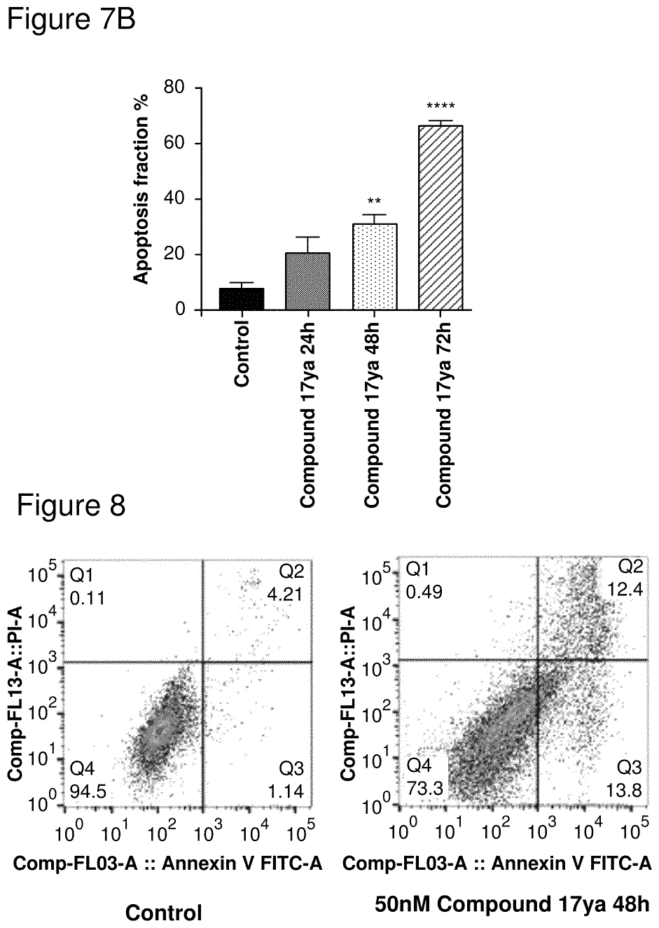

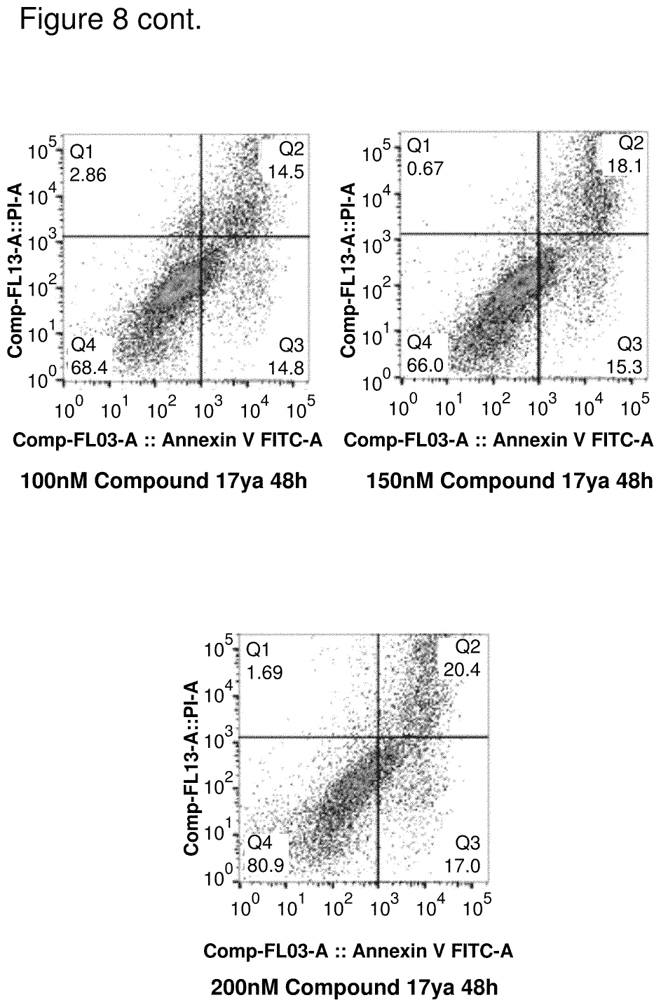

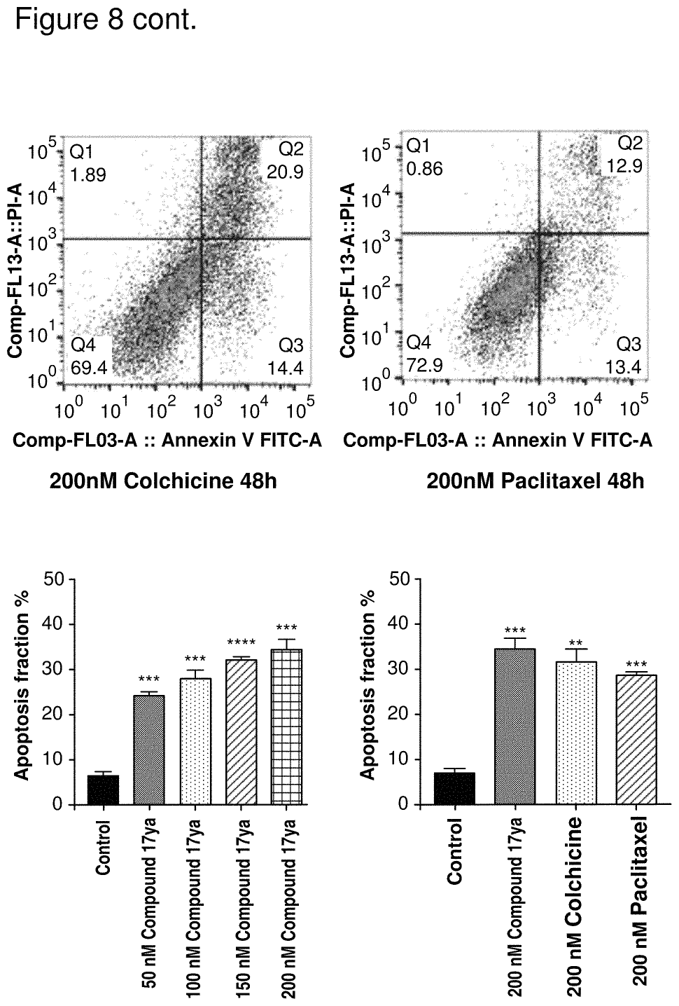

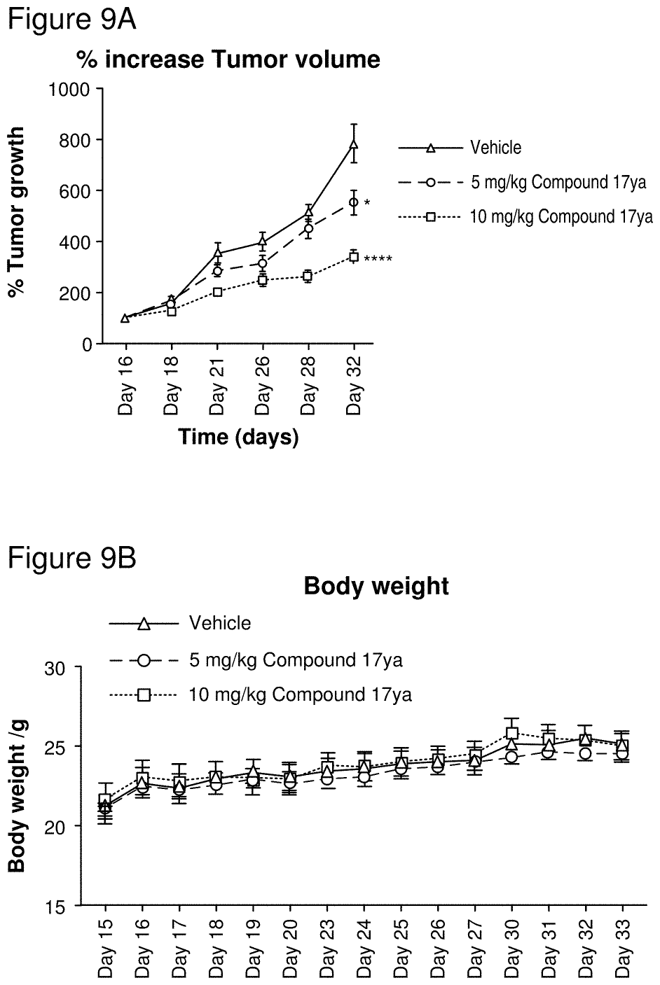

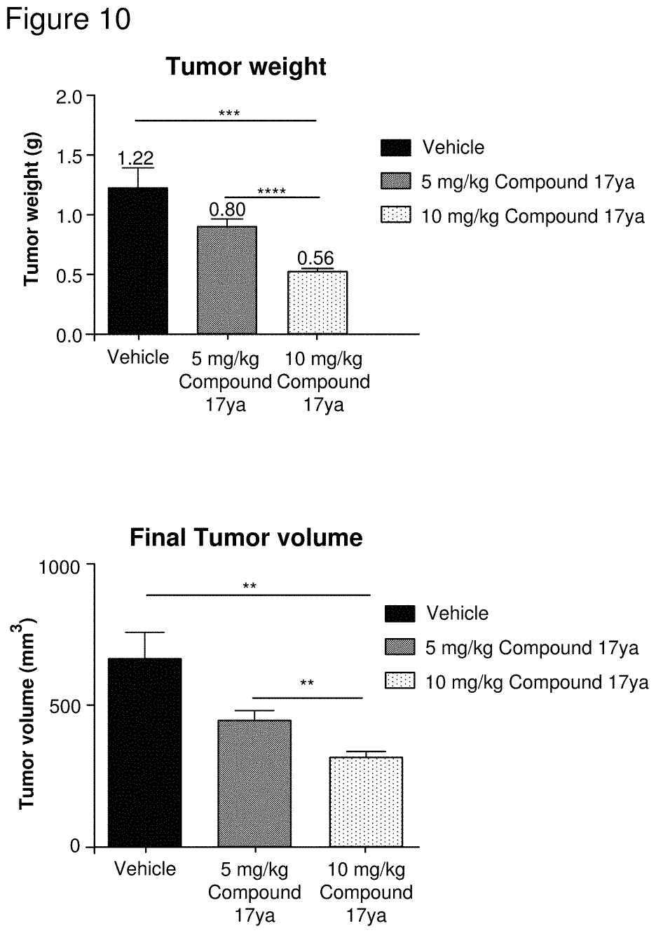

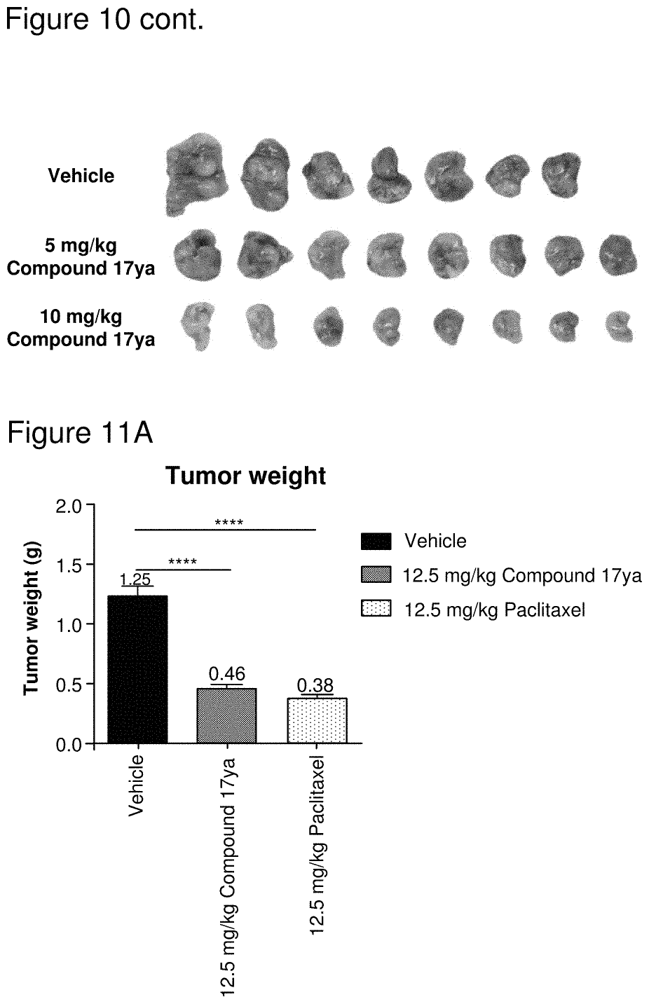

The cell apoptosis induction of compound 17ya on TNBC cells was also determined as compared against a control were 100 nM compound 17ya was studied at 24 hours, 48 hours, and 72 hours. See FIG. 7 for illustration. Compound 17ya induced TNBC cell apoptosis in a dose and time-dependent manner as compared to a control where compound 17ya was studied at 50 nm, 100 nM, 150 nM, and 200 nM for 48 hours and compared to colchicine (200 nM, 48 hours) and paclitaxel (200 nM, 48 hours). The results are illustrated in FIG. 8. The anti-cancer active of compound 17ya was studied in vivo at 5 mg/kg and 10 mg/kg and compared to a control and it was determined that compound 17ya inhibited TNBC tumor growth in a dose dependent manner without interfering with body weight. FIG. 9 illustrates the percent tumor growth and body weight (g) comparison of compound 17ya at 5 mg/kg and 10 mg/kg to a control. FIG. 10 illustrates the size comparison of tumors as compound 17ya inhibited TNBC tumor growth in a dose dependent manner.

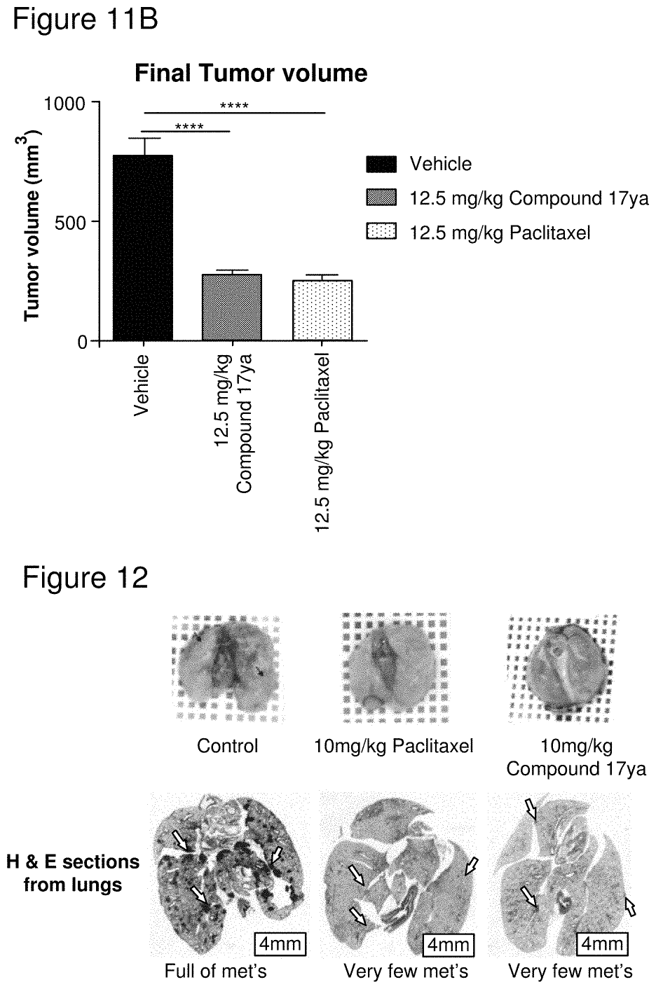

The anti-cancer activity of compound 17ya was compared against paclitaxel. It was determined that compound 17ya inhibited TNBC tumor growth significantly over the control and similarly to paclitaxel treatment. FIG. 11 illustrates the graphical comparison between vehicle, compound 17ya (12.5 g/kg), and paclitaxel (12.5 g/kg) effect on tumor weight (g) and final tumor volume (mm³).

The anti-metastatic activity of compound 17ya in vivo was also studied. The activity of compound 17ya (10 mg/kg) was compared to a control and paclitaxel (10 mg/kg) in H 7 E sections from lungs. FIG. 12 illustrates the results of this study, where compound 17ya resulted in very few metastases similarly to paclitaxel but in contrast to the control that had many metastases.

The efficacy of compound 17ya in vitro was determined using an orthotopic ovarian cancer mouse model. Two weeks after transplantation of SKOV3 cells into the left-side ovaries in NSG female mice, the mice were treated with vehicle or compound 17ya (10 mg/kg) orally for 4 weeks (5 treatments per week). To test the activity of compound 17ya in SKOV3 and OVCAR3 cells, cell survival ability was studied by performing colony formation assay. Cell migration and invasion were examined by using a modified transwell chamber. Precoated matrigel on the transwell inserts were used to test cells invasion ability.

Treatment with compound 17ya significantly inhibited SKOV3 ovarian tumor growth and metastasis to major organs (liver and spleen) in vivo, compared with the vehicle control (Table 1). Upon treatment with compound 17ya for 2 weeks at 10 nM or 30 nM concentration, cell growth in both SKOV3 and OVCAR3 was significantly reduced. Consistent with this finding, abilities of ovarian cancer cell migration and invasion were substantially inhibited with 20 nM of compound 17ya treatment in both SKOV3 and OVCAR3 cells. The results are summarized in the following Table 1:

TABLE 1

| | | | % | % |
|---|---|---|---|---|
| | | | Inhibition | Increase |
| | | Compound | (vs. | (vs. |
| Catalogue | Vehicle | 17ya | control) | control) |
| Tumor | 175.8 ± 35.9 mg | 24.4 ± 7.2 mg | 86.10 | |
| weight | | | | |
| Mice weight | 1.86 ± 0.65 g | 2.46 ± 1.32 g | | 70.96 |
| alternation | | | | |
| Liver | 5/5* | 0/5 | | |
| metastases | | | | |
| Spleen | 3/5* | 0/5* | | |
| metastases | | | | |

*Note:
for these dataset, the denominator is the number of tested mice, the numerator is the number of metastasized mice.

The test results demonstrated that orally available compound 17ya effectively inhibited tumor growth and metastasis in orthotopic ovarian cancer mouse model without acute toxicity and reduce ovarian cancer cells survival, migration and invasion abilities and concluding that compound 17ya is a tubulin inhibitor for the treatment of ovarian cancer.

In one embodiment, this invention provides methods for treating triple negative breast cancer and/or ovarian cancer comprising administering to the subject at least one compound described above and/or an isomer, metabolite, pharmaceutically acceptable salt, pharmaceutical product, tautomer, hydrate, N-oxide, polymorph, or crystal of said compound, or any combination thereof in a therapeutically effective amount to treat the triple negative breast cancer.

The invention encompasses a method of treating a subject suffering from triple negative breast cancer and/or ovarian cancer comprising administering to the subject at least one compound described above, or its isomer, metabolite, pharmaceutically acceptable salt, pharmaceutical product, tautomer, hydrate, N-oxide, polymorph, crystal or any combination thereof in an amount effective to treat triple negative breast cancer in the subject. In another embodiment, the compound is compound 12db. In another embodiment, the compound is compound 11cb. In another embodiment, the compound is compound 11fb. In another embodiment, the compound is compound 12da. In another embodiment, the compound is compound 12fa. In another embodiment, the compound is compound 12fb. In another embodiment, the compound is compound 12cb. In another embodiment, the compound is compound 55. In another embodiment, the compound is compound 6b. In another embodiment, the compound is compound 17ya.

A still further aspect of the present invention relates to a method of treating or preventing a cancerous condition that includes: providing at least one compound described above and then administering an effective amount of the compound to a patient in a manner effective to treat or prevent a cancerous condition.

According to one embodiment, the patient to be treated is characterized by the presence of a precancerous condition, and the administering of the compound is effective to prevent development of the precancerous condition into the cancerous condition. This can occur by destroying the precancerous cell prior to or concurrent with its further development into a cancerous state.

According to another embodiment, the patient to be treated is characterized by the presence of a cancerous condition, and the administering of the compound is effective either to cause regression of the cancerous condition or to inhibit growth of the cancerous condition, i.e., stopping its growth altogether or reducing its rate of growth. This preferably occurs by destroying cancer cells, regardless of their location in the patient body. That is, whether the cancer cells are located at a primary tumor site or whether the cancer cells have metastasized and created secondary tumors within the patient body.

As used herein, subject or patient refers to any mammalian patient, including without limitation, humans and other primates, dogs, cats, horses, cows, sheep, pigs, rats, mice, and other rodents. In one embodiment, the subject is male. In another embodiment, the subject is female. In some embodiments, while the methods as described herein may be useful for treating either males or females.

When administering the compounds, they can be administered systemically or, alternatively, they can be administered directly to a specific site where cancer cells or precancerous cells are present. Thus, administering can be accomplished in any manner effective for delivering the compounds or the pharmaceutical compositions to the cancer cells or precancerous cells. Exemplary modes of administration include, without limitation, administering the compounds or compositions orally, topically, transdermally, parenterally, subcutaneously, intravenously, intramuscularly, intraperitoneally, by intranasal instillation, by intracavitary or intravesical instillation, intraocularly, intraarterially, intralesionally, or by application to mucous membranes, such as, that of the nose, throat, and bronchial tubes.

The method encompasses administering at least one compound in combination with an anti-cancer agent by administering the compounds as herein described, alone or in combination with other agents.

When the compounds or pharmaceutical compositions of the present invention are administered to treat, suppress, reduce the severity, reduce the risk, or inhibit a cancerous condition, the pharmaceutical composition can also contain, or can be administered in conjunction with, other therapeutic agents or treatment regimen presently known or hereafter developed for the treatment of various types of cancer. Examples of other therapeutic agents or treatment regimen include, without limitation, radiation therapy, immunotherapy, chemotherapy, surgical intervention, and combinations thereof.

The following examples are presented to more fully illustrate the preferred embodiments of the invention. They should in no way, however, be construed as limiting the broad scope of the invention.

### EXAMPLES

The Examples set forth below are for illustrative purposes only and are not intended to limit, in any way, the scope of the present invention.

### Materials and Methods:

Cell culture. Ovarian cancer cell lines, SKOV3 and OVCAR3 were obtained from ATCC (American Type Culture Collection, Manassas, Va., USA) and cultured in Dulbecco's Modified Eagle Medium (DMEM) supplemented with 10% FBS (MIDSCI; St. Louis, USA), 100 U/ml penicillin, and 100 μg/ml streptomycin (Invitrogen; Carlsbad, Calif.). Cells were cultured in 5% carbon dioxide (CO₂) and 37° C. incubator.

General.

All reagents were purchased from Sigma-Aldrich Chemical Co., Fisher Scientific (Pittsburgh, Pa.), AK Scientific (Mountain View, Calif.), Oakwood Products (West Columbia, S.C.), etc. and were used without further purification. Moisture-sensitive reactions were carried under an argon atmosphere. ABT-751 was prepared according methods reported by Yoshino et al.²⁶ Routine thin layer chromatography (TLC) was performed on aluminum backed Uniplates (Analtech, Newark, Del.). Melting points were measured with Fisher-Johns melting point apparatus (uncorrected). NMR spectra were obtained on a Bruker AX 300 (Billerica, Mass.) spectrometer or Varian Inova-500 (Vernon Hills, Ill.) spectrometer. Chemical shifts are reported as parts per million (ppm) relative to TMS in CDCl₃. Mass spectral data was collected on a Bruker ESQUIRE electrospray/ion trap instrument in positive and negative ion modes. Elemental analyses were performed by Atlantic Microlab Inc.

### Example 1

### Ovarian Cancer Tumor Growth Suppression

Cell clonogenic survival assay. 350 SKOV3 or OVCAR3 cells were seeded on 6-well plates and cultured for 3 days with DMEM containing 10% FBS (i.e., growth media). On the third day of culture, the media was replaced with fresh growth media containing varying concentrations of compound 17ya ranging from 0, 1.25, 2.5, 5, 10 and 30 nM. The media was replaced every 3 days with fresh growth media containing compound 17ya until the 13th day of culture. Cells were then fixed with 70% ethanol and stained with crystal violet. Colonies from triplicate wells were counted for statistical analysis.

Cell migration assay. The cell migration assay was performed using a modified transwell chamber (BD FALCON, San Jose, Calif.). The chambers were inserted into 24-well cell culture plates. 3×104 SKOV3 or OVCAR3 cells with compound 17ya (20 nM) and vehicle treatment in 300 μl serum-free DMEM were added to the upper chamber. DMEM containing 10% FBS (serving as the chemoattractant) was added into the lower chamber of each well and incubated for 8 h. The medium and nonmigrated cells in the upper chamber were removed, while the migrated cells on the lower side of the membranes were fixed with methanol and stained with crystal violet. Pictures were taken at 10× magnification, and cells from at least three different fields were counted.

Cell invasion assay. SKOV3 or OVCAR3 (5×10⁵) cells with compound 17ya (20 nM) and vehicle treatment were seeded in 300 μl serum-free DMEM onto inserts precoated with Matrigel (BD BIOCOAT using 24-well Tumor Invasion System (BD BioSciences, San Jose, Calif.). DMEM containing 10% FBS was added to the bottom chamber of the invasion system as the chemoattractant and incubated for 24 h. The medium and nonmigrated cells in the upper chamber were removed, while the migrated cells on the lower side of the membranes were fixed with methanol and stained for 5 min with hematoxylin and eosin. Pictures were taken at 10× magnification. Invaded cells were counted in at least three different fields.

Orthotopic ovarian cancer mouse model. 5×105 SKOV3 cells labeled with a lentiviral vector expressing luciferase (pLenti-UBC-Luc2-T2A-mKate) were intrabursally injected into 10 two-month old NOD.Cg-Prkdcscid Il2rgtmlWjl/SzJ severely immunocompromised female mice (NSG). Two weeks after transplantation of SKOV3 cells into the left-side ovaries in NSG female mice, the mice were treated with vehicle or compound 17ya (10 mg/kg) orally for 4 weeks (5 treatments per week). Five mice were used for each group. Tumor initiation and metastasis were monitored once a week using a Xenogen imaging system. All mice were sacrificed at two months; the ovaries and metastasized organs were harvested and imaged using Xenogen imaging system; the tumors were weighed and tissues were processed for H&E staining.

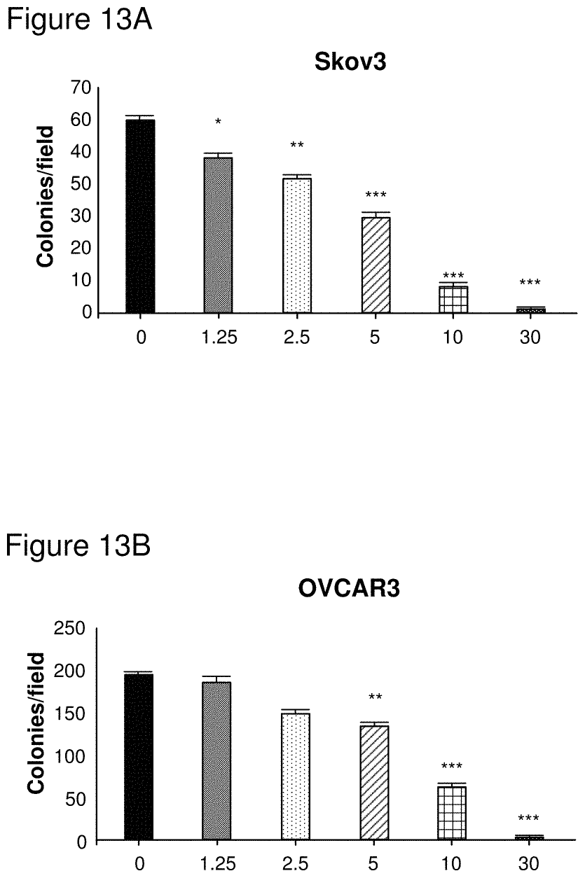

The treatment with compound 17ya inhibited ovarian cancer cell survival. To test the effect of compound 17ya on ovarian cancer cells, cell survival was examined by assaying cell colony formation, as described above. 350 SKOV3 and OVCAR3 ovarian cells were cultured in 6-well plates and treated with compound 17ya at six doses of: 0, 1.25, 2.5, 5, 10 and 30 nM. On the 13th day, the colonies were stained with crystal violet. Compound 17ya significantly inhibited cell survival in both SKOV3 and OVCAR3 cells as illustrated in FIG. 13 (**p<0.01, ***p<0.001).

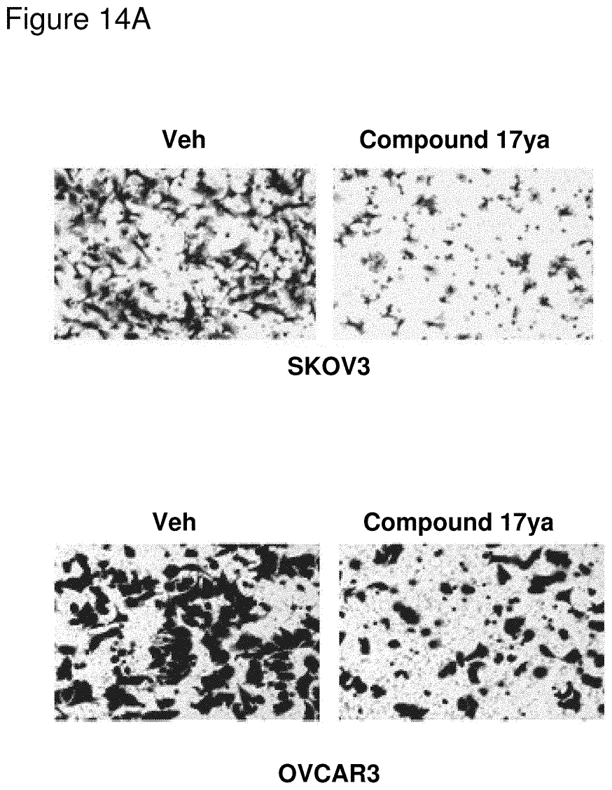

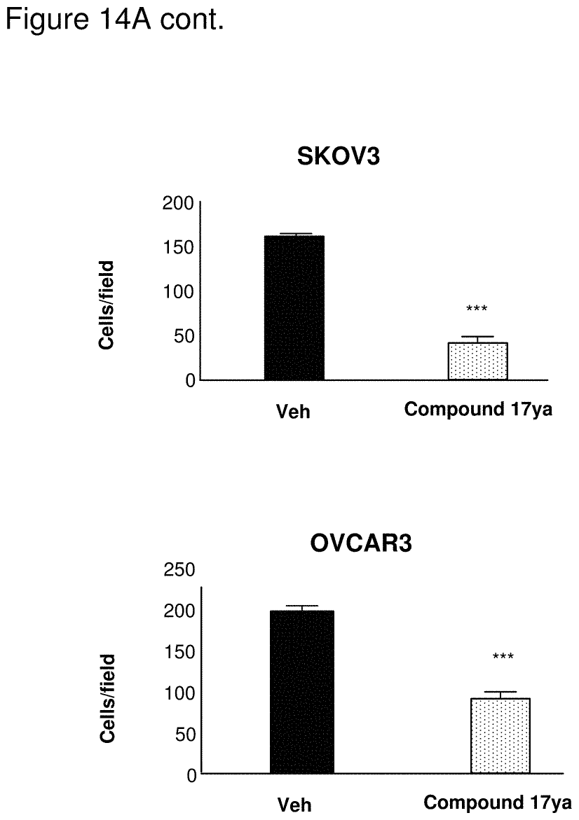

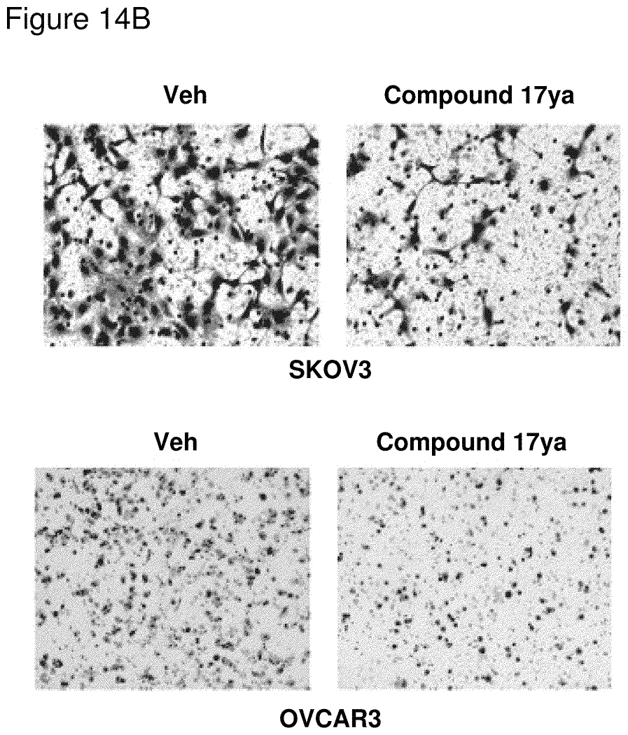

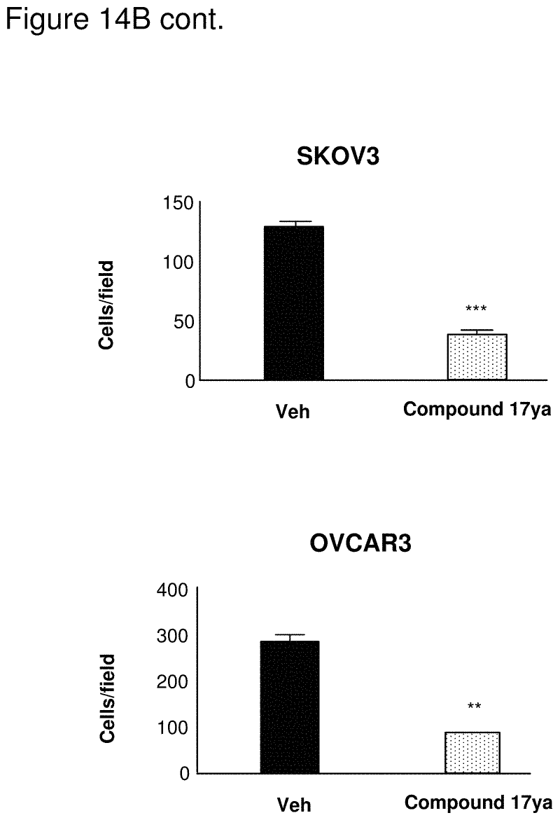

Treatment with compound 17ya inhibited ovarian cancer cells migration and invasion. Using transwell plates, cell migration was tested against compound 17ya and vehicle treatment cells. It was found that cell migration was significantly reduced in both SKOV3 and OVCAR3 cells with compound 17ya as illustrated in FIG. 14A. Cell invasion was assessed using Matrigel-coated transwells. Treatment with compound 17ya significantly reduced cells compared to the vehicle cells in both SKOV3 and OVCAR3, as illustrated in FIG. 14B.

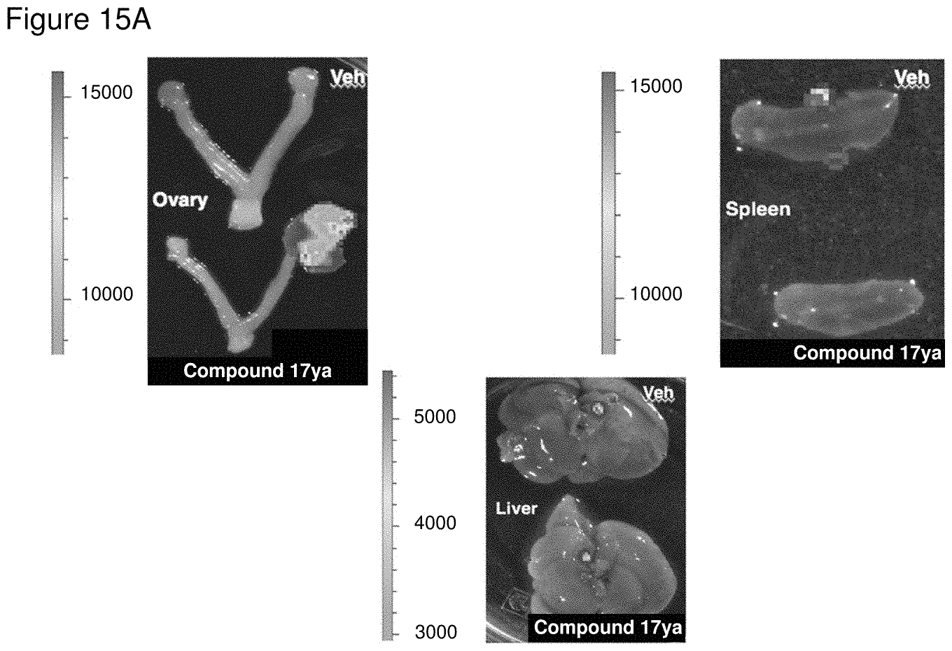

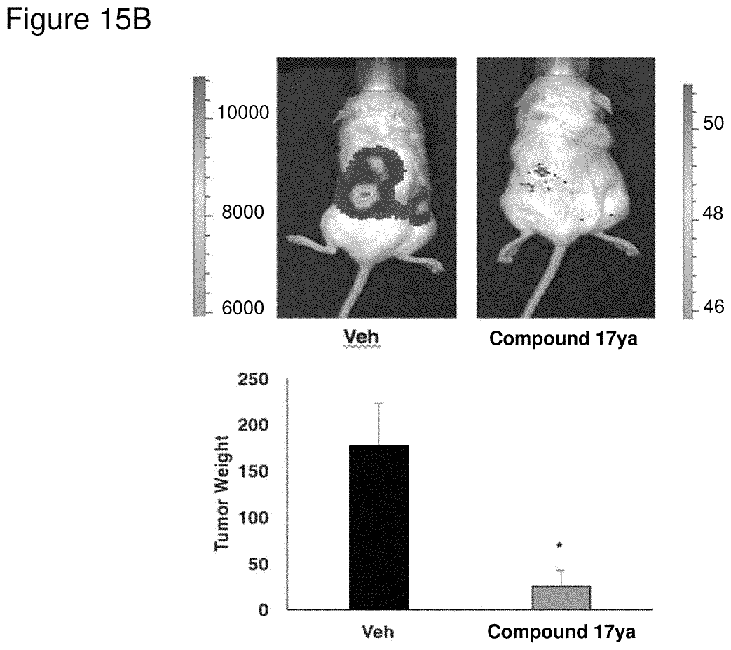

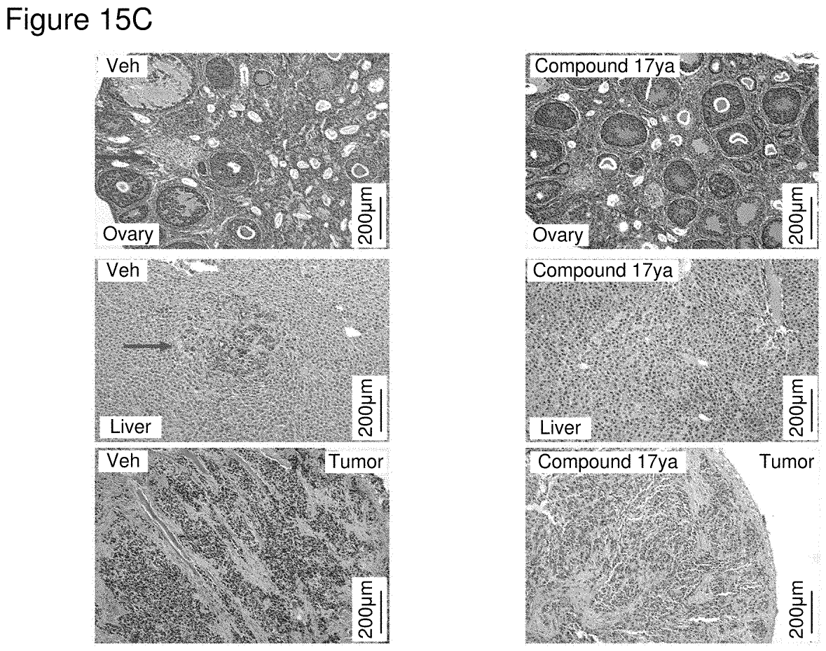

Compound 17ya inhibited ovarian tumor growth and metastasis in vivo. An orthotopic ovarian cancer mouse model was established. 5×105 Wildtype SKOV3-Luc2 cells were intrabursally injected into 2 month old NSG female mice and mice were treated five days a week for 4 weeks. Tumors were collected and imaged using Xenogen system. SKOV3 cells transduced with a lentiviral luciferase vector were injected into the left-side ovaries in two-month NSG mice, the mice were treated with vehicle or compound 17ya (10 mg/kg) for 4 weeks. Treatment with compound 17ya significantly inhibited SKOV3 ovarian tumor growth as illustrated in FIG. 15B. Treating with compound 17ya inhibited metastasis to major organs (liver and spleen) in vivo as compared to the vehicle control as illustrated in FIG. 15A. The H.E staining on ovaries, tumors and livers showed that treatment with compound 17ya inhibited ovarian tumor growth and metastasis, as illustrated in FIG. 15C. Tumors were not visible in ovaries, liver and spleen of mice treated with Compound 17ya.

### Example 2

Materials and Methods

Chemical compounds and cell lines. Colchicine was purchased from Sigma (St. Louse, Mo.). Paclitaxel was purchased from LC Laboratories (Woburn, Mass.). Compound 17ya was synthesized as described (Chen et al., “Discovery of novel 2-aryl-4-benzoyl-imidazole (ABI-III) analogues targeting tubulin polymerization as antiproliferative agents.” J. Med. Chem., 2012, 55. 7285-7289.), with purity (>98%) and identity verified by HPLC, HR-MS (Waters, Milford, Mass.) and proton nuclear magnetic resonance (Bruker, Billerica, Mass.). Two human triple negative breast cancer (TNBC) cell lines were used in this study: MDA-MB-231 and MDA-MB-468 purchased from ATCC (Manassas, Va.) and authenticated prior to use for this study. These cells were cultured in DMEM medium (Mediatech, Inc., Manassas, Va.) supplemented with 10% fetal bovine serum (Atlanta Biologicals, Lawrenceville, Ga.) and 1% antibiotic-antimycotic Solution (Sigma-Aldrich, St. Louis, Mo.) at 37° C. in a humidified atmosphere containing 5% CO₂.

Cell viability assay. The anti-proliferative effect of compound 17ya was investigated in human melanoma (A375 and M14), human HER2-positive breast (MDA-MB-453 and SKBR3) and TNBC (MDA-MB-231 and MDA-MB-468) cancer cell lines using routine MTS assay as described by Li et al., “A Potent, Metabolically Stable Tubulin Inhibitor Targets Colchicine Binding Site and Overcomes Taxane Resistance,” Cancer Res., 2018, 78, 265-277. IC₅₀ (50% of cell growth inhibition) values were calculated by GraphPad Prism 7 software using nonlinear regression.

Colony formation assay. MDA-MB-231 or MDA-MB-468 cells were seeded into 12-well plates with cell density of 200 cells/well and incubated for 24 h. Cells were then treated with colchicine, paclitaxel and Compound 17ya at different concentrations. The medium of each group was renewed once a week. After culture for 7 days (MDA-MB-231) and 14 days (MDA-MB-468), the cells were washed with PBS, fixed with methanol and stained with 0.5% crystal violet. The morphology of colonies was captured under a microscope and colony area was quantified using ImageJ software (NIH, Bethesda, Md.). The drug treatment was performed in triplicate.

Caspase 3/7 activity assay. Apoptosis induced by compound 17ya was measured using Caspase Glo 3/7 assay system (Promega, Madison Wis.) according to the manufacturer's instructions as described in Li et al., “Design, Synthesis and Structure-Activity Relationship Studies of Novel Surviving Inhibitors with Potent Anti-Proliferative Properties,” PLoS One, 2015, 10, e0129807. 5000 cells were seeded in each well of a 96-well plate and treated with compound 17ya 20 nM for 24 h in triplicate. The caspase 3/7 activity was normalized by total protein content in each sample.

Cell migration and invasion assay. Chemotactic cell migration was carried out using Transwell 96-well plate contained a membrane insert (pore size 8 μm) and a tray (BD Biosciences, CA), and the effect of compound 17ya on cell invasion was performed using matrigel invasion chamber (Corning, N.Y.). In both assays, MDA-MB-231 and MDA-MB-468 cells in serum-free medium were starved for 24 h, followed by suspending the cells in serum-free medium containing 16 nM colchicine and compound 17ya and plating them in the top chamber of the membrane insert or matrigel-coated membrane in triplicate. Medium containing serum was added in the lower chamber as a chemoattractant. After 24 h incubation for MDA-MB-231 cells and 48 h incubation for MDA-MB-468 cells, the cells that did not migrate through the membrane or invade through the matrigel were removed by cotton swabs while the cells that had migrated or invaded to the bottom surface of the chamber were fixed in 4% buffered formalin phosphate solution, stained with 0.5% crystal violet solution and imaged by a microscope. The number of cells migrated or invaded were counted manually using ImageJ software.

Cell migration was also analyzed by the scratch assay. Briefly, MDA-MB-231 cells (10⁵ cells/well) and MDA-MB-468 (2×10⁵ cells/well) cells were seeded in 12-well plates and incubated overnight. The following day, a scratch was made in the cell monolayer by using a sterile 200-μl pipette tip. After washing away the floating cells, the cell culture medium was replaced by medium containing vehicle DMSO, colchicine, paclitaxel or compound 17ya at specific concentration (e.g., 16 nM). After 12 h, 24 h and 48 h, the wound width was determined and imaged with Evos Fl Imaging System (Life Technologies, Carlsbad, Calif.). The extent of wound closure was shown as the percentage of decrease of the original scratch width at each measured time point. Experiments were done in triplicate.

Immunofluorescence staining. 10⁵ MDA-MB-231 cells or 2×10⁵ MDA-MB-468 cells were seeded in 6-well plates on sterile coverslips for 24 h prior to treatment with 32 nM colchicine, paclitaxel and compound 17ya for 18 h. For tubulin visualization, cells were washed three times with PBS, fixed with 4% paraformaldehyde for 15 min and permeabilized with 0.2% Triton X-100 in PBS for 15 min. Then the cells were blocked in 1.5% bovine serum albumin (BSA), 0.1% Tween 20 in PBS for 1 h and incubated anti-α-tubulin antibody (Thermo Fisher Scientific, Waltham Mass.) in 1% bovine serum albumin (BSA), 0.1% Tween 20 in PBS overnight at 4° C. The following day, cells were washed and incubated with Alexa Fluor 647 goat anti-mouse IgG (Molecular Probes, Eugene Oreg.) in dark for 1 h at room temperature, followed by the addition of Prolong Diamond Antifade reagent with DAPI (Invitrogen, Carlsbad, Calif.) and subsequent mounting with slides. Images depicted in figures were obtained with a Keyence BZ-X700 microscope (Keyence, Osaka Japan).

Detection of apoptosis. MDA-MB-231 and MDA-MB-468 Cells were seeded in 6-well plates (2×10⁵/well). After incubating overnight, cells were treated with 100 nM compound 17ya for 24 h, 48 h and 72 h. A dose dependent investigation was carried out by treating cells with increasing doses for 48 h. Cells were then washed twice with PBS and 10⁵ cells were suspended in 200 ul Annexin V-FITC binding buffer (eBioscience, Grand Island, N.Y.). 185 μl cell suspension were added 5 μl Annexin V-FITC and 10 μl propidium iodide cell suspension, after mixture and incubation for 10 min at room temperature, the cells were analyzed by a Bio-Rad ZE5 cell analyzer (Bio-rad, Hercules, Calif.).

Cell Cycle Analysis, Western Blotting, In Vivo Orthotopic Xenograft Model.

All animal studies were carried out in adherence to the NIH Principles of Laboratory Animal Care and protocols approved by the Institutional Animal Care and Use Committee at the University of Tennessee Health Science Center. Female Nod-Scid-γ (NSG) mice at 5-6 weeks of age were housed in a specific pathogen free environment with a 12:12-hour light-dark cycle. Temperature was maintained at 20-26° C. and the relative humidity was maintained at 30-70%. 2.5×10⁵ MDA-MB-231 cells in 10 μl of HBSS were surgically inoculated into the left and right inguinal mammary gland fat pads of NSG mice as described by Pfeffer et al., “Comprehensive analysis of microRNA (miRNA) targets in breast cancer cells,” J. Biol. Chem., 2013, 288, 27480-27493, hereby incorporated by reference. Mice were inspected weekly for tumor appearance until the average tumor size reached 100 mm³. Then the mice were randomly divided into 3 groups (n=5 per group) and drug treatment was started. The control group was administered with vehicle (1:1 ratio of PEG 300:water) orally, drug treatment groups were administered 5 mg/kg compound 17ya or 10 mg/kg compound 17ya orally five times a week, respectively. Primary tumor size was monitored twice a week using a digital caliper and body weight of the mice was recorded during the treatment. The tumor volume was calculated using the formula volume=(width²×length)/2. After 33 days treatment, when the tumor size in the vehicle group reached 1000 mm³, tumors and major organs were imaged and collected in 10% buffered formalin phosphate solution for histological analysis. Another similar orthotopic xenograft model for comparing the efficacy of compound 17ya with paclitaxel was performed as described above. The mice were randomized into 3 groups with 8 mice per group. Identically, the control group was administered with vehicle (1:1 ratio of PEG 300:water) orally, paclitaxel group was administered 12.5 mg/kg paclitaxel via intraperitoneal injection every other day and compound 17ya group was administered 12.5 mg/kg compound 17ya orally five times a week, and tumors and major organs were collected in 10% buffered formalin phosphate solution for histological analysis when the tumor size in the vehicle group reached 1000 mm³.

Experimental lung metastasis model: 7-8-week-old NSG mice were used to investigate the efficacy of compound 17ya to inhibit the metastasis of TNBC. 2×10⁵ MDA-MB-231 cells in 100 μl of HBSS was inoculated into each mouse via tail vein injection. Vehicle, 10 mg/kg compound 17ya and 10 mg/kg paclitaxel treatments with the same dose frequency of the orthotopic xenograft model were started after 24 h. Animal health and body weight were monitored weekly during the treatment. After 23 days, the mice were sacrificed and all major organs were imaged and collected in 10% buffered formalin phosphate solution for subsequent histology and immunohistochemistry analysis.

Histology and immunohistochemistry (IHC) analysis: Fixed tumors and organs were embedded in paraffin and several section slides were cut for further Hematoxylin/eosin (H&E) staining and IHC staining. H&E staining and IHC staining were carried out as previously described. Primary antibodies used in IHC staining included rabbit anti-Ki67 (1:400), rabbit anti-CD31 (1:100), rabbit anti-cleaved Parp (1:50) and rabbit anti-cleaved caspase 3 (1:200) (#9027; #77699; #5625; #9661, Cell Signal Technology, Danvers Mass.), and biotinylated horse anti-rabbit IgG antibody (BA-1100, Vector Laboratories Inc., Burlingame, Calif.) was used as the secondary antibody. Anti-mitochondria IHC staining was performed to visualize the metastasis of MDA-MB-231 cells in experimental lung metastasis model. Images were acquired with a Keyence BZ-X700 microscope.

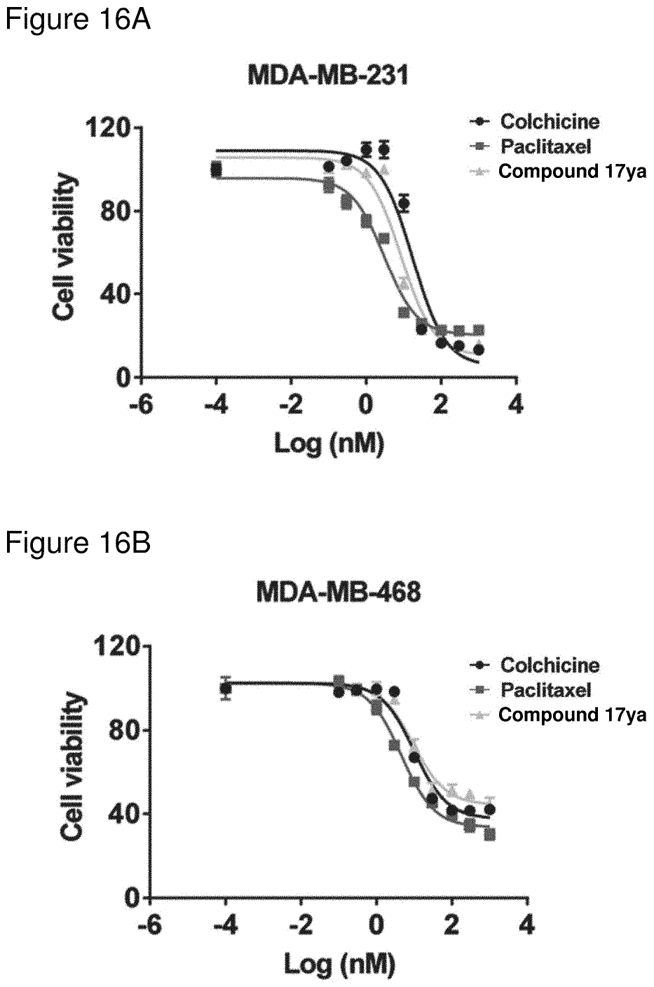

Compound 17ya extenuates the proliferation of different breast cancer cells. Compound 17ya previously was tested in a panel of melanoma cancer cell lines with an IC₅₀ of 10 nM as described by Li et al., “Discovery of novel 2-aryl-4-benzoyl-imidazole (ABI-III) analogues targeting tubulin polymerization as antiproliferative agents,” J. Med. Chem., 2012, 55 7285-7289. Compound 17ya was evaluated to determine whether it could also inhibit the growth of breast cancer cells using MTS assay. Table 2 showed that compound 17ya had anti-proliferative effect against breast cancer cell lines, especially in TNBC. The testing incorporated two well-known tubulin inhibitors, colchicine and paclitaxel, to compare the efficacy of compound 17ya against TNBC cell growth. The results of the test are illustrated in FIG. 16. All three tubulin inhibitors were potent in inhibiting the proliferation of TNBC. Colony formation assay is always used for assessing the cell proliferation by determining the colony growth from microcolonies to macrocolonies. The colony formation results showed that compound 17ya attenuated the capacity of proliferation of MDA-MB-231 and MDA-MB-468 cells in a dose-dependent manner.

TABLE 2
Compound 17ya extenuates proliferation of different breast cancers

| Tumor Type | Cell Line | IC₅₀ ± SEM (nM) |
|---|---|---|
| Melanoma | M14 | 8.86 ± 1.97 |
| | RPMI7951 | 8.76 ± 2.24 |
| Breast | MDA-MB-453 | 13.59 ± 2.42 |
| | SKBR3 | 13.29 ± 1.41 |
| | MDA-MB-231 | 8.23 ± 2.39 |
| | MDA-MB-468 | 9.59 ± 2.36 |

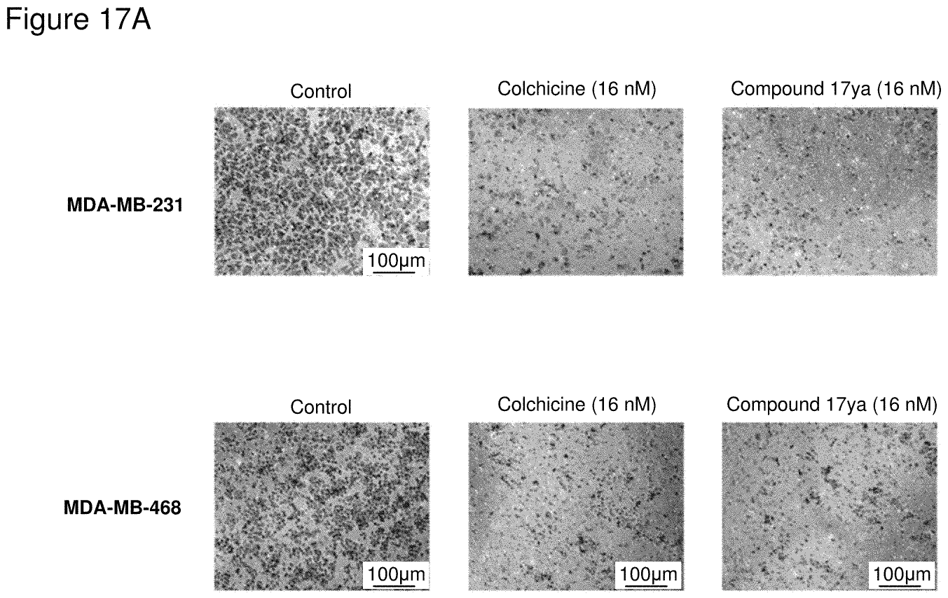

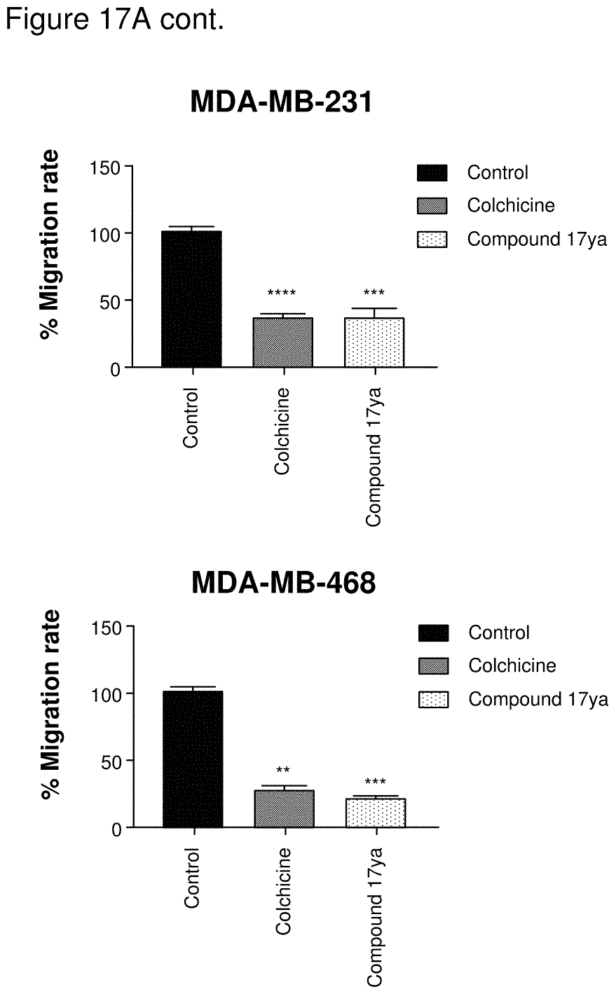

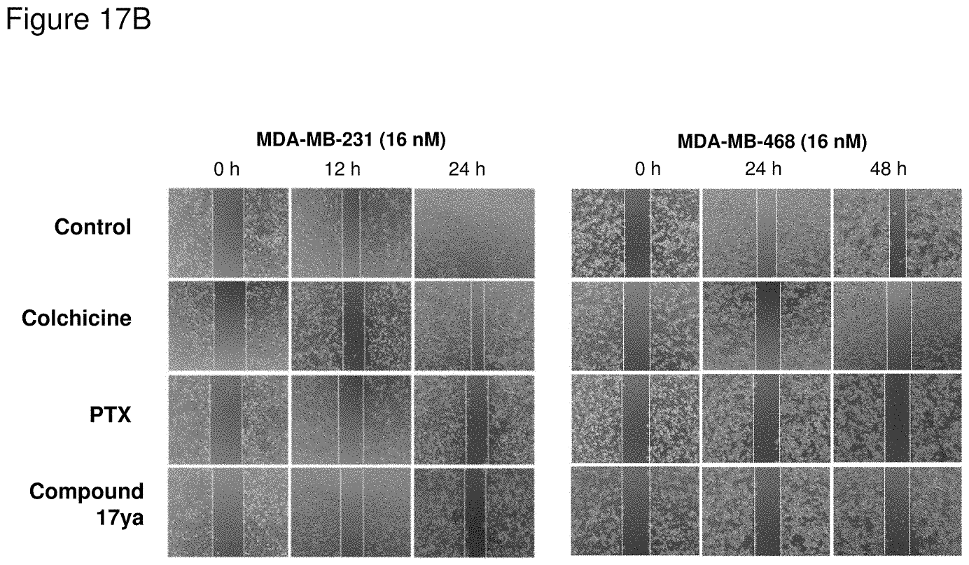

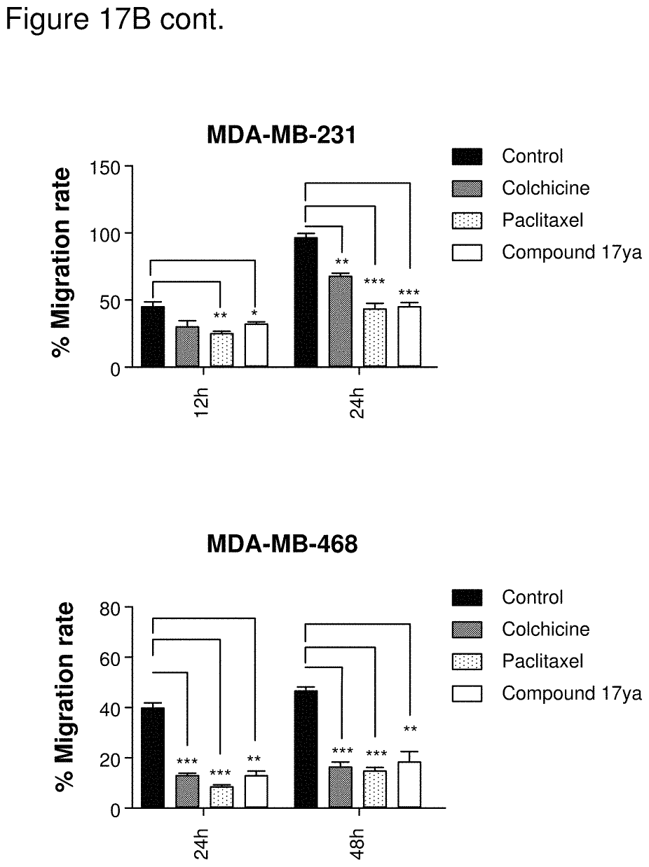

Compound 17ya inhibited TNBC cells migration and invasion. The effect of compound 17ya on the migration and invasion of TNBC cells after 24 or 48 hours of drug treatment was investigated. FIG. 17 illustrates the results. Compound 17ya inhibited the cells ability to migrate through a membrane insert in the presence of 16 nM concentration, showing the similar potency as of colchicine. Likewise, Compound 17ya reduced the TNBC cells capacity to invade through the matrigel-coated membrane. To further confirm these results, a scratch assay was performed using paclitaxel and colchicine as positive control. At a dose of 16 nM, Compound 17ya, colchicine and paclitaxel showed effective inhibition of cell migration. Based on these findings, we concluded that Compound 17ya suppressed cell migration significantly, emphasizing a potential role Compound 17ya plays in inhibiting the TNBC metastasis.

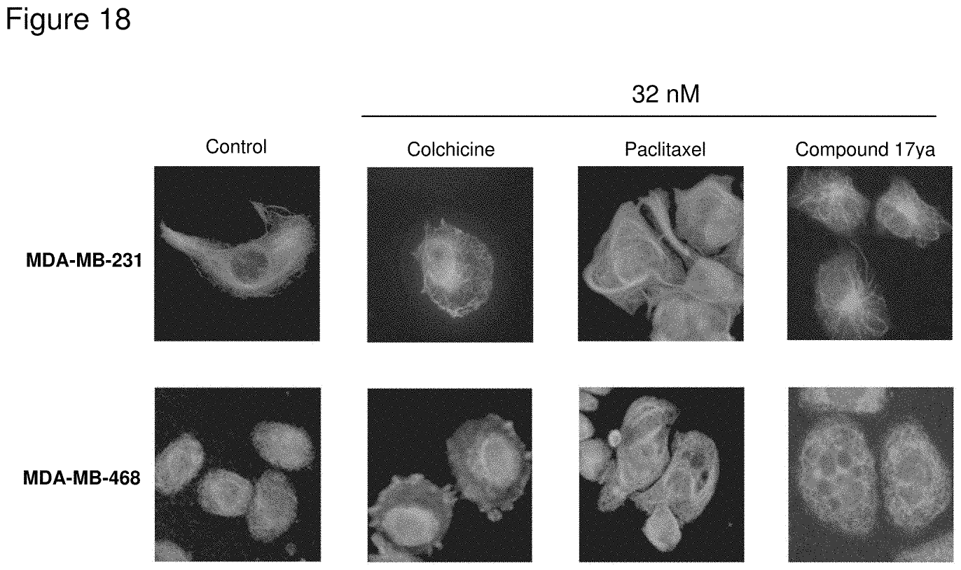

Compound 17ya interfered with microtubule assembly and mitotic spindle organization: Compound 17ya was used with immunofluorescence staining to visualize the microtubule network and compared with known microtubule-destabilizing agent colchicine and microtubule-stabilizing agent paclitaxel. TNBC cells in negative control group showed intact microtubule fibers and organization of microtubules. FIG. 18 illustrates these results. Treatment with paclitaxel resulted multipolar spindles with highly condensed chromosomes due to the enhance of tubulin polymerization in TNBC cells. Like the treatment of colchicine, cells treated with Compound 17ya had shrunken and the cell shape was changed from spindle to round and irregular, which confirmed that Compound 17ya targeted the tubulin and interfere the tubulin polymerization.

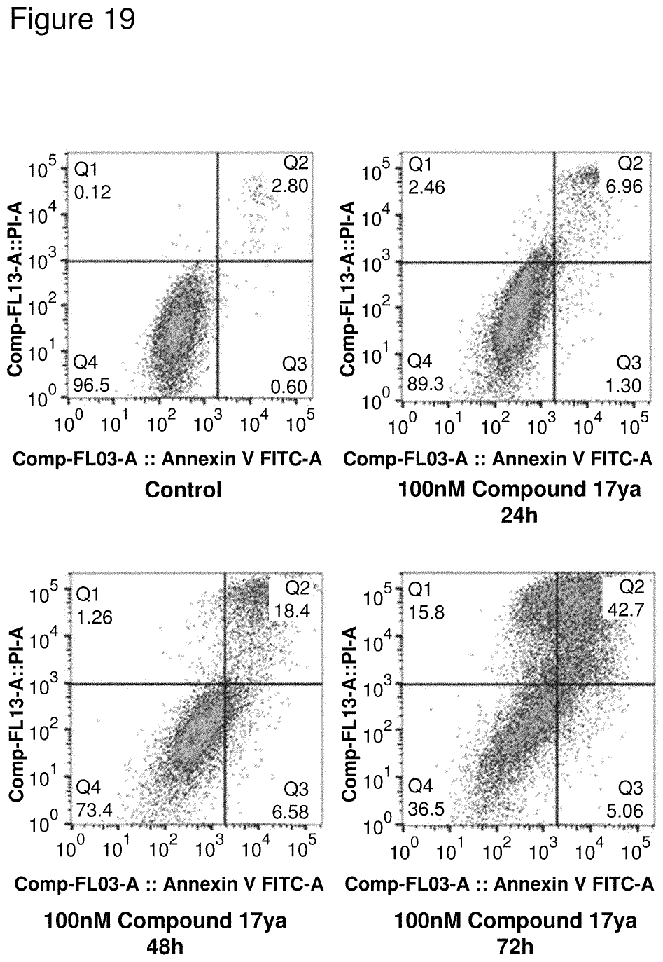

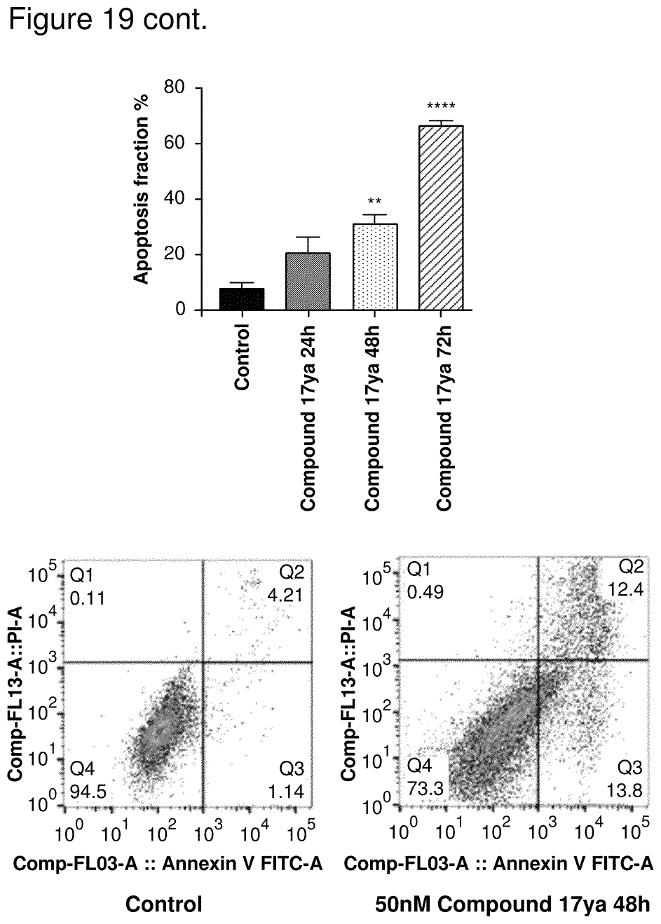

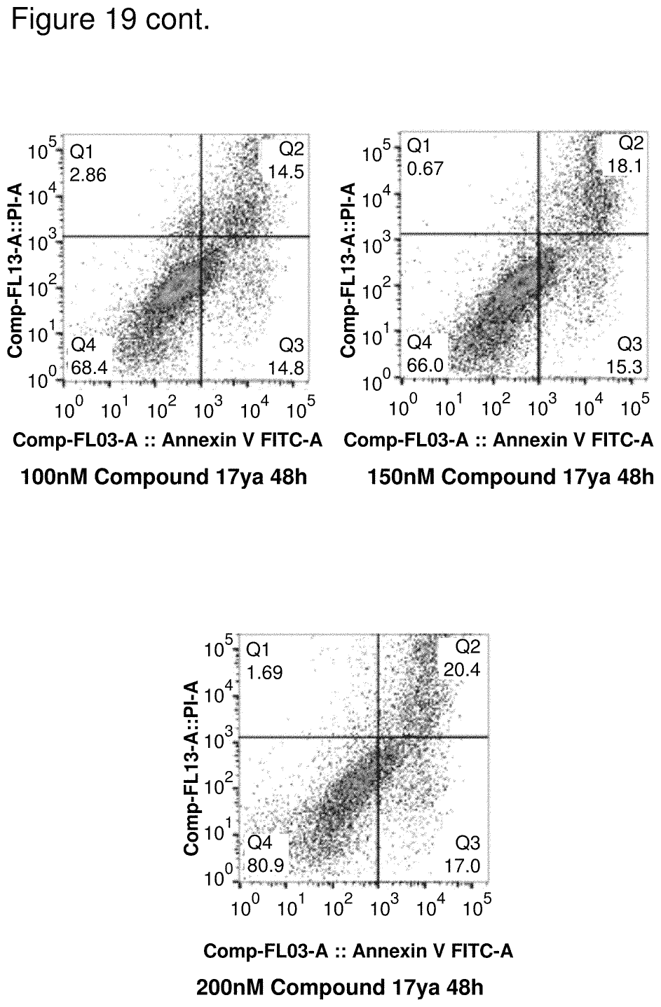

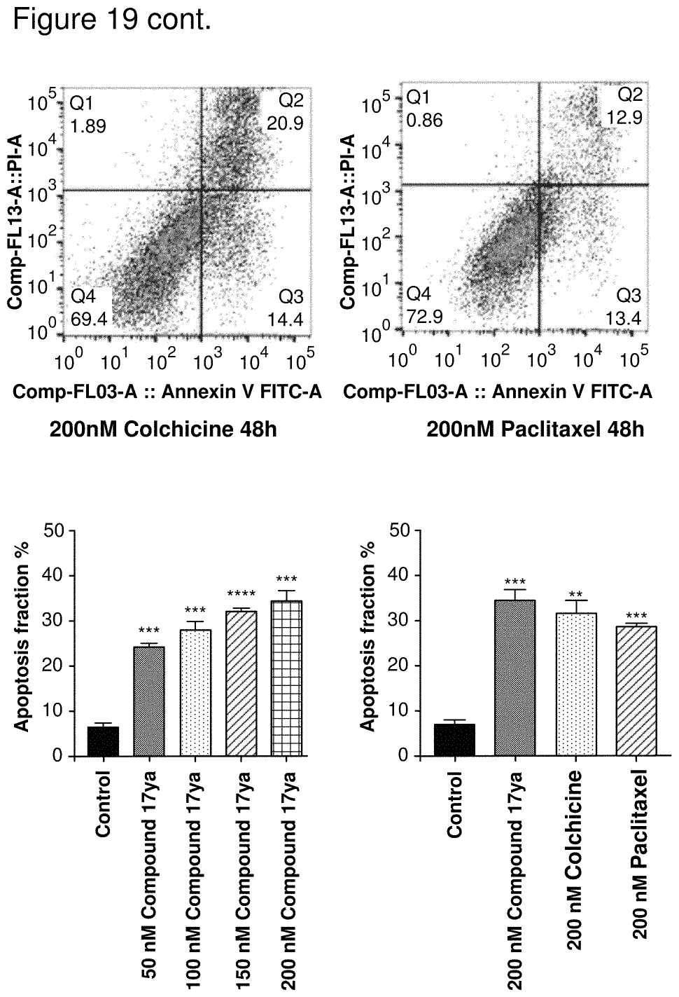

Compound 17ya induced increased apoptosis in TNBC cells: Since many tubulin inhibitors were reported to have pro-apoptotic effect on cancer cells, compound 17ya was studied to determine the effect on apoptosis induction in TNBC cells. MDA-MB-231 cells were treated with 100 nM Compound 17ya in a time-dependent manner. Compound 17ya induced the cells to apoptosis as illustrated in FIG. 19. Cells were treated with increasing concentrations of compound 17ya for 48 h. Compound 17ya initiated apoptotic cell death in a dose-dependent manner, and thus caused apoptosis of TNBC cells.

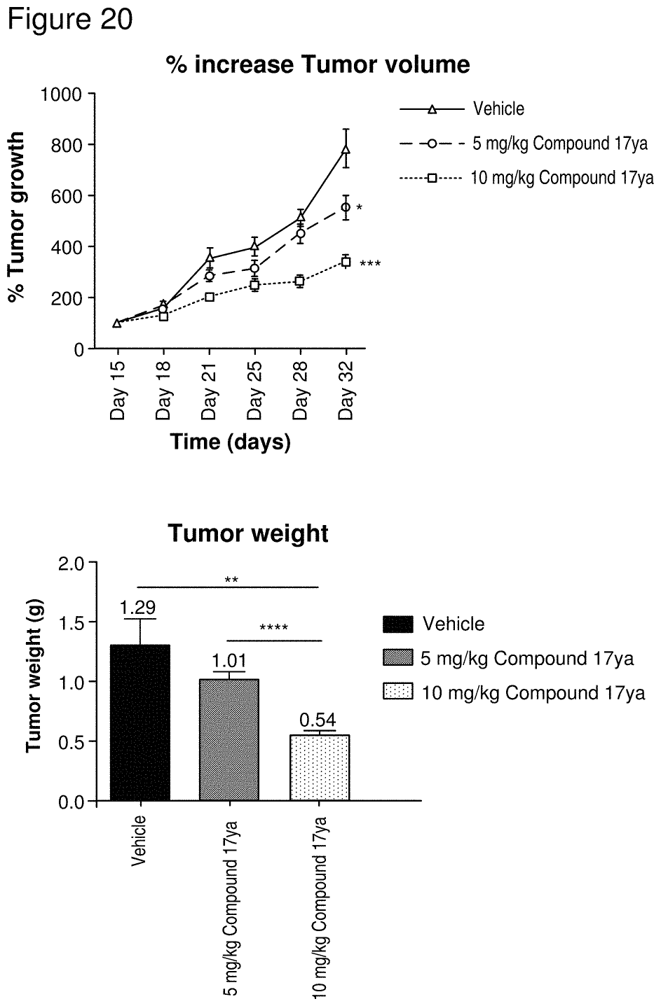

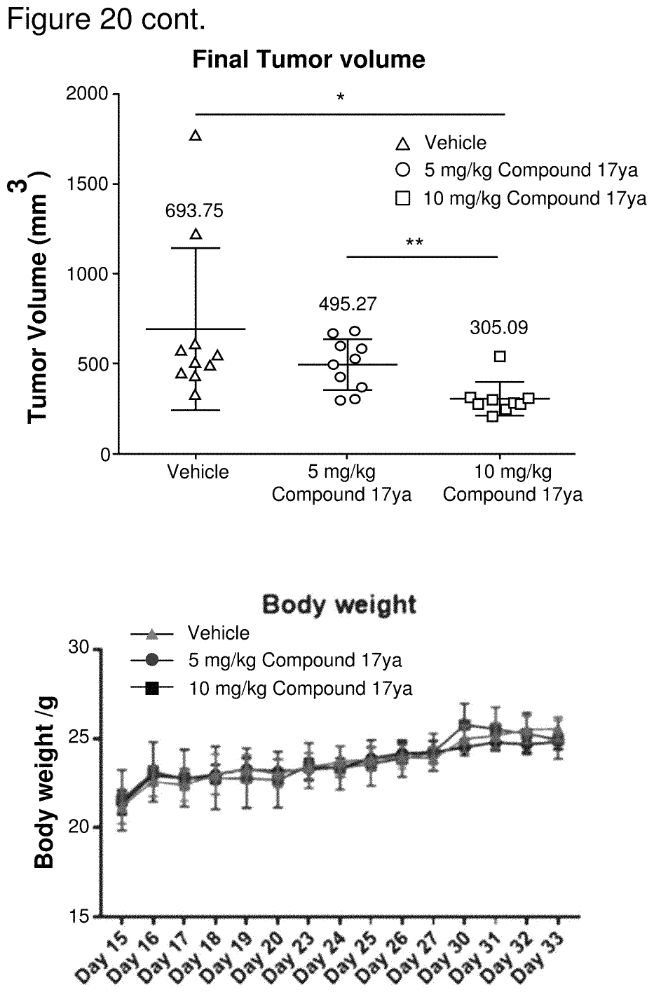

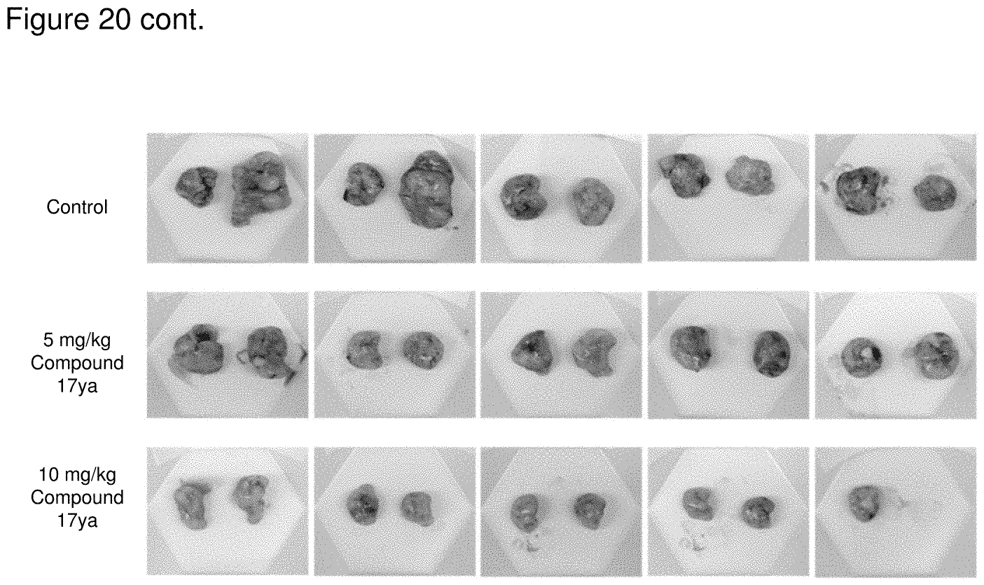

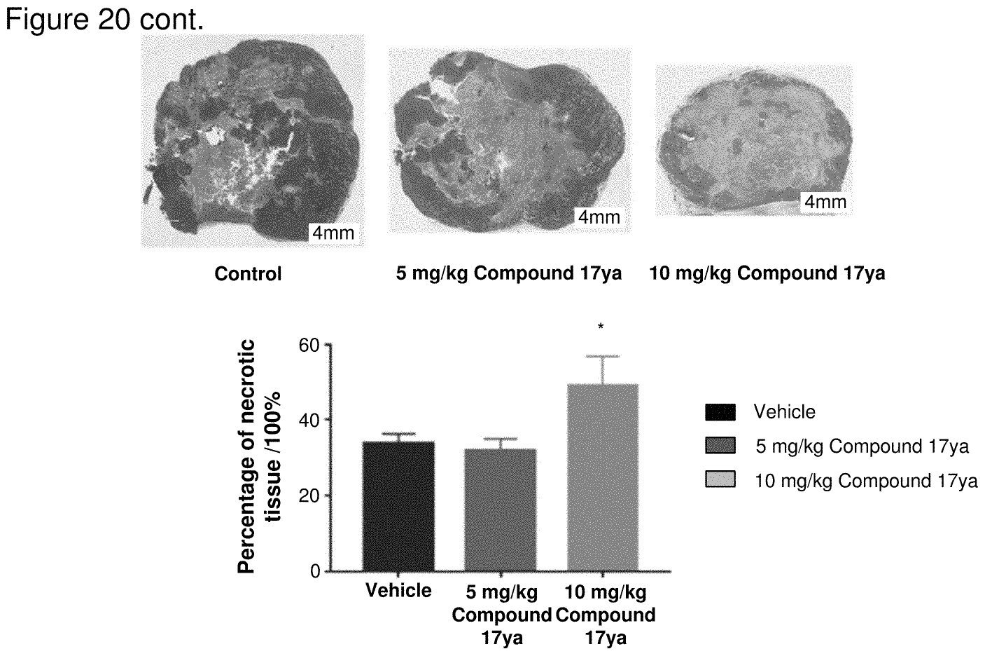

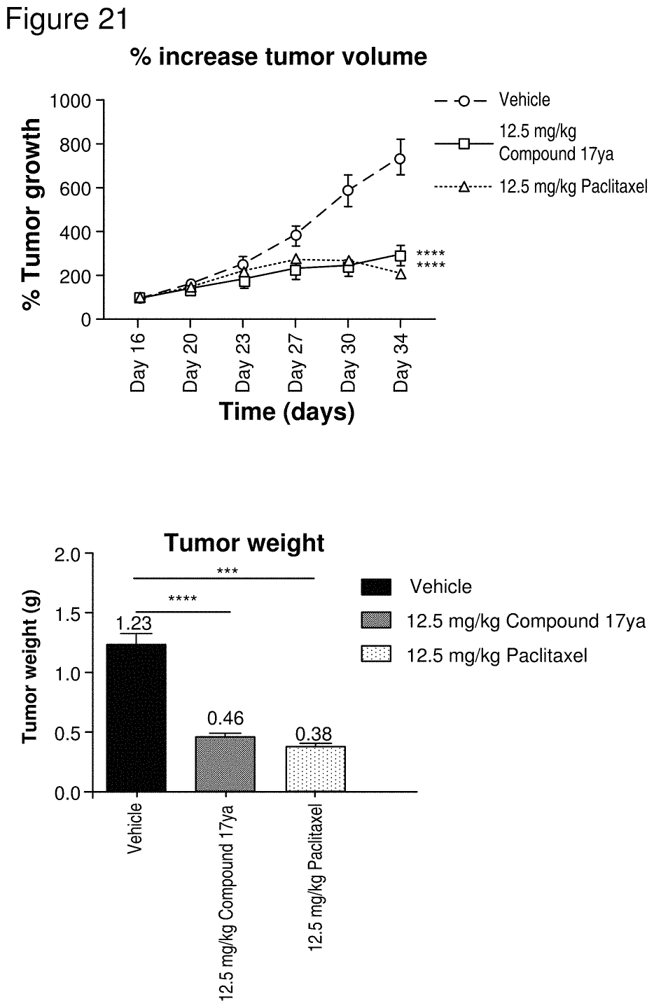

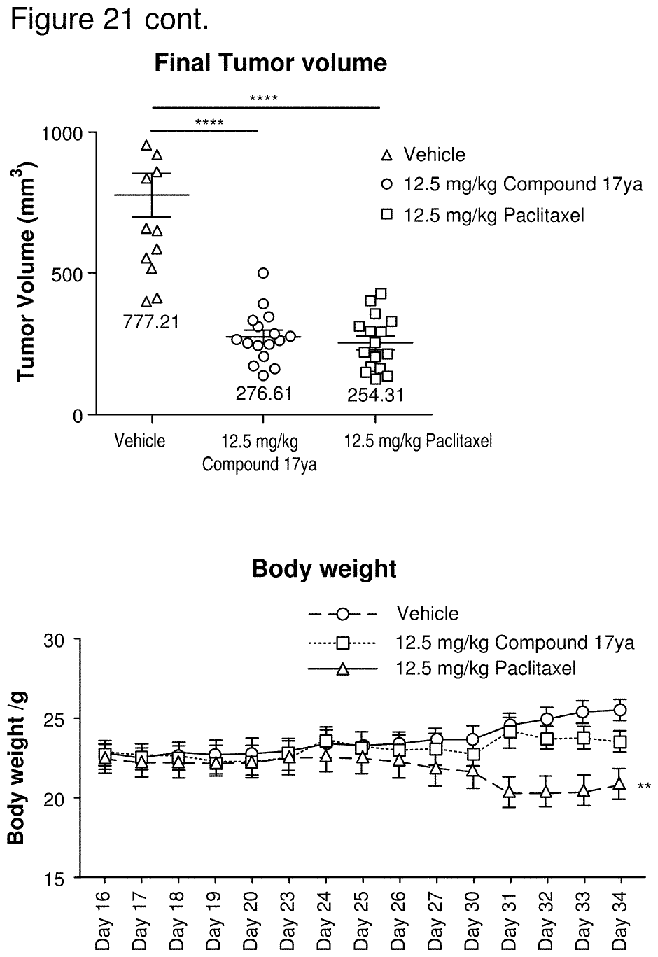

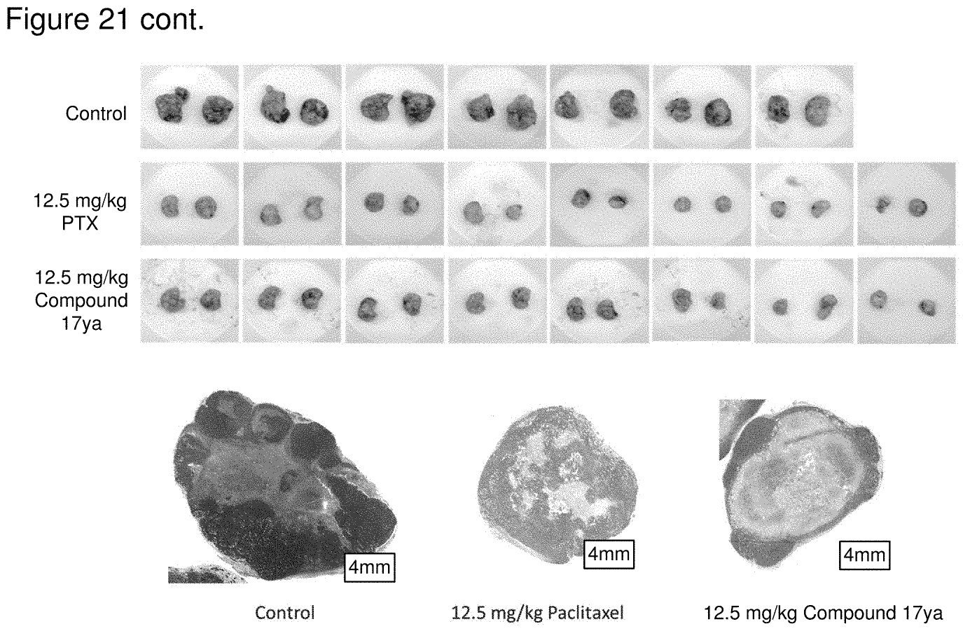

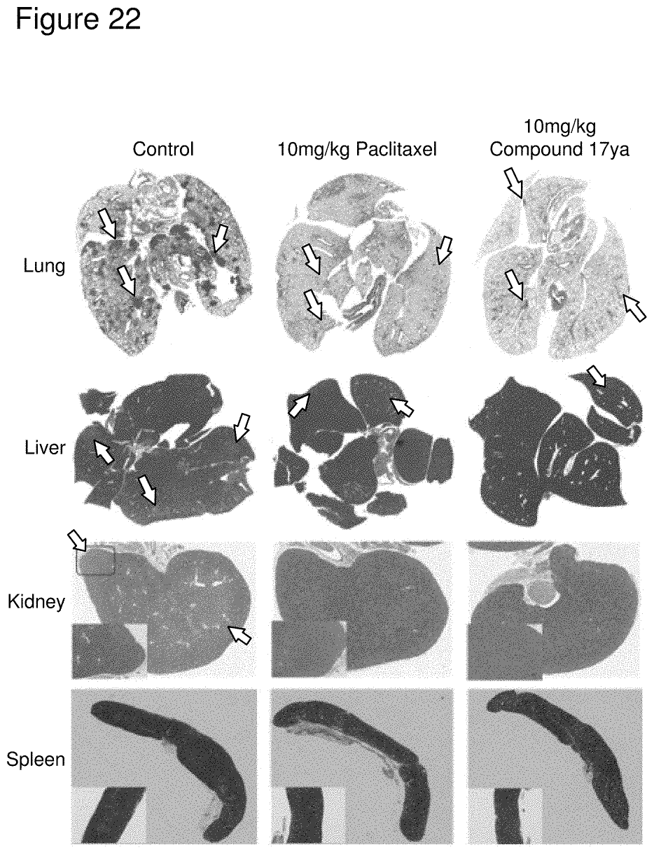

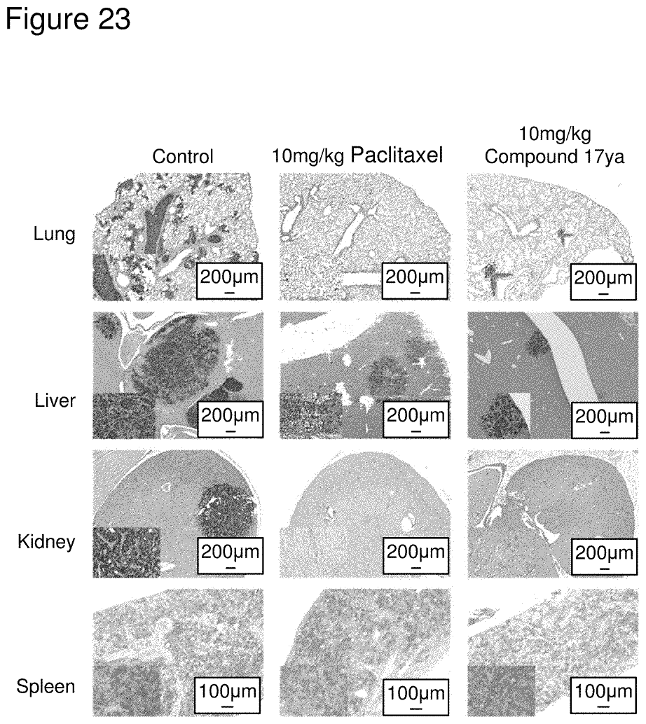

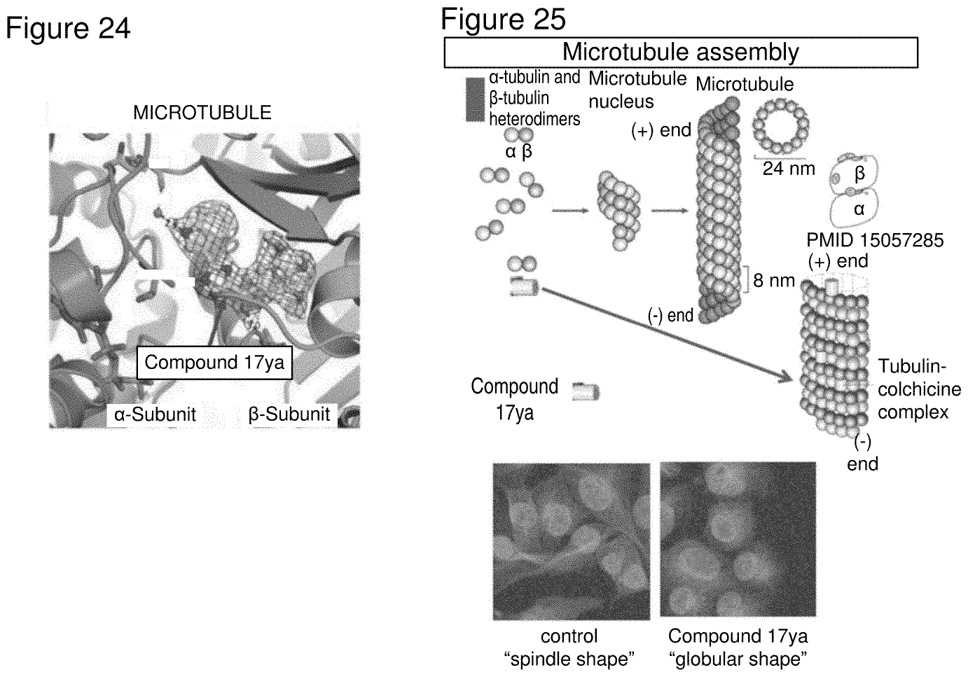

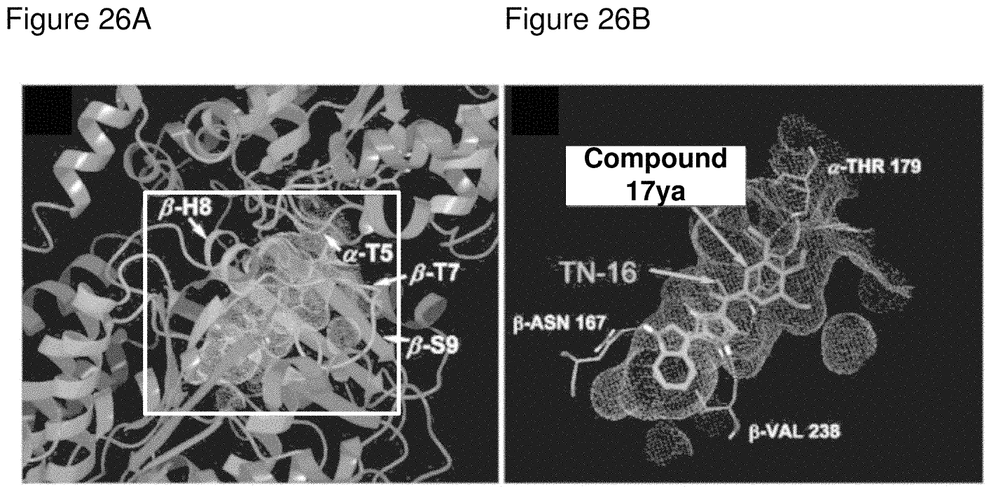

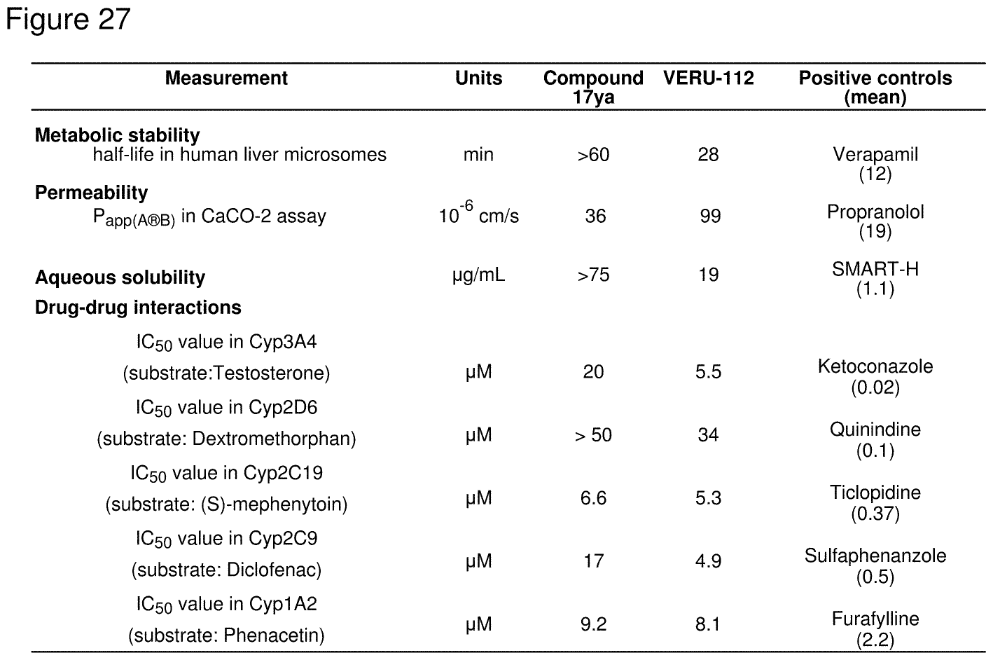

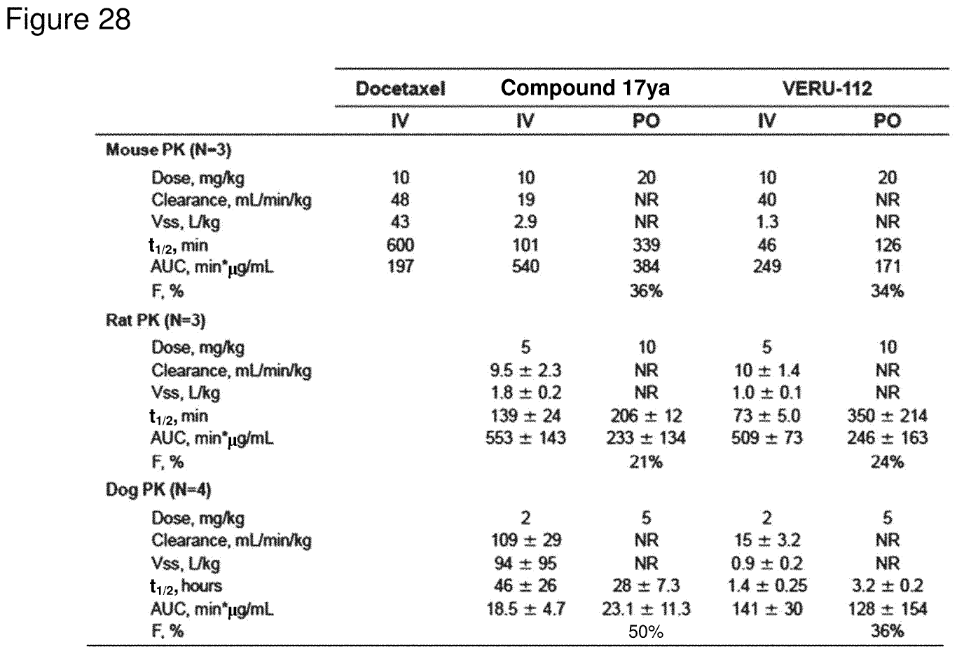

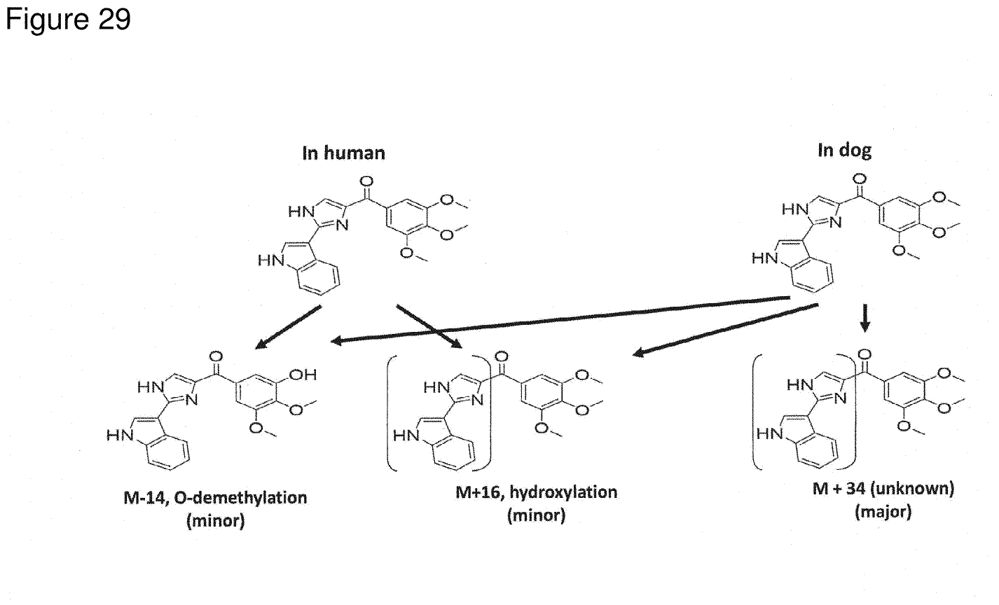

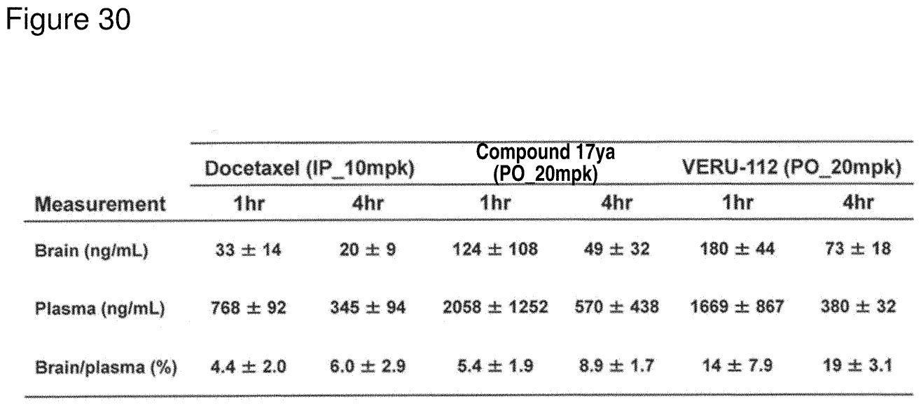

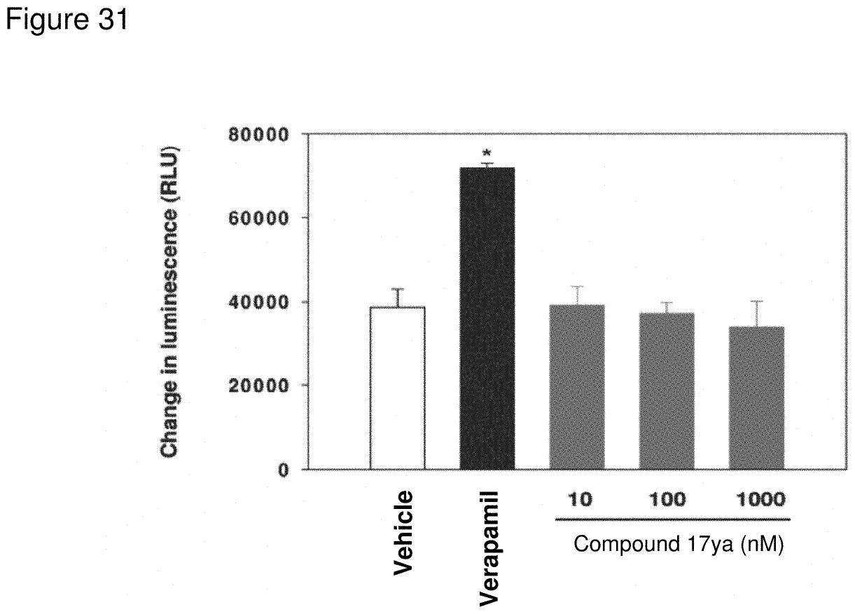

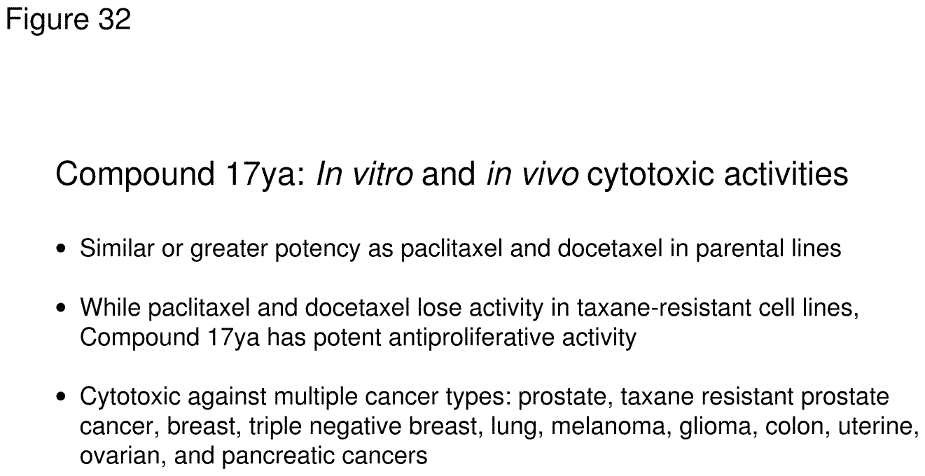

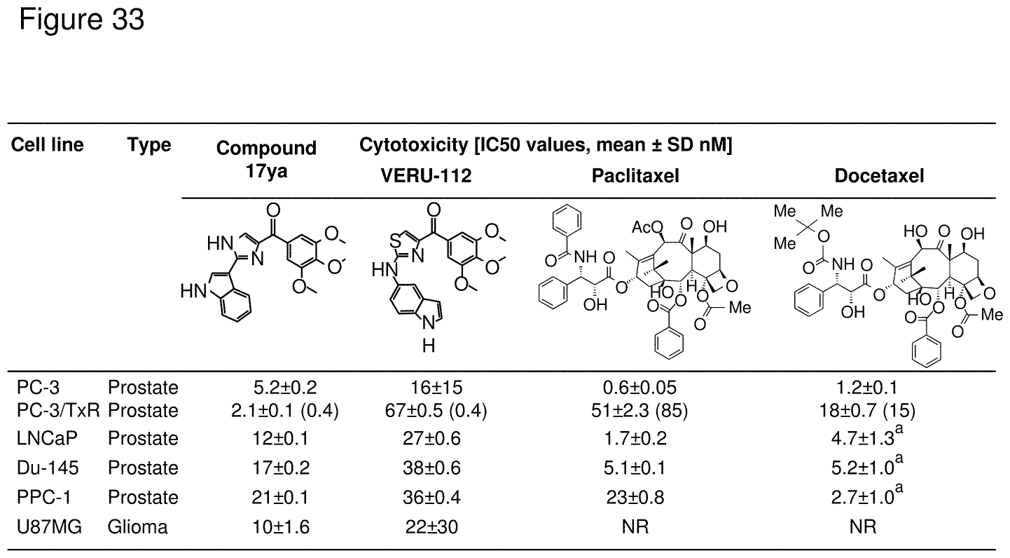

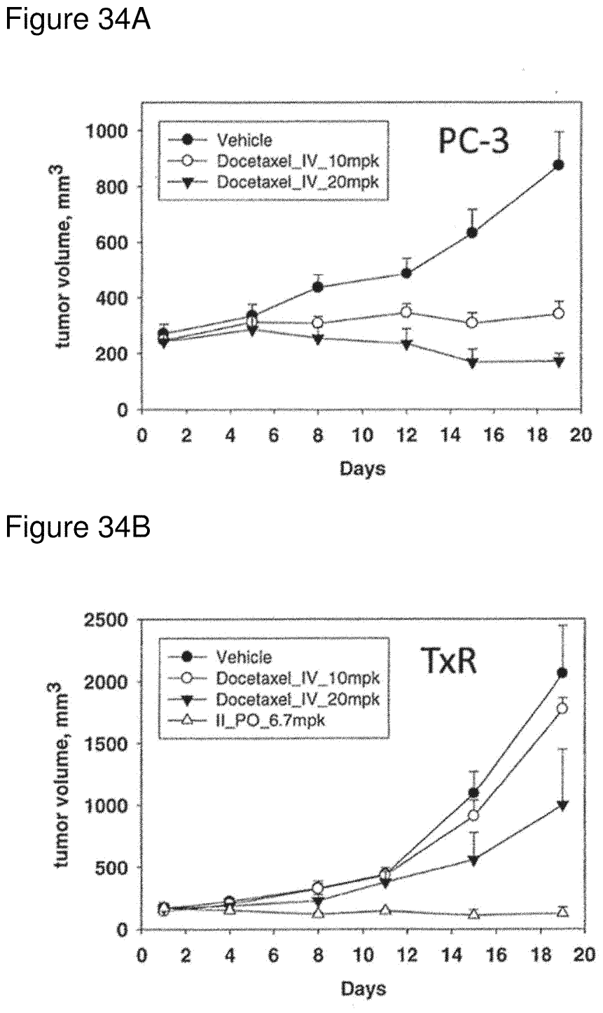

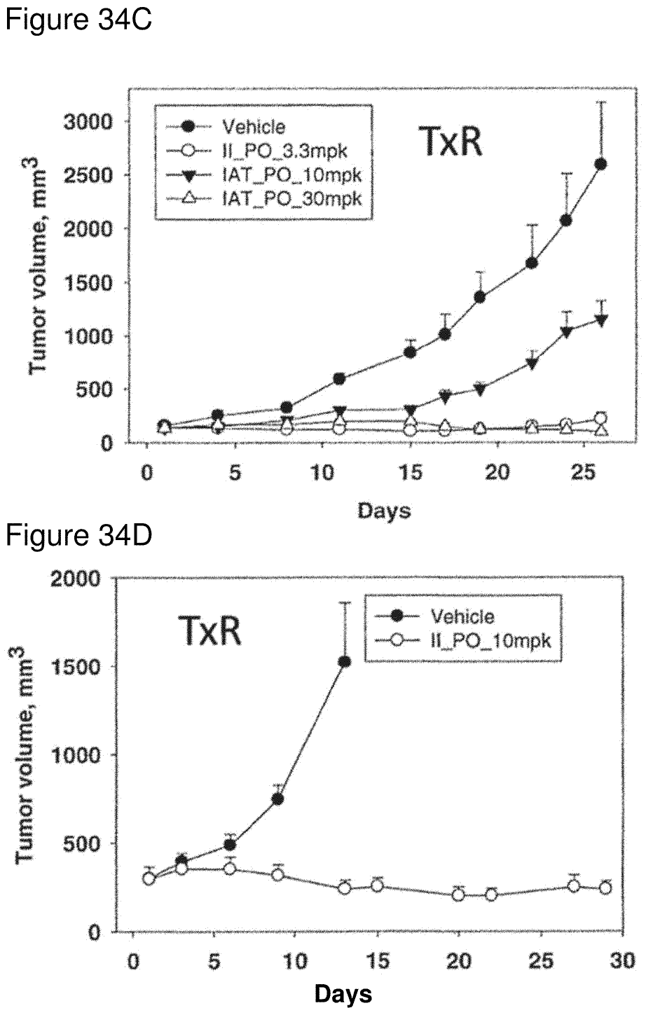

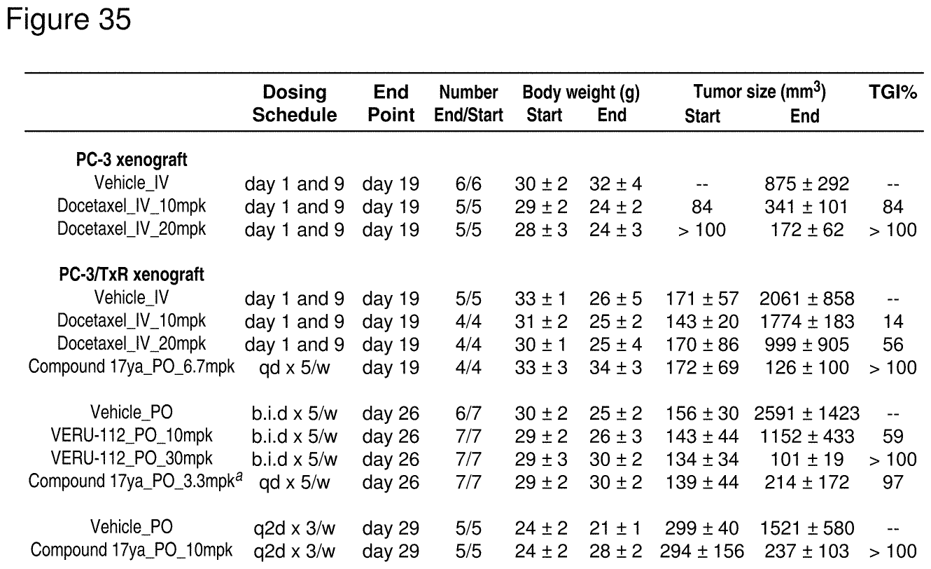

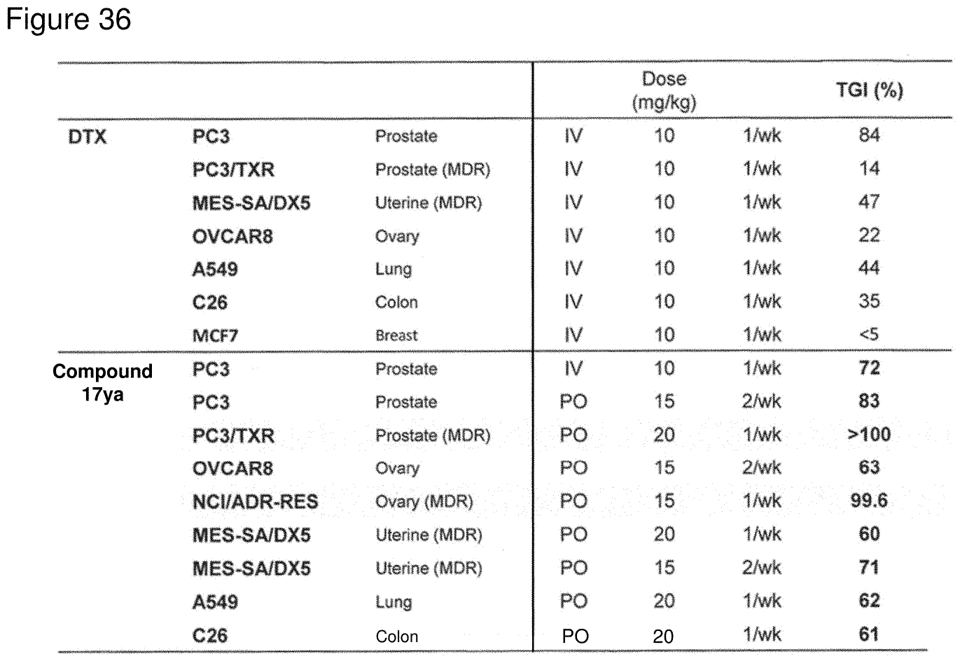

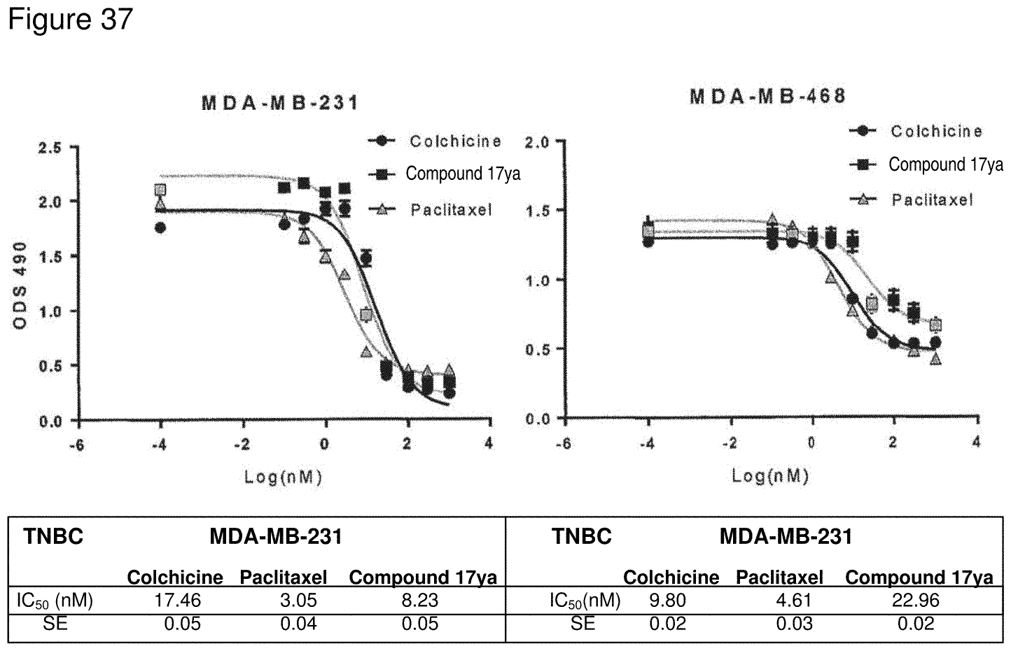

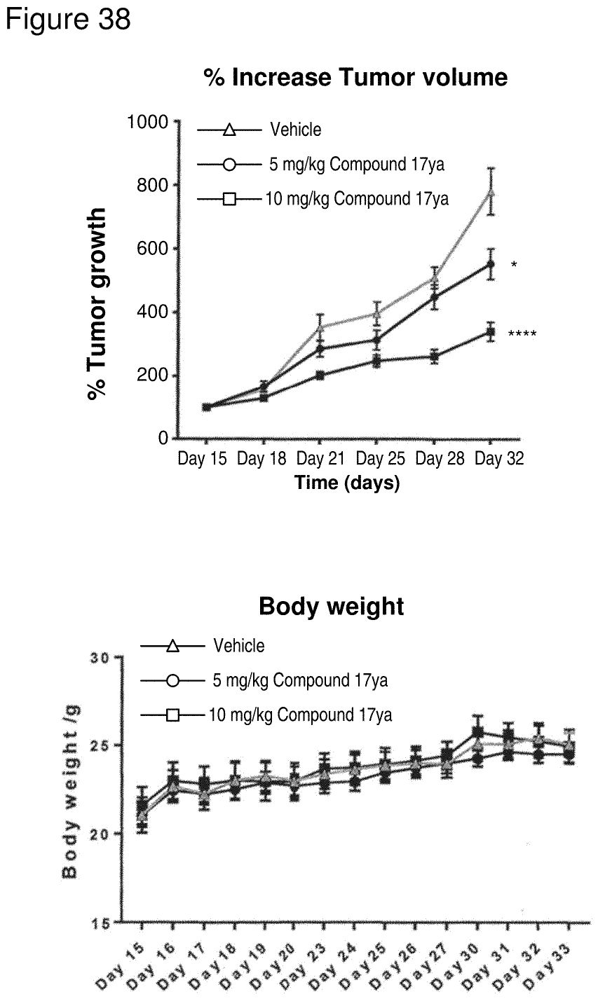

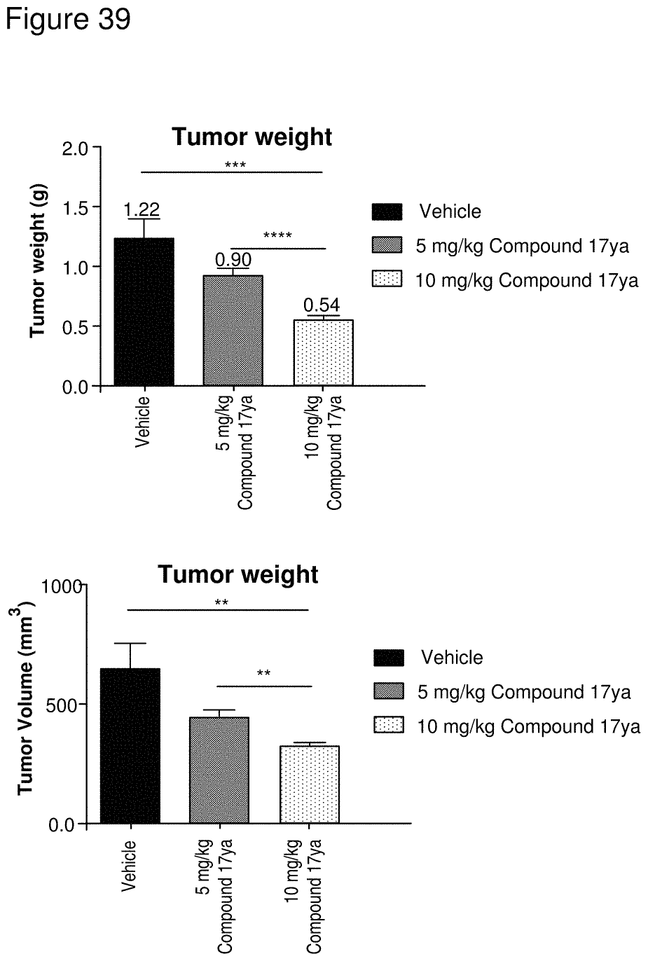

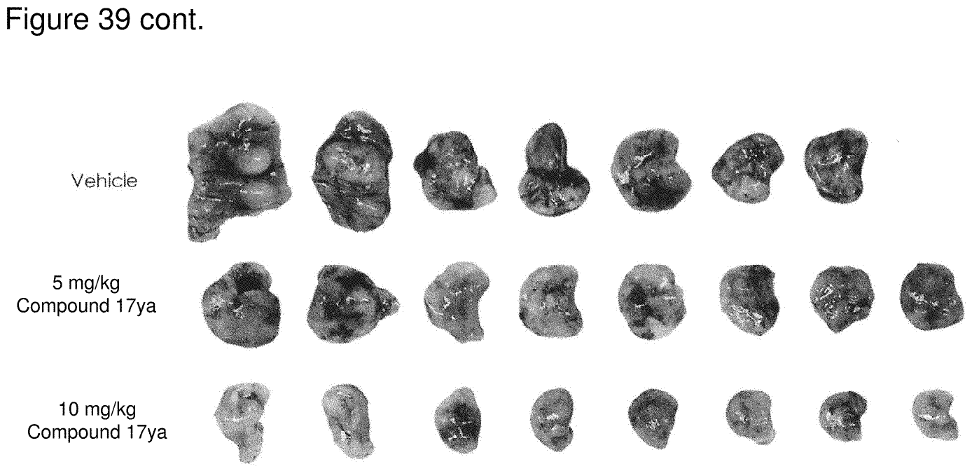

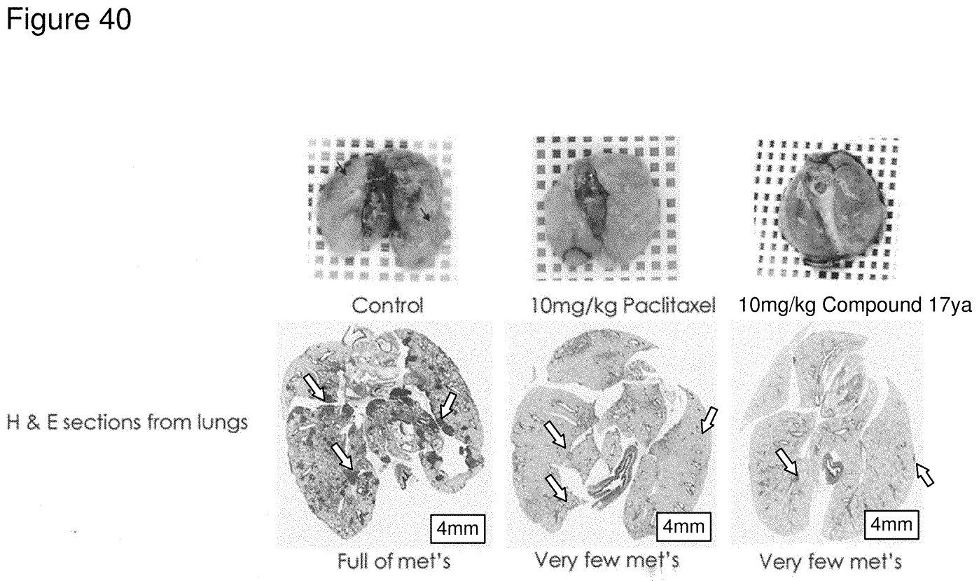

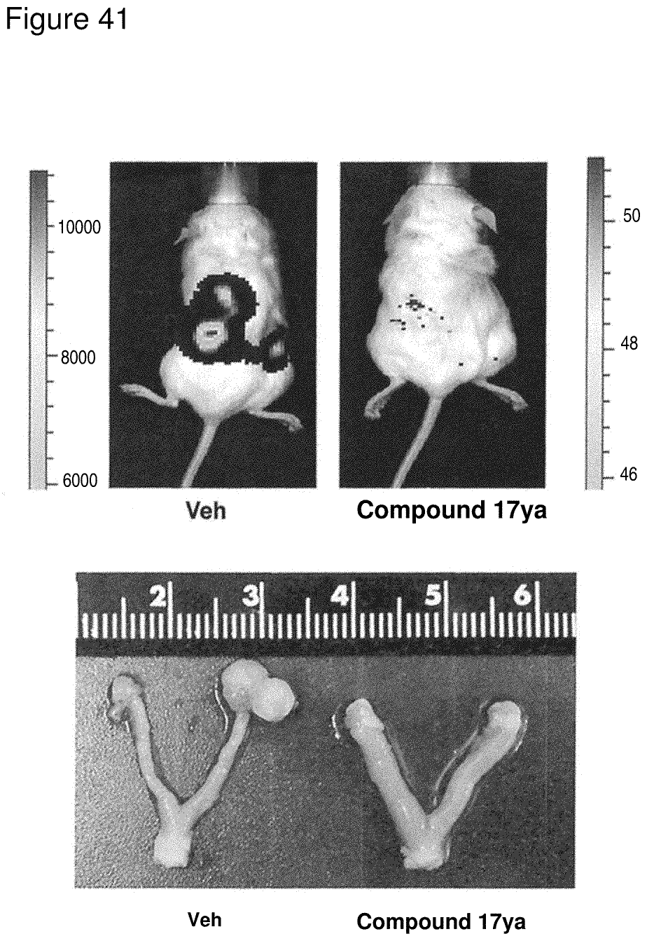

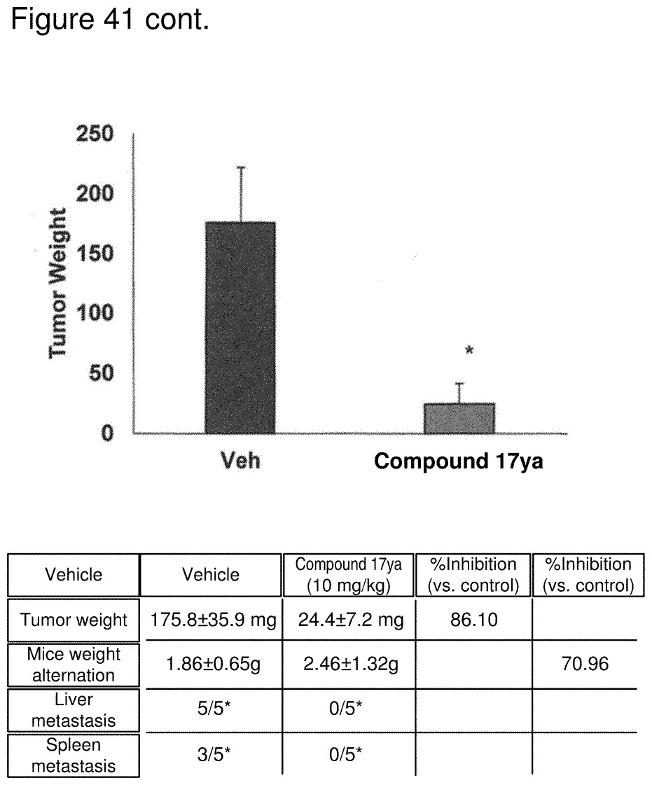

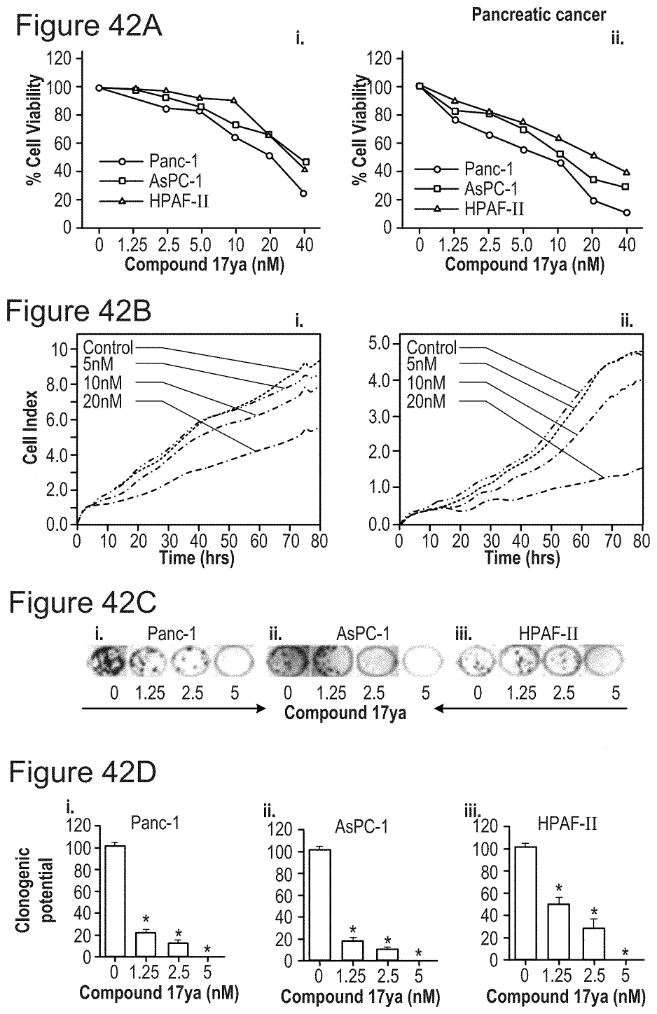

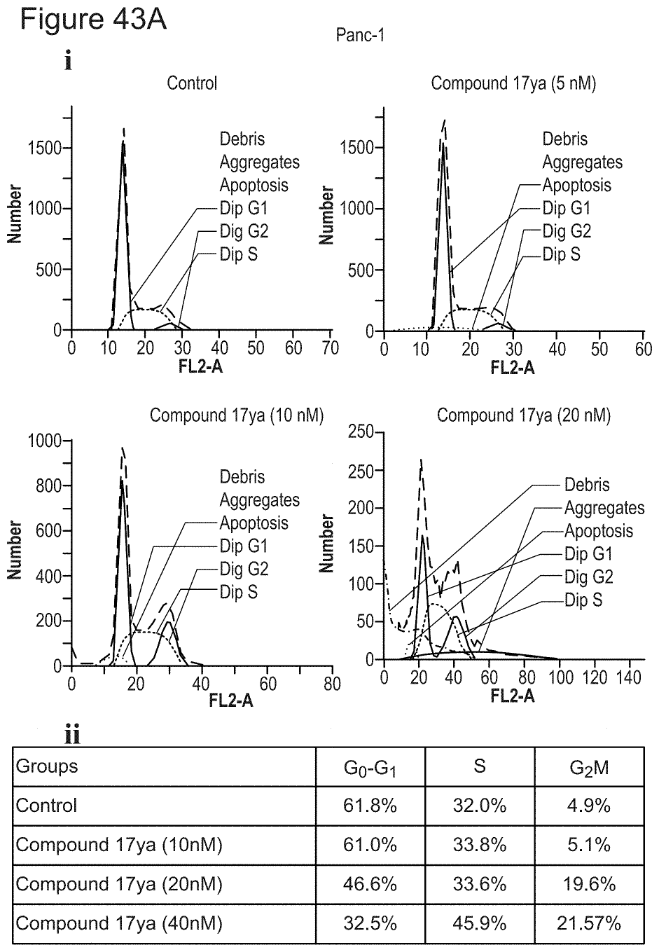

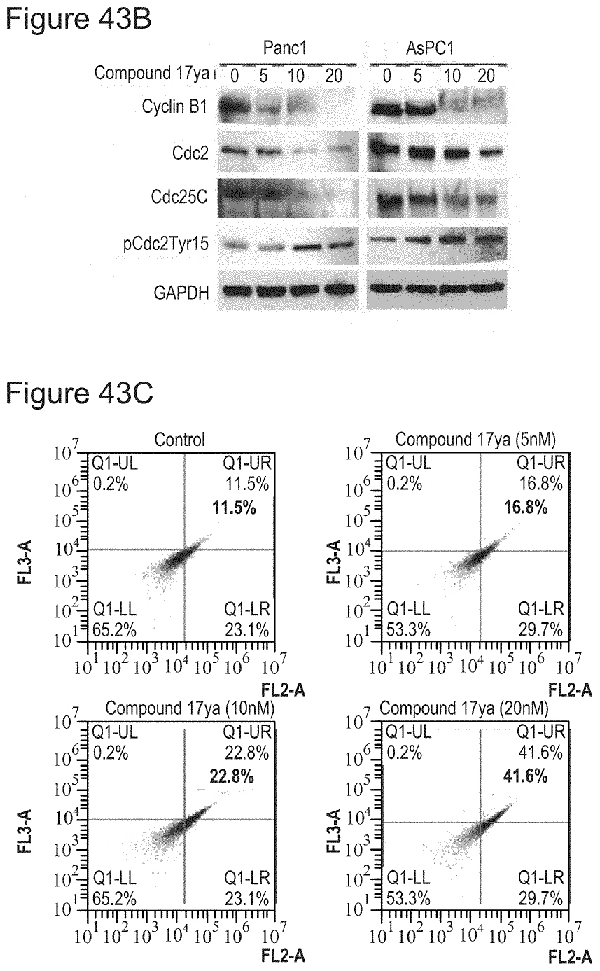

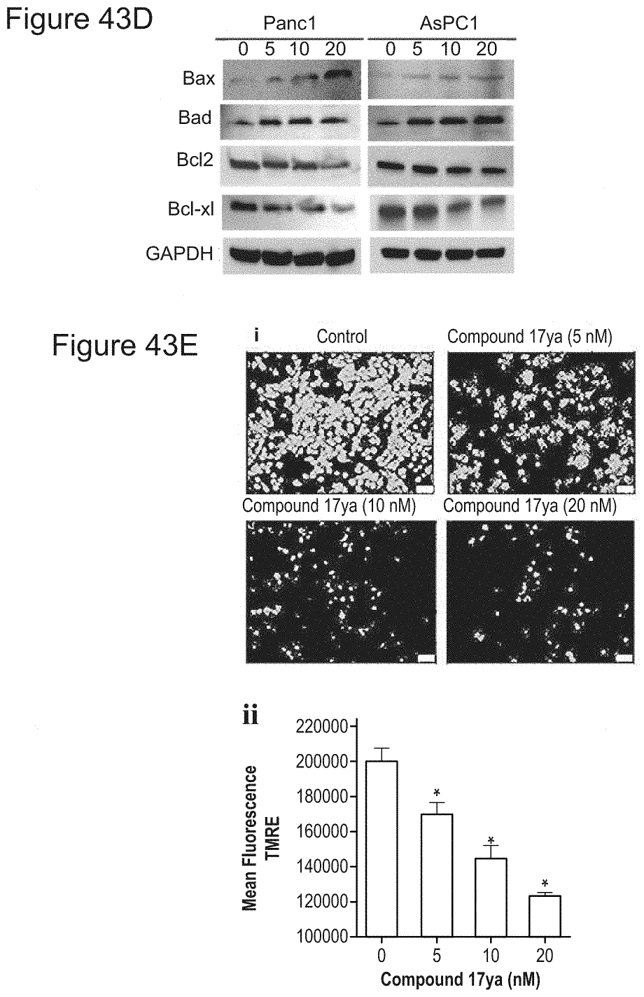

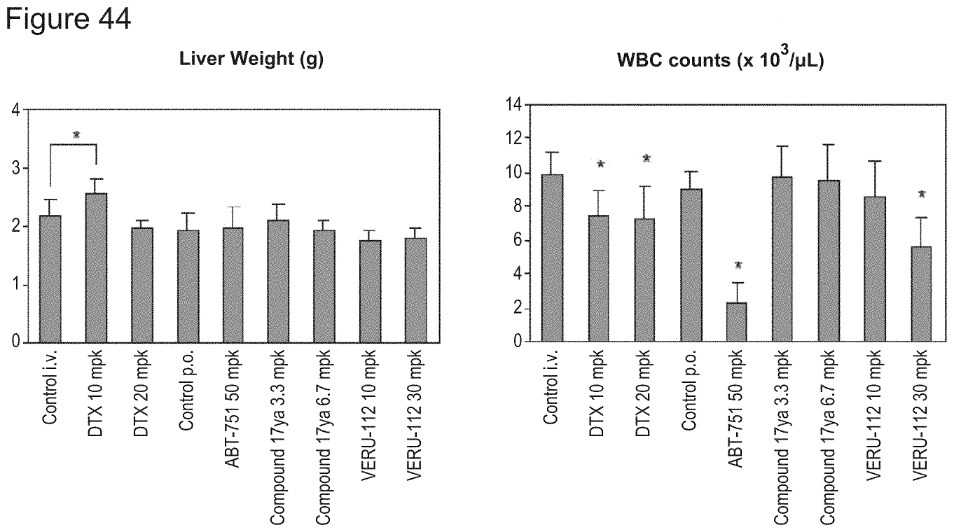

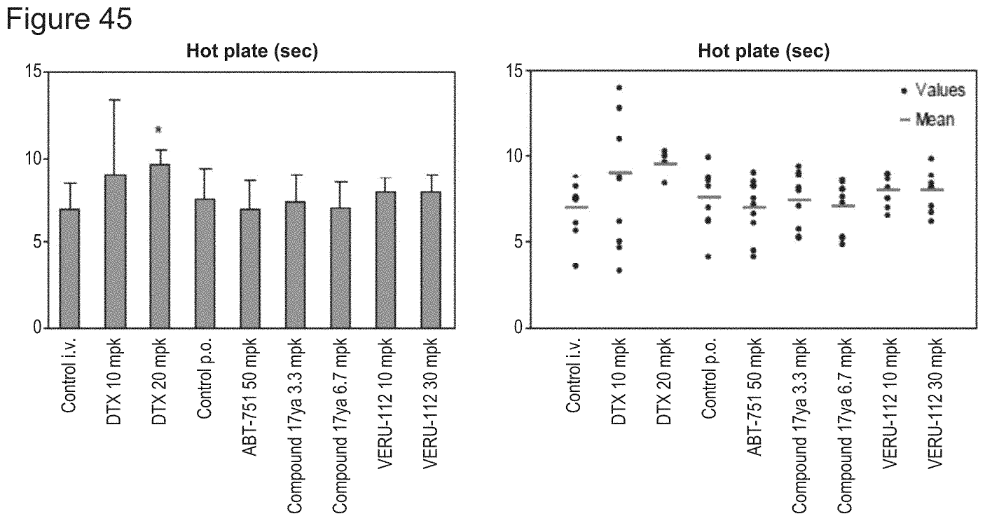

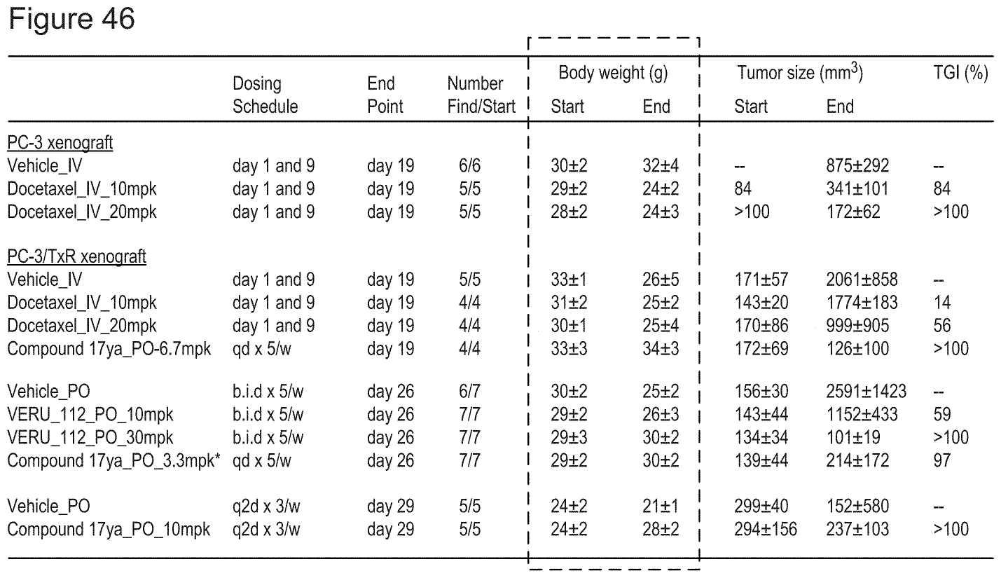

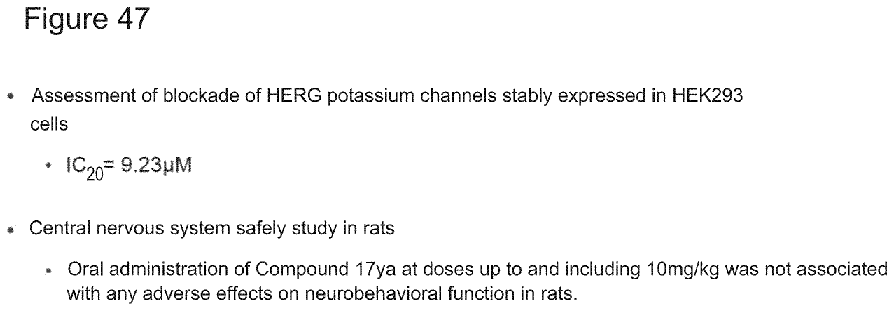

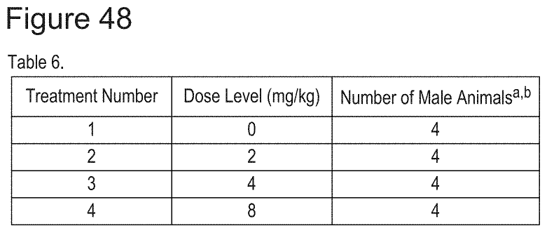

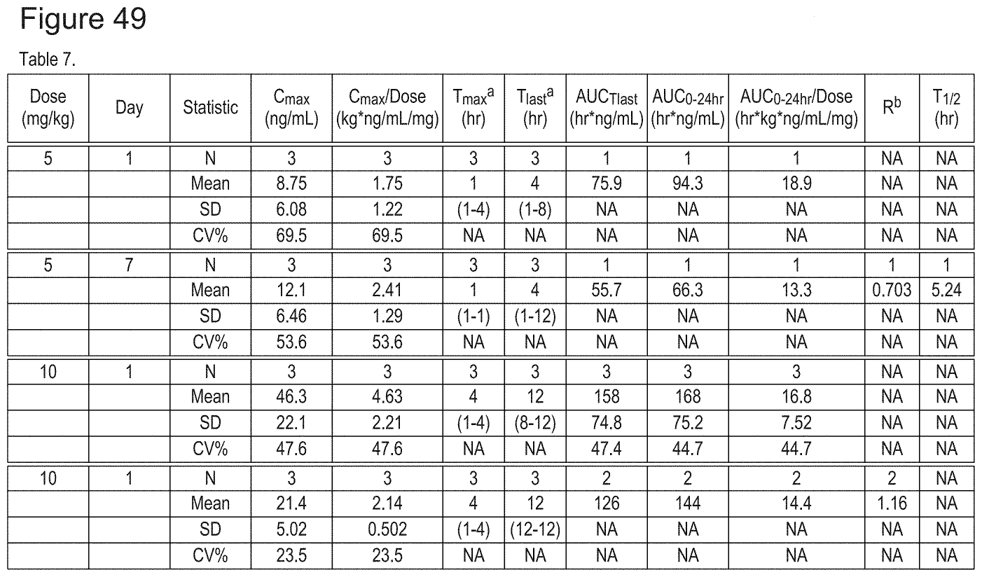

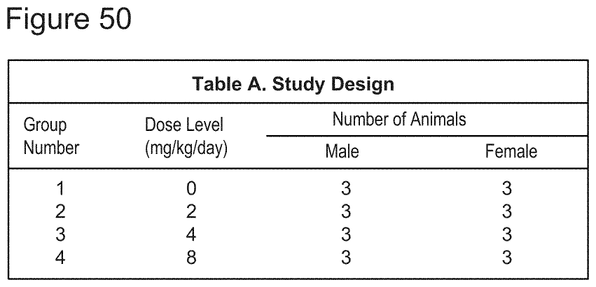

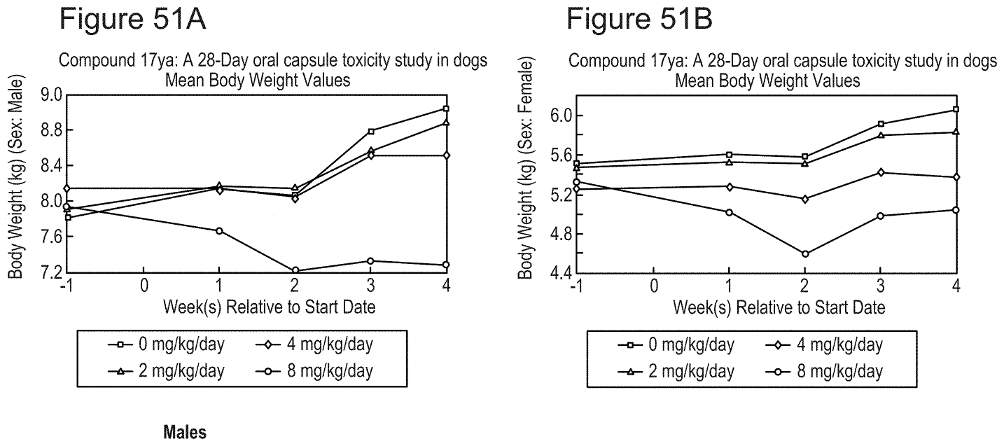

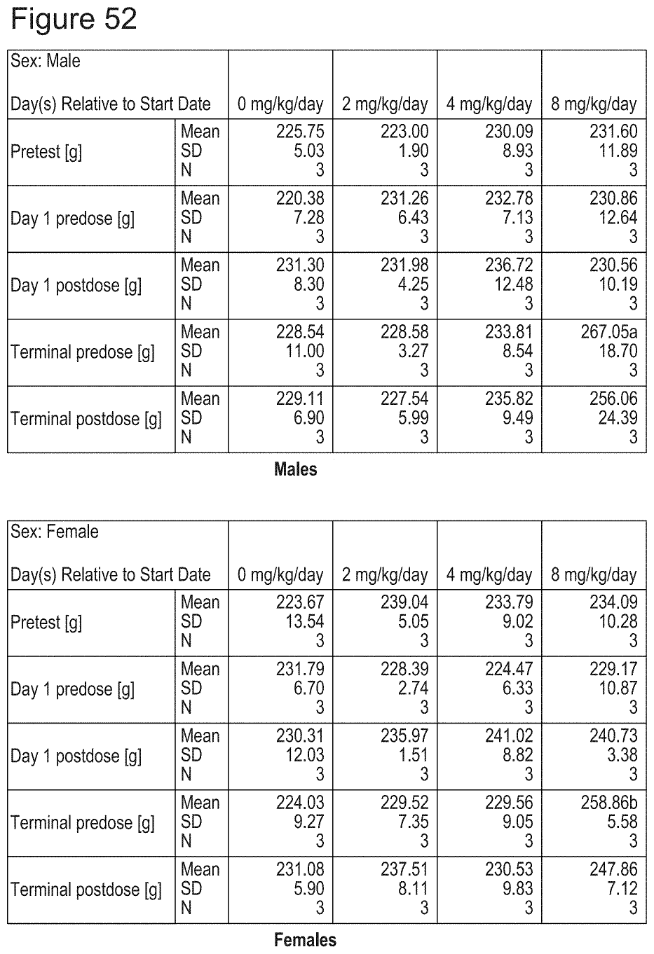

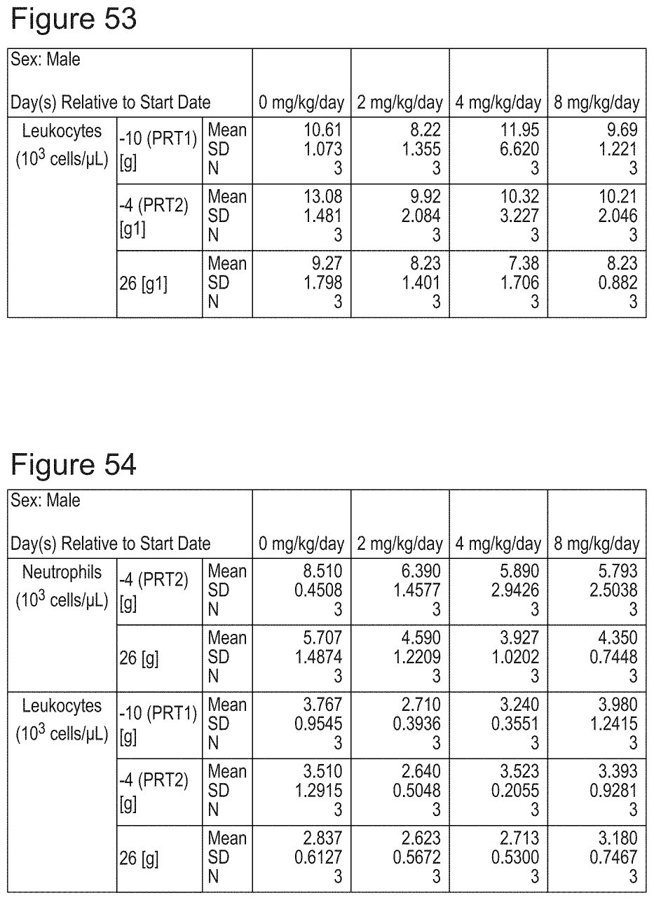

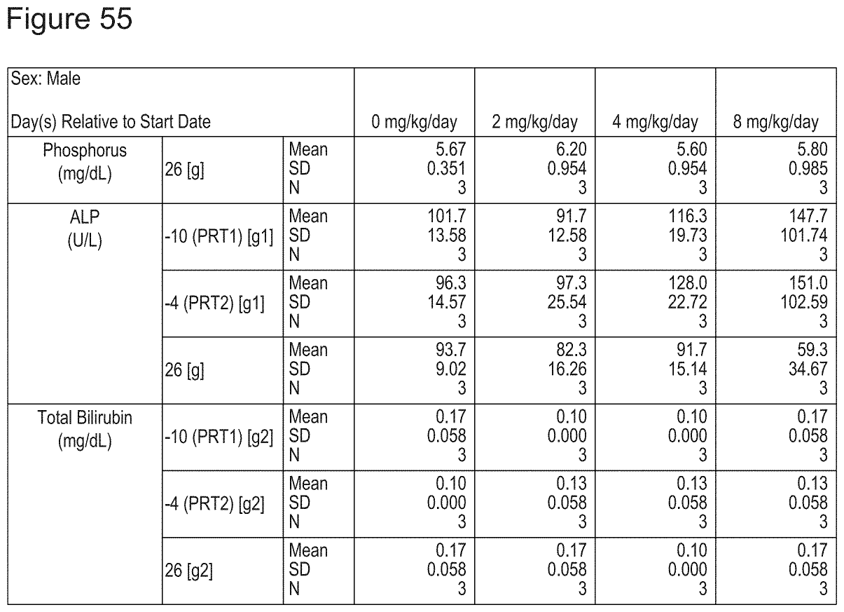

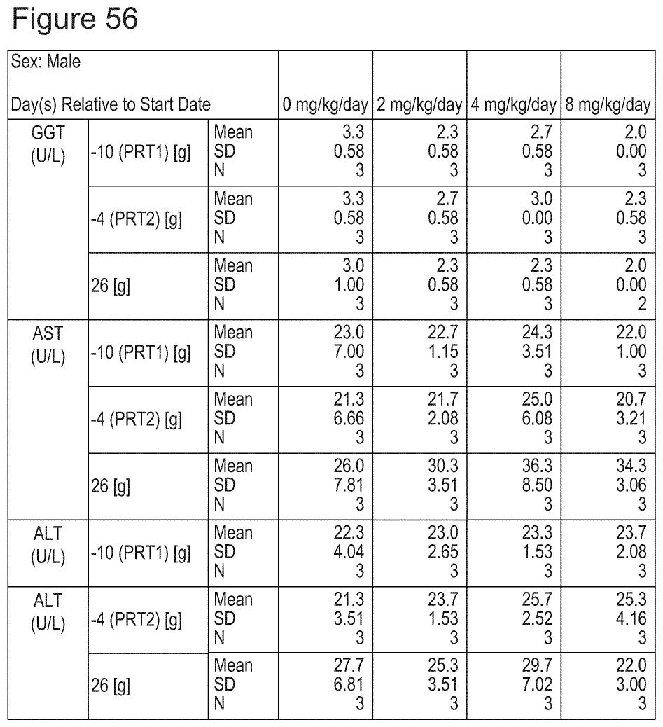

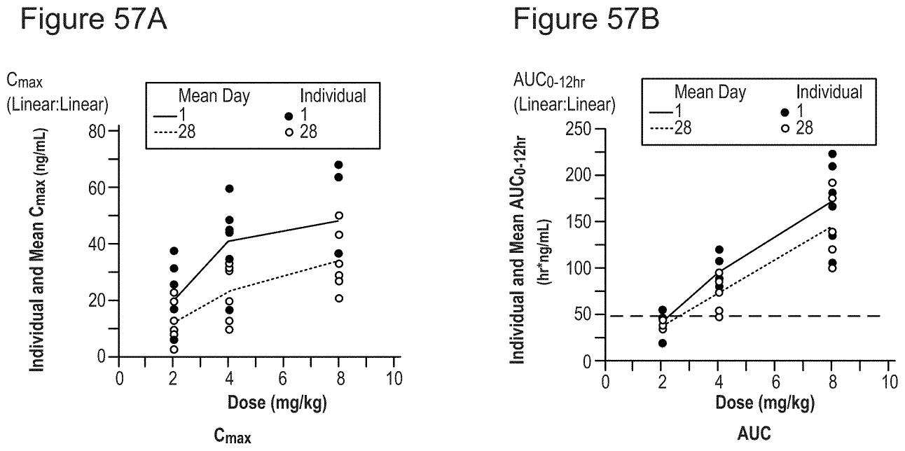

Compound 17ya inhibited TNBC tumor growth and metastasis in vivo. To validate in vitro results, the anti-cancer activity of compound 17ya in orthotopic TNBC mouse model was studied to determine if the potent effect of compound 17ya in vitro could be observed in vivo. After 33 days of treatment, treatment with compound 17ya inhibited TNBC tumor growth in a dose dependent manner without interfering with the body weight of mice. The results are illustrated in FIG. 20. The efficacy of compound 17ya in mice bearing TNBC tumors was shown by reduced tumor size and tumor weight compared with the control (vehicle treated) mice. The shape of all tumors in these three groups furthered showed that tumor size decreased with as the dose of compound 17ya increased. The H & E staining of the tumor sections determined that compound 17ya induced TNBC tumor necrosis, which is similar to what was observed in vitro. A study compared the efficacy of compound 17ya with paclitaxel in the same model since paclitaxel is one of the standard cares for TNBC treatment in clinic. Both compound 17ya and paclitaxel significantly regressed the tumor size and tumor weight. The results are illustrated in FIG. 21. The overall tumor picture confirmed that tumor size decreased in both compound 17ya and paclitaxel treated mice. Although compound 17ya was less potent than paclitaxel in regressing the tumor growth, it was better to dissolve and administer in the treatment of cancer compared to the paclitaxel. H & E staining of tumors in a comparison study further suggested that both compound 17ya and paclitaxel induced TNBC tumor necrosis. A tail vein study determined the efficacy of compound 17ya in anti-metastasis of TNBC in vivo. FIG. 22 and FIG. 23 demonstrated that the lungs in vehicle group were full of metastasis (indicated by yellow arrow), while the lungs in compound 17ya and paclitaxel group had little, which suggested that compound 17ya significantly reduced metastasis of TNBC. Similar results were found in liver, kidney and spleen tissues, which further demonstrated that compound 17ya inhibited the metastasis of TNBC.

### Example 3

In addition to the materials and methods described above, the following examples utilized the following procedures.

Cell cycle analysis. To determine the cell cycle profile in the mitotic phase (especially G2 and M phase), cells were treated with colchicine, paclitaxel, and compound 17ya. The cells were harvested by trypsinization, fixed, permeabilized, stained with anti-phospho-Histone H3-AlexaFluor® 488 antibody on ice for 1 h in the dark and incubated with freshly prepared propidium iodide/Rnase solution for 30 min at room temperature in the dark according to the manufacturer's protocol (#FCCH0225103, EMD Millipore Corp., Burlington Mass.). Stained cells were then analyzed by Bio-Rad ZE5 cell analyzer (Bio-Rad, Hercules, Calif.). Data were processed, and graphs generated using FlowJo (FlowJo, LLC, Ashland, Oreg.).

Western Blotting. Cells were incubated with increasing doses of compound 17ya and 100 nM colchicine and paclitaxel for 24 h, or 100 nM compound 17ya for 24 h, 48 h, and 72 h as time-dependent investigation. Then the cells were harvested, washed with ice-cold PBS, lysed in RIPA buffer (25 nM Tris pH 7.6, 150 nM NaCl, 1% NP-40, 1% sodium deoxycholate, 0.1% SDS) with Halt™ protease and phosphatase inhibitor (Thermo Fischer Scientific) and then centrifuged at 13000 rpm at 4° C. for 10 min. Protein in the supernatant was determined by BCA Protein Assay (Thermo Fischer Scientific). Equal amounts of each denatured protein sample were loaded and separated by SDS-PAGE gradient gels (Bio-Rad, #456-1083). Protein was wet-box transferred to PVDF membranes. The membranes were then blocked in 5% non-fat milk in TBST solution at room temperature for 1 h, incubated with primary antibodies overnight at 4° C. and bound with secondary antibody for 1 h subsequently. The following primary antibodies were used: rabbit anti-Poly (ADP-ribose) polymerase (PARP, 1:1000), rabbit anti-cleaved PARP (1:1000), rabbit anti-cleaved-capase-3 (1:1000) and rabbit anti-GAPDH HRP conjugate (#9532; #5625; #9661; #3683, Cell Signal Technology, Danvers, Mass.). Bound proteins were detected using Clarity™ Western ECL Substrate (Bio-Rad, #1705060) and visualized by ChemiDoc-It2 Imager system (UVP, LCC, Upland, Ca).

In vivo orthotoxic xenograft model. All animal studies were carried out in adherence to the NIH Principles of Laboratory Animal Care and protocols approved by the Institutional Animal Care and Use Committee at the University of Tennessee Health Science Center. Female Nod-Scid-γ (NSG) mice at 5-6 weeks of age were housed in a specific pathogen free environment with a 12:12-hour light-dark cycle. Temperature was maintained at 20-26° C. and the relative humidity was maintained at 30-70%. 2.5×10⁵ MDA-MB-231 cells in 10 μl of HBSS were surgically inoculated into the left and right inguinal mammary gland fat pads of NSG mice as described by Pfeffer et al., “Comprehensive analysis of microRNA (miRNA) targets in breast cancer cells,” J. Biol. Chem., 2013, 288, 27480-27493, hereby incorporated by reference. Mice were inspected weekly for tumor appearance until the average tumor size reached 100 mm³. Then the mice were randomly divided into 5 groups (n=14 in vehicle control group, n=8 in drug treated group) and drug treatment was started. The control group was administered with vehicle (1:1 ratio of PEG 300:water) orally, drug treatment groups were administered 5 mg/kg compound 17ya, 10 mg/kg compound 17ya, 12.5 mg/kg compound 17ya orally five times a week, and 12.5 mg/kg paclitaxel via intraperitoneal injection every other day, respectively. Primary tumor size was monitored twice a week using a digital caliper and body weight of the mice was recorded during the treatment. The tumor volume was calculated using the formula volume=(width²×length)/2. After 18 days treatment, when the tumor size in the vehicle group reached 1000 mm³, tumors and major organs were imaged and collected in 10% buffered formalin phosphate solution for histological analysis.

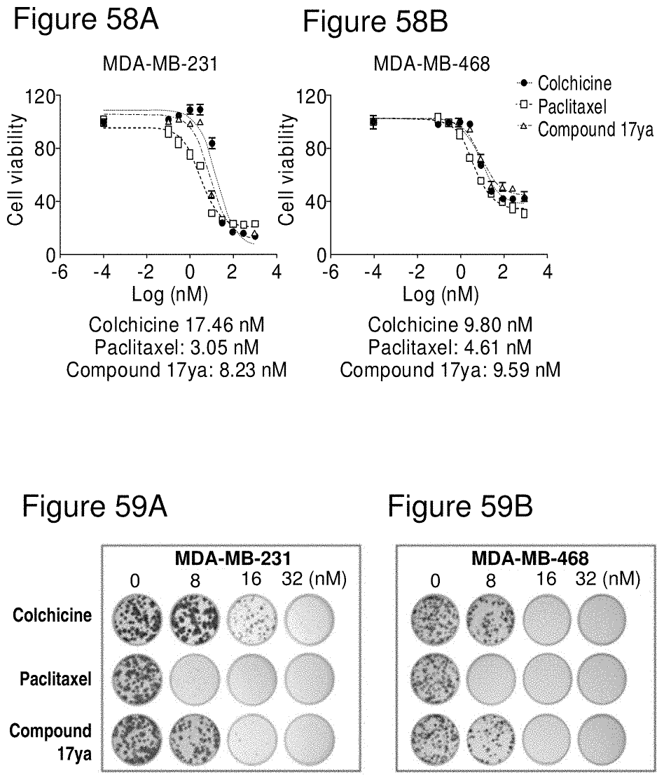

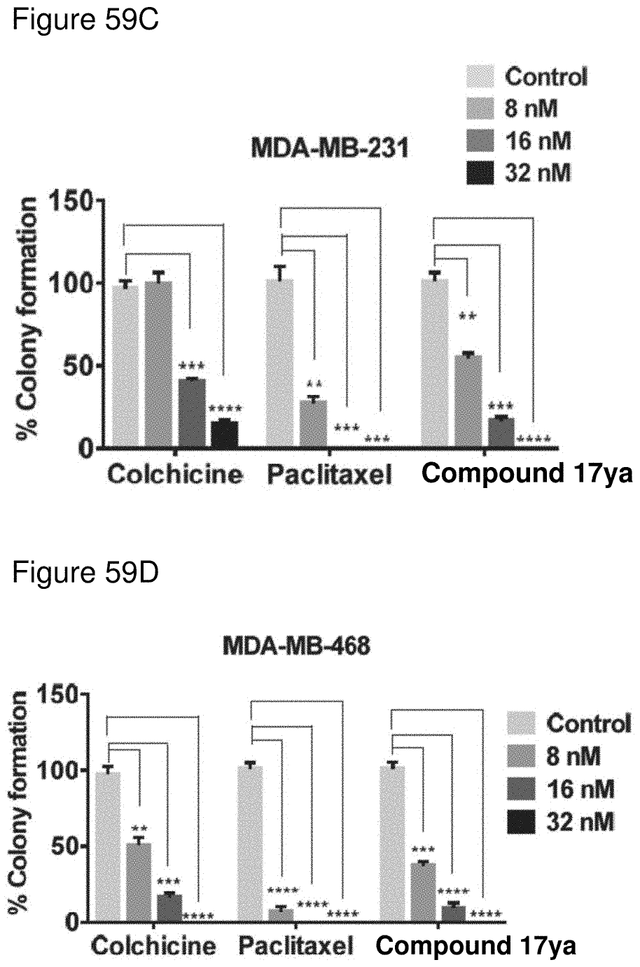

Compound 17ya extenuated the proliferation of different breast cancer cells and interfered microtubule assembly and mitotic spindle organization. Compound 17ya previously was tested in a panel of melanoma cancer cell lines with an average IC₅₀ of 4 nM, and now was evaluated to determine whether compound 17ya could inhibit the growth of breast cancer cells using MTS assay. The results demonstrated that compound 17ya had an anti-proliferative effect against breast cancer cell lines, with IC₅₀ value of 14 nM in HER2-positive breast cancer cells and 8 nM in TNBC cells. Tubulin-destabilizing colchicine and tubulin-stabilizing agent paclitaxel were incorporated to compare the efficacy of colchicine, paclitaxel, and compound 17ya against TNBC cell growth. (See FIG. 58A for MDA-MB-231 and FIG. 58B for MDA-MB-486). All three tubulins inhibitors were potent in inhibiting the proliferation of TNBC, where IC₅₀ values of compound 17ya ranged from 8.2-9.6 nM. Colony formation assay results demonstrated that compound 17ya attenuated the capacity of the proliferation of TNBC cells in a dose dependent manner. (See FIG. 59A for MDA-MB-231 and FIG. 59B for MDA-MB-486). Paclitaxel showed the most remarkable effect with relative percentage of colonies covering 27.5% compared to colchicine (100%) at 8 nM and compound 17ya (55%) was more potent than colchicine. In MDA-MB-468 cells, reductions in colony formation were observed in colchicine (51%), paclitaxel (7.4%), and compound 17ya (37.7%) at the dose of 8 nM, which indicated that all three tubulin inhibitors inhibited the colony formation of TNBC cells.

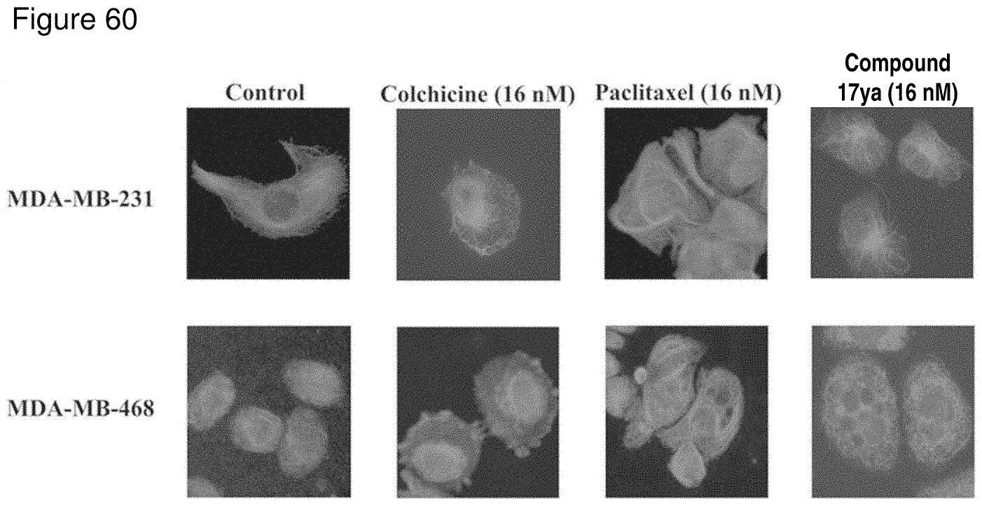

Immunofluorescence staining was used to visualize the microtubule network in comparison with colchicine and paclitaxel. TNBC cells in the negative control group showed intact microtubule fibers and organization of microtubules, as illustrated in FIG. 60. Treatment with paclitaxel resulted in multipolar spindles with highly condensed chromosomes due to the increase of tubulin polymerization in TNBC cells. Like the treatment of colchicine, cells treated with compound 17ya has shrunken and the cell shape was changed from spindle to round and irregular, that confirmed compound 17ya targeted tubulin and interfered with tubulin polymerization.

### Example 4

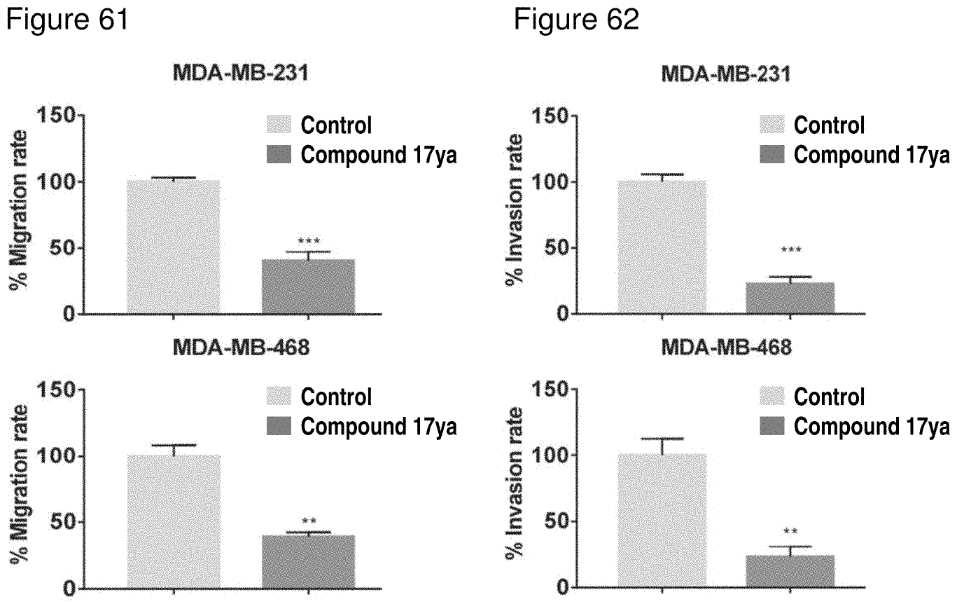

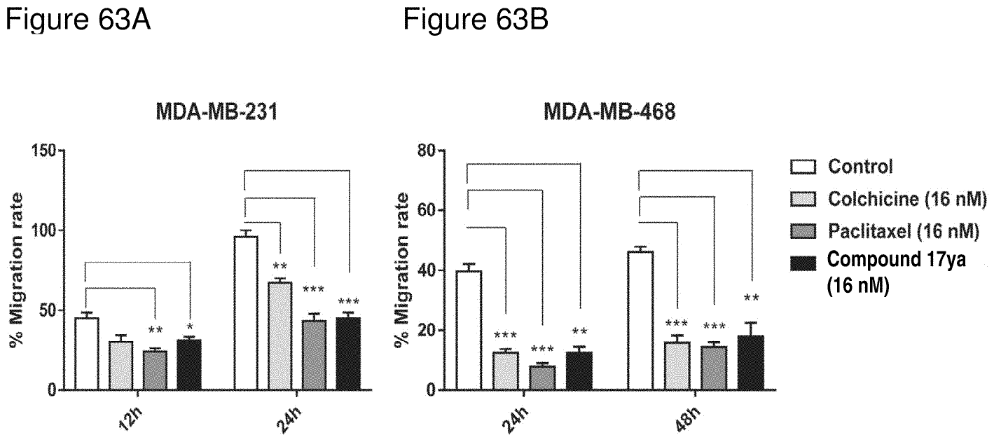

Compound 17ya inhibited TNBC cell migration and invasion. The example focused on the effect of compound 17ya on the migration and invasion of TNBC cells after 24 or 48 hours of treatment. Compound 17ya inhibited TNBC cells ability to migrate through a membrane insert in the presence of 16 nM concentration by an average migration rate of 40% in MDA-MB-231 cells and 34% in MDA-MB-468 cells as compared to a control group (migration rate 100%) as illustrated in FIG. 61. Compound 17ya reduced the TNBC cells capacity to invade through the Matrigel-coated membrane with an average invasion rate of 55% and 36% in MDA-MB-231 and MDA-MB-468 cells, respectively, when invasion rate of the control group was set as 100%. The results are illustrated in FIG. 62. A scratch assay was performed using paclitaxel and colchicine as positive controls. At a dose of 16 nM, compound 17ya, colchicine, and paclitaxel showed effective inhibition of cell migration as illustrated in FIG. 63A for MDA-MB-231 and FIG. 63B for MDA-MB-486. After 24 hours, the average migration rates of DMSO, colchicine, paclitaxel, and compound 17ya treated MDA-MB-231 cells were 100%, 67.3%, 13.3%, and 44.9%, respectively. Similarly, after 48 hours treatment with colchicine, paclitaxel, and compound 17ya were able to reduce the migration of MDA-MB-231 cells with average migration rates of 15.8% m 14.5%, and 17.9%, respectively. The findings concluded that compound 17ya significantly suppressed cell migration.

### Example 5

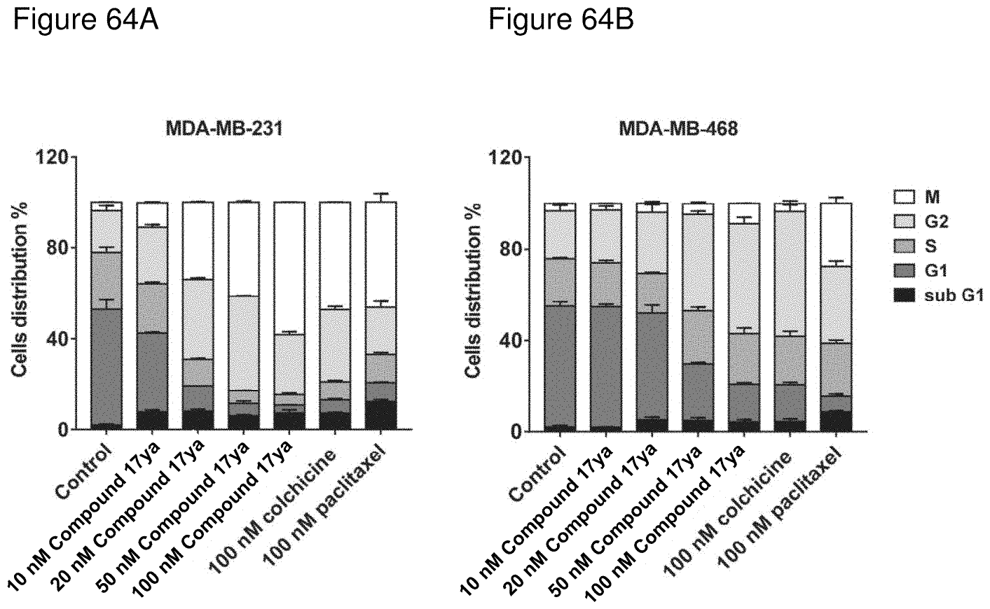

Compound 17ya blocks TNBC cells in G2/M phase and induced cell apoptosis. Microtubule dynamics play a significant role in cell division. Its disruption may lead to the mitotic arrest of growing cells in metaphase and ultimately cause cell death. In this example it was determined that compound 17ya can affect cell cycle arrest. A flow cytometry analysis was conducted of cells treated for 24 hours with 100 nM colchicine, 100 nM paclitaxel, and different concentrations of compound 17ya. Different compounds showed divergent effects on the cell cycle progression in different cells lines. Compound 17ya treatment induced the accumulation of MDA-MB-231 cells in the G2 and M phase with a reduction in the population of cells in the G1 and S phase in a dose dependent manner. Colchicine and paclitaxel, employed as positive controls, also arrested MDA-MB-231 cells in G2/M phase as illustrated in FIG. 64A. Compound 17ya induced a G2 phase arrest in MDA-MB-468 cells and reduced the cell population of the G1 phase, while it had little effect in the percentage of cells in S phase as illustrated in FIG. 64B. A small increase of cells in the M phase was observed. Colchicine arrested MDA-MB-468 cells in G2 phase, paclitaxel arrested cells in both G2 and M phase as illustrated in FIG. 64B. Compound 17ya concentration resulted in a large accumulation of cells in G2/M phase starting with 20 nM in MDA-MB-231 cells, 50 nM in MDA-MB-468 cells, with maximum accumulation observed by 100 nM. Compound 17ya thus induced G2/M phase arrest of TNBC cells, leading to growth inhibition.

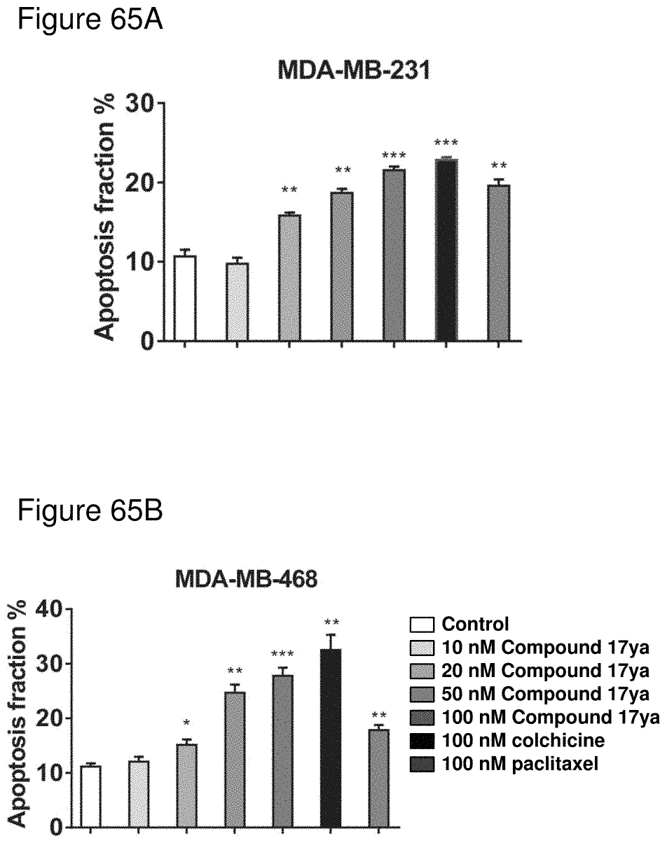

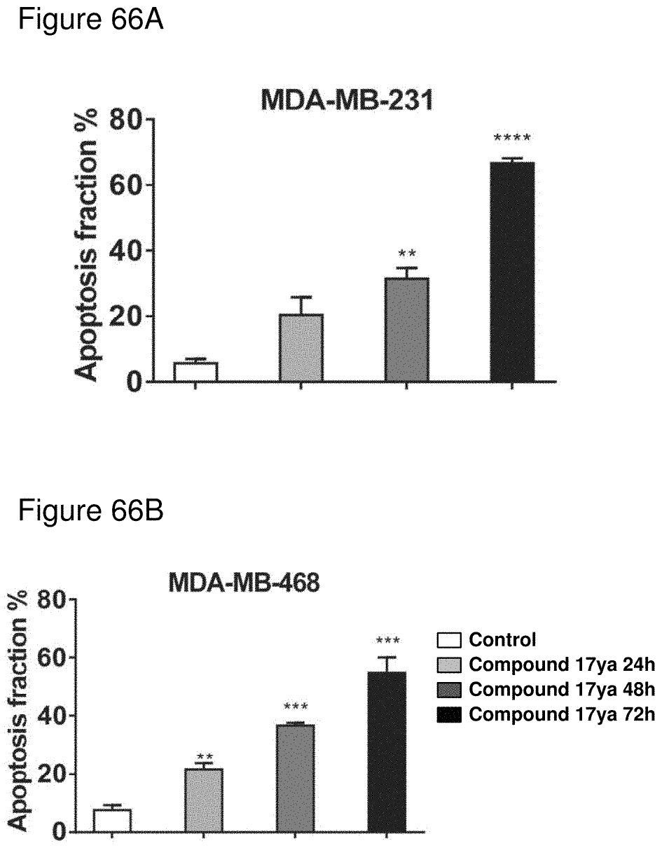

The pro-apoptotic effect on TNBC cells by compound 17ya was studied. The effect of compound 17ya on apoptosis induction in TNBC cells was studied using Annexn V-FITC-PI double staining method. MDA-MB-231 and MDA-MB-468 cells were treated with increasing concentrations of compound 17ya for 24 h. Quantitatively, compound 17ya initiated apoptotic cell death in a dose dependent manner, indicated by the appearance of Annexin-V⁺/PI⁻ cells, Annexin-V⁺/PI⁺ cells and Annexin-V⁻/PI⁺ cells shown in the representative histograms of FIGS. 65A-B. The potency of compound 17ya to induce TNBC cell apoptosis was identical to colchicine, but higher than paclitaxel. The results illustrated that MDA-MB-231 (FIG. 66A) and MDA-MB-468 (FIG. 66B) cell treated with 100 nM of compound 17ya for 24, 48, and 72 hours underwent apoptosis in a time-dependent manner.

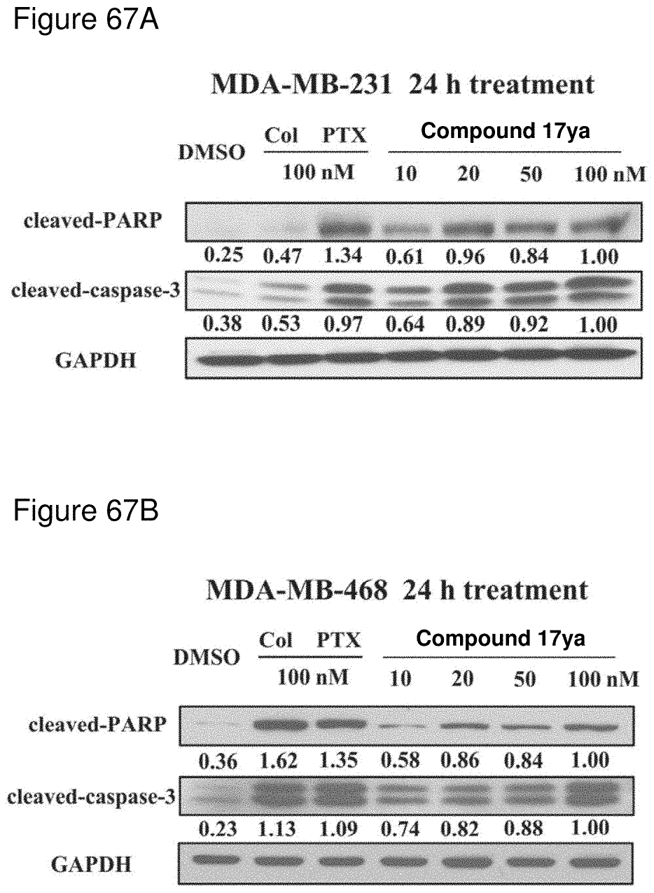

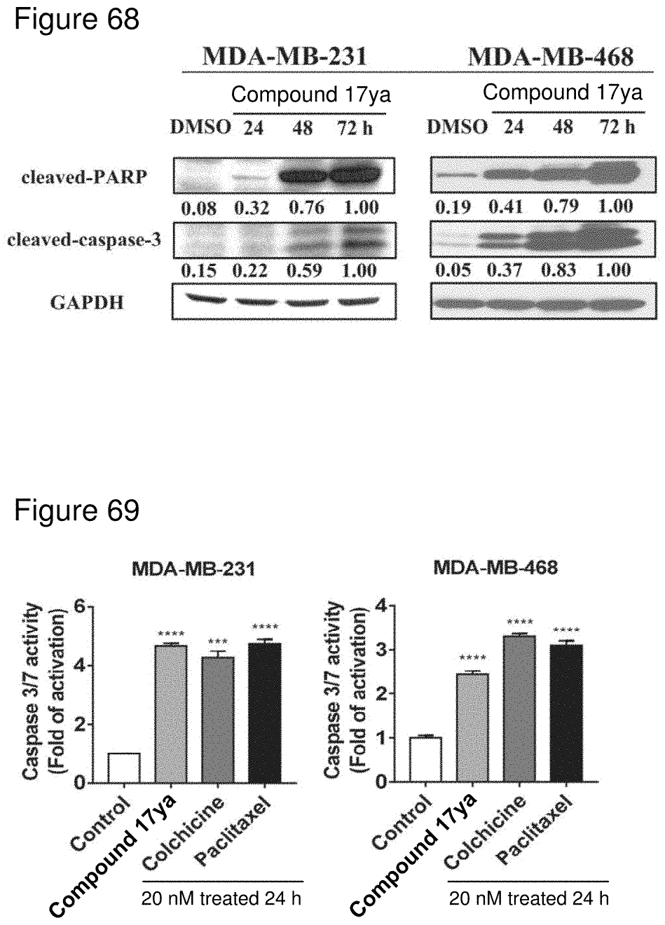

Caspases and PARP play an important role in the initiation and execution of programmed cell death. We determined the effect of compound 17ya on whether it triggered apoptotic cell death through regulating caspase-3/PARP pathway, expression of cleaved-caspase-3, and cleaved PARP in TNBC cells treated with compound 17ya as analyzed by Western blotting. The results demonstrated that the expression of cleaved-caspase-3 and cleaved-PARP were increased in a dose dependent manner after 24 hours of treatment of compound 17ya, although their expression were lower than those in paclitaxel-treated group both in MDA-MB-231 (FIG. 67A) and MDA-MB-468 (FIG. 67B) cells. Colchicine was also able to induce the upregulation of cleaved-caspase-3 and cleaved-PARP in MDA-MB-468 cells after 24 hours of treatment. Compound 17ya increased cleaved-caspase-3 and cleaved-PARP in a time dependent manner as illustrated in FIG. 68. To confirm the effect of compound 17ya on the expression of cleaved-caspase-3 and cleaved-PARP in the protein level, the caspase 3/7 activity was evaluated on MDA-MB-231 and MDA-MB-468 cells using the Caspase Glo 3/7 assay system. The results are illustrated in FIG. 69. Colchicine and paclitaxel were used as positive controls. Compared to the cells in the control group, compound 17ya, colchicine, and paclitaxel displaced up to 4-fold higher caspase 3/7 activity, that is consistent with enhanced apoptosis induction of TNBC cells.

### Example 5

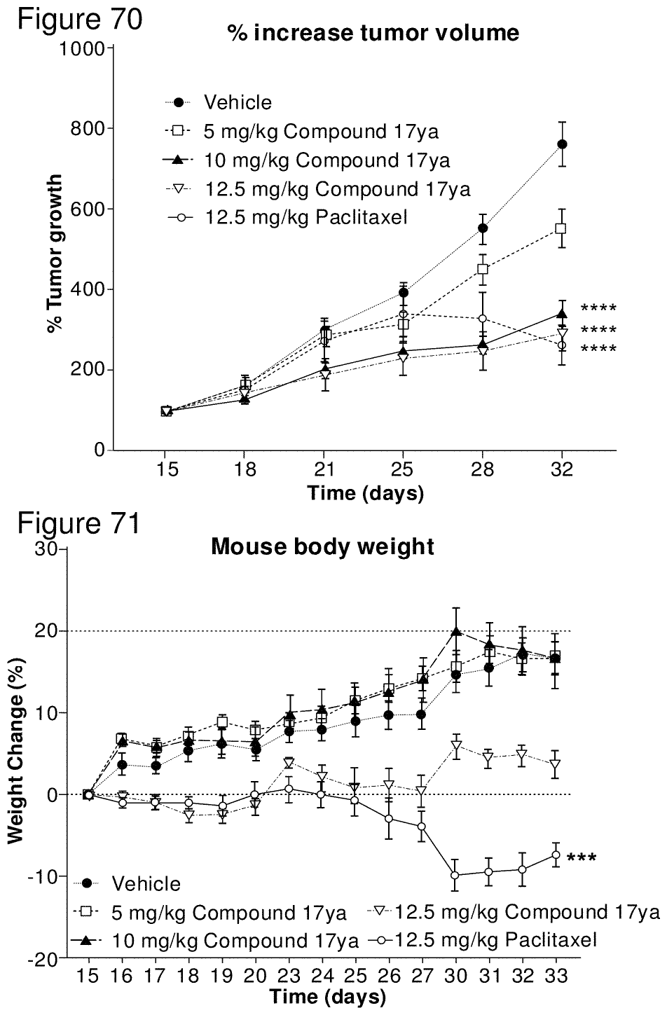

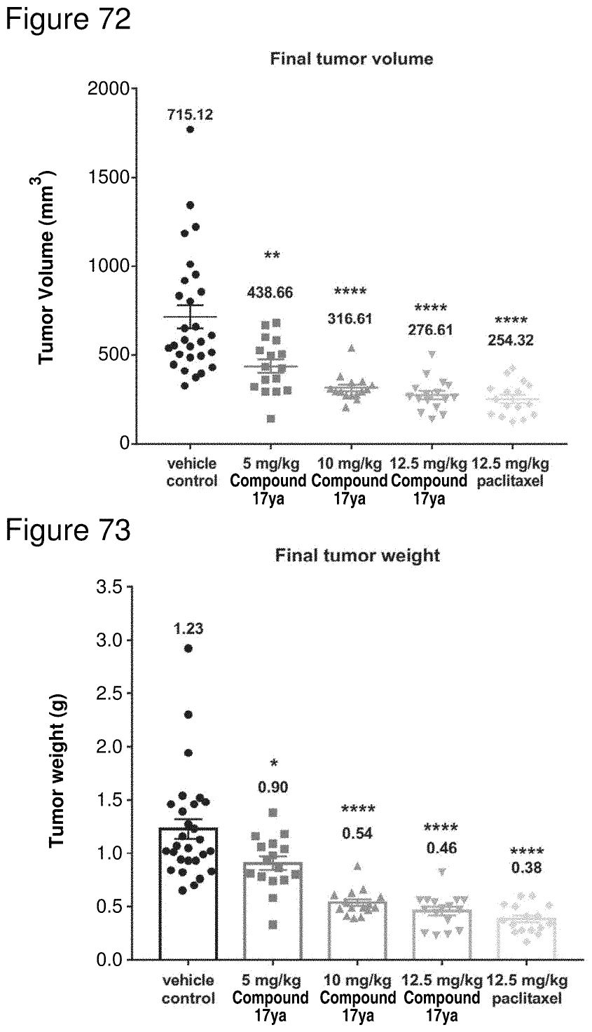

Compound 17ya inhibits TNBC tumor growth in vivo. Compound 17ya affects the in vivo growth of human cancer cell lines, as investigated by the anticancer activity in orthotopic TNBC mouse model. Since paclitaxel is one of the widely used chemotherapeutics for TNBC treatment in the clinic; it was incorporated as a comparison. NSG mice bearing MDA-MB-231 xenografts were treating with vehicle, 5 mg/kg Compound 17ya, 10 mg/kg compound 17ya, 12.5 mg/kg compound 17ya, and 12.5 mg/kg paclitaxel for 18 days. As compared to the vehicle treated group, the percentage increase of tumor size was significantly decreased in 10 mg/kg and 12.5 mg/kg compound 17ya and paclitaxel-treated groups, while the 5 mg/kg oral administration showed relatively weak tumor growth inhibition as illustrated in FIG. 70. During treatment, no loss of body weight was observed for compound 17ya treated groups an indication of lack of toxicity. While 12.5 mg/kg paclitaxel treatment decreased mice body weight significantly, suggesting accumulated toxicity of paclitaxel during the treatment as illustrated in FIG. 71. Compared to the vehicle-treated control group, compound 17ya at 5 mg/kg reduced average tumor volume and tumor weight by 38.66% and 26.83%, respectively. Compound 17ya at 10 mg/kg decreased average tumor volume and weight by 55.73% and 56.10%, respectively, and a dose of 12.5 mg/kg decreased average tumor volume and tumor weight by 61.32% and 62.6%, respectively. The results indicated that compound 17ya inhibited TNBC tumor growth in a dose dependent manner as illustrated in FIGS. 72 and 73. The group treated with 12.5 mg/kg compound 17ya was comparable to paclitaxel in average final tumor weight (0.46 g vs. 0.38 g). The efficacies of compound 17ya and paclitaxel against tumors were evident on tumor images, where treatment displayed significant reduction in tumor volume compared to the vehicle group.

### Example 6

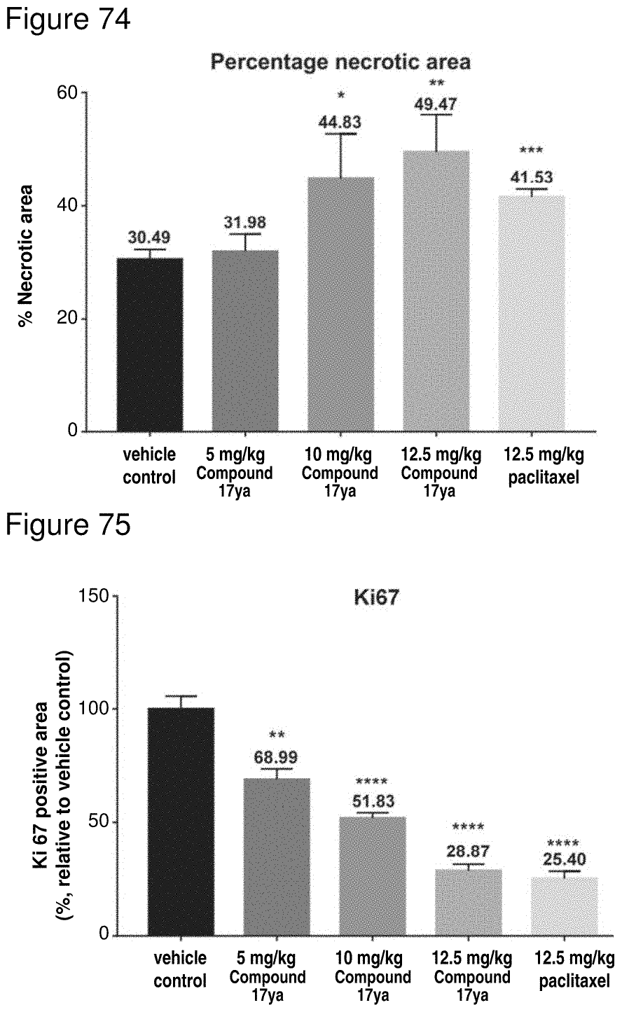

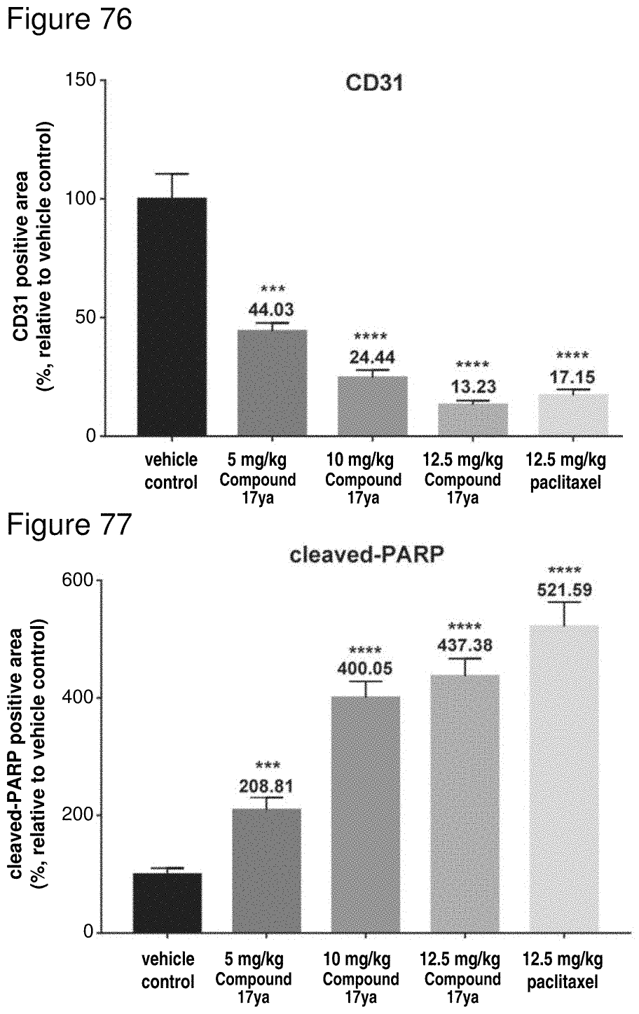

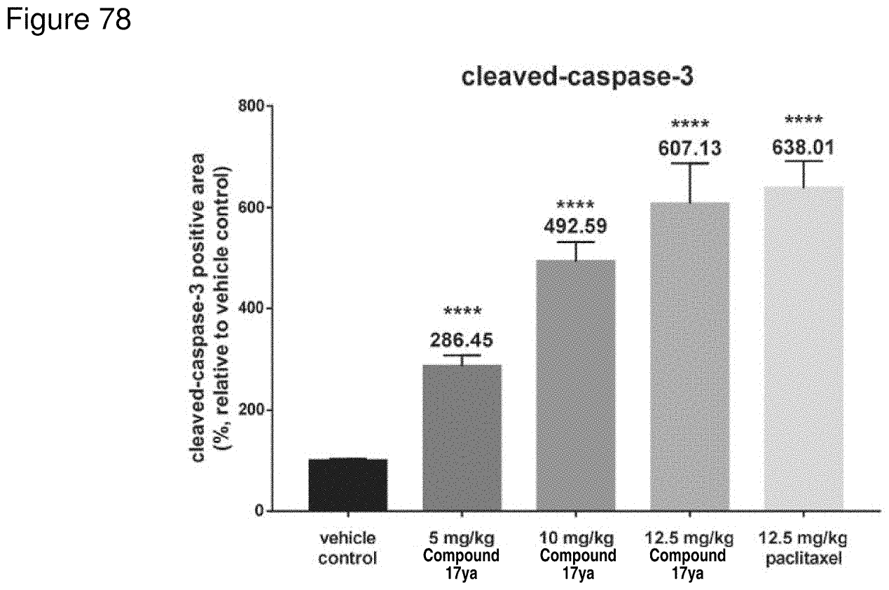

Compound 17ya induces tumor necrosis, anti-angiogenesis, and apoptosis in vivo. Tumors were excised stained with H&E and the expression of cell proliferation marker Ki67, prognostic angiogenic marker CD31, apoptotic markers cleaved-PARP, and cleaved-caspase-3 were determined through IHC staining. Natural necrosis happens inside the tumor caused by internal hypoxia, both H&E and IHC were imaged near the margin of the tumor. Both compound 17ya and paclitaxel treatment increased the number of necrotic tumor cells with pyknosis, indicated by nuclear shrinkage. Necrotic cells increased with the dose of compound 17ya from 5 mg/kg to 12.5 mg/kg. An increased percentage of necrotic area of whole tumors was observed with the compound 17ya treated group as compared to the vehicle treated counterpart as illustrated in FIG. 74. Tumors in the compound 17ya (12.5 mg/kg) treated group had 49.5% of necrotic area, comparable to the percentage of paclitaxel induced tumor necrosis (41.5%), that demonstrated the potency of compound 17ya to induce tumor necrosis, which was more efficacious to induce tumor necrosis than paclitaxel. IHC analysis demonstrated that compound 17ya treatment significantly decreased the number of Ki67-positive cells (FIG. 75) and CD31-positive cells (FIG. 76) in tumor tissues, demonstrated by 71% and 87% decrease in compound 17ya (12.5 mg/kg) treated tumors compared to the tumors in the vehicle control group, respectively, which suggested that compound 17ya inhibited the proliferation of TNBC cells and disrupt tumor vasculature in vivo. The results demonstrated that there was a clear dose-dependent increase of cells expressing the cleaved-PARP (FIG. 77) and cleaved-caspase-3 (FIG. 78), that confirmed the increased apoptosis due to treatment with compound 17ya. Tumor growth suppression, tumor vasculature disruption, and apoptotic cell death induction after paclitaxel treatment were evident from IHC histological results and the anticancer activity of paclitaxel was similar to compound 17ya. Taken together, the results demonstrated that compound 17ya exhibit similar anticancer activity as paclitaxel that suppressed tumor growth significantly at the in vivo level.

### Example 7

Compound 17ya inhibited TNBC spontaneous metastasis and cancer in lung metastasis mouse model. The experiment tested the inhibition of spontaneous lung metastasis of mice in vehicle, compound 17ya at 5 mg/kg, 10 mg/kg, and 12.5 mg/kg, and paclitaxel 12.5 mg/kg. In the vehicle group, the lung metastasis foci were increased in the lung lobe (four mice had large metastasis, 10 mice had more than five lung metastases). Compound 17ya inhibited the spontaneous metastasis of TNBC cells with some few metastasis foci in 5 mg/kg treated group (six mice with few metastases, two mice without metastases), one or two small metastasis foci in 10 mg/kg treated group (four mice with few metastases, four mice without metastases), a metastasis or none observed in the 12.5 mg/kg treated group (one mouse with one lung metastasis, seven with no metastasis), and no metastasis was observed in the 12.5 mg/kg paclitaxel treated group (all eight mice had no metastasis), indicating the significant role of compound to inhibit metastasis of TNBC.

Since compound 17ya decreased the number and size of TNBC metastases in an orthotopic mouse model, an experimental lung metastasis model was used to evaluate the anti-metastasis effect of the compound. Due to the weakness of mice after tail vein inoculation, 10 mg/kg of paclitaxel and 10 mg/kg of compound 17ya were chosen as the dosage for this study. After 22 days of treatment, mice were euthanized and lungs, livers, kidneys, and spleens were harvested, fixed, and examined by anti-mitochondria IHC and H&E staining. The lungs in the vehicle group were full of metastasis (indicated by brown dots), while the lungs in the compound 17ya and paclitaxel treated groups exhibited significantly inhibited lung metastasis of TNBC. Results of the liver and spleen tissues were similar. Fever metastases were detected in the kidneys of vehicle mice, while the kidneys of the compound 17ya and paclitaxel treated groups were clear, that demonstrated that compound 17ya inhibited metastases of TNBC. Body weights and physical activities of mice were normal in the compound 17ya treated group, while body weights and physical activities of mice in the paclitaxel treated group were slightly decreased, demonstrating the toxicity of paclitaxel during long term treatment. H&E staining showed that multiple metastases with varying sized were observed in the lung, liver, kidney, and spleen of vehicle mice, whereas the metastases of compound 17ya and paclitaxel treated mice were sparse and smaller. The lung results indicated that compound 17ya showed comparable efficacy to paclitaxel in suppressing metastasis of TNBC cells without significant toxicity in mice.

All of the features described herein (including any accompanying claims, abstract and drawings), and/or all of the steps of any method or process so disclosed, may be combined with any of the above aspects in any combination, except combinations where at least some of such features and/or steps are mutually exclusive. Although preferred embodiments have been depicted and described in detail herein, it will be apparent to those skilled in the relevant art that various modifications, additions, substitutions, and the like can be made without departing from the spirit of the invention and these are therefore considered to be within the scope of the invention as defined in the claims which follow.

What is claimed:

1. A method of treating triple negative breast cancer and/or ovarian cancer in a subject in need thereof comprising administering to the subject a therapeutically effective amount of a compound represented by the structure of formula XI: 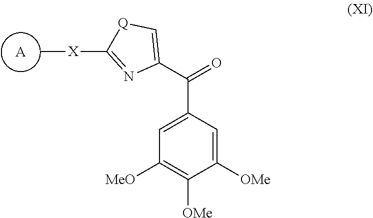
wherein
X is a bond, NH or S;
Q is O, NH or S; and
A is substituted or unsubstituted single-, fused- or multiple-ring, aryl or (hetero)cyclic ring systems; substituted or unsubstituted, saturated or unsaturated N-heterocycles; substituted or unsubstituted, saturated or unsaturated S-heterocycles; substituted or unsubstituted, saturated or unsaturated O-heterocycles; substituted or unsubstituted, saturated or unsaturated cyclic hydrocarbons; or substituted or unsubstituted or saturated or unsaturated mixed heterocycles;
wherein said A ring is optionally substituted by 1-5 substituents which are independently O-alkyl, O-haloalkyl, F, Cl, Br, I, haloalkyl, CF₃, CN, —CH₂CN, NH₂, hydroxyl, —(CH₂)ᵢNHCH₃, —(CH₂)ᵢNH₂, —(CH₂)ᵢN(CH₃)₂, —OC(O)CF₃, C₁-C₅ linear or branched alkyl, haloalkyl, alkylamino, aminoalkyl, —OCH₂Ph, —NHCO-alkyl, COOH, —C(O)Ph, C(O)O— alkyl, C(O)H, —C(O)NH₂ or NO₂; and
i is an integer between 0-5;
wherein if Q is S, then X is not a bond, or an isomer, pharmaceutically acceptable salt, pharmaceutical product, tautomer, hydrate, N-oxide, or combinations thereof, to treat the triple negative breast cancer and/or ovarian cancer.

2. The method according to claim 1, wherein said compound is represented by the structure of formula VIII: 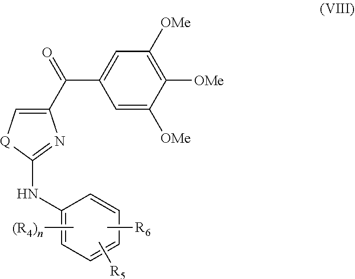
R₄, R₅ and R₆ are independently hydrogen, O-alkyl, O-haloalkyl, F, Cl, Br, I, haloalkyl, CF₃, CN, —CH₂CN, NH₂, hydroxyl, —(CH₂)ᵢNHCH₃, —(CH₂)ᵢNH₂, —(CH₂)ᵢN(CH₃)₂, —OC(O)CF₃, C₁-C₅ linear or branched alkyl, haloalkyl, alkylamino, aminoalkyl, —OCH₂Ph, —NHCO-alkyl, COOH, —C(O)Ph, C(O)O-alkyl, C(O)H, —C(O)NH₂ or NO₂;
Q is S, O or NH;
i is an integer between 0-5; and
n is an integer between 1-3.

3. The method according to claim 1, wherein said compound is represented by the structure of formula XI(b): 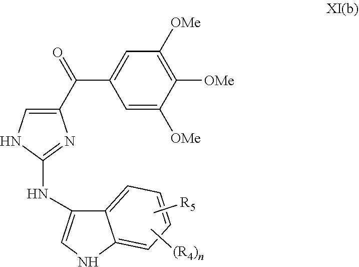
wherein R₄ and R₅ are independently hydrogen, O-alkyl, O-haloalkyl, F, Cl, Br, I, haloalkyl, CF₃, CN, —CH₂CN, NH₂, hydroxyl, —(CH₂)ᵢNHCH₃, —(CH₂)ᵢNH₂, —(CH₂)ᵢN(CH₃)₂, —OC(O)CF₃, C₁-C₅ linear or branched alkyl, haloalkyl, alkylamino, aminoalkyl, —OCH₂Ph, —NHCO-alkyl, COOH, —C(O)Ph, C(O)O-alkyl, C(O)H, —C(O)NH₂ or NO₂;
i is an integer from 0-5; and
n is an integer between 1-4.

4. The method according to claim 1, wherein said compound is represented by the structure of formula XI(c): 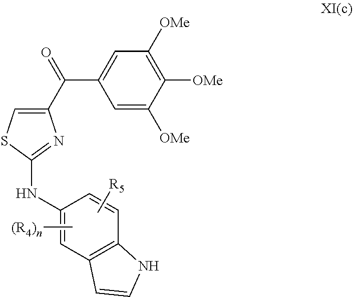
wherein R₄ and R₅ are independently hydrogen, O-alkyl, O-haloalkyl, F, Cl, Br, I, haloalkyl, CF₃, CN, —CH₂CN, NH₂, hydroxyl, —(CH₂)ᵢNHCH₃, —(CH₂)ᵢNH₂, —(CH₂)ᵢN(CH₃)₂, —OC(O)CF₃, C₁-C₅ linear or branched alkyl, alkylamino, aminoalkyl, —OCH₂Ph, —NHCO-alkyl, COOH, —C(O)Ph, C(O)O-alkyl, C(O)H, —C(O)NH₂ or NO₂;
i is an integer from 0-5; and
n is an integer between 1-4.

5. The method according to claim 4, wherein said compound is compound 55, represented by the structure:

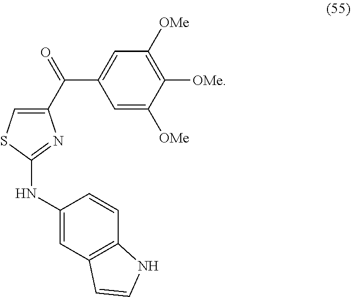 [SMILES: COc1cc(C(=O)c2csc(Nc3ccc4[nH]ccc4c3)n2)cc(OC)c1OC]

6. The method according to claim 2, wherein said compound is (2-(phenylamino)thiazol-4-yl)(3,4,5-trimethoxyphenyl)methanone (5a), (2-(p-tolylamino)thiazol-4-yl)(3,4,5-trimethoxyphenyl)methanone (5b), (2-(p-fluorophenylamino)thiazol-4-yl)(3,4,5-trimethoxyphenyl)methanone (5c), (2-(4-chlorophenylamino)thiazol-4-yl)(3,4,5-trimethoxyphenyl)methanone (5d), or (2-(phenylamino)-1H-imidazol-4-yl)(3,4,5-trimethoxyphenyl)methanone (5e).

7. The method according to claim 1, wherein the compound is combined with a pharmaceutically acceptable carrier.

8. The method according to claim 1, further comprising administering an additional cancer therapy.

9. A method of treating triple negative breast cancer and/or ovarian cancer in a subject in need thereof comprising administering to the subject a therapeutically effective amount of a compound represented by the structure of formula XI(e): 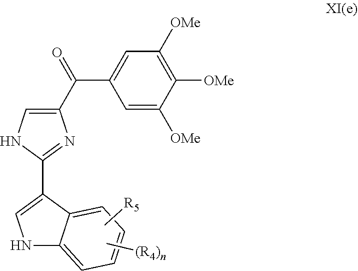
wherein R₄ and R₅ are independently hydrogen, O-alkyl, O-haloalkyl, F, Cl, Br, I, haloalkyl, CF₃, CN, —CH₂CN, NH₂, hydroxyl, —(CH₂)ᵢNHCH₃, —(CH₂)ᵢNH₂, —(CH₂)ᵢN(CH₃)₂, —OC(O)CF₃, C₁-C₅ linear or branched alkyl, alkylamino, aminoalkyl, —OCH₂Ph, —NHCO-alkyl, COOH, —C(O)Ph, C(O)O-alkyl, C(O)H, —C(O)NH₂ or NO₂;
i is an integer from 0-5; and
n is an integer between 1-4, or an isomer, pharmaceutically acceptable salt, pharmaceutical product, tautomer, hydrate, N-oxide, or combinations thereof, to a treat the triple negative breast cancer and/or ovarian cancer.

10. The method according to claim 9, wherein said compound is compound 17ya represented by the structure:

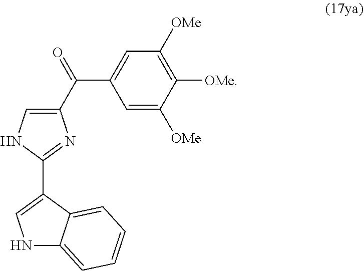 [SMILES: COc1cc(C(=O)c2c[nH]c(-c3c[nH]c4ccccc34)n2)cc(OC)c1OC]

11. The method according to claim 9 further comprising an additional cancer therapy.
